(19) United States
(12) Patent Application Publication
(10) Pub. No.: US 2025/0049960 A1
(43) Pub. Date: Feb. 13, 2025

(54) Multicomponent systems for site-specific genome modifications

(71) Applicant: The Regents of the University of California, Oakland, CA (US)

(72) Inventors: Kathleen Collins, Berkeley, CA (US); Xiaozhu Zhang, Berkeley, CA (US); Briana Van Treeck, Berkeley, CA (US); Heather E. Upton, Berkeley, CA (US); Sarah Palm, Berkeley, CA (US); Jeremy McIntyre, Berkeley, CA (US)

(73) Assignee: The Regents of the University of California, Oakland, CA (US)

(21) Appl. No.: 18/928,020

(22) Filed: Oct. 26, 2024

Related U.S. Application Data
(63) Continuation of application No. PCT/US23/66470, filed on May 2, 2023.
(60) Provisional application No. 63/337,564, filed on May 2, 2022.

(51) Int. Cl. A61K 48/00; C12N 9/12; C12N 9/22; C12N 15/11; C12N 15/90

(52) U.S. Cl. CPC A61K 48/0058; C12N 9/1276; C12N 9/22; C12N 15/11; C12N 15/907; C12Y 207/07049; C12N 2310/20

(57) ABSTRACT

The invention includes systems, compositions, and methods for the making of modular gene editing through reverse transcriptase related processes. Systems and methods that use modified nucleotides and peptides are specifically provided.

### CROSS-REFERENCES TO RELATED APPLICATIONS

This application is a continuation of PCT/US23/66470, filed May 2, 2023, which claims priority to U.S. Provisional Application No. 63/337,564, filed May 2, 2022, the disclosures of which are hereby incorporated by reference in entirety for all purposes.

### STATEMENT OF GOVERNMENT SUPPORT

This invention was made with government support under Grant Number GM139306 and HL156819 awarded by the National Institutes of Health. The government has certain rights in the invention.

### REFERENCE TO SEQUENCE LISTING

The present application is being filed with a Sequence Listing in electronic format. The Sequence Listing file, entitled B22-079.xml, was created on Jun. 2, 2023, and is 637,400 bytes in size. The information in electronic format of the Sequence Listing is incorporated herein by reference in its entirety.

### BACKGROUND OF THE DISCLOSURE

Transgene introduction into eukaryotic genomes offers vast opportunities to improve, correct and/or alter genetic expression, and concomitantly serve to treat or ameliorate disease symptoms. Successful transgene insertion would allow for rescue from loss-of-function mutations, inhibition of gain-of-function mutations, the exogenous control of RNA and/or protein expression, the introduction of isoform expression specificity, engineered gene and protein expression, and other useful outcomes.

Current methods that introduce genetic material to cells for insertion into the genome still have major hurdles to overcome. For example, methods which deliver DNA to target cells require the DNA pass through the cell's cytoplasm, which often induces a destructive or deleterious immune response. Further, methods for site-specific integration of DNA introduced into the genome by homologous recombination (HR) introduce a potentially mutagenic double-stranded DNA break and disrupt the subject genome and epigenome at the site of integration. This DNA integration is often not site-specific in higher eukaryotes, particularly in post-mitotic cells, because HR is suppressed in favor of non-homologous end-joining throughout most of the cell cycle.

A means for effective and site-specific transgene insertion into a live-cell genome, with flexibility as to the length of DNA, accomplished without potential for DNA in the cytoplasm, would be a tremendous contribution to human, animal, microorganism, and plant biology, with powerful research and clinical applications.

One approach would be to introduce a transgene sequence as an RNA that could serve as a template for complementary DNA (cDNA) synthesis by a reverse transcriptase (RT). Currently, however, molecular signals that could allow RNA introduced to mammalian cells to be copied as a template for transgene insertion into the genome have not been identified.

A class of genes known as non-long terminal repeat (LTR) retroelements (RE) or equivalently non-LTR retrotransposons, present an exciting potential solution. These genes are capable of self-amplification within their host-genome. They act by expressing a non-LTR retrotransposon RT protein (RT), which binds to and synthesizes cDNA using its own retroelement transcript RNA as a template and a nick in the genomic DNA (catalyzed by an endonuclease (EN) domain of the RT protein) as a primer for cDNA synthesis initiation (RT Primer Extension). This process, known as target-primed reverse transcription (TPRT), adds another copy of a double-stranded DNA retroelement in the genome.

WO2022/155055 describes a two-component system for site-specific safe-harbor transgene insertion to the human genome. The two components are a non-LTR retroelement reverse transcriptase (RT), and a template RNA matched to that RT engineered to enable full-length transgene insertion instead of the native retroelement propensity to 5′ insertion truncation. The mechanism for synthesis of the first inserted DNA strand is target-primed reverse transcription (TPRT), directed by the template RNA 3′ module and is enhanced by the part of that 3′ module that is a non-native 3′ tail. The 5′ module functions to provide template RNA biostability, increase template RNA bioavailability to bind the RT protein, and direct second-strand synthesis.

By creating biopolymer constructs derived in part from retroelement sequences the instant disclosure provides compositions and methods for the insertion and expression of transgenes into eukaryotic, in particular human, cell genomes.

### SUMMARY OF THE DISCLOSURE

The invention provides compositions, methods, and/or uses of proteins and nucleotides, as well as modified proteins and polynucleotides, to effect target primed reverse transcription (TPRT) transgene insertion into a subject genome using components derived from non-long terminal repeat (non-LTR) retrotransposons.

The invention provides a system for genome editing comprising (i) at least one reverse transcriptase construct (RTC), said RTC comprising a polynucleotide encoding a polypeptide having enzymatic activity for reverse transcription of a polynucleotide template, and (ii) at least one gene insertion construct (GIC), said GIC comprising at least one polynucleotide template suitable for reverse transcription by a polypeptide encoded by the at least one RTC.

In some embodiments, the system for genome editing comprises:
(i) at least one reverse transcriptase construct (RTC), said RTC comprising at least one reverse transcriptase module (RTC: RT-module) comprising an mRNA encoding a reverse transcriptase (RT), at least one reverse transcriptase construct 5′ module (RTC: 5′ module), and/or at least one reverse transcriptase construct 3′ module (RTC: 3′ module), and
(ii) at least one gene insertion construct (GIC), said GIC comprising at least one RNA template suitable for reverse transcription by a polypeptide encoded by the at least one RTC, wherein the at least one gene insertion construct comprises at least one GIC: 5′ module, at least one GIC: payload module, and/or at least one GIC: 3′ module.

In some embodiments, the RT-module comprises an mRNA encoding a RT from an organism selected from birds, arthropods, fish, tunicates, or other animals including mammals and humans.

In some embodiments, the system for genome editing comprises:
i) a RTC 5′ module comprising a 5′ untranslated region (5′-UTR), a Kozak sequence, a non-native translation start codon, and/or a 5′ cap;
ii) a RT-module comprising an mRNA encoding a RT from an organism selected from the group consisting of Zonotrichia albicollis (ZoAl), Taeniopygia guttata (TaGu), Tinamus guttatus (TiGu), Oryzias latipes (OrLa), and Tribolium castaneum (lineage B) (TriCasB);
iii) a RTC 3′ module comprising a reverse transcriptase translation stop codon, a 3′ untranslated region (3′ UTR), and a poly-A tail;
iv) a GIC: 5′ module comprising a sequence derived from a native retroelement 5′ region, an rRNA sequence, a ribozyme sequence, a folding motif sequence, and/or an RNA polymerase I terminator sequence;
(v) a GIC: payload module comprising at least one transgene ORF or non-coding RNA (ncRNA) sequence, a transgene promoter sequence or an an internal ribosome entry site (IRES), a transgene 5′ untranslated sequence, a transgene 3′ untranslated sequence, a transgene polyadenylation signal sequence, and/or a transgene ncRNA processing sequence; and
(iv) a GIC: 3′ module comprising a reverse transcriptase recognition sequence, a rRNA sequence, and/or an A-Tract sequence.

In some embodiments, at least one reverse transcriptase construct comprises at least one biopolymer, said biopolymer comprising at least one nucleic acid, at least one amino acid, and any combination thereof. In some embodiments, the RTC polynucleotide of (i) above comprises an mRNA encoding a reverse transcriptase. In some embodiments, the GIC polynucleotide template of (ii) above comprises an RNA. In some embodiments, the polynucletide of (i) above comprises an mRNA encoding a reverse transcriptase and the GIC polynucleotide template of (ii) above comprises a separate (different) RNA. In some embodiments, the GIC comprises an RNA template that is different than the mRNA encoding the RT of (i).

In some embodiments, the at least one reverse transcriptase construct comprises at least one reverse transcriptase open reading frame (ORF) module (RTC: RT-module), optionally at least one reverse transcriptase construct 5′ untranslated region (UTR) module (RTC: 5′ module), optionally at least one reverse transcriptase construct 3′ UTR module (RTC: 3′ module), and any combination thereof.

In some embodiments, at least one reverse transcriptase module comprises or encodes at least one reverse transcriptase.

In some embodiments, the at least one reverse transcriptase module comprises or encodes at least one reverse transcriptase derived from a non-long terminal repeat (non-LTR) retroelement.

In some embodiments, the at least one reverse transcriptase comprises or encodes a non-native translation start codon.

In some embodiments, the at least one reverse transcriptase comprises at least one DNA binding domain, at least one RNA binding domain, at least one cDNA synthesis domain, at least one endonuclease domain, and any combination thereof.

In some embodiments, the at least one of the at least one reverse transcriptase domain, at least one subject DNA binding domain, at least one template RNA binding domain, and at least one endonuclease domain, and any combination thereof, are derived from a species of reverse transcriptase which is different than at least one of the other at least one reverse transcriptase domain, at least one subject DNA binding domain, at least one template RNA binding domain, and at least one endonuclease domain.

In some embodiments, the at least one reverse transcriptase construct 5′ module comprises or encodes at least one RNA polymerase promoter, at least one 5′ untranslated region (5′-UTR), at least one Kozak sequence, at least one 5′ cap and any combination thereof.

In some embodiments, the at least one reverse transcriptase construct 3′ module comprises or encodes at least one reverse transcriptase translation stop codon, at least one 3′ untranslated region (3′ UTR), at least one poly-A tract and/or tail, and any combination thereof.

In some embodiments, the at least one reverse transcription module comprises or encodes at least one structure illustrated in FIGS. 2-5 or any combination thereof.

In some embodiments, the at least one reverse transcriptase construct comprises, encodes, or is encoded by at least one of SEQ ID NOS 1-57. In some embodiments, the at least one reverse transcriptase construct comprises an mRNA encoding an RT protein from a species selected from the group consisting of TriCasB, NaViB, OrLa, ZoAl, TiGu, TaGu, GeFo, DroSi, BoMo. DrMerc, DrMe, GaAc, PuPu, AdVa, HyMaA, CiIn, LiPo, TriCan, LeCo, and any combination thereof.

In some embodiments, the at least one gene insertion construct comprises or encodes at least one nucleic acid biopolymer. In some embodiments, the gene insertion construct comprises a template RNA.

In some embodiments, the at least one gene insertion construct comprises or encodes at least one optional GIC: 5′ module, at least one GIC: payload module, at least one optional GIC: 3′ module, and any combination thereof.

In some embodiments, the at least one GIC: 5′ module comprises or encodes at least one sequence derived from a native retroelement 5′ region, optionally at least one GIC: 5′ module rRNA sequence, optionally at least one GIC: 5′ module ribozyme (RZ) sequence, optionally at least one GIC: 5′ module folding motif sequence, or any combination thereof.

In some embodiments, the optional at least one GIC: 5′ module rRNA sequence comprises or encodes between 1 and 30 nt of subject rRNA.

In some embodiments, the optional at least one GIC: 5′ module ribozyme sequence comprises or encodes at least one self-cleaving ribozyme, optionally wherein said self-cleaving ribozyme comprises a hepatitis delta virus (HDV) ribozyme.

In some embodiments, the optional at least one GIC: 5′ module ribozyme sequence comprises or encodes a ribozyme derived from the 5′ region of at least one non-long terminal repeat retroelement. In some embodiments, the optional at least one GIC: 5′ module folding motif sequence comprises or encodes at least one autonomous folding RNA sequence motif, optionally wherein said autonomous folding RNA sequence motif comprises at least one hairpin motif, at least one stem-loop motif, at least one paired stem motif, within the RZ, or any combination thereof.

In some embodiments, the GIC: 5′ module comprises or encodes at least one of SEQ ID NOS 60-153, or a sequence having at least 90% identity (e.g., at least 90, 91, 92, 93, 94, 95, 96, 97, 98, 99 or 100% identity) to at least one of SEQ ID NOS 60-153. In some embodiments, the GIC: 5′ module comprises a sequence from a species selected from the group consisting of OrLa, TriCasB, TriCasA, ZoAl, TiGu, DroSi, LeCo, CiIn, FoRa, TriCan, HDV-28, HDV-24, HDV-21, HDV-13, HDV-36, or any combination thereof.

In some embodiments, the at least one GIC: 3′ module comprises or encodes at least one GIC: 3′ module reverse transcriptase recognition sequence, optionally at least one GIC: 3′ module rRNA sequence, optionally at least one GIC: 3′ module A-Tract sequence, or any combination thereof.

In some embodiments, the at least one GIC: 3′ module reverse transcriptase recognition sequence comprises or encodes at least one sequence which interacts with at least one reverse transcriptase. In some embodiments, the at least one GIC: 3′ module reverse transcriptase recognition sequence comprises a sequence selected from the group consisting of SEQ ID NOs 154-178.

In some embodiments, the at least one GIC: 3′ module reverse transcriptase recognition sequence is derived from the 3′ region of a native retroelement.

In some embodiments, the optional at least one GIC: 3′ module rRNA sequence comprises or encodes between 1 and 30 nt of rRNA.

In some embodiments, the optional at least one GIC: 3′ module A-Tract sequence comprises or encodes a sequence of between 1 and 50 adenine bases.

In some embodiments, the at least one GIC: 3′ module comprises or encodes at least one of SEQ ID NOS 154-178 or at least one of SEQ ID NOS 225-253. In some embodiments, the GIC: 3′ module comprises a sequence from a species selected from the group consisting of OrLa, TriCasB, TaGu, GeFo, ZoAl, NaViB, DroSi, PuPu, LiPo, BoMo, GaAc, LeCo, CiIn, DrMe, DrNa, DrMer, TriCan, AdVa, HyMaA, or any combination thereof.

In some embodiments, the at least one GIC: payload module comprises or encodes at least one transgene ORF sequence, optionally at least one transgene promoter sequence, optionally at least one transgene 5′ untranslated sequence, optionally at least one transgene 3′ untranslated sequence, optionally at least one transgene polyadenylation signal sequence, optionally at least one transgene non-coding RNA (ncRNA), optionally at least one ncRNA processing sequence and/or other alternative 3′ end processing or stabilization signal, or any combination thereof.

In some embodiments, the at least one transgene sequence comprises or encodes at least one sequence of interest for insertion into a subject genome.

In some embodiments, at least one transgene promoter sequence comprises or encodes at least one sequence which promotes expression of a transgene in a subject genome.

In some embodiments, the at least one GIC: payload module comprises or encodes at least one transgene 5′ untranslated sequence that comprises or encodes at least one transgene mRNA 5′ untranslated region.

In some embodiments, at least one transgene 3′ untranslated sequence comprises or encodes at least one transgene mRNA 3′ untranslated region.

In some embodiments, at least one transgene polyadenylation signal sequence comprises or encodes at least one transgene polyadenylation signal.

In some embodiments, at least one transgene non-coding RNA (ncRNA) processing sequence and/or other alternative 3′ end processing or stabilization signal comprises or encodes at least one termination signal, at least one 3′ processing signal, and any combination thereof for at least one transgene expressed ncRNA.

In some embodiments, the at least one GIC: payload module comprises or encodes a sequence having at least 90% identity (e.g., at least 90, 91, 92, 93, 94, 95, 96, 97, 98, 99 or 100% identity) to at least one of SEQ ID NOS 284-295 or SEQ ID NOS 296-332 or any combination thereof.

In some embodiments, at least one of the at least one GIC: 5′ module and at least one GIC: 3′ module comprise or encode at least one sequence derived from a species of non-long terminal repeat retroelement different from at least one of the other at least one GIC: 5′ module and at least one GIC: 3′ module.

In some embodiments, the at least one gene insertion construct comprises or encodes at least one structure illustrated in the Figures, e.g., FIGS. 6-9 and any combination thereof.

In some embodiments, the system comprises: (i) at least one reverse transcriptase construct, wherein the at least one reverse transcriptase construct comprises, encodes, or is encoded by at least one sequence having at least 90% identity (e.g., at least 90, 91, 92, 93, 94, 95, 96, 97, 98, 99 or 100% identity) to a sequence selected from the group consisting of SEQ ID NOS 1-57 and, (ii) at least one gene insertion construct, wherein at least one gene insertion construct comprises at least one sequence having at least 90% identity (e.g., at least 90, 91, 92, 93, 94, 95, 96, 97, 98, 99 or 100% identity) to a sequence selected from the group consisting of SEQ ID NOS 60-153, 179-205, 206-207, 208-217, 225-253, 275-278, 279-281, 284-295, or 296-332. In some embodiments, mRNA sequences transfected to produce RT proteins are split out from plasmid and encoded protein amino acid sequences.

In some embodiments, the system comprises:
(i) at least one reverse transcriptase construct, wherein the at least one reverse transcriptase construct comprises or is encoded by at least one sequence having at least 90% identity (e.g., at least 90, 91, 92, 93, 94, 95, 96, 97, 98, 99 or 100% identity) to a sequence selected from the group consisting of SEQ ID NOS 1-57; and
(ii) at least one gene insertion construct, wherein the at least one gene insertion construct comprises:
a GIC: 5′ module comprising a sequence having at least 90% identity (e.g., at least 90, 91, 92, 93, 94, 95, 96, 97, 98, 99 or 100% identity) to a sequence selected from the group consisting of SEQ ID NOs: 60-153;
a rRNA sequence comprising a sequence selected from the group consisting of SEQ ID NOs: 179-205, or a sequence having one, two or three nucleotide changes relative to a sequence selected from the group consisting of SEQ ID NOs: 179-205; or does not comprise a rRNA sequence;
a GIC: payload module comprising at least one transgene sequence; and
a GIC: 3′ module comprising a sequence having at least 90% identity (e.g., at least 90, 91, 92, 93, 94, 95, 96, 97, 98, 99 or 100% identity) to a sequence selected from the group consisting of SEQ ID NOS 225-253;
a GIC: 3′ module reverse transcriptase recognition sequence comprising a sequence having at least 90% identity (e.g., at least 90, 91, 92, 93, 94, 95, 96, 97, 98, 99 or 100% identity) to a sequence selected from the group consisting of SEQ ID NOS 154-178;
a GIC: 3′ module rRNA sequence selected from the group consisting of SEQ ID NOS 208-217, or a sequence comprising one, two, or three nucleotide substitutions thereof; and
a GIC: 3′ module A-Tract sequence comprising 1 to 100 adenine bases.

In some embodiments, the RTC 5′ module 5′ UTR comprises a sequence having at least 90% identity (e.g., at least 90, 91, 92, 93, 94, 95, 96, 97, 98, 99 or 100% identity) to SEQ ID NO:58.

In some embodiments, the RTC 3′ module 3′ UTR comprises a sequence having at least 90% identity (e.g., at least 90, 91, 92, 93, 94, 95, 96, 97, 98, 99 or 100% identity) to SEQ ID NO:59.

In some embodiments, the system comprises a gene insertion construct synthesis construct (GIC: synthesis construct) which comprises or encodes at least one of the gene insertion constructs described herein.

In some embodiments, at least one of the at least one reverse transcriptase construct and at least one gene insertion construct comprise or encode at least one sequence derived from a different species of retroelement than at least one of the other at least one reverse transcriptase construct and at least one gene insertion construct.

In some embodiments, the system for genome editing comprises at least one combination of, (i) at least one reverse transcriptase construct described herein, and (ii) at least one gene insertion construct described herein.

Also provided is a method for inserting at least one transgene into a subject genome comprising administering an effective amount of at least one of the gene insertion systems (GIS) of the disclosure to the subject.

In some embodiments, the transgene is inserted at one or more target sites in the subject genome, optionally wherein the one or more target sites comprise at least one safe harbor site.

In some embodiments, the optional at least one safe harbor site comprises at least one ribosomal DNA (rDNA) sequence, optionally wherein the at least one ribosomal DNA sequence comprises at least one 28 S rDNA sequence.

In some embodiments, at least one method comprises administering at least one of the gene insertion systems formulated with at least one delivery agent.

In some embodiments, the at least one delivery agent is at least one nanoparticle, optionally wherein the at least one nanoparticle comprises at least one lipid nanoparticle.

Also provided is a pharmaceutical composition comprising at least one of the gene insertion system of claims and, optionally at least one of at least one excipient, at least one delivery agent, at least one adjuvant, and any combination thereof.

Also provided is a method of treating a therapeutic indication in a subject in need thereof comprising administering an effective amount of at least one of the gene insertion systems of the disclosure or at least one of the pharmaceutical compositions of the disclosure to the subject.

In some embodiments, the therapeutic indication is caused by loss of telomerase activity.

In some embodiments, the at least one gene insertion system comprises at least one TERT transgene.

Also provided is a kit for making a gene insertion system of the disclosure. In some embodiments, the kit comprises a pharmaceutical composition of the disclosure. In some embodiments, the kit optionally further comprises buffers, DNA plasmids, or protocols to make said gene insertion systems or pharmaceutical composition.

Also provided is a method comprising de novo design of a 5′ module that recruits host machinery for second strand nicking and thus second strand synthesis. In embodiments this method provides efficiency of insertion gain by de novo design of the 5′ module to (a) include a predetermined length and position of rRNA (described herein), (b) have enhanced RZ folding, and/or (c) recruit host cell machinery.

In another aspect, the disclosure provides a method for inserting at least one transgene into a genome of a cell comprising contacting the cell with at least one of the gene insertion systems (GIS) of the disclosure.

In some embodiments, the transgene is inserted at one or more target sites in the subject genome, optionally wherein the one or more target sites comprise at least one safe harbor site. In some embodiments, the optional at least one safe harbor site comprises at least one ribosomal DNA (rDNA) sequence, optionally wherein the at least one ribosomal DNA sequence comprises at least one 28 S rDNA sequence.

In some embodiments, the method comprises administering at least one of the gene insertion systems formulated with at least one delivery agent. In some embodiments, the at least one delivery agent is at least one nanoparticle, optionally wherein the at least one nanoparticle comprises at least one lipid nanoparticle.

In some embodiments, the transgene is inserted with a target site-specificity of greater than 90% on-target (e.g., a target site-specificity greater than 90, 91, 92, 93, 94, 95, 96, 97, 98, or 99%).

In some embodiments, the RTC comprises an RNA encoding an RT from Zonotrichia albicollis (ZoAl), Taeniopygia guttata (TaGu) or Tinamus guttatus (TiGU), or comprises an amino acid sequence having at least 90% identity to SEQ ID NO:18, SEQ ID NO:20, SEQ ID NO:27, SEQ ID NO:29, or SEQ ID NO:25.

In some embodiments, the transgene is expressed at the target site for 3 months or more.

In some embodiments, the cell is contacted with the GIS wherein the molar ratio of the RTC to GIC is from about 10:1 to 1:20.

In some embodiments, the method is an in vitro method, an ex vivo method, or an in vivo method.

In some embodiments, the cell is selected from the group consisting of a primary cell, a transformed cell, an epithelial cell, a fibroblast, a human cell, a monkey cell and a mouse cell.

In some embodiments, wherein the cell is an allogenic cell or autologous cell. In some embodiments, the autologous cell is an HLA-matched cell.

The invention encompasses all combinations of the particular embodiments recited herein, as if each combination had been laboriously recited.

### BRIEF DESCRIPTION OF THE DRAWINGS

FIG. 1 is a diagram illustrating an example subject genome including a target insertion site and native retroelement. The expanded view (bottom) illustrates the shows the exemplary component structure of an R2 native retroelement.

FIG. 2 is a diagram illustrating the structure of an example reverse transcriptase construct (RTC).

FIG. 3 is a diagram illustrating exemplary domains of an RT protein of the invention.

FIG. 4 is an illustration depicting exemplary source organisms for RT protein domains including DNA binding domains (DB), RNA binding domains (RB), reverse transcriptase (RT) domains, and endonuclease (EN) domains. Also illustrated are diagrams depicting a small set of example combinations of RT protein domains. Domain identity is defined by the organism the wild-type RT is found in such that A1 is Zonotrichia albicollis, A2 is Taeniopygia guttata, A3 is Tinamus guttatus, A4 Geospiza fortis, B1 is Pungitis pungitis, B2 is Oryzias latipes, B3 is Gasterosteus aculeatus, C1 is Nasonia vitripennis, C2 is Drosophila melanogaster, C3 is Tribolium castaneum (lineage B), C4 is Bombyx mori, C5 is Drosophila simulans, C6 is Drosophila mercatorum, D1 is Lepidurus couseii, D2 is Triops cancriformis, E1 is Hydra magnipapillata, E2 is Limulus polyphemus, E3 is Adineta vaga, and E4 is Ciona intestinalis.

FIG. 5 is a set of diagrams illustrating a series of exemplary RTCs of the invention which includes a sequence which includes or encodes for an RT protein (RT) including an RT translation start codon (M). RTCs may include a 5′ untranslated sequence (5′-UTR), a translation stop codon (SC), and/or a 3′ untranslated sequence (3′-UTR).

FIG. 6 is a diagram illustrating the structure of an example gene insertion construct (middle). Expanded views show the structure of an example 5′ module (bottom left), 3′ module (bottom right), and payload module (top).

FIG. 7 is an illustration depicting exemplary source organisms for GIC 5′ module (5′ M) components, 3′ module (3′ M) components, and RTC RT module (RT) components. Also illustrated are diagrams depicting a small set of possible example GICs with potential combinations of 5′ and 3′ modules flanking a payload module with a paired Reverse Transcriptase Construct (Paired RT). Module identity is defined by the organism the wild-type retroelement and/or reverse transcriptase is found in such that A1 is Zonotrichia albicollis, A2 is Taeniopygia guttata, A3 is Tinamus guttatus, A4 Geospiza fortis, B1 is Pungitis pungitis, B2 is Oryzias latipes, B3 is Gasterosteus aculeatus, C1 is Nasonia vitripennis, C2 is Drosophila melanogaster, C3 is Tribolium castaneum, C4 is Bombyx mori, C5 is Drosophila simulans, C6 is Drosophila mercatorum, D1 is Lepidurus couseii, D2 is Triops cancriformis, E1 is Hydra magnipapillata, E2 is Limulus polyphemus, E3 is Adineta vaga, and E4 is Ciona intestinalis.

FIG. 8 is a diagram illustrating the structure of an example subject genome after insertion of a transgene by a Gene Insertion System (GIS) of the invention.

FIG. 9 is a diagram illustrating the structure of an example GIC synthesis construct.

FIG. 10 is an image of radioactive DNA synthesis products resolved by denaturing PAGE gel. The solid black box indicates the gel region with the expected product lengths. Lane numbers correspond to the various RT proteins tested as detailed in Table 3 of Example 10. Lane 1 reaction contained a negative control purification from cells that did not express RT protein.

FIG. 11 A is a cartoon depicting an example experimental design for testing RT protein specificity for binding template RNAs from cognate and non-cognate R2 element 3′UTR. FIG. 11 B Shows the spot blot results of assaying for the selectivity of B. mori, D. simulans, and O. latipes RT for the cognate and non-cognate template 3′ UTRs.

FIG. 12 A & FIG. 12 B shows the results of a denaturing PAGE gel of TPRT reaction products. The arrow indicates size expected for the correct TPRT product. Lane B contained the reaction product of B. mori RT, lane D contained the reaction product of D. simulans RT, lane O contained the reaction product of O. latipes, and lane N contained the reaction product of no enzyme. FIG. 12 A shows the results of reactions that contained the reaction product of the indicated RT protein with a template containing D. simulans template 3′UTR (lanes labeled alone) or with a template containing D. simulans template 3′UTR with 4 nt of rRNA (lanes labeled with R4). FIG. 12 B shows the results of reactions that contained the reaction product of the indicated RT protein with a template containing O. latipes template 3′UTR (lanes labeled alone) or with a template containing O. latipes template 3′UTR with 4 nt of rRNA (lanes labeled with R4).

FIG. 13 shows the results of a denaturing PAGE gel of TPRT reaction products from B. mori RT with indicated templates. The arrow indicates size expected for the correct TPRT product, the circle marks the length of products resulting from internal initiation.

FIG. 14 A & FIG. 14 B show the results of a denaturing PAGE gels of TPRT reaction products from O. latipes RT with indicated templates.

FIG. 15 shows the results of a denaturing PAGE gels of TPRT reaction products from T. castaneum RT with indicated templates. Intended TPRT product length indicated by arrow.

FIG. 16 shows the results the results of a denaturing PAGE gel of TPRT reaction products from Z. albicollis derived RT proteins. Table 8 in Example 17 gives the GIC identity used for each of the indicated lanes. Expected length of TPRT products is indicated by the solid box (Top), expected length of the precipitation recovery control is indicated by the box with a dashed outline (middle), the expected length of the radiolabeled target site oligonucleotide is indicated by the box outlined in a dot-dot-dash pattern (bottom).

FIG. 17 shows the results the results of a denaturing PAGE gel of TPRT reaction products from T. guttata derived RT proteins. Lane 1 contained the length reference ladder, Lane 2 contained only the RT protein (no template RNA) and Table 11 in Example 19 gives the GIC identity used for each of the other indicated lanes. Expected length of TPRT products is indicated by the solid box (Top), expected length of the precipitation recovery control is indicated by the box with a dashed outline (middle), the expected length of the radiolabeled target site oligonucleotide is indicated by the box outlined in a dot-dot-dash pattern (bottom).

FIG. 18 A & FIG. 18 B show PCR amplification products of genomic DNA following templated transgene insertion by T. castaneum RT proteins with indicated templates. In FIG. 18 A the expected product lengths are indicated by the box. All correct insertion PCR products should be the same size. In FIG. 18 B the expected product lengths are indicated by the arrows. Correct insertion PCR product lengths differ for the template with no 5′ module (3) versus with a 5′ module (5_3).

FIG. 19 shows the results PCR amplification of genomic DNA. The Top panel corresponds to amplification of the expected 3′ junction and the bottom panel the expected 5′ junction. Lanes marked “L” contained a reference length ladder, Lanes marked 1 and 9 contained PCR products without transfection of either TriCasB-derived RT expressing plasmid or GIC, 2-8 contained PCR products after transfection of a GIC as described in Example 21 Table 13 without an RT expressing plasmid, while Lanes marked 10-16 contained PCR products after transfection of both a GIC as described in Example 21 Table 13 and an RT expressing plasmid. Some expected PCR product lengths are marked with asterisks. See SuppFIGS for all asterisks included.

FIG. 20 shows the results PCR amplification of genomic DNA. Lanes marked A-J contained PCR products with size as expected for detection of the intended 5′ junction after co-transfection of an RTC mRNA and GIS RNA as indicated in Example 24 Table 16.

FIG. 21 shows exemplary FACS analysis results for a transgene GFP-negative clonal cell population (Top 2 Panels) and a transgene GFP-positive clonal cell population (Bottom 2 panels).

### DETAILED DESCRIPTION OF THE DISCLOSURE

### I. Introduction

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

The invention provides systems and methods for genome editing and/or gene modifications, including the insertion of a transgene into a subject genome. The systems, referred to herein as gene insertion systems (GIS) may include at least 2 components (i.e., a 2-component GIS), (a) at least one reverse transcriptase (RT) construct (RTC) which comprises or encodes a at least one reverse transcriptase and (b) at least one separately expressed gene insertion construct (GIC) which comprises or encodes an RNA construct to be used as a template for reverse transcription. As used herein, the term “construct” may refer to any artificially designed or synthesized biopolymer. Said biopolymers may, for example, be comprised of nucleic acids (e.g., DNA or RNA), amino acids, or any combination thereof. In some embodiments, both (a) and (b) are RNA constructs. In some embodiments, (a) is an amino acid construct (i.e., a protein) and (b) is an RNA construct.

Also provided are engineered RTCs capable of target primed reverse transcription (TPRT). As used herein, the term “target primed reverse transcription” refers to any process where a reverse transcriptase uses an available DNA 3′ end at the target site as the primer to initiate cDNA synthesis.

Further, the systems and methods provided may allow for insertion of a transgene at a sequence-specific location in the subject DNA (referred to herein as a target site), such as a safe harbor site. As used herein, the terms “safe harbor,” and “safe harbor site,” refer to any site in a subject genome where disruption of the subject DNA sequence, for example by insertion of a heterologous sequence, does not negatively impact the function of the subject cell. An exemplary safe harbor site utilized herein is within the portion of the subject genome that encodes for ribosomal RNA (rRNA), including the rRNA precursor transcribed by RNA Polymerase I that is encoded by what is referred to herein as a ribosomal DNA (rDNA) locus, containing sequences that encode for 5.8 S, 18 S, or 28 S rRNA.

The disclosure demonstrates that delivery of RNA alone can program the insertion of a DNA transgene into a safe-harbor location of the genome of a cell, e.g., a human cell. In some embodiments, both an RNA template encoding the transgene to be inserted, and a messenger RNA encoding the reverse transcriptase enzyme necessary to convert the RNA template into genomic DNA are delivered to cells. It is expected that RNA-only delivery will more readily translate to gene therapy in humans by exploiting ongoing innovations of non-toxic, highly efficient, cell-type-targeted RNA delivery mechanisms.

In some aspects, plasmid-based expression of reverse transriptase (RT) is combined with a transfected RNA template. In some embodiments, the transgene template 5′ module comprising native or natural parts of R2 retroelement sequences is used in heterologous combinations with the RT, which provides the advantage of full-length site-specific sequence insertion rather than a truncated retroelement sequence insertion. In some embodiments, the template RNA comprises 3′ modules with retroelement 3′UTR sequences from the same species as the RT. In some embodiments, the 3′ UTR further comprises a 3′ poly-A tract that increases target site-specific insertion efficiency.

The disclosure provides the following improvements and advantages compared to prior systems and methods. The inventors demonstrated:
(i) RT proteins from birds are remarkably active for transgene insertion, such that more than 20% of transfected cells have a functionally expressed transgene. Bird RTs are hyper-selective for copying a template RNA comprising a bird 3′ UTR followed by a 3′ poly-A tract;
(ii) heterologous combinations of bird R2 retroelement 3′ UTR and RT protein can be more effective that native combinations;
(iii) non-native, de novo created and optimized 5′ modules can be more effective, resulting in one or more orders of magnitude increase in site-specific insertion efficiency.
(iv) native 5′ modules from red flour beetles (TriCasA) (TCA, TCA5, TCARZ, and the like), which are from an R2 retroelement of a completely different clade than the bird RT proteins, can be more effective;
(v) transgene insertion delivery with co-transfected 2-RNA system rather than plasmid expression of RT followed by transfection of template RNA;
(vi) 2-RNA transfection can insert multiple transgenes per cell, enabling multiplexing of gene delivery in a single RNA administration. This allows multiple therapeutic transgenes to be inserted into the genome of the same cell, including transgenes that encode for therapeutic proteins or separate subunits of therapeutic proteins, or a combination of therapeutic proteins and RNAs;
(vii) 2-RNA delivery results in transgene expression across a broad range of cell types including primary cell lines and non-dividing or slowly dividing cells, including mouse and monkey as well as human cells;
(viii) genome sequencing demonstrates site specificity of insertion; and
(ix) the inserted transgene expression cassette has multiple-month expression stability.

### Retroelement Originating Components

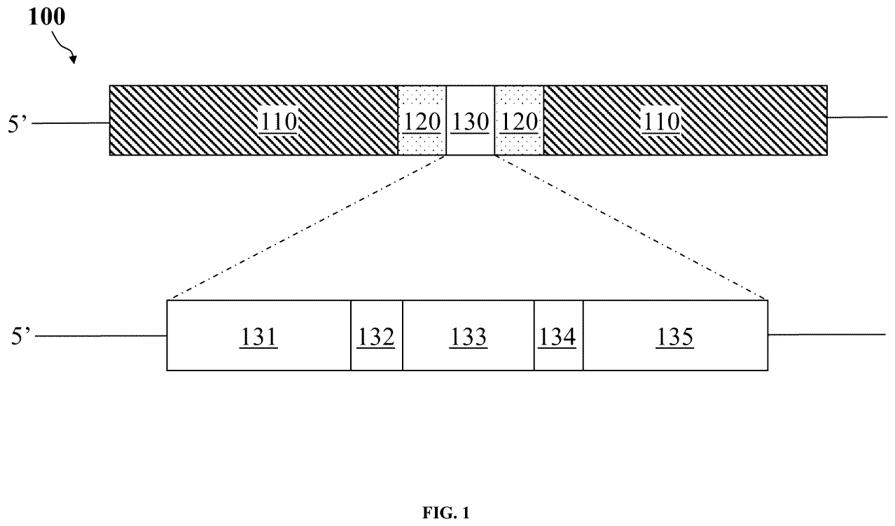

The RTCs and/or GICs of the invention may include components (interchangeably referred to as modules) which may be derived from portions of at least one non-long terminal repeat retroelement (non-LTR) and/or are not known in nature. Without wishing to be bound by theory FIG. 1 illustrates (top) a subject genome including a native retroelement 100 in this case a non-long terminal repeat retroelement (non-LTR) retroelement. As may be seen from the illustration, subject DNA 110 may include at least one target insertion site 120, and at the target insertion site a native retroelement 130, may be present. The architecture of an example native retroelement may be further examined in the expanded view (bottom). Here, the retroelement 5′ region 131 precedes the translation start site 132. The retroelement 5′ region is generally not translated into an amino acid biopolymer and may include sequences of nucleic acids that are recognized by the retroelement RT and/or, affect second strand synthesis of the native retroelement during later insertion. The translation start site 132 is the first nucleotide that will be translated into an amino acid. The retroelement reverse transcriptase open reading 133 frame encodes a reverse transcriptase which can recognize, bind, and use retroelement RNA transcript as a template for reverse transcription. The retroelement reverse transcriptase open reading frame extends to but excludes the translation stop site 134. The retroelement 3′ region 135 is generally not translated into an amino acid biopolymer and may include nucleic acid sequences which are recognized by the native retroelement RT. Regions 131 and 135 may or may not be present and if present may include sequences that duplicate the surrounding target site sequence and/or are not encoded by the retroelement RNA template.

Suitable retroelements from which GIS components may be derived include but are not limited to non-LTR retroelements, for example of the RLE-type or APE-type or Penelope type. An RLE-type non-LTR retrotransposon may be from any one of many clades, including but not limited to R2, R4, CRE, Genie, HERO, NeSL. An APE-type non-LTR retrotransposon may be from any one of many clades, including but not limited to I, R1, L1, Tx1, CR1, Rex1, Jockey, L2, Tad, RTE, RTEX, Ingi, Vingi, TRAS, SART, or any combination thereof. In some embodiments, GIS components may be derived from retroelements that insert into rDNA, i.e., the so-called R elements, such as retroelements of the R1 or R2 clade. In some embodiments, the R2 clade retroelement may have canonical R2 retroelement insertion site specificity or may be derived from an R8 and/or R9 retroelement in the larger R2 clade that have changed target sequence relative to the canonical R2 retroelements or may be derived from R2NS retroelements that appear to have lost target site specificity.

GIS components may be derived from portions or domains of retroelements found in any species, including those of distant evolutionary relation to the subject. For example, suitable retroelements from which GIS components may be derived may include those found in birds (e.g., Zonotrichia albicollis, Taeniopygia guttata, Tinamus guttatus, and Geospiza fortis), fish (e.g., Pungitis pungitis, Oryzias latipes, Danio rerio, Oryzias melastigmaa, Petromyzon marinus, Salmo trutta, Salmo salar, or Gasterosteus aculeatus), insects (e.g., Drosophila mercatorum, Drosophila melanogaster, Nasonia vitripennis, Tribolium castaneum, Drosophila simulans, Apis cerana, and Bombyx mori), crustaceans (e.g., Lepidurus couesii, and Triops cancriformis), other invertebrates (e.g., Limulus polyphemus, Hydra magnipapillata, or Adineta vaga), chordates (e.g., Ciona intestinalis) including mammals, and any combination thereof.

In some embodiments, GIS components may be derived from portions or domains of any sequence disclosed herein.

### II. Gene Insertion System (Gis) Compositions

The systems of the invention for the insertion of genetic material (e.g., transgenes) into a subject genome are referred to throughout this disclosure as gene insertion systems (GIS). A GIS may be comprised of a plurality of biopolymer constructs which are co-administered to carry out insertion of at least one transgene via target primed reverse transcription (TPRT). These biopolymer constructs may be amino acid biopolymers, nucleic acid biopolymers, hybrid biopolymers containing both amino and nucleic acids, or any combination thereof. In some examples a GIS consists of at least 2 biopolymer constructs, at least one reverse transcriptase construct (RTC) and at least one gene insertion construct (GIC). In such an example, the RTC comprises the means for carrying out reverse transcription, such as by comprising or encoding a reverse transcriptase, and the GIC comprises or encodes at least one RNA sequence which may be used as a template by the RTC for cDNA synthesis.

The biopolymer constructs of the invention are themselves comprised of a plurality of modules such that the modules may be combined as needed to alter the system for desired functions. As used herein, the term “module” refers to a portion of a construct defined either by its function (e.g., the functional domains of a protein), or by its sequence (e.g., an amino acid or nucleic acid sequence).

### Reverse Transcriptase Construct (RTC)

A GIS of the invention comprises at least one RTC which includes or encodes an active RT protein, such as an RT derived from a non-LTR retroelement. As used herein, the term “RTC” refers to a biopolymer construct which includes or encodes at least one reverse transcriptase (RT). In some embodiments, at least one RTC for use in a GIS of the invention may include an amino acid biopolymer, including but not limited to a polypeptide, a protein, pro-protein, or any combination thereof. In some embodiments, at least one RTC for use in a GIS of the invention may include a nucleic acid biopolymer, including but not limited to RNA, DNA, or any combination thereof. In some embodiments, at least one RTC may comprise at least one mRNA construct.

### RTC Architecture

An RTC of the invention may comprise at least one RTC: reverse transcriptase module (RTC: RT-module), at least one optional reverse transcriptase construct 5′ module (RTC: 5′ module), at least one optional reverse transcriptase construct 3′ module (RTC: 3′ module), and any combination thereof. In some examples of an RTC, the RTC: 5′ module and RTC: 3′ module may be optional and one or both may not be present. In some embodiments, at least one RTC may comprise, or be delivered to a subject as, a linear RNA biopolymer. In some embodiments, at least one RTC may comprise, or be delivered to a subject as, an mRNA biopolymer.

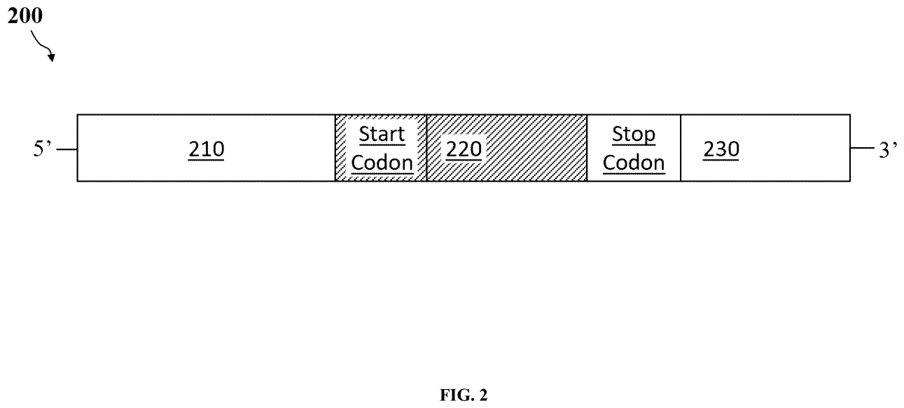

Turning now to FIG. 2, the architecture of an exemplary linear RNA biopolymer (e.g., mRNA) RTC 200 is provided. As illustrated, for an mRNA biopolymer RTC, the RTC: 5′ module 210, is an optional component of an RTC which, when present, may include sequences to alter the immunogenicity of the RTC and/or control expression of the RTC: RT-module 220. For example, the RTC: 5′ module may include or encode at least one 5′ cap (for example TriLink Clean Cap AG, m7(3′OMeG)(5′)ppp(5′)(2′OMeA)pG), at least one 5′ untranslated region (5′-UTR), at least one Kozak sequence, at least one promoter and any combination thereof. The start codon, a 3-nucleotide sequence of nucleic acids known to initiate translation, marks the 5′ end of the RTC: RT-module. The RTC: RT-module (detailed below) includes and extends from the start codon to and excludes the stop codon. The optional RTC: 3′ module 230, when present, includes and extends from the stop codon to the RTC 3′ end. The RTC: 3′ module, when present, may include sequences to alter the immunogenicity of the RTC and/or control expression of the RTC: RT-module. For example, the RTC: 3′ module may include or encode a translation stop codon, a 3′ UTR, polyadenosine sequence(s), a polyadenylation signal, or any combination thereof.

In some embodiments, at least one RTC may comprise, or be delivered to a subject as, a plasmid. In some embodiments, at least one RTC may comprise, or be delivered to a subject as, an mRNA, or pro-mRNA. In some embodiments, at least one RTC may comprise, or be delivered to a subject as, a protein. In some embodiments, at least one RTC may comprise, or be delivered to a subject as, a pro-protein.

### RTC: RT-Modules

The RT-module of an RTC comprises or encodes at least one compound or composition with reverse transcription activity, a specific but non-limiting example of which are a class of enzymatic proteins known as reverse transcriptases (RTs). In some embodiments, the RT-module may include or encode a biopolymer derived from at least one RT found in a retroelement gene (i.e., a retroelement RT). In some embodiments, the RTC: RT-module comprises or encodes at least one reverse transcriptase derived from a non-long terminal repeat retroelement.

### Reverse Transcriptases

As used herein, the term “Reverse Transcriptase (RT)” is used in its broadest sense to refer to any biopolymer with reverse transcription activity. In some embodiments, an RT for use in the invention may be or be derived from a non-LTR RT from the Zonotrichia albicollis, Taeniopygia guttata, Tinamus guttatus, Geospiza fortis, Pungitis pungitis, Oryzias latipes, Danio rerio, Oryzias melastigma, Petromyzon marinus, Salmo trutta, Salmo salar, or Gasterosteus aculeatus, Drosophila mercatorum, Drosophila melanogaster, Nasonia vitripennis, Tribolium castaneum, Drosophila simulans, Apis cerana, Bombyx mori, Lepidurus couesii, Triops cancriformis, Limulus polyphemus, Hydra magnipapillata, Adineta vaga, Ciona intestinalis, other birds, other arthropods, other fish, other tunicates, other animals (including mammals and humans) or the like's genomes.

In some embodiments, at least one RTC: RT-module for use in a GIS of this disclosure may comprise, encode, or be encoded by at least one of SEQ ID NOS 1-57. In some embodiments, at least one RTC: RT-module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 1-57. In some embodiments, the RTC: RT-module comprises a non-native or non-natural sequence having at least 90%, 91%, 92%, 93%, 94%, 95%, 96%, 97%, 98%, or 99% identity to SEQ ID NOS 1-57.

In some embodiments, at least one RTC: RT-module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOs 17-21 (a ZoA1 RT sequence)..

In some embodiments, at least one RTC: RT-module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID Nos 26-29 (a TaGu RT sequence).

In some embodiments, at least one RTC: RT-module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID Nos 1-5 (a TriCasB RT sequence).

In some embodiments, an RTC: RT-module may comprise or encode a protein shown to be active for TPRT via a suitable TPRT assay. A non-limiting example of a suitable TPRT assay includes (i) transfecting a population of cells with expression plasmids encoding the RT protein with a suitable tag for affinity purification (e.g., a FLAG tag), (ii) lysing the cell population and collecting and purifying the expressed protein product through an appropriate method known in the art, (iii) preparing recombinant template RNA by any method known in the art (e.g., T7 RNA polymerase) (iv) combining purified RT proteins, recombinant templates, and a nucleotide solution including a target site oligonucleotide duplex DNA with an end-radiolabeled bottom strand in a medium which promotes reverse transcription by the RT, and (v) collecting and analyzing products by any suitable method known in the art (e.g., denaturing PAGE).

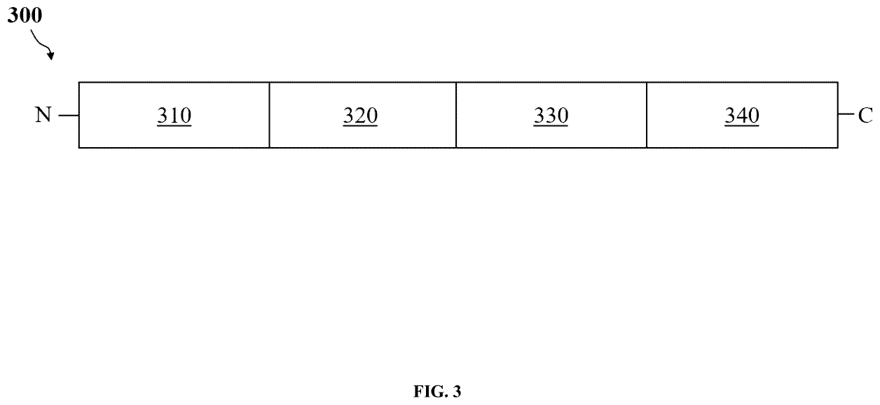

RTs suitable for use in the invention may be comprised of a plurality of functional domains. In some embodiments, such as is illustrated in FIG. 3 at least one reverse transcriptase 300 comprises at least one DNA binding domain 310, at least one RNA binding domain 320, at least one cDNA synthesis domain 330, at least one endonuclease domain 340, and any combination thereof. Note, for this illustration only one possible configuration of domains is presented. In some embodiments, any of the depicted domains may be present in a different frequency in the RT and/or the domains may be present in any order. In some embodiments, the DNA and RNA binding domains might be from a different type of polypeptide than an RT or of sequence not known to be in a eukaryotic genome (e.g., de novo engineered DNA or RNA binding domain).

### Start Codon

At least one non-native translation start codon may be added to a nucleic acid sequence encoding an RT by various methods known in the art. The non-native translation start codon may be added to a sequence derived from a non-LTR retroelement at any position which produces a functional RT. For example, at least one non-native start codon may be added at about 1, 10, 50, 100, 150, 200, 250, 300, 350, 400, 450, 500, 550, 600, 650, 700, 750, 800, 850, 900, 950, 1000, or more bases from a known reference point in the wild-type non-LTR retroelement (e.g., from an amino acid sequence motif in the native retroelement RT ORF). The positioning of a translation start codon may be selected as the result of optimization of polypeptide length, sequence composition, activities, biological stability, lack of aggregation, or localization, and/or to give the mRNA encoding the protein improved biological stability, among other considerations evident to those practiced in the art of engineering optimal or regulated protein expression in the target cells of interest.

The translation start codon may be any 3 nucleotides known to initiate translation by a ribosome, dependent on or independent of another sequence or structure in the mRNA. In some embodiments, the non-native translation start codon is AUG.

### RTC: 5′ Module

An RTC of the invention may comprises at least one RTC: 5′ module. In general, the RTC: 5′ module comprises untranslated biopolymer components which may, by way of non-limiting examples, alter the immunogenicity of the GIC, aid in localizing the GIC to targeted intracellular regions, control or alter expression of a GIC's RTC: RT-module, label a GIC for identification, assist in purification of a GIC, control degradation of a GIC, allow for exogenous or endogenous regulation of GIC activity and/or function, and any combinations thereof.

In some embodiments, at least one RTC: 5′ module may include or encode at least one 5′ UTR. In some embodiments, at least one RTC: 5′ module may include or encode at least one 5′ cap. In some embodiments, at least one RTC: 5′ module may include or encode at least one microRNA binding sequence. In some embodiments, at least one RTC: 5′ module may include or encode at least one RNA polymerase promoter.

In some embodiments, at least one RTC: 5′ module for use in a GIS of this disclosure comprises a 5′ UTR of SEQ ID NO 58.

In embodiments we used one 5′ and one 3′ UTR for the transfected mRNAs, which were taken from the BioNTech vaccine sequence as reported to WHO. We also used their template-encoded polyA region (instead of using polyA polymerase post-transcription), which is composed of A30-10 nt Linker—A70 and followed by a TypellS restriction site to cleave template for mRNA transcription without any extra 3′ nt. All mRNAs were capped with TriLink AG clean cap m7(3′OMeG)(5′)ppp(5′)(2′OMeA)pG). The UTRs are selected for tissue-specific RT expression, for example to impose cell type specific translational control.

In some embodiments, an RTC: 5′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 58.

### RTC: 3′Module

An RTC of the invention may comprises at least one RTC: 3′ module. In general, the RTC: 3′ module comprises untranslated biopolymer components which may, by way of non-limiting examples, alter the immunogenicity of the GIC, aid in localizing the GIC to targeted intracellular regions, control or alter expression of a GIC's RTC: RT-module, label a GIC for identification, assist in purification of a GIC, control degradation of a GIC, allow for exogenous or endogenous regulation of GIC activity and/or function, and any combinations thereof.

In some embodiments, at least one RTC: 3′ module may include at least one 3′ UTR. In some embodiments, at least one RTC: 3′ module may include or encode at least one poly-A tract or poly-A tail. In some embodiments, at least one RTC: 3′ module may include or encode at least one microRNA binding sequence.

In some embodiments, at least one RTC: 3′ module for use in a GIS of this disclosure comprises a 3′ UTR and poly-A tail of SEQ ID NO 59.

In some embodiments, an RTC: 3′ module comprises a 3′ UTR with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 59.

### Modularity of the RTC

RTCs of the invention may be designed for a desired function or activity by combining any combination of at least one RTC: RT-module, optionally at least one RTC: 5′ module, and/or optionally at least one RTC: 3′ module. In some embodiments, the RTC comprises at least one RTC: 5′ module. In some embodiments, the RTC comprises at least one RTC: 3′ module. In some embodiments, the RTC comprises at least one RTC: RT-module. In some embodiments, the RTC comprises at least one RTC: 5′ module, at least one RTC: RT-module, and at least one RTC: 3′ module. In some embodiments, the RTC comprises at least one RTC: 5′ module, and at least one RTC: RT-module. In some embodiments, the RTC comprises at least one RTC: RT-module, and at least one RTC: 3′ module.

In some embodiments, an RTC of the invention may not include at least one RTC: 5′ module, and at least one RTC: 3′ module. In some embodiments, an RTC of the invention may not include at least one RTC: 5′ module, or at least one RTC: 3′ module. In some embodiments, an RTC of the invention may not include at least one RTC: 5′ module. In some embodiments, an RTC of the invention may not include at least one RTC: 3′ module.

In some embodiments, at least one RTC may comprise any combination of: (a) at least one RTC: 5′module selected from, encoding, or encoded by any one of SEQ ID NO 58, (b) at least one RTC: RT-module selected from, encoding, or encoded by any one of SEQ ID NOS 1-57, and/or (c) at least one RTC: 3′ module selected from, encoding, or encoded by any one of SEQ ID NO 59.

### Exemplary RTCs

RTCs for use in the invention may comprise, encode, or be encoded by at least one of SEQ ID NOS 1-57. In some embodiments, an RTC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 1-57.

In some embodiments, at least one RTC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 17-21.

In some embodiments, at least one RTC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 26-29.

In some embodiments, at least one RTC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 24-25.

In some embodiments, at least one RTC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 1-5.

In some embodiments, at least one RTC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 35-37.

In some embodiments, at least one RTC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 32-34.

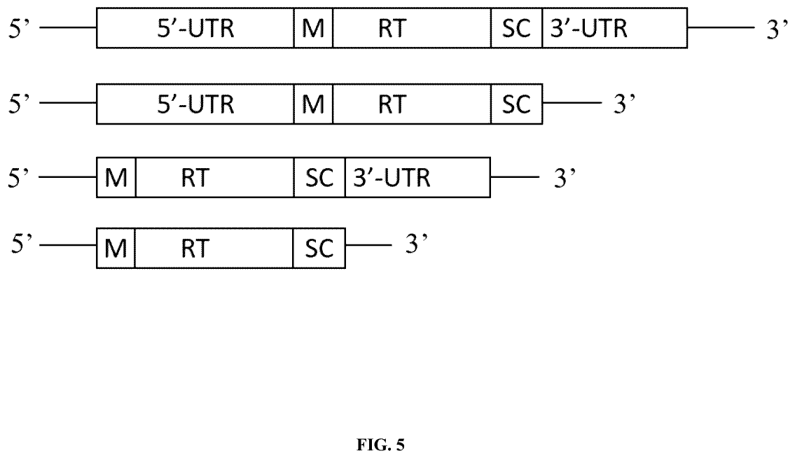

In some embodiments, at least one RTC comprises a structure illustrated in FIG. 5. RTC Regulatory Elements

The RTCs of the invention may further comprise any number of regulatory elements, which may be located within any of the RTC modules. As used herein, the term “regulatory element” refers to any sequence, region, or domain that allows for control of expression or activity of the biopolymer it is part of.

For example, an RNA based RTC may contain any number of micro-RNA (miRNA) or small interfering RNA (siRNA) binding sites. Without wishing to be bound by theory, the presence of these RNA interference (RNAi) binding sites may prevent expression of the RT protein in specific cell types, based on the RNAi transcriptome present. In this way, a GIS of the invention can be de-targeted from a subject cell type. As used herein, the term “miRNA or siRNA binding site” refers to a sequence of RNA that is complimentary to at least one miRNA or siRNA respectively.

In some embodiments, an RTC may comprise at least one miRNA and/or siRNA binding site that is complementary to at least one miRNA and/or siRNA comprised in or encoded by a transgene to be inserted by the GIS. In general, this may enable a GIS of the invention to self-regulate the number of transgene insertions made by a single administration of the GIS and/or prevent repeat insertion of transgenes after the initial administration. In this way, a GIS may have increased capacity for re-dosing or co-dosing to a given subject.

### Gene Insertion Construct (GIC)

A GIS of the invention comprises at least one GIC, which, in general includes or encodes at least one sequence of interest intended for insertion into a subject genome (i.e., a “payload sequence”). As used herein, the term “GIC” refers to any biopolymer construct which includes or encodes at least one RNA sequence, such that the RNA sequence is recognized by at least one RT comprised or encoded by at least one RTC: RT-module and can serve as a template for reverse transcription. In some embodiments, at least one GIC for use in a GIS of the invention may include a nucleic acid biopolymer, including but not limited to RNA, DNA, or any combination thereof.

### GIC Architecture

Gene insertion constructs (GICs) of the invention may comprise or encode at least one GIC: 5′ module, at least one GIC: payload module, at least one GIC: 3′ module, and any combination thereof. In some embodiments, at least one GIC may comprise, or be delivered to a subject as, a plasmid. In some embodiments, at least one GIC may comprise, or be delivered to a subject as, a linear RNA.

In some embodiments, the at least one GIC: 5′ module is optional. In some embodiments, the at least one GIC: 3′ module may be optional. In some embodiments, a GIC of the invention may comprise or encode at least one GIC: payload module and does not comprise or encode at least one GIC: 5′ module and/or at least one GIC: 3′ module.

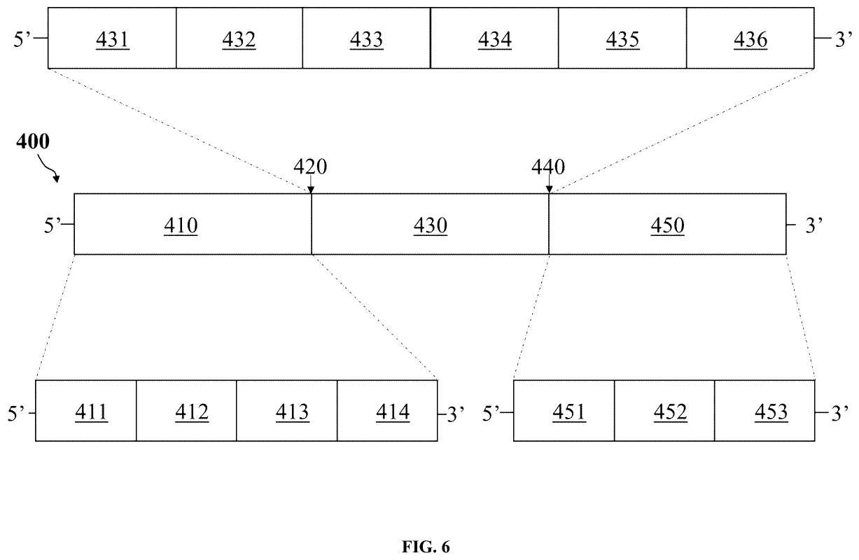

As can be seen in FIG. 6, which depicts an exemplary linear RNA GIC 400, the optional GIC: 5′ module 410 extends from the 5′ GIC sequence terminus to the GIC: 5′ module terminus 420. The GIC: payload module 430 is oriented 3′ to the GIC: 5′ module (when present) and extends to the GIC: payload module terminus 440. Finally, the GIC: 3′ module 450 extends to the 3′ GIC terminus. Each of these features are discussed in detail below.

### GIC: 5′ Module

GIC: 5′ modules for use in a GIC of this disclosure may comprise or encode at least one sequence derived from a native retroelement 5′ region. Without wishing to be bound by theory, the 5′ module may comprise or encode RNA sequences which interact with at least one RNA binding domain of an RT, effect second strand synthesis during transgene insertion, decrease immunogenicity of the GIC, provide features useful for GIC stability and/or purification, and any combination thereof.

### GIC: 5′Module Architecture

In embodiments the 5′ module comprises or contains a 5′ rRNA sequence and a ribozyme (RZ) sequence. In some embodiments, the 5′ rRNA sequence and RZ sequence are not necessarily entirely separate. In some embodiments, the 5′ module comprises a ‘folding sequence’, which may be separate from the RZ sequence. In some embodiments, a GIC: 5′ module may optionally comprise or encode at least one GIC: 5′ module rRNA sequence (or other target site sequence), optionally at least one GIC: 5′ module ribozyme (RZ) sequence, optionally at least one GIC: 5′ module folding sequence, and any combination thereof.

Turning back to FIG. 6, the expanded view (bottom left) of a GIC: 5′ module 410 illustrates the architecture of one exemplary GIC: 5′module. The GIC: 5′ rRNA sequence 411, when present at the 5′ end of the 5′ module, may include or encode an RNA sequence which is complementary to a sequence of subject DNA located 5′ to the target insertion site or otherwise near the target insertion site. The GIC: 5′ module ribozyme (RZ) sequence 412, when present, may include at least one RNA sequence with the fold of a self-cleaving RZ, which may or may not self-cleave to release the functional GIC from a transcribed 5′ leader sequence. The GIC: 5′ module RZ sequence will fold and when active will cleave such that the GIC: 5′ rRNA sequence is included as part of the RZ at or near the 5′ end of the GIC. The optional GIC: 5′ module folding motif sequence 413 may include at least one RNA sequence with predicted or demonstrated autonomous folding, which may be useful to physically and/or kinetically separate folding of the GIC: 5′ module RZ from folding of the payload sequence. Additionally, within region 414 or at position 420, which is between the GIC 5′ module 410 and payload module 430, GIC sequence may be added to terminate or otherwise regulate transcription initiated from endogenous cellular promoter sequence(s) flanking the target site. In some embodiments, endogenous cellular promoter sequence(s) flanking the target site may be used for payload expression, which is one example of a situation in which GIC sequence(s) may be added at position 420 and/or 440 to modulate payload expression (for example, to initiate translation or terminate transcription of a host promoter RNA transcript containing the payload sequence). In addition, region 414 may contain an RNA polymerase (RNAP) termination sequence to prevent RNA polymerase readthrough from genes at the target insertion site. In some embodiments, the RNAP is RNAP I (Pol I), and the termination sequence prevents Pol I readthrough transcription when the GIC payload module is integrated into a ribosomal DNA gene target site. In some embodiments, the RNAP terminator sequence comprises the sequence 5′

(SEQ ID NO: 333)
5′-AGGTCGACCAGATGTCCGAGGTCGACCAGTTGTCCG-3′.

GIC: 5′Module rRNA Sequence

The at least one GIC: 5′ module rRNA sequence is an optional component of a GIC: 5′ module. When present, it may include or encode a sequence of human ribosomal RNA (rRNA) or other sequences homologous and/or complimentary to at least one subject DNA sequence located 5′ to the target insertion site. Without wishing to be bound by theory, this sequence of rRNA may direct second strand synthesis of the inserted cDNA transgene by recruiting at least one endogenous DNA repair mechanism. In some embodiments, the GIC: 5′ module rRNA sequence is located 5′ of the GIC: 5′ module RZ sequence. In some embodiments, the GIC: 5′ module does not comprise a sequence including an rRNA genomic sequence.

In some embodiments, the at least one GIC: 5′ module rRNA sequence may comprise or encode between about 1 and 36 nt of rRNA. In some embodiments, the at least one GIC: 5′ module rRNA sequence may comprise or encode between about 1 and 30 nt of rRNA. In some embodiments, the at least one GIC: 5′ module rRNA sequence may comprise or encode between about 1 and 28 nt of rRNA. In some embodiments, the at least one GIC: 5′ module rRNA sequence may comprise or encode between about 1 and 26 nt of rRNA. In some embodiments, the at least one GIC: 5′ module rRNA sequence may comprise or encode between about 1 and 13 nt of rRNA. In some embodiments, the at least one GIC: 5′ module rRNA sequence may comprise or encode between about 1 and 11 nt of rRNA.

In some embodiments, the at least one GIC: 5′ module rRNA sequence may comprise or encode about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 nt of rRNA.

In some embodiments, the at least one GIC: 5′ module rRNA sequence may comprise or encode about 30 nt of rRNA. In some embodiments, the at least one GIC: 5′ module rRNA sequence may comprise or encode about 36 nt of rRNA. In some embodiments, the at least one GIC: 5′ module rRNA sequence may comprise or encode about 28 nt of rRNA. In some embodiments, the at least one GIC: 5′ module rRNA sequence may comprise or encode about 26 nt of rRNA. In some embodiments, the at least one GIC: 5′ module rRNA sequence may comprise or encode about 13 nt of rRNA. In some embodiments, the at least one GIC: 5′ module rRNA sequence may comprise or encode about 11 nt of rRNA. In some embodiments, the GIC: 5′ module rRNA sequence comprises a 5′ G nucleotide.

In some embodiments, at least one GIC: 5′ module rRNA sequence may comprise, encode, or be encoded by at least one of SEQ ID NOS 179-205. In some embodiments, the at least one GIC: 5′ module rRNA sequence may comprise, encode, or be encoded by a sequence with at least 100%, 99%, 98%, 97%, 96%, 95%, 90%, 85%, 80%, 75%, 70% homology to at least one of SEQ ID NOS 179-205. In some embodiments, the at least one GIC: 5′ module rRNA sequence comprises a sequence having one, two or three nucleotide changes or substitutions relative to a sequence selected from the group consisting of SEQ ID NOs: 179-205.

In some embodiments, at least one GIC: 5′ module rRNA sequence may comprise, encode, or be encoded by a sequence with at least 100%, 99%, 98%, 97%, 96%, 95%, 90%, 85%, 80%, 75%, 70% homology to at least one of SEQ ID NO 181. In some embodiments, the at least one GIC: 5′ module rRNA sequence comprises a sequence having one, two or three nucleotide changes relative to SEQ ID NO 181.

In some embodiments, at least one GIC: 5′ module rRNA sequence may comprise, encode, or be encoded by a sequence with at least 100%, 99%, 98%, 97%, 96%, 95%, 90%, 85%, 80%, 75%, 70% homology to at least one of SEQ ID NO 183. In some embodiments, the at least one GIC: 5′ module rRNA sequence comprises a sequence having one, two or three nucleotide changes relative to SEQ ID NO 183.

In some embodiments, at least one GIC: 5′ module rRNA sequence may comprise, encode, or be encoded by a sequence with at least 100%, 99%, 98%, 97%, 96%, 95%, 90%, 85%, 80%, 75%, 70% homology to at least one of SEQ ID NO 184. In some embodiments, the at least one GIC: 5′ module rRNA sequence comprises a sequence having one, two or three nucleotide changes relative to SEQ ID NO 184.

### GIC: 5′ Module RZ Sequence

The GIC: 5′ module RZ sequence is an optional component of a GIC: 5′ module that, when present comprises or encodes at least one self-cleaving ribozyme or sequence with the fold of a self-cleaving ribozyme (together described as RZ). Without wishing to be bound by theory, this motif may bury the 5′ OH terminus of the GIC, such as the 5′ terminus resulting from self-cleavage, in a stable tertiary structure, which may decrease innate immune response to an exogenous RNA, decrease decay of the GIC by 5′-3′ exonucleases dependent on 5′ monophosphate to initiate cleavage, and lower the chances of the subject cell recognizing the GIC as an mRNA or other undesired RNA type instead of as a template RNA.

In some embodiments, the at least one GIC: 5′ module RZ sequence comprises or encodes a ribozyme derived from the 5′ region of at least one non-LTR retroelement. In some embodiments, the at least one GIC: 5′ module RZ sequence comprises or encodes a ribozyme derived from the 5′ region of a non-LTR retroelement from G. aculeatus, L. polyphemus, P. pungitis, N. vitripennis, G. fortis, O. latipes, Z. albicollis, T. guttata, T. castaneum (for example from R2 lineage A or B), T. guttatus, other birds, other arthropods, other fish, other tunicates, other animals, or the like's genome.

In some embodiments, the GIC: 5′ module RZ sequence comprises or encodes an RZ with potential to form the Hepatitis Delta Virus (HDV) RZ secondary and tertiary structure, which may be modified from sequences found in nature and/or designed de novo without use of known genome sequences. In some embodiments, the HDV-fold RZ sequence bridging paired stems P1 and P2, which can be described as Junction (J) 1/2, is comprised in part or whole by a desired length of target site sequence, for example 5′ rRNA, or by the desired target site sequence additionally protected by formation of a stem-loop. In some embodiments, the HDV-fold RZ paired stem 4 (P4) design may enable non-denaturing GIC purification, for example by binding to a native or modified sequence of PP7 or MS2 phage coat protein. In some embodiments, the sequence of the RZ is designed and optimized to minimize or eliminate alternative non-productive folding. In some embodiments, the sequence of the RZ is designed and optimized to minimize the number of uridine nucleotides. In some embodiments, the sequence of the RZ is designed and optimized to enable replacement of a canonical ribonucleotide, in complete or part, by a nucleotide analog incorporated during template RNA synthesis.

In some embodiments, at least one GIC: 5′ module RZ sequence may comprise, encode, or be encoded by at least one of SEQ ID NOS 60-153. In some embodiments, the RZ sequence spontaneously folds as an active RZ. In some embodiments, the RZ sequence comprises an internal rRNA sequence at the 5′ end. In some embodiments, the RZ sequence is extended 5′ or 3′. In some embodiments, the RZ sequence comprises a catalytically inactive RZ sequence. In some embodiments, at least one GIC: 5′ module RZ sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 60-153. In some embodiments, the GIC: 5′ module RZ sequence comprises a non-native or non-natural sequence having at least 90%, 91%, 92%, 93%, 94%, 95%, 96%, 97%, 98%, or 99% identity to SEQ ID NOS 60-153.

In some embodiments, at least one GIC: 5′ module RZ sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 60.

In some embodiments, at least one GIC: 5′ module RZ sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 64.

In some embodiments, at least one GIC: 5′ module RZ sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 67.

In some embodiments, at least one GIC: 5′ module RZ sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 100.

In some embodiments, at least one GIC: 5′ module RZ sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 120.

In some embodiments, at least one GIC: 5′ module RZ sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 121.

In some embodiments, at least one GIC: 5′ module RZ sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 136.

### GIC: 5′ Module Folding Sequence

The GIC: 5′ module folding sequence is an optional component of the 5′ module that, when present, comprises at least one RNA sequence motif with a specific designed structure. In some embodiments, an autonomous folding RNA sequence motif comprises at least one hairpin motif, which, for example, may be present after the RZ to insulate RZ sequence from misfolding by base-pairing with the subsequently transcribed payload region. In some embodiments, the 5′ module region designed to improve productive template RNA folding may base-pair or otherwise interact, directly or indirectly, with another template RNA region in the payload module or 3′ module. In some embodiments the at least one RNA sequence motif directing template RNA folding may comprise at least one stem-loop motif that binds a protein bridge to another stem-loop motif. In some embodiments, the 5′ module folding sequence may favor pairing of the template RNA with the RT-encoding mRNA, for example to promote a 1:1 stoichiometry of co-packaged of RT-encoding mRNA and template RNA in an individual delivery vehicle. In some embodiments, the 5′ module folding sequence may favor pairing of the template RNA with an endogenous target cell RNA, for example for purposes of template RNA stabilization, localization, and/or other useful outcomes.

In some embodiments, at least one GIC: 5′ module folding sequence may comprise, encode, or be encoded by at least one of SEQ ID NOS 206-207. In some embodiments, at least one GIC: 5′ module folding sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 206-207. In some embodiments, the GIC: 5′ module folding sequence comprises a non-native or non-natural sequence having at least 90%, 91%, 92%, 93%, 94%, 95%, 96%, 97%, 98%, or 99% identity to SEQ ID NOS 206-207.

In some embodiments, at least one GIC: 5′ module folding sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 206.

In some embodiments, at least one GIC: 5′ module folding sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 207.

### Modularity of the GIC: 5′ Module

The disclosed 5′ module components may be used interchangeably with each other in a combinatorial manner to design a 5′ module with the required or desired functionality for a particular GIS.

In some embodiments, the at least one GIC: 5′ module comprises at least one GIC: 5′ Module rRNA sequence. In some embodiments, the at least one GIC: 5′ module comprises at least one GIC: 5′ module RZ sequence. In some embodiments, the at least one GIC: 5′ module comprises at least one GIC: 5′ module folding sequence. In some embodiments, the at least one GIC: 5′ module comprises at least one GIC: 5′ Module rRNA sequence and at least one GIC: 5′ module RZ sequence. In some embodiments, the at least one GIC: 5′ module comprises at least one GIC: 5′ Module rRNA sequence and at least one GIC: 5′ module RZ sequence and at least one GIC: 5′ module folding sequence.

In some embodiments, at least one GIC: 5′ module may comprise any combination of: (a) at least one GIC: 5′ Module rRNA sequence selected from, encoding, or encoded by any one of SEQ ID NOS 179-205, (c) at least one GIC: 5′ module RZ sequence selected from, encoding, or encoded by any one of SEQ ID NOS 60-153, and/or (d) at least one GIC: 5′ module folding sequence selected from, encoding, or encoded by any one of SEQ ID NOS 206-207.

### Exemplary GIC: 5′ Modules

In some embodiments, at least one GIC: 5′ module may comprise, encode, or be encoded by at least one of SEQ ID NOS 60-153. In some embodiments, at least one GIC: 5′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 60-153. In some embodiments, the GIC: 5′ module comprises a non-native or non-natural sequence having at least 90%, 91%, 92%, 93%, 94%, 95%, 96%, 97%, 98%, or 99% identity to SEQ ID NOS 60-153.

In some embodiments, at least one GIC: 5′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 60, 61, 77, and 79-83.

In some embodiments, at least one GIC: 5′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 62 and 63.

In some embodiments, at least one GIC: 5′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 120.

In some embodiments, at least one GIC: 5′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 116-118.

### GIC: 3′Module

3′ modules for use in a GIC of this disclosure may comprises or encodes at least one sequence derived from a native retroelement 3′ UTR. In general, the 3′ module includes components which promote recognition and binding of the GIC by an RT, position the payload module for reverse transcription, and stabilize the GIC RNA.

### GIC: 3′ Module Architecture

In some embodiments, a GIC: 3′ module may comprise or encode at least one GIC: 3′ module RT recognition sequence, optionally at least one GIC: 3′ module rRNA sequence, optionally at least one GIC: 3′ module A-Tract sequence, and any combination thereof.

Turning once again to FIG. 6. The expanded view (bottom right) illustrates the architecture of an example GIC: 3′ module 450. At the 5′ end of the GIC: 3′ module is the GIC: 3′ module RT recognition sequence 451, which may contain or encode a sequence which is recognized or bound by at least one RT. When present, the GIC: 3′ module rRNA sequence 452 may be 3′ to the GIC: 3′ module RT recognition sequence and may comprise or encode a sequence homologous to the target site region, for example 28S rRNA nucleotides that could base-pair with a TPRT primer 3′ end. Finally, when present, the GIC: 3′ module A-Tract sequence 453 may include an adenosine-rich or tandem adenosine sequence that may be of constrained length, for example between 10 and 60 nt, and may be at the 3′ end of the GIC: 3′ module.

### GIC: 3′ Module RT Recognition Sequence

The GIC: 3′ module RT recognition sequence may comprise or encode at least one sequence which interacts with, or is recognized by, at least one reverse transcriptase. Without wishing to be bound by theory, at least one sequence of RNA in the GIC: 3′ module RT recognition sequence may bind, at least temporarily, with at least one template RNA binding domain of an RT, such as a retroelement RT. The length and sequence identity of the GIC: 3′ module RT recognition sequence may also function to position the RT on the GIC such that the first nucleotide reverse transcribed by the RT is the intended 3′ end of the transgene to be inserted. It will be understood that the GIC: 3′ module RT recognition sequence can be referred to herein as a GIC: 3′ module 3′UTR.

In some embodiments, the at least one GIC: 3′ module RT recognition sequence is derived from or comprises the 3′ region of a native retroelement. In some embodiments, the at least one GIC: 3′ module RT recognition sequence is derived from the 3′ region of a non-LTR retroelement from G. aculeatus, D. melanogaster, L. polyphemus, P. pungitis, N. vitripennis, G. fortis, O. latipes, Z. albicollis, T. guttata, T. castaneum, T. guttatus, D. simulans, B. mori, A. vaga, other birds, other arthropods, other fish, other tunicates, other animals, or the like's genome. In some embodiments, the at least one GIC: 3′ module RT recognition sequence is modified from the 3′ region of a native retroelement by increasing the stability or homogeneity of folding. In some embodiments, the at least one GIC: 3′ module RT recognition sequence is designed and/or selected for a desired affinity and/or specificity of RT interaction, or for another mechanism that confers desired function as a template for reverse transcription. In some embodiments, the at least one GIC: 3′ module RT recognition sequence is designed and/or selected to not interact with or affect endogenous target cell components and/or have deleterious impact on the host cell.

In some embodiments, the at least one GIC: 3′ module RT recognition sequence (or GIC: 3′ module 3′UTR sequence) may comprise, encode, or be encoded by at least one of SEQ IDNOS 200-224. In some embodiments, the at least one GIC: 3′ module RT recognition sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 154-175. In some embodiments, the GIC: 3′ module RT recognition sequence is a non-native or non-natural sequence having at least 90%, 91%, 92%, 93%, 94%, 95%, 96%, 97%, 98%, or 99% identity to SEQ ID NOS 154-178.

In some embodiments, at least one GIC: 3′ module RT recognition sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 156.

In some embodiments, at least one GIC: 3′ module RT recognition sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 158, 176, 177, or 178.

In some embodiments, at least one GIC: 3′ module RT recognition sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 157.

In some embodiments, the GIC: 3′ module comprises a RT recognition sequence that is from a different species than the RT encoded by the RTC construct. For example, in some embodiments, the RT recognition sequence can be from one species of bird, and the RT can be from another species of bird. In some embodiments, the RT recognition sequence is from a bird selected from one of Zonotrichia albicollis, Taeniopygia guttata, Tinamus guttatus, or Geospiza fortis, and the RT is selected from a different bird species (e.g., Zonotrichia albicollis, Taeniopygia guttata, Tinamus guttatus, or Geospiza fortis). In some embodiments, RT encoded by the RTC construct is selected from one of Zonotrichia albicollis, Taeniopygia guttata, Tinamus guttatus, or Geospiza fortis, and the RT recognition sequence is selected from a different bird species (e.g., Zonotrichia albicollis, Taeniopygia guttata, Tinamus guttatus, or Geospiza fortis). In some embodiments, the RT encoded by the RTC construct is selected from an amino acid sequence having at least 90%, 91%, 92%, 93%, 94%, 95%, 96%, 97%, 98%, 99% or 100% identity to SEQ ID NOS 18 or 20 and the RT recognition sequence is selected from an amino acid sequence having at least 90%, 91%, 92%, 93%, 94%, 95%, 96%, 97%, 98%, 99% or 100% identity to SEQ ID NOS 157, 158, 159, or 176-178. In some embodiments, the RT encoded by the RTC construct is selected from an amino acid sequence having at least 90%, 91%, 92%, 93%, 94%, 95%, 96%, 97%, 98%, 99% or 100% identity to SEQ ID NOS: 27 or 29, and the RT recognition sequence is selected from an amino acid sequence having at least 90%, 91%, 92%, 93%, 94%, 95%, 96%, 97%, 98%, 99% or 100% identity to SEQ ID NOS 156, 158, 159, or 176-178. In some embodiments, the RT encoded by the RTC construct is selected from an amino acid sequence having at least 90%, 91%, 92%, 93%, 94%, 95%, 96%, 97%, 98%, 99% or 100% identity to SEQ ID NO 25, and the RT recognition sequence is selected from an amino acid sequence having at least 90%, 91%, 92%, 93%, 94%, 95%, 96%, 97%, 98%, 99% or 100% identity to SEQ ID NOS 156, 157, 158 or 176-178. In some embodiments, the RT encoded by the RTC construct is selected from an amino acid sequence having at least 90%, 91%, 92%, 93%, 94%, 95%, 96%, 97%, 98%, 99% or 100% identity to SEQ ID NO 31, and the RT recognition sequence is selected from an amino acid sequence having at least 90%, 91%, 92%, 93%, 94%, 95%, 96%, 97%, 98%, 99% or 100% identity to SEQ ID NOS 156, 157, or 159.

GIC: 3′ Module rRNA Sequence

The GIC: 3′ module rRNA sequence, or at a non-rDNA target site the sequence that would base-pair with TPRT primer immediately downstream of the target site nick, is an optional component of the 3′ module which, when present, may comprise a sequence of human ribosomal RNA (rRNA). Without wishing to be bound by theory, the length and sequence identity of the GIC: 3′ module rRNA sequence affects how accurately and efficiently a GIS disclosed herein inserts a transgene into a subject genome. For example, selection of some GIC: 3′ module rRNA sequence lengths may result in internal initiation of reverse transcription, effectively shortening the inserted transgene, or could enable insertion at an off-target site, both of which would decrease the efficiency and specificity of transgene insertion at the intended target site. The RTC and GIC are engineered to require a specific length of base-pairing of the GIC: 3′ module rRNA sequence to the primer sequence immediately downstream of the target site nick. This builds in additional fidelity in target site use and additional efficiency of precise transgene insertion junctions. The optimal length of GIC: 3′ rRNA is less than 20 nt, in specific 4 nt, with strong stimulation from formation of all 4 bp at the target site nick. Therefore, if the RTC were to nick randomly, with 4 nt GIC: 3′ rRNA, only 1/256 nicks would have optimal transgene insertion.

In some embodiments, the at least one GIC: 3′ module rRNA sequence may comprise or encode between about 1 and 30 nt of rRNA. In some embodiments, the at least one GIC: 3′ module rRNA sequence may comprise or encode between about 1 and 20 nt of rRNA. In some embodiments, the at least one GIC: 3′ module rRNA sequence may comprise or encode between about 1 and 10 nt of rRNA. In some embodiments, the at least one GIC: 3′ module rRNA sequence may comprise or encode between about 1 and 5 nt of rRNA.

In some embodiments, the at least one GIC: 3′ module rRNA sequence may comprise or encode a portion of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nt of rRNA.

In some embodiments, the at least one GIC: 3′ module rRNA sequence may comprise or encode about 20 nt of rRNA. In some embodiments, the at least one GIC: 3′ module rRNA sequence may comprise or encode about 4 nt of rRNA. In some embodiments, the at least one GIC: 3′ module rRNA sequence may comprise or encode about 10 nt of rRNA.

In some embodiments, at least one GIC: 3′ module rRNA sequence may comprises at least one of SEQ ID NOS 208-213. In some embodiments, the at least one GIC: 3′ module rRNA sequence is selected from the group consisting of SEQ ID NOs 208-217, or a sequence comprising one, two, or three nucleotide substitutions thereof.

### GIC: 3′Module A-Tract Sequence

The GIC: 3′ module A-Tract sequence is an optional component of the 3′ module which, when present comprises a terminal sequence tract with tandem adenosines (A). Without wishing to be bound by theory, the GIC: 3′ module A-Tract sequence may stabilize or protect the GIC from further 3′ processing and nonetheless disfavor the recognition, ribonucleoprotein assembly, trafficking, and translation-linked decay of the GIC as a mRNA by the cell. Furthermore, at least one GIC: 3′ module A-tract sequence may protect a GIC from binding by general single-stranded RNA binding proteins and aid in positioning of the GIC: 3′ rRNA sequence to base-pair with the target-site primer. As a matter of clarity, the A-Tract sequence is not equivalent to the native mRNA poly-A tail sequence, which is typically about greater than 100-200 nt of tandem A.

In some embodiments, the optional at least one GIC: 3′ module A-Tract sequence comprises or encodes a sequence of between about 1 and 50 adenosines. For example, the optional GIC: 3′ module A-Tract sequence may comprise or encode a sequence of about 1 to 50 adenosines, about 5 to 50 adenosines, about 10 to 50 adenosines, about 15 to 50 adenosines, about 20 to 50 adenosines, about 25 to 50 adenosines, about 30 to 50 adenosines, about 35 to 50 adenosines, about 40 to 50 adenosines, about 45 to 50 adenosines, about 1 to 45 adenosines, about 5 to 45 adenosines, about 10 to 45 adenosines, about 15 to 45 adenosines, about 20 to 45 adenosines, about 25 to 45 adenosines, about 30 to 45 adenosines, about 35 to 45 adenosines, about 40 to 45 adenosines, about 1 to 40 adenosines, about 5 to 40 adenosines, about 10 to 40 adenosines, about 15 to 40 adenosines, about 20 to 40 adenosines, about 25 to 40 adenosines, about 30 to 40 adenosines, about 35 to 40 adenosines, about 1 to 35 adenosines, about 5 to 35 adenosines, about 10 to 35 adenosines, about 15 to 35 adenosines, about 20 to 35 adenosines, about 25 to 35 adenosines, about 30 to 35 adenosines, about 1 to 30 adenosines, about 5 to 30 adenosines, about 10 to 30 adenosines, about 15 to 30 adenosines, about 20 to 30 adenosines, about 25 to 30 adenosines, about 1 to 25 adenosines, about 5 to 25 adenosines, about 10 to 25 adenosines, about 15 to 25 adenosines, about 20 to 25 adenosines, about 1 to 20 adenosines, about 5 to 20 adenosines, about 10 to 20 adenosines, about 15 to 20 adenosines, about 1 to 15 adenosines, about 5 to 15 adenosines, about 10 to 15 adenosines, about 1 to 10 adenosines, about 5 to 10 adenosines, or about 1 to 5 adenosines. In some embodiments, the GIC: 3′ module A-Tract sequence comprises between about 1 to 100, 1 to 90, 1 to 80, 1 to 70, or 1 to 60 adenosines.

In some embodiments, the optional at least one GIC: 3′ module A-Tract sequence comprises or encodes a sequence of between about 20 and 25 adenosines.

In some embodiments, the optional at least one GIC: 3′ module A-Tract sequence comprises or encodes a sequence of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 adenosines. In some embodiments, the GIC: 3′ module A-Tract sequence comprises 22 adenosines.

### Modularity of the GIC: 3′ Module

The disclosed 3′ module components may be used interchangeably with each other in a combinatorial manner to design a 3′ module with the required or desired functionality for a particular GIS.

In some embodiments, the at least one GIC: 3′ module comprises at least GIC: 3′ module RT recognition sequence. In some embodiments, the at least one GIC: 3′ module comprises at least one GIC: 3′ module rRNA sequence. In some embodiments, the at least one GIC: 3′ module comprises at least one GIC: 3′ module A-Tract sequence. In some embodiments, the at least one GIC: 3′ module comprises at least GIC: 3′ module RT recognition sequence and at least one GIC: 3′ module rRNA sequence. In some embodiments, the at least one GIC: 3′ module comprises at least one GIC: 3′ module RT recognition sequence and at least one GIC: 3′ module A-Tract sequence. In some embodiments, the at least one GIC: 3′ module comprises at least one GIC: 3′ module RT recognition sequence, at least one GIC: 3′ module rRNA sequence, and at least one GIC: 3′ module A-Tract sequence.

In some embodiments, at least one GIC: 3′ module may comprise any combination of: (a) at least one GIC: 3′ module RT recognition sequence selected from, encoding, or encoded by any one of SEQ ID NOS 154-175, (b) at least one GIC: 3′ module rRNA sequence selected from, encoding, or encoded by any one of SEQ ID NOS 208-217, and/or (c) at least one GIC: 3′ module A-Tract sequence.

### Exemplary GIC: 3′ Modules

In some embodiments, at least one GIC: 3′ module may comprise, encode, or be encoded by at least one of SEQ ID NOS 225-253. In some embodiments, at least one 3′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one sequence selected from the group consisting of SEQ ID NOS 225-253. In some embodiments, the at least one GIC: 3′ module comprises a sequence having at least 90% identity (e.g., at least 90%, 91%, 92%, 93%, 94%, 95%, 96%, 97%, 98%, or 99% identity) to a sequence selected from the group consisting of SEQ ID NOS 225-253, or any combination thereof. In some embodiments, the GIC: 3′ module comprises a non-native or non-natural sequence having at least 90%, 91%, 92%, 93%, 94%, 95%, 96%, 97%, 98%, or 99% identity to SEQ ID NOS 225-253.

In some embodiments, at least one GIC: 3′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 238-244.

In some embodiments, the at least one GIC: 3′ module may comprise a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to a sequence selected from the group consisting of “GACGGTAGC TAGGTTCGCA AGGCAGCCAC AAGCCAAAGA TAGGTAGGGT GCTCATAGTG AGTAGGGACA GTGCCTTTTG ATTCACAACG CGTCAATACC ATCTGACACG GATACCCTTA CCGGACTTGT CATGATCTCC CAGACTTGTC CAAGGTGGAC GGGCCACCTT TACTTAACCC GGAAAAGGAA CATATATTAA TTATATGTGT TCGGAAAA” (SEQ ID NO:176), “CCGGACTTGT CATGATCTCC CAGACTTGTC CAAGGTGGAC GGGCCACCTT TACTTAACCC GGAAAAGGAA CATATATTAA TTATATGTGT TCGGAAAA” (SEQ ID N:177), and “CAAGGTGGAC GGGCCACCTT TACTTAACCC GGAAAAGGAA CATATATTAA TTATATGTGT TCGGAAAA”(SEQ ID NO:178). In some embodiments, these sequences further include a 3′ sequence TAGCaaaaaaaaaaaaaaaaaaaaaa (SEQ ID NO: 334).

In some embodiments, at least one GIC: 3′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 239.

In some embodiments, at least one GIC: 3′ module may comprise, encode, or be encoded by a sequence with at least 95%, 90%, 85%, 80%, 75%, 70%, 65%, 60%, 55%, 50%, 45%, 40%, 35%, 30%, 25%, 20%, 10%, or 5% homology to SEQ ID NO 232.

In some embodiments, at least one GIC: 3′ module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 240.

### GIC: Payload Module

GIC: payload modules for use in a GIC of the invention comprise or encode at least one payload sequence that will serve as part of the template for reverse transcription and insertion into the subject genome by a GIS disclosed herein. As used herein, the term “payload sequence” or simply “payload” refers to any biopolymer sequence intended for insertion into a target genome by at least one GIS of the invention. A payload sequence of the invention may include at least one transgene.

As used herein, the term “transgene” is used in its broadest sense to refer to any genetic sequence inserted into a subject genome by a GIS of the invention. For example, transgenes may include sequences not normally found in the subject genome or sequences normally found in the subject genome but not at the target insertion site. Transgenes may include, without limitation, sequences which comprise or encode a desired expression product (e.g., at least one mRNA, microRNA, siRNA, rRNA, tRNA, long non-coding RNA, small cytoplasmic RNA, small nuclear RNA, small nucleolar RNA, small Cajal body RNA, circular RNA, peptide, polypeptide, and/or protein) and/or sequences which control expression of at least one transgene. In some embodiments, the transgene encodes a protein selected from telomerase reverse transcriptase (TERT, e.g., human TERT), phenylalanine hydroxylase (PAH, e.g., human PAH), Factor VIII (e.g., human Factor VIII), a mutant Factor VIII having variable size B domains (e.g., hFactor VIII N6, and hFactor VIII N6mutant), or Factor IX (e.g, human Factor IX). In some embodiments, the transgene encodes a regulatory RNA. In some embodiments, the transgene encodes an inhibitor of another protein. In some embodiments, the inhibitor is single chain antibody. In some embodiments, the transgene encodes a protein that can be used to treat a disease selected from a gene in Table X.

TABLE X
Representative Transgenes.

| Disease | Locus | Gene name |
|---|---|---|
| Achromatopsia (ACHM) | CNGB3 | beta 3 subunit of a cyclic nucleotide-gated ion |
| | | channel |
| Achromatopsia (ACHM) | CNGA3 | alpha 3 subunit of a cyclic nucleotide-gated ion |
| | | channel |
| Adrenoleukodystrophy | ABCD1 | ALDP protein |
| Albinism, oculocutaneous, type II | OCA2 | Oculocutaneous albinism II (OCA2) |
| Beta thalassemia | HBB | hemoglobin subunit beta |
| Brugada Syndrome | SCN5A | Sodium Voltage-Gated Channel Alpha Subunit 5 |
| Canavan disease | ASPA | aspartoacylase |
| Charcot-Marie-Tooth Disease | PMP22 | Peripheral Myelin Protein 22 |
| Choroideremia (CHM) | REP1 | Rab escort protein 1 |
| Chronic granulomatous disease (CGD) | CYBA | p22-phox (phagocyte oxidase): alpha subunit |
| CILD1, with or without situs inversus (Kartagener | DNAI1 | Dynein, axonemal, intermediate chain 1 |
| syndrome) | | |
| Classical Ehlers Danlos (cEDS) | COL5A1/2 | Type V collagen |
| Cleidocranial Dysplasia (CCD) | RUNX2 | RUNX Family Transcription Factor 2 |
| Congenital deafness (presents at birth) | GJB2 | Gap Junction Protein Beta 2 |
| Crigler-Najjar syndrome, type I | UGT1A1 | bilirubin uridine diphosphate glucuronosyl |
| | | transferase |
| Cystic fibrosis | CFTR | CF transmembrane conductance regulator |
| Familial Adenomatous Polyposis | APC | APC Regulator Of WNT Signaling Pathway |
| Fanconi anemia | FANCE | FA Complementation Group E |
| Fragile X syndrome | FMR1 | fragile X messenger ribonucleoprotein 1 |
| Gaucher disease Type 1 | GBA | glucosylceramidase beta 1 |
| Hemochromatosis (iron overload) | HFE | Homeostatic Iron Regulator |
| Hemophilia A | F8 | Coagulation factor VIII |
| Huntington's disease | HTT | Huntingtin (HTT) |
| Hypercholesterolemia, type B | APOB | apolipoprotein B |
| Hypophosphatemic rickets | PHEX | Phosphate-regulating endopeptidase |
| | | homologue, X-linked |
| Kneist Syndrome | COL2A1 | Alpha-1 chain of type II collagen |
| Leber congenital amaurosis (LCA) | CEP290 | centrosomal protein 290 kDa |
| Leber congenital amaurosis (LCA) | CRB1 | crumbs family member 1, photoreceptor |
| | | morphogenesis associated |
| Leber congenital amaurosis (LCA) | GUCY2D | guanylate cyclase 2D, membrane (retina- |
| | | specific) |
| Leber Hereditary Optic Neuropathy (LHON) | ND4 | NADH dehydrogenase 4 |
| Leber Hereditary Optic Neuropathy (LHON) | ND1 | NADH dehydrogenase 1 |
| Lesch-Nyhan syndrome (LNS) | HPRT1 | Hypoxanthine-guanine |
| | | phosphoribosyltransferase |
| Marfan syndrome | FBN1 | Fibrillin 1 |
| Medium-chain acyl-CoA dehydrogenase deficiency | ACADM | Medium-Chain Acyl-CoA Dehydrogenase |
| Mucopolysaccharidoses (MPS) | IDUA | Alpha-L-Iduronidase |
| Muscular dystrophy, Becker type | DMD | Dystrophin |
| Muscular dystrophy, Duchenne type | DMD | Dystrophin |
| Myotonic dystrophy type 1 | DMPK | Dystrophia myotonica-protein kinase |
| Myotonic dystrophy type 2 | CNBP | CCHC-type zinc finger nucleic acid binding |
| | | protein |
| Neurofibromatosis types II | NF2 | Moesin-Ezrin-Radixin Like (MERLIN) Tumor |
| | | Suppressor |
| Neurofibromatosis, type 1 | NF1 | Neurofibromin 1 (NF1) |
| Niemann-Pick disease type A and B | SMPD1 | Sphingomyelinase |
| Parkison's Disease | GBA | glucosylceramidase beta 1 |
| Phenylketonuria (PKU) | PAH | Phenylalanine hydroxylase (PAH) |
| Polycystic kidney disease 1 and 2 | PKD2 | Polycystic kidney disease 2 |
| Respiratory distress syndrome, Surfactant protein-B | SFTPC | Surfactant, pulmonary-associated protein C |
| (SP-B) deficiency | | |
| Retinitis pigmentosa visual field | EYS | Eyes Shut Homolog |
| Rett's syndrome | MECP2 | Methyl-CpG-binding protein 2 |
| Rhodopsin-mediated autosomal dominant retinitis | PRPH2 | Peripherin 2 |
| pigmentosa (RHO-adRP) | | |
| Rhodopsin-mediated autosomal dominant retinitis | PRPF31 | Pre-MRNA Processing Factor 31 |
| pigmentosa (RHO-adRP) | | |
| Rhodopsin-mediated autosomal dominant retinitis | RHO | Rhodopsin |
| pigmentosa (RHO-adRP) | | |
| Sickle-cell anemia | HBB | hemoglobin subunit beta |
| Spermatogenic failure, nonobstructive | USP9Y | Ubiquitin-specific peptidase 9Y |
| Spinal muscular atrophy | SMN1 | Survival Of Motor Neuron 1, Telomeric |
| Stargardt disease | ABCA4 | ATP-binding cassette sub-family A member 4 |
| Tay-Sachs disease | HEXA | Hexosaminidase A |
| Usher Syndrome | MYO7A | myosin VIIA |
| vitelliform macular dystrophy (Best) | BEST1 | bestrophin-1 |
| Von Hippel-Lindau (VHL) | VHL | von Hippel-Lindau ubiquitination complex |
| X-linked retinitis pigmentosa (XLRP) | RPGR | retinitis pigmentosa GTPase regulator |
| X-linked retinitis pigmentosa (XLRP) | RP2 | retinitis pigmentosa 2 |
| X-linked retinoschisis (XLRS) | RS1 | retinoschisin |
| α1-antitrypsin deficiency (COPD, emphysema, liver | SERPINA1 | α1-antitrypsin |
| disease) | | |

### GIC: Payload Module Architecture

A GIC: payload module may comprise at least one (e.g., one, two or three or more) transgene sequence and may also comprise, optionally at least one transgene promoter sequence, optionally at least one transgene 5′ untranslated sequence, optionally at least one transgene 3′ untranslated sequence, optionally at least one transgene polyadenylation signal or poly-A tail sequence, optionally at least one transgene non-coding RNA (ncRNA) processing sequence, and any combination thereof.

Turning once more to FIG. 6, the architecture of an exemplary payload module 430 is illustrated in the top expanded view. When present, the optional transgene promoter sequence 431 may include or encode at least one promoter which may control expression of the inserted transgene by the subject cell. The optional transgene 5′ UTR sequence 432, may include or encode sequences that, when the inserted transgene is expressed, encode a 5′ UTR for the transgene mRNA. The transgene sequence 433 of the payload module may comprise at least one transgene sequence for reverse transcription and insertion by a disclosed GIS, for example this sequence may comprise or encode the ORF of a gene of interest. The optional transgene 3′ UTR sequence 434 may include or encode at least one 3′ UTR for an expressed transgene's mRNA. Similarly, the optional transgene polyadenylation signal sequence 435 may include or encode a polyadenylation signal for an expressed transgene's mRNA. Finally, the optional transgene non-coding RNA (ncRNA) processing sequence 436 may include or encode termination and/or 3′ processing signals for transgene expressed nrRNAs.

### Transgene Promoter Sequence and RNAP II 5′ UTR Sequences

When present, the transgene promoter sequence may comprise or encode at least one promoter sequence which comprises the means to promote expression of a transgene in a subject genome. Many such means of promoting expression of a gene and/or transgene are known in the art, including inserting a known promoter sequence 5′ to the gene of interest. It will be understood by those skilled in the art that the identity of a promoter sequence may be selected based on the identity of the transgene and other use specific factors and therefore, any suitable promoter may be utilized in the practice of this disclosure.

Exemplary promoters for use in this disclosure may be constitutive or inducible. In some embodiments, the transgene promoter sequence may comprise or encode at least one promoter for RNA polymerases I-III (RNAP I, RNAP II or III). In some embodiments, instead of or in addition to a promoter, the same region of at least one transgene may comprise or encode at least one ribozyme or other motif to enable liberation of a transgene RNA transcript from host cell rDNA RNAP I transcription.

In some embodiments, the at least one transgene promoter sequence comprises or encodes at least one human U1 snRNA promoter. In some embodiments, the at least one transgene promoter sequence comprises or encodes at least one human U3 snRNA promoter. In some embodiments, the at least one transgene promoter sequence comprises or encodes at least one human U6 snRNA promoter. In some embodiments, the at least one transgene promoter sequence comprises or encodes at least one human tRNA promoter.

When present, the transgene 5′ UTR sequence comprises or encodes at least one mRNA 5′ UTR for the inserted transgene. In general, this sequence comprises or encodes a sequence that, when the inserted transgene is expressed by the cell, is not translated into an amino acid biopolymer by the cell ribosome. These sequences include for example, a 5′ UTR natively associated with the transgene, a 5′ UTR which is non-native to the transgene (including sequences derived from the 5′ sequence of retroelements), a “synthetic” 5′ UTR which may not be found associated with any known wild-type gene, and any combinations thereof,

It will be understood by those skilled in the art that the selection of the transgene 5′ UTR sequence will depend on the identity of the transgene and other use specific factors and therefore any known or discovered 5′ UTR sequence may be suitable for use in a transgene 5′ sequence of a payload module.

In some embodiments, at least one transgene promoter sequence may comprise, encode, or be encoded by at least one of SEQ ID NOS 275-278 or 282-283. In some embodiments, at least one transgene promoter sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 275-278 or 282-283.

In some embodiments, at least one transgene promoter sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 275.

In some embodiments, at least one transgene promoter sequence may comprise, encode, or be encoded by a sequence with at least 95%, 90%, 85%, 80%, 75%, 70%, 65%, 60%, 55%, 50%, 45%, 40%, 35%, 30%, 25%, 20%, 10%, or 5% homology to SEQ ID NO 276.

In some embodiments, at least one transgene promoter sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 277.

In some embodiments, at least one transgene promoter sequence comprises a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 278.

In some embodiments, at least one transgene promoter sequence comprises a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 282.

In some embodiments, at least one transgene promoter sequence comprises a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 283.

In some embodiments, the GIC: payload module comprises an RNA polymerase (RNAP) terminator sequence located 5′ of the transgene promoter sequence. In some embodiments, the RNAP is RNAP I (Pol I), and the termination sequence prevents Pol I readthrough transcription when the GIC payload module is integrated into a ribosomal DNA gene target site. In some embodiments, the RNAP terminator sequence comprises the sequence 5′-AGGTCGACCAGATGTCCGAGGTCGACCAGTTGTCCG-3′ (SEQ ID NO:333).

### Transgene Sequence

The transgene sequence of the payload module comprises or encodes at least one sequence of interest for insertion into a subject genome. As used herein, the term “sequence of interest” refers to a biopolymer sequence comprising or encoding at least one desired expression product. In some embodiments, the transgene encodes a protein selected from hTERT, hPAH, hFactor VIII, a mutant hFactor VIII having variable size B domains (e.g., hFactor VIII N6, and hFactor VIII N6mutant), or Factor IX (e.g, human Factor IX). In some embodiments, the transgene encodes a regulatory RNA. In some embodiments, the transgene encodes an inhibitor of another protein. In some embodiments, the inhibitor is single chain antibody. In some embodiments, the transgene encodes a protein that can be used to treat a disease selected from a gene in Table X.

Any sequence of interest may be suitable for the practice of this disclosure, without limitation to the origin from which the sequence was derived (i.e., its species of origin or if the sequence is natural or artificial), or the length of the sequence.

In some embodiments, at least one transgene sequence may comprise, encode, or be encoded by at least one of SEQ ID NOS 284-295. In some embodiments, at least one transgene sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 284-295.

In some embodiments, at least one transgene sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 292 or 293.

In some embodiments, at least one transgene sequence may comprise, encode, or be encoded by a sequence with at least 95%, 90%, 85%, 80%, 75%, 70%, 65%, 60%, 55%, 50%, 45%, 40%, 35%, 30%, 25%, 20%, 10%, or 5% homology to at least one of SEQ ID NO 294-295.

In some embodiments, at least one transgene sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NO 314-332.

### Transgene 3′ UTR Sequence and Polyadenylation Signal

When present, the transgene 3′ UTR sequence comprises or encodes at least one mRNA 3′ UTR for the inserted transgene. In general, this sequence comprises or encodes a sequence that when the inserted transgene is expressed by the cell is not translated into an amino acid biopolymer by the cell ribosome. These sequences can include for example, a 3′ UTR natively associated with the transgene, a 3′ UTR which is non-native to the transgene (including sequences derived from the 3′ sequence of retroelements), a “synthetic” 3′ UTR which is not associated with any known wild-type gene, and any combinations thereof.

It will be understood by those skilled in the art that the selection of the transgene 3′ UTR sequence will depend on the identity of the transgene and other use specific factors and therefore any known or discovered 3′ UTR sequence may be suitable for use in a transgene 3′ sequence of a payload module.

When present the transgene polyadenylation signal sequence comprises or encodes at least one transgene mRNA polyadenylation signal. Any suitable polyadenylation signal known or discovered may be used in a template module of this disclosure. For the sake of clarity, the at least one transgene polyadenylation signal present in or encoded within the inserted transgene provides for RNAP II to append a poly-A tail on an mRNA or ncRNA expression product of the transgene.

In some embodiments, the at least one transgene 3′ UTR sequence may comprise a sequence selected from at least one of SEQ ID NOS 279-281. In some embodiments, the at least one transgene 3′ UTR sequence may comprise a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one SEQ ID NOS 279-281.

In some embodiments, at least one transgene 3′ UTR sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 279.

In some embodiments, at least one transgene 3′ UTR sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 280.

In some embodiments, at least one transgene 3′ UTR sequence may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NO 281.

Transgene Non-Coding RNA (ncRNA) Processing Sequence

When present, the transgene ncRNA processing sequence comprises or encodes sequences which control expression or processing of transgene expressed ncRNA, such as transfer RNAs (tRNAs), rRNAs, microRNAs, siRNAs, snRNAs, and the like. In some embodiments, the at least one non-coding RNA (ncRNA) processing sequence comprises or encodes at least one termination signal, at least one 3′ processing signal, and any combination thereof for at least one transgene expressed ncRNA.

In some embodiments, at least one transgene ncRNA processing sequence comprises or encodes at least one MALAT1 3′ processing and/or protection signal. In some embodiments, at least one transgene ncRNA processing sequence comprises or encodes at least one RNA triplex-forming end-protection structure. In some embodiments, at least one transgene ncRNA processing sequence comprises or encodes at least one endonuclease recruitment structure, site, or motif. In some embodiments, at least one transgene ncRNA processing sequence comprises or encodes at least one poly-thymidine tract. In some embodiments, at least one transgene RNA 3′ termination and/or processing sequence includes a SalI termination box for RNAP I.

### Modularity of the Payload Module

The disclosed GIC: payload module components may be used interchangeably with each other in a combinatorial manner to design a 3′ module with the required or desired functionality for a particular GIS.

In some embodiments, at least one GIC: payload module may comprise or encode at least one transgene sequence. In some embodiments, at least one GIC: payload module may optionally comprise or encode at least one transgene promoter sequence. In some embodiments, at least one GIC: payload module may optionally comprise or encode at least one transgene 5′ UTR sequence. In some embodiments, at least one GIC: payload module may optionally comprise or encode at least one transgene 3′ UTR sequence. In some embodiments, at least one GIC: payload module may optionally comprise or encode at least one transgene polyadenylation signal sequence. In some embodiments, at least one GIC: payload module may optionally comprise or encode at least one transgene ncRNA processing sequence.

In some embodiments, at least one GIC: payload module may comprise or encode at least one transgene sequence, at least one transgene promoter sequence, at least one transgene 5′ UTR sequence, at least one transgene 3′ UTR sequence, at least one transgene polyadenylation signal sequence, and/or at least one ncRNA processing sequence.

In some embodiments, at least one GIC: payload module may comprise any combination of: (a) at least one transgene promoter sequence and 5′ UTR sequence selected from any one of SEQ ID NOS 275-278, or a sequence having at least 90% identity (e.g., at least 90, 91, 92, 93, 94, 95, 96, 97, 98, 99 or 100% identity) to any one of SEQ ID NOS 275-278, (b) at least one transgene sequence selected from, encoding, or encoded by any one of SEQ ID NOS 284-295 or SEQ ID NOS 296-332, or a sequence having at least 90% identity (e.g., at least 90, 91, 92, 93, 94, 95, 96, 97, 98, 99 or 100% identity) to any one of SEQ ID NOS 284-295 and 296-332, and (c) at least one transgene 3′ UTR sequence and polyadenylation signal selected from SEQ ID NOS 279-281, or a sequence having at least 90% identity (e.g., at least 90, 91, 92, 93, 94, 95, 96, 97, 98, 99 or 100% identity) to SEQ ID NOS 279-281.

### Exemplary GIC: Payload Modules

In some embodiments, at least one GIC: payload module may comprise, encode, or be encoded by at least one sequence selected from SEQ ID NOS 296-332. In some embodiments, at least one GIC: payload module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one sequence selected from SEQ ID NOS 296-332.

In some embodiments, at least one GIC: payload module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NOS 292, 293, 314, or 315.

In some embodiments, at least one GIC: payload module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NOS 294, 295, 316, or 317.

In some embodiments, at least one GIC: payload module may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NOS 318, 319, 320, or 321.

### Modularity of the GIC

The disclosed GIC components (i.e., GIC: 5′ modules, GIC: 3′ modules, and GIC: payload modules) may be used interchangeably with each other in a combinatorial manner to design a GIC with the required or desired functionality for a particular GIS.

In some embodiments, at least one GIC comprises at least one GIC: 5′ module. In some embodiments, at least one GIC comprises at least one GIC: payload module. In some embodiments, at least one GIC comprises at least one GIC: 3′ module. In some embodiments, at least one GIC comprises at least one GIC: 5′ module and at least one GIC: payload module. In some embodiments, at least one GIC comprises at least one GIC: 5′ module and at least one GIC: 3′ module. In some embodiments, at least one GIC comprises at least one GIC: 5′ module, at least one GIC: payload module, and at least one GIC: 3′ module.

In some embodiments, at least one GIC comprises at least one GIC: 5′ module comprising a GIC: 5′ module RE sequence derived from the same species of retroelement as the GIC: 3′ module RT recognition sequence. In some embodiments, at least one GIC comprises at least one GIC: 5′ module comprising a GIC: 5′ module RE sequence derived from a different species of retroelement as the GIC: 3′ module RT recognition sequence. In some embodiments, at least one GIC comprises at least one GIC: 5′ module comprising a GIC: 5′ module sequence not native to eukaryotic biology and generally useful for at least one GIC containing any GIC: 3′ module RT recognition sequence.

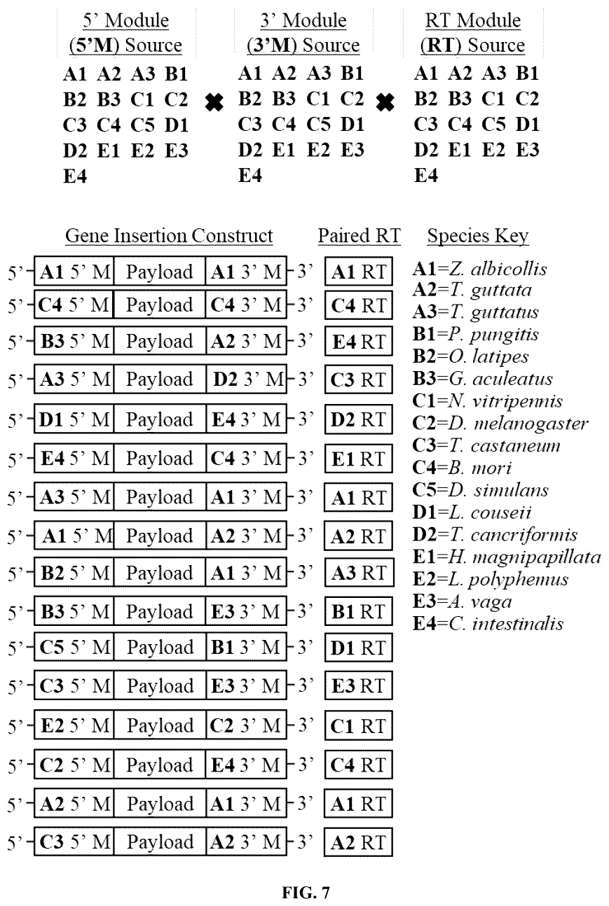

In some embodiments, the GIC comprises a combination of GIC: 5′ module sequence sources and GIC: 3′ module sequence sources illustrated in FIG. 7. In FIG. 7, A1 is Zonotrichia albicollis, A2 is Taeniopygia guttata, A3 is Tinamus guttatus, A4 Geospiza fortis, B1 is Pungitis pungitis, B2 is Oryzias latipes, B3 is Gasterosteus aculeatus, C1 is Nasonia vitripennis, C2 is Drosophila melanogaster, C3 is Tribolium castaneum, C4 is Bombyx mori, C5 is Drosophila simulans, C6 is Drosophila mercatorum, D1 is Lepidurus couseii, D2 is Triops cancriformis, E1 is Hydra magnipapillata, E2 is Limulus polyphemus, E3 is Adineta vaga, and E4 is Ciona intestinalis.

In some embodiments, at least one GIC may comprise, encode, or be encoded by any combination of: (a) at least one GIC: 5′ module selected from, encoding, or encoded by any sequence selected from SEQ ID NOS 179-205, or a sequence having one, two or three nucleotide changes or substitutions relative to SEQ ID NOs: 179-205, SEQ ID NOS 60-153, or a sequence having at least 90% identity (e.g., at least 90, 91, 92, 93, 94, 95, 96, 97, 98, 99 or 100% identity) to SEQ ID NOS 60-153, SEQ ID NOS 206-207, or a sequence having at least 90% identity (e.g., at least 90, 91, 92, 93, 94, 95, 96, 97, 98, 99 or 100% identity) to SEQ ID NOS 206-207, (b) at least one GIC: payload module selected from, encoding, or encoded by any sequence selected from one of SEQ ID NOS 284-295, or 499-525, or a sequence having at least 90% identity (e.g., at least 90, 91, 92, 93, 94, 95, 96, 97, 98, 99 or 100% identity) to SEQ ID NOS 284-295, or 296-318, and/or (c) at least one GIC: 3′ module selected from, encoding, or encoded by any sequence selected from one of SEQ ID NOS 225-253, or a sequence having at least 90% identity (e.g., at least 90, 91, 92, 93, 94, 95, 96, 97, 98, 99 or 100% identity) to SEQ ID NOS 225-253. Exemplary GIC

In some embodiments, at least one GIC may comprise, encode, or be encoded by at least one of SEQ ID NOS 284-295, or 499-525. In some embodiments, at least one GIC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to at least one of SEQ ID NOS 284-295, or 296-332.

In some embodiments, at least one GIC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NOS 292, 293, 314, or 315.

In some embodiments, at least one GIC may comprise, encode, or be encoded by a sequence with at least 100%, 99%, 98%, 97%, 96%, 95%, 90%, 85%, 80%, 75%, 70%, 65%, 60%, 55%, 50%, 45%, 40%, 35%, 30%, 25%, 20%, 10%, or 5% homology to SEQ ID NOS 294, 295, 316, or 317.

In some embodiments, at least one GIC may comprise, encode, or be encoded by a sequence with at least 95%, 90%, 85%, 80%, 75%, 70%, 65%, 60%, 55%, 50%, 45%, 40%, 35%, 30%, 25%, 20%, 10%, or 5% homology to SEQ ID NOS 318, 319, 320, or 321.

### GIS Design and Modularity

The disclosed GIS components (i.e., RTCs and GICs) may be used interchangeably with each other in a combinatorial manner to design a GIS with the required or desired functionality.

In some embodiments, at least one GIS may comprise at least one RTC. In some embodiments, at least one GIS may comprise at least one GIC. In some embodiments, at least one GIS may comprise at least RTC and at least one GIC.

### Composition of GIS Biopolymers

The composition of biopolymers comprising the GIS components may be selects from those disclosed herein in a combinatorial manner to design a GIS with the required or desired functionality.

In some embodiments, at least one RTC may be introduced to at least one subject as an RNA biopolymer. In some embodiments, at least one RTC may be introduced to at least one subject as an mRNA biopolymer.

In some embodiments, at least one GIC may be introduced to at least one subject as an RNA biopolymer. In some embodiments, at least one GIC may be introduced to at least one subject as a linear RNA biopolymer.

In some embodiments, at least one RTC may be introduced to at least one subject as an RNA biopolymer and at least one GIC may be introduced to at least one subject as an RNA biopolymer.

In some embodiments, at least one RTC may be introduced to at least one subject as an mRNA biopolymer and at least one GIC may be introduced to at least one subject as an RNA biopolymer.

In some embodiments, at least one RTC and/or at least one GIC may be introduced to at least one subject as a DNA biopolymer. In some embodiments, at least one RTC and/or at least one GIC may be introduced to at least one subject as a plasmid.

In some embodiments, at least one RTC may be introduced to at least one subject as an amino acid biopolymer. In some embodiments, at least one RTC may be introduced to at least one subject as a protein.

In some embodiments, at least one RTC may be introduced to at least one subject as an amino acid biopolymer and at least one GIC may be introduced to at least one subject as an RNA biopolymer. In some embodiments, at least one RTC may be introduced to at least one subject as a plasmid and at least one GIC may be introduced to at least one subject as an RNA biopolymer.

In some embodiments, at least one RTC may be introduced to at least one subject as a plasmid and at least one GIC may be introduced to at least one subject as a plasmid. In some embodiments, at least one RTC may be introduced to at least one subject as an RNA (e.g., an mRNA) and at least one GIC may be introduced to at least one subject as plasmid.

### Paired-RTs

A GIS of the invention may be optimized for a desired function by designing or selecting the composition of at least one of the GIS's GICs, RTCs, or both to control interaction between the GIC and RTC. For example, altering the compositions of the GIC and/or RTC may allow for the changes in the efficiency, rate, and/or fidelity of full-length payload insertion as monitored by detection of insertions using PCR, sequencing, and/or by payload transgene expression; the sequence specificity and/or chromosome location of target site selection for payload insertion as monitored by sequencing, hybridization, or other visualization of genomic locations of inserted DNA; the selectivity for which an RTC utilizes only the administered GIC as a reverse transcription template; and the like. The term “paired RT” is used herein to refer to the particular RTC: RT-module sequence administered in combination with a particular GIC sequence.

Without wishing to be bound by theory, altering the interaction of an RTC and GIC may be accomplished through the selection of the RTC: RT-module and the GIC: 5′ module and/or GIC: 3′ module. For example, specificity of an RTC for a GIC may be altered by selecting components derived from the same or different species of retroelements. As used herein, two GIS components are said to be homologous if they are derived from the same species of retroelement. Conversely, two GIS components are said to be heterologous if they are derived from different species of retroelement.

In some embodiments, at least one of the RTC: RT-modules comprise or encode at least one sequence derived from a different species of retroelement than at least one of retroelement derived GIC: 5′ module and/or GIC: 3′ module sequences (referred to herein as a “heterologous paired RT”).

In some embodiments, all the sequences derived from a retroelement in both the RTC and GIC are derived from the same species of retroelement (referred to herein as a “homologous paired RT”).

In some embodiments, heterologous paired RTs may have increased specificity as compared to homologous paired RTs.

As used herein, the term “specificity” refers to the likelihood with which a paired RT will efficiently and/or preferentially utilize the intended template RNA for transgene insertion.

In some embodiments, at least one GIS may comprise at least one combination of GIC, and paired RT as illustrated in FIG. 7.

### Exemplary GIS

In some embodiments, at least one GIS may comprise, encode, or be encoded by any combination of: (a) at least one RTC selected from, encoding, or encoded by any sequence selected from one of SEQ ID NOS 1-59, or a sequence having at least 90% identity (e.g., at least 90, 91, 92, 93, 94, 95, 96, 97, 98, 99 or 100% identity) to one of SEQ ID NOS 1-59 and (b) at least one GIC selected from, encoding, or encoded by any sequence comprising one of SEQ ID NOS 179-205, or a sequence having one, two or three nucleotide changes or substitutions relative to SEQ ID NOs: 179-205; SEQ ID NOS 60-153, or a sequence having at least 90% identity (e.g., at least 90, 91, 92, 93, 94, 95, 96, 97, 98, 99 or 100% identity) to SEQ ID NOS 60-153, SEQ ID NOS 206-207, or a sequence having at least 90% identity (e.g., at least 90, 91, 92, 93, 94, 95, 96, 97, 98, 99 or 100% identity) to SEQ ID NOS 206-207; SEQ ID NOS 284-295, or 296-332, or a sequence having at least 90% identity (e.g., at least 90, 91, 92, 93, 94, 95, 96, 97, 98, 99 or 100% identity) to SEQ ID NOS 284-295, or 296-332; and/or SEQ ID NOS 225-253, or a sequence having at least 90% identity (e.g., at least 90, 91, 92, 93, 94, 95, 96, 97, 98, 99 or 100% identity) to SEQ ID NOS 225-253.

### III. FORMULATIONS AND DELIVERY MECHANISMS

### Nucleic Acids

In some embodiments, the RTC constructs or GIC constructs may contain one or more modified nucleotides such as, but not limited to, nucleobase modifications, sugar modified nucleotides, and/or backbone modifications. In some embodiments, the RTC constructs or GIC constructs may contain combined modifications, for example, combined nucleobase and backbone modifications.

In some embodiments, the modified nucleotide may be a nucleobase-modified nucleotide. Modified bases refer to nucleotide bases such as, but not limited to, adenine, cytosine, thymine, guanine, uracil, xanthine, inosine, and queuosine that have been modified by the replacement or addition of one or more groups or atoms. In some embodiments, the modified nucleotide may be a backbone-modified nucleotide.

The RTC constructs and/or GIC constructs may include one or more substitutions, insertions and/or additions, deletions, and covalent modifications with respect to reference sequences, in particular, the sequence of interest, are included within the scope of this invention.

In some embodiments, the RTC constructs and/or GIC constructs includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.).

The RTC constructs and/or GIC constructs may include any useful modification, such as to the sugar, the nucleobase, or the internucleoside linkage (e.g., to a linking phosphate/to a phosphodiester linkage/to the phosphodiester backbone).

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the RNA may enhance immune evasion. The RNA may be synthesized and/or modified by methods well established in the art.

In some embodiments, at least one RNA construct may comprise at least one modified uracil. Examples of uracil modifications include 5-methyl-uridine, 5-methoxy-uridine, pseudouridine, N1-methyl-pseudouridine, and/or 2-thiouridine. In some embodiments, at least one RNA construct may comprise at least one modified adenosine. Examples of adenosine modification include 2,6-diaminopurine deoxynucleotide.

In some embodiments, sugar modifications (e.g., at the 2′ position or 4′ position) or replacement of the sugar one or more RNA may, as well as backbone modifications, include modification or replacement of the phosphodiester linkages.

### Delivery Mechanisms

Gene Insertion Systems (GIS) of the invention may be introduced to a subject via any delivery mechanism known in the art. As used herein, “delivery mechanism” refers to a method or composition used to introduce the GIS, a component of the GIS, or a product of the GIS to a subject. Non-limiting examples of delivery mechanisms include delivery vehicles, direct transfection (such as with a transfection agent), implantation of cells previously transfected with the GIS, and any combination thereof.

### Delivery Vehicles

In some embodiments, a GIS of the invention may be formulated in delivery vehicles. In general, delivery vehicles may facilitate in vivo or in vitro transfection of subject cells by protecting GIS components from degradation in the extracellular environment, facilitating uptake by subject cells, enhancing endosomal escape, and any combination thereof. Delivery vehicle may include but are not limited to nanoparticles including lipid-based nanoparticles (e.g., lipid nanoparticles (LNPs), liposomes, and micelles) and non-lipid nanoparticles (e.g., virus like particles (VLPs) and polymeric delivery particles).

### Nanoparticles

In some embodiments, delivery vehicles may include at least one nanoparticle. In general, the term “nanoparticle” as used herein may refer to any particle ranging in size from 10-1000 nm, for example a particle may be 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 nm.

### Lipid Based Particles

In some embodiments, delivery vehicles may comprise at least one lipid-based nanoparticles including, but not limited to lipid nanoparticles (LNPs), liposomes, micelles, and any combination thereof.

### Lipid Nanoparticles

In some embodiments, the delivery vehicle may be a lipid nanoparticle (LNP). In general, LNPs possess an exterior lipid layer including a hydrophilic exterior surface that is exposed to the non-LNP environment, non-aqueous or an aqueous interior space (i.e., micelle like and vesicle like LNPs respectively), and at least one hydrophobic inter-membrane space. LNP membranes may be non-lamellar or lamellar and may be comprised of 1, 2, 3, 4, 5 or more than 5 layers. LNPs may be solid or semi-solid. In some embodiments at least one cargo or a payload (such as the GIS) may be present in the interior space, the inter membrane space, on the exterior surface, or any combination thereof of the LNP.

LNPs useful herein are known in the art and generally comprise an ionizable (cationic) lipid, a phospholipid, cholesterol, and a polymer-conjugated lipid. Without wishing to be bound by theory, cholesterol promotes membrane fusion and aids in LNP stability, a phospholipids may aid in endosomal escape and provide structure to the LNP bilayer, polymer-conjugated lipids reduce LNP aggregation and “protects” the LNP from non-specific endocytosis by immune cells, and the ionizable (cationic) lipid enhances endosomal escape and complexes negatively charged cargo (such as polynucleotides of the GIS).

In some embodiments, the GIS of the invention may be incorporated into LNPs. In some embodiments a lipid nanoparticle may be comprised of at least one cationic lipid (e.g., an ionizable cationic lipid), at least one non-cationic lipid (e.g., a phospholipid), at least one sterol (e.g., cholesterol), at least polymer-conjugated lipid (e.g., a PEG-lipid), or any combination thereof. In some embodiments a lipid nanoparticle may be comprised of at least one cationic lipid (e.g., an ionizable cationic lipid), at least one non-cationic lipid (e.g., a phospholipid), at least one sterol (e.g., cholesterol), and at least one polymer-conjugated lipid (e.g., a PEG-lipid). In some embodiments, the LNP may be comprised of at least one cationic lipid (e.g., an ionizable cationic lipid), at least one non-cationic lipid, and at least one sterol (e.g., cholesterol). In some embodiments, the LNP may be comprised of at least one cationic lipid (e.g., an ionizable cationic lipid), at least one non-cationic lipid (e.g., a phospholipid), and at least one polymer-conjugated lipid (e.g., a PEG-lipid). In some embodiments, the LNP may be comprised of at least one non-cationic lipid (e.g., a phospholipid), at least one sterol (e.g., cholesterol), and at least one polymer-conjugated lipid (e.g., a PEG-lipid). In some embodiments, the LNP may be comprised of at least one cationic lipid (e.g., an ionizable cationic lipid) and at least one non-cationic lipid (e.g., a phospholipid). In some embodiments, the LNP may be comprised of at least one cationic lipid (e.g., an ionizable cationic lipid) and at least one sterol. In some embodiments, the LNP may be comprised of at least one cationic lipid (e.g., an ionizable cationic lipid) and at least one polymer-conjugated lipid (e.g., a PEG-lipid). In some embodiments, the LNP may be comprised of at least one non-cationic lipid (e.g., a phospholipid) and at least one sterol (e.g., cholesterol). In some embodiments, the LNP may be comprised of at least one non-cationic lipid (e.g., a phospholipid) and at least one polymer-conjugated lipid (e.g., a PEG-lipid). In some embodiments, the LNP may be comprised of at least one sterol (e.g., cholesterol) and at least one polymer-conjugated lipid (e.g., a PEG-lipid). In some embodiments, the LNP may be comprised of at least one cationic lipid (e.g., an ionizable cationic lipid). In some embodiments, the LNP may be comprised of at least one non-cationic lipid (e.g., a phospholipid). In some embodiments, a LNP may be comprised of a sterol (e.g., cholesterol). In some embodiments, the LNP may be comprised of a polymer-conjugated lipid (e.g., a PEG-lipid).

The LNPs described herein may be formed using techniques known in the art. As a non-limiting example, an organic solution containing the lipids is mixed together with an acidic aqueous solution containing the GIS in a microfluidic channel resulting in the formation of a GIS loaded delivery vehicle.

### Micelles

In some embodiments, the delivery vehicles comprise of at least one micelle. In some embodiments, micelles may be comprised of any or all the same components as a lipid-nanoparticle, differing principally in their method of manufacture. As used herein, “micelles” refer to small particles which do not have an aqueous intra-particle space. Without wishing to be bound by theory, the intra-particle space of micelles does not include any additional lipid-head groups, and rather is occupied by the hydrophobic tails of the lipids comprising the micelle membrane and possible associated GIS.

### Liposomes

In some embodiments, the delivery vehicles comprise of at least one liposome. In some embodiments, liposomes may be comprised of any or all the same components and same component amounts as a lipid nanoparticle, differing principally in their method of manufacture. As used herein, “liposomes” refer to small vesicles comprised of at least one lipid bilayer membrane surrounding an aqueous inner-nanoparticle space. Further, liposomes differ from extracellular vesicles in that they are generally not derived from a progenitor/host cell. Liposomes can be potentially hundreds of nanometers in diameter comprising a series of concentric bilayers separated by narrow aqueous spaces (i.e., (large) multilamellar vesicles (MLV)), potentially smaller than 50 nm in diameter (small unicellular vesicles (SUV)), and potentially between 50 and 500 nm in diameter (large unilamellar vesicles (LUV)).

### Exosomes

In some embodiments, the delivery vehicle comprises at least one exosome. In general, “exosomes” refer to small, membrane bound, extracellular vesicles with an endocytic origin. Exosome membranes are generally composed of a bilayer of lipids and lamellar, with an aqueous inter-nanoparticle space. Exosomes will tend to include components of the host/progenitor membrane they are derived from in addition to designed components. Without wishing to be bound by theory, exosomes are generally released into an extracellular environment from host/progenitor cells post fusion of multivesicular bodies the cellular plasma membrane.

### Virus-Like Particles

In some embodiments, the delivery vehicle comprises at least one virus like particle (VLP). In general, virus like particles are a non-infectious vesicle comprised predominantly of a protein capsid, coat, shell, or sheath (all to be understood as equivalent used interchangeably herein) derived from a virus which can be loaded with the GIS. In some embodiments, VLP's may be synthesized using cellular machinery to express viral capsid protein sequences, which then self-assemble and incorporate the GIS. In some embodiments, VLPs may be formed by providing the capsid and GIS components without expression related cellular machinery and allowing them to self-assemble.

Non-limiting examples of viral families and species from which VLPs may be derived include, Parvoviridae, Retroviridae, Flaviviridae, Paramyxoviridae, adeno-associated virus, HIV, Hepatitis C virus, HPV, bacteriophages. or any combination thereof.

### Polymeric Delivery Particles

In some embodiments, the delivery vehicle may comprise at least one polymeric delivery particle. As used herein, “polymeric delivery particles” refer to non-aggregating delivery particles comprised of soluble polymers conjugated to GIS moieties via various linkage groups. In some embodiments, polymeric delivery agents may comprise any of the polymers described herein.

In some embodiments, the delivery vehicle may comprise a nucleic acid nanoparticle (NANP). In general, “nucleic acid nanoparticles” are small particles formed from non-coding nucleic acid sequences which interact to form 3-dimensional structures capable of carrying a cargo (e.g., GIS components).

### Encapsulation

In some embodiments, the delivery vehicle may fully encapsulate a GIS disclosed herein. In some embodiments, the delivery vehicle may partially encapsulate a GIS disclosed herein. In some embodiments, essentially 0% of the GIS present is exposed to the environment outside of the delivery vehicle in the final formulation (i.e., the GIS is fully encapsulated). In some embodiments, the GIS is associated with the delivery vehicle but is at least partially exposed to the environment outside of the delivery vehicle.

In some embodiments, the delivery vehicle may be characterized by the encapsulation efficiency, i.e., the % of the GIS not exposed to the environment outside of the delivery vehicle. For the sake of clarity, an encapsulation efficiency of about 100% refers to a delivery vehicle formulation where essentially all the GIS is fully encapsulated by the delivery vehicle, while an encapsulation rate of about 0% refers to a delivery vehicle where essential none of the GIS is encapsulated in the delivery vehicle, such as with a delivery vehicle where the GIS is bound to the external surface of the delivery vehicle. On some embodiments, and delivery vehicle may have an encapsulation efficiency of less than about 100%, less than about 95%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15% less than about 10%, or less than 5%. In some embodiments, an delivery vehicle may have an encapsulation efficiency of between about 90 to 100%, 80 to 100%, 70 to 100%, 60 to 100%, 50 to 100%, 40 to 100%, 30 to 100%, 20 to 100%, 10 to 100%, 80 to 90%, 70 to 90%, 60 to 90%, 50 to 90%, 40 to 90%, 30 to 90%, 20 to 90%, 10 to 90%, 70 to 80%, 60 to 80%, 50 to 80%, 40 to 80%, 30 to 80%, 20 to 80%, 10 to 80%, 60 to 70%, 50 to 70%, 40 to 70%, 30 to 70%, 20 to 70%, 10 to 70%, 40 to 50%, 30 to 50%, 20 to 50%, 10 to 50%, 30 to 40%, 20 to 40%, 10 to 40%, 20 to 30%, 10 to 30%, and 10 to 20%.

### Physical Characteristics of Delivery Vehicle Nanoparticles

In some embodiments, the delivery vehicles can be characterized by their shape. In some embodiments, the delivery vehicles may be, but are not limited to being essentially spherical, essentially rod-shaped (i.e., cylindrical), or essentially disk shaped.

In some embodiments, the delivery vehicles can be characterized by their size. In some embodiments, the size of a delivery vehicle can be defined as its diameter. As used hereinvin relation to delivery vehicle size, “diameter” refers to the diameter of its largest circular cross section of the delivery vehicle. In some embodiments the delivery vehicles may have a diameter between 30 nm to about 150 nm. For example, the delivery vehicle may have diameters ranging between about 40 to 150 nm 50 to 150 nm, 60 to 150 nm, about 70 to 150 nm, or 80 to 150 nm, 90 to 150 nm, 100 to nm, 110 to 150 nm, 120 to 150 nm, 130 to 150 nm, 140 to 150 nm, 30 to 30 to 140 nm, 40 to 140 nm, 50 to 140 nm, 60 to 140 nm, 70 to 140 nm, 80 to 140 nm, 90 to 140 nm, 100 to 140 nm, 110 to 140 nm, 120 to 140 nm, 130 to 140 nm, 140 to 140 nm, 30 to 140 nm, 40 to 130 nm, 50 to 130 nm, 60 to 130 nm, 70 to 130 nm, 80 to 130 nm, 90 to 130 nm, 100 to 130 nm, 110 to 130 nm, 120 to 130 nm, 30 to 120 nm, 40 to 120 nm, 50 to 120 nm, 60 to 120 nm, 70 to 120 nm, 80 to 120 nm, 90 to 120 nm, 100 to 120 nm, 110 to 120 nm, 30 to 110 nm, 40 to 110 nm, 50 to 110 nm, 60 to 110 nm, 70 to 110 nm, 80 to 110 nm, 90 to 110 nm, 100 to 110 nm, 30 to 100 nm, 40 to 100 nm, 50 to 100 nm, 60 to 100 nm, 70 to 100 nm, 80 to 100 nm, 90 to 100 nm, 30 to 90 nm, 40 to 90 nm, 50 to 90 nm, 60 to 90 nm, 70 to 90 nm, 80 to 90 nm, 30 to 80 nm, 40 to 80 nm, 50 to 80 nm, 60 to 80 nm, 70 to 80 nm, 30 to 70 nm, 40 to 70 nm, 50 to 70 nm, 60 to 70 nm, 30 to 60 nm, 40 to 60 nm, 50 to 60 nm, 30 to 50 nm, 40 to 50 nm, and 30 to 40 nm.

In some embodiments, a population of delivery vehicles, for example all delivery vehicles resulting from the same formulation, may be characterized by measuring the uniformity of physical characteristics (e.g., size, shape, or mass) of the particles in the population. In some embodiments, uniformity may be expressed as the polydispersity index (PI) of the population. In some embodiments uniformity may be expressed as the disparity (Ð) of the population. As used herein, the terms “polydispersity index” and “disparity” are understood to be equivalent and may be used interchangeably.

In some embodiments, a population of delivery vehicles resulting from a given formulation will have a PI of between about 0.1 and 1. In some embodiments, a population of delivery vehicles resulting from a given formulation will have a PI of between about 0.1 to 1, 0.1 to 0.8, 0.1 to 0.6, 0.1 to 0.4, 0.1 to 0.2, 0.2 to 1, 0.2 to 0.8, 0.2 to 0.6, 0.2 to 0.4, 0.4 to 1, 0.4 to 0.8, 0.4 to 0.6, 0.6 to 1, 0.6 to 0.8, and 0.8 to 1. In some embodiments, a population of delivery vehicles resulting from a giving formulation will have a PI of less than about 1, less than about 0.5, less than about 0.4, less than about 0.3, less than about 0.2, less than about 0.1.

### Delivery Targeting

In some embodiments, delivery vehicles formulated with the GIS may promote localization of the GIS to any of the targeted areas, tissues, cells, or physiological systems described herein (i.e., the delivery vehicle “targets” the specified location). In some embodiments, targeting may be achieved by a given formulation of delivery vehicle structural components. In some embodiments, delivery vehicles may comprise targeting agents.

### Targeting Agents

In some embodiments, the delivery vehicle may comprise at least one targeting agent. As used herein, the term targeting agent may refer in some embodiments to a moiety, compound, antibody, etc. that specifically binds a particular type or category of cell and/or other particular type of compounds, (e.g., a moiety that targets a specific cell or type of cell). In some embodiments, a targeting agent may have an affinity for the surface of certain target cells (i.e., be specific for), a target cell surface antigen, a target cell receptor, or a combination thereof.

In some embodiments, a targeting agent may refer to an agent that has a particular action (e.g., cleaves) when exposed to a particular type or category of substances and/or cells, and this action can drive the delivery vehicle to target a particular type or category of cell.

In some embodiments, the term targeting agent can refer to an agent that may be part of the delivery vehicle and plays a role in the delivery vehicle's specificity for a target, although the agent itself may or may not be specific for the particular type or category of cell itself.

In some embodiments, the presence of at least one targeting agent in the delivery vehicle may increase the efficiency (e.g., total amount or rate) of cellular uptake of the GIS delivered by the delivery vehicle. In some embodiments, the presence of at least one targeting agent in the delivery vehicle may increase the specificity (e.g., total amount or rate) of cellular uptake of the GIS delivered by the delivery vehicle. As used herein, “specificity” refers to a higher efficiency of cellular uptake by target cells than by non-target cells

In some embodiments, suitable targeting agents may include, but are not limited to, one or more small molecule targeting agents (e.g., carbohydrate moieties), antibodies, antibody-like molecules, peptides, vitamins (e.g., folate), sugars (e.g., lactose and galactose), artificial affinity molecules (e.g., a peptidomimetic or an aptamer), antibody fragments, single chain variable fragments (scFv), cell surface receptors (e.g., T cell receptor (TCR), B cell receptor (BCR), or chimeric antigen receptor (CAR)), and any combination thereof.

In some embodiments, cell surface antigens which may be targeted by targeting agents may include any cell surface molecule of the target cell. Examples of suitable cell surface molecules include, but are not limited to, a protein, sugar, lipid, or other antigen on the cell surface. In some embodiments, the cell surface antigen undergoes internalization.

In some specific embodiments, the delivery vehicle can comprise more than one targeting agents.

In some embodiments, at least one targeting agent may be incorporated into the lipid membrane of the nanoparticle. In some embodiments, at least one targeting agent may be presented on the external surface of the nanoparticle. In some embodiments, at least one targeting agent may be conjugated to a lipid-component of the nanoparticle. In some embodiments, at least one targeting agent may be conjugated to a polymer component of the nanoparticle. In some embodiments, a monomer comprising a targeting agent residue (e.g., a polymerizable derivative of a targeting agent such as an (alkyl) acrylic acid derivative of a peptide) can be co-polymerized to form the polymer-conjugated lipid forming the delivery vehicle. In some embodiments, at least one targeting agent may be anchored to the nanoparticle via hydrophobic and hydrophilic interactions among at least one targeting agent, the nanoparticle membrane, and the aqueous environments inside or outside the nanoparticle. In some embodiments, at least one targeting agent is conjugated to a peptide/protein component of the nanoparticle membrane. In some embodiments, at least one targeting agent is conjugated to a suitable linker moiety which is conjugated to a component of the nanoparticle membrane. In some embodiments, any combination of forces and bonds can result in the targeting agent being associated with the nanoparticle.

In some embodiments, one or more targeting agents may be coupled to at least one polymer of the delivery vehicles through a linking moiety. In some embodiments, the linking moiety may be a cleavable linking moiety (e.g., comprises a cleavable bond). In some embodiments, the linking moiety may comprise a bond that may be cleaved by a specific enzyme (e.g., a phosphatase, or a protease). In some embodiments, the linking moiety may comprise a bond that may be cleavable upon a change in intracellular pH, redox potential, or other intracellular parameter. In some embodiments, a linking moiety may comprise a bond that may be cleaved upon exposure to a matrix metalloproteinase (MMP).

### Direct Transfection

In some embodiments, GIS disclosed herein may be directly transfected into target cells without the use of a delivery vehicle. In some embodiments, GIS disclosed herein may be transfected into a target cell using any technique known in the art. Such techniques may include but are not limited to chemical transfection methods (e.g., calcium phosphate exposure), physical transfection methods (e.g., electroporation, microinjection, and biolistic particle delivery). In some embodiments, direct transfection may be carried out utilizing lipid mediated transfection agents, such as but not limited to, lipofectamine, lipofectamine 2000, and any combination thereof.

### Implantation of Transfected Cells

In some embodiments, the GIS of the invention may be introduced to a population of cells (e.g., via direct transfection as described herein) in vitro for latter implantation to a subject. In some embodiments, the population of cells for implantation may be stem cells. In some embodiments, the population of cells for implantation may be derived from the subject. In some embodiments, implantation may be carried out via any method known in the art.

### IV. Pharmaceutical Composition and Route of Administration

The invention provides pharmaceutical compositions for administration of the GIS to a subject. In some embodiments, the invention provides pharmaceutical compositions for use as a medicament in the treatment of a therapeutic indication. In some embodiments, the pharmaceutical composition comprises at least one active ingredient (e.g., the GIS of the invention) and at least one pharmaceutically acceptable excipient, adjuvant, carrier, dilutant, or any combination thereof. In some embodiments, the pharmaceutical composition is formulated for at least one rout of administration. In some embodiments, the pharmaceutical composition is formulated for delivering a specified dose, optionally on a specified schedule, of at least one active ingredient (e.g., the GIS).

As used herein the term “pharmaceutical composition” refers to compositions comprising at least one active ingredient and optionally one or more pharmaceutically acceptable excipients. As used herein, the phrase “active ingredient” generally refers to any of, the GIS, a gene payload carried by the GIS for insertion into the subject genome, or the expression product of a gene payload carried by the GIS as described herein.

### Pharmaceutical Formulations and Compositions

The GIS may be formulated using one or more excipients to: (1) increase stability of the GIS or a delivery mechanism comprising the GIS; (2) increase cell transfection or transduction; (3) permit the sustained or delayed introduction of the GIS to the subject's cells; (4) alter the biodistribution (e.g., target the GIS to specific tissues or cell types); (5) increase the expression of encoded genes; (6) alter the release profile of encoded protein; and/or (7) allow for regulatable expression of the GIS and/or the GIS payload.

Without limitation, formulations can include saline, liposomes, lipid nanoparticles, polymers, peptides, proteins, cells transfected with the GIS (e.g., for transfer or transplantation into a subject) and any combinations thereof.

In some embodiments, formulations of the pharmaceutical compositions described herein may be prepared by any method known or hereafter developed in the art of pharmacology. In general, such preparatory methods include the step of associating the active ingredient with an excipient and/or one or more other accessory ingredients.

Formulations of the GIS and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition as described herein may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, an excipient is approved for use for humans and for veterinary use. In some embodiments, an excipient may be approved by United States Food and Drug Administration. In some embodiments, an excipient may meet the standards of the United States Pharmacopoeia (USP), the European Pharmacopoeia (EP), the British Pharmacopoeia, and/or the International Pharmacopoeia. In some embodiments, a pharmaceutically acceptable excipient may be at least 100%, at least 99%, at least 98%, at least 97%, at least 96%, or 95% pure. In some embodiments, an excipient may be of pharmaceutical grade.

In some embodiments relative amounts of the pharmaceutically acceptable excipient, the active ingredient, and/or any additional ingredients may vary in pharmaceutical compositions of the invention. In some embodiments, the relative amounts may vary depending upon the size, condition, and/or identity of the subject being treated. In some embodiments, the relative amounts may vary depending upon the route by which the composition is to be administered. For example, the composition may comprise between 0.1% and 100%, (e.g., between 0.1% and 99%, between 0.5 and 50%, between 1-30%, between 5-80%, or at least 80% (w/w)) of the active ingredient.

### Excipients, Diluents, and Inactive Ingredients

In some embodiments, the pharmaceutical composition may include any excipient know or discovered in the art. Examples of suitable excipients include, but are not limited to, any and all preservatives, isotonic agents, thickening or emulsifying agents, solvents, dispersion media, diluents or other liquid vehicles, dispersion or suspension aids, surface active agents, and combinations thereof. In some embodiments, excipients may be chosen based on their suitability for the particular dosage form desired.

In some embodiments, formulations described herein may comprise at least one inactive ingredient. As used herein, the term “inactive ingredient” refers to one or more agents included in formulations that do not contribute to the activity of the active ingredient of the pharmaceutical composition. In some embodiments, none, some, or all of the inactive ingredients in the pharmaceutical composition may be approved by the US Food and Drug Administration (FDA).

In some embodiments, pharmaceutical formulations disclosed herein may include cations or anions. In some embodiments, the pharmaceutical formulations include metal cations such as, but not limited to, Ca2+, Zn2+, Mn2+, Cu2+, Mg+ and any combinations thereof. In some embodiments, pharmaceutical formulations may include polymers complexed with a metal cation.

In some embodiments, pharmaceutical compositions may include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Pharmaceutically acceptable salts of the invention include, for example, the conventional non-toxic salts of any parent compound formed, from non-toxic inorganic or organic acids. Pharmaceutically acceptable salts include, but are not limited to, alkali or organic salts of acidic residues such as carboxylic acids; and mineral or organic acid salts of basic residues such as amines.

In some embodiments, the pharmaceutical composition may include at least one solvent. In some embodiments, when water is the solvent, the solvate is generally referred to as a “hydrate.”

### Routes of Administration

The GIS, including pharmaceutical compositions comprising the GIS described herein may be administered by any delivery route which results in successful integration of the GIS into subject cells. Acceptable routes of administration include, but are not limited to, auricular (in or by way of the ear), biliary perfusion, buccal (directed toward the cheek), cardiac perfusion, caudal block, conjunctival, cutaneous, dental (to a tooth or teeth), dental intracoronal, diagnostic, ear drops, electro-osmosis, endocervical, endosinusial, endotracheal, enema, enteral (into the intestine), epicutaneous (application onto the skin), epidural (into the dura mater), extra-amniotic administration, extracorporeal, eye drops (onto the conjunctiva), gastroenteral, hemodialysis, infiltration, insufflation (snorting), interstitial, intra-abdominal, intra-amniotic, intra-arterial (into an artery), intra-articular, intrabiliary, intrabronchial, intrabursal, intracardiac (into the heart), intracartilaginous (within a cartilage), intracaudal (within the cauda equine), intracavernous injection (into a pathologic cavity) intracavitary (into the base of the penis), intracerebral (into the cerebrum), intracerebroventricular (into the cerebral ventricles), intracisternal (within the cisterna magna cerebellomedularis), intracorneal (within the cornea), intracoronary (within the coronary arteries), intracorporus cavernosum (within the dilatable spaces of the corporus cavernosa of the penis), intradermal (into the skin itself), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramuscular (into a muscle), intramyocardial (within the myocardium), intraocular (within the eye), intraosseous infusion (into the bone marrow), intraovarian (within the ovary), intraparenchymal (into brain tissue), intrapericardial (within the pericardium), intraperitoneal (infusion or injection into the peritoneum), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (into the spinal canal), intrathecal (within the cerebrospinal fluid at any level of the cerebrospinal axis), intrathoracic (within the thorax), intratubular (within the tubules of an organ), intratumor (within a tumor), intratympanic (within the aurus media), intrauterine, intravaginal administration, intravascular (within a vessel or vessels), intravenous (into a vein), intravenous bolus, intravenous drip, intraventricular (within a ventricle), intravesical infusion, intravitreal (through the eye), iontophoresis (by means of electric current where ions of soluble salts migrate into the tissues of the body), irrigation (to bathe or flush open wounds or body cavities), laryngeal (directly upon the larynx), nasal administration (through the nose), nasogastric (through the nose and into the stomach), nerve block, occlusive dressing technique (topical route administration which is then covered by a dressing which occludes the area), ophthalmic (to the external eye), oral (by way of the mouth), oropharyngeal (directly to the mouth and pharynx), parenteral, percutaneous, periarticular, peridural, perineural, periodontal, photopheresis, rectal, respiratory (within the respiratory tract by inhaling orally or nasally for local or systemic effect), retrobulbar (behind the pons or behind the eyeball), soft tissue, subarachnoid, subconjunctival, subcutaneous (under the skin), sublabial, sublingual, submucosal, topical, transdermal, transdermal (diffusion through the intact skin for systemic distribution), transmucosal (diffusion through a mucous membrane), transplacental (through or across the placenta), transtracheal (through the wall of the trachea), transtympanic (across or through the tympanic cavity), transvaginal, ureteral (to the ureter), urethral (to the urethra), vaginal, and spinal.

In some embodiments, pharmaceutical compositions may be administered in a way which allows them to cross the vascular barrier, the blood-brain barrier, or other epithelial barriers. The GIS may be administered in any suitable form, including, but not limited to, a liquid solution, a suspension, a solid form, a solid form suitable for dissolution in a liquid solution, a solid form capable of suspension in a liquid solution, and any combination thereof.

In some embodiments, the GIS may be delivered to a subject via a multi-site route of administration. A subject may be administered at 2, 3, 4, 5, or more than 5 sites.

In some embodiments, the GIS may be delivered to a subject via a single route administration.

In some embodiments, a subject may be administered the GIS using a bolus infusion.

In some embodiments, a subject may be administered the GIS using methods of sustained delivery (i.e., infusion) over a period of minutes, hours, or days. The infusion rate may be changed depending on any delivery parameters including, but not limited to, the nature of the subject, desired distribution, the formulation used, and so on.

In some embodiment, the GIS may be delivered by intramuscular delivery route including, but not limited to, subcutaneous injection or an intravenous injection.

In some embodiments, the GIS may be delivered by oral administration including, but not limited to, a digestive tract administration or a buccal administration.

In some embodiments, the GIS may be delivered by intraocular delivery route including, but not limited to, an intravitreal injection or application of eye drops.

In some embodiment, the GIS may be delivered by intranasal delivery route including, but not limited to, nasal drops or nasal sprays.

In some embodiments, the GIS may be administered to a subject by peripheral injections including, but not limited to, intramuscular, intraperitoneal, intravenous, conjunctival, or joint injection.

In some embodiments, the GIS may be delivered by injection into the cerebrospinal fluid route including, but not limited to, intrathecal and intracerebroventricular administration.

In some embodiments, the GIS may be delivered by systemic delivery route including, but not limited to, intravascular administration.

In some embodiments, the GIS may be administered to a subject by intraparenchymal administration.

In some embodiments, the GIS may be administered to a subject by topical administration.

In some embodiments, the GIS may be administered to a subject by intracranial delivery.

In some embodiments, the GIS may be administered to a subject by intramuscular administration.

In some embodiments, the GIS may be administered to a subject by intravenous administration.

In some embodiments, the GIS may be administered to a subject by subcutaneous administration.

In some embodiments, the GIS may be delivered by more than one route of administration.

### Injectable and Parenteral Administration

In some embodiments, pharmaceutical compositions described herein may be administered parenterally. Liquid dosage forms for parenteral and oral administration include, but are not limited to, pharmaceutically acceptable solutions, emulsions, microemulsions, elixirs, suspensions, and/or syrups. In addition to active ingredients, liquid dosage forms may comprise inert diluents commonly used in the art such as, for example, solubilizing agents, water or other solvents, and emulsifiers (e.g., polyethylene glycols, propylene glycol, 1,3-butylene glycol, tetrahydrofurfuryl alcohol, isopropyl alcohol, ethyl alcohol, ethyl carbonate, ethyl acetate, benzyl alcohol, benzyl benzoate, dimethylformamide, oils, glycerol, and fatty acid esters of sorbitan), and any combination thereof. Exemplary oils may include cottonseed, groundnut, corn, germ, olive, castor, and sesame oils and mixtures thereof. In some embodiments, pharmaceutical compositions comprise solubilizing agents such as alcohols, oils, glycols, CREMOPHOR®, modified oils, polysorbates, polymers, cyclodextrins, and/or combinations thereof. In some embodiments, surfactants are included such as hydroxypropylcellulose.

In some embodiments, injectable preparations may include sterile injectable aqueous or oleaginous suspensions. Sterile solutions for injection may be formulated according to the known art using suitable wetting agents, dispersing agents, and/or suspending agents. Sterile injectable preparations may be sterile injectable suspensions, solutions, and/or emulsions in nontoxic, parenterally acceptable, diluents and/or solvents. In some embodiments, sterile injectable preparation may be a solution in 1,3-butanediol. In some embodiments, acceptable vehicles and solvents include, but are not limited to, Ringer's solution, U.S.P., water, isotonic sodium chloride solution, and sterile, fixed oils. In some embodiments, fixed oils may include any bland fixed oil (e.g., synthetic mono- or diglycerides). In some embodiments, fatty acids, such as oleic acid, can be used in the preparation of injectables.

In some embodiments, injectable formulations may be sterilized by filtration through a bacterial-retaining filter, and/or by incorporating sterilizing agents. In some embodiments, sterilizing agents may be in the form of sterile solid compositions which can be dissolved or dispersed in a sterile injectable medium, such as sterile water, prior to use.

It is often desirable to slow the absorption of active ingredients from subcutaneous or intramuscular injections in order to prolong the effect of active ingredients. In some embodiments, delayed absorption of a parenterally administered pharmaceutical compositions is accomplished by dissolving or suspending the pharmaceutical composition in an oil vehicle. In some embodiments, slowing the absorption of active ingredients may be accomplished by the use of liquid suspensions of amorphous or crystalline material with poor water solubility. The rate of absorption of active ingredients depends upon the rate of dissolution which, in turn, may depend upon crystal size and crystalline form.

### Oral Administration

In some embodiments, pharmaceutical compositions and/or formulations described herein may be administered orally. Solid dosage forms for oral administration include tablets, capsules, powders, pills, and granules. In general, for solid dosage forms, an active ingredient is mixed with at least one inert, pharmaceutically acceptable excipient including, but not limited to, dicalcium phosphate or sodium citrate, binders (e.g. carboxymethylcellulose, alginates, gelatin, polyvinylpyrrolidinone, sucrose, and acacia), fillers or extenders (e.g. starches, lactose, sucrose, glucose, mannitol, and silicic acid), disintegrating agents (e.g. agar, calcium carbonate, potato or tapioca starch, alginic acid, certain silicates, and sodium carbonate), absorption accelerators (e.g. quaternary ammonium compounds), humectants (e.g. glycerol), solution retarding agents (e.g. paraffin), absorbents (e.g. kaolin and bentonite clay), wetting agents (e.g. cetyl alcohol and glycerol monostearate), lubricants (e.g. talc, calcium stearate, magnesium stearate, solid polyethylene glycols, sodium lauryl sulfate), and any combination thereof. In the case of tablets, capsules, and pills, the dosage form may comprise buffering agents.

Liquid dosage forms for oral administration may include those described for parenteral administration above. Besides inert diluents, oral compositions may include adjuvants such as emulsifying agents, wetting agents, suspending agents, flavoring agents, sweetening agents, and/or perfuming agents.

### Topical or Transdermal Administration

In some embodiments, pharmaceutical compositions and/or formulations described herein may be formulated for administration topically. The skin may be an ideal target site for delivery as it is readily accessible. In some embodiments, routes to deliver pharmaceutical compositions described herein to or through the skin include, but are not limited to, topical application (e.g., for cosmetic applications and/or local/regional treatment), intradermal injection (e.g., for cosmetic applications and/or local/regional treatment), and systemic delivery (e.g., for treatment of dermatologic diseases that affect both cutaneous and extracutaneous regions).

In some embodiments, pharmaceutical compositions and/or formulations described herein may be delivered using a variety of dressings bandages (e.g., adhesive bandages) or (e.g., wound dressings) for effectively and/or conveniently carrying out methods described herein. In some embodiments, dressing or bandages may comprise sufficient amounts of pharmaceutical compositions described herein to allow users to perform multiple treatments.

Dosage forms for topical and/or transdermal administration may include lotions, creams, ointments, gels, sprays, pastes, powders, solutions, inhalants and/or patches. Generally, topical and/or transdermal administration may be formulated by admixing active ingredients under sterile conditions with pharmaceutically acceptable excipients, buffers, and/or any needed preservatives.

In some embodiments, transdermal patches may be used. Transdermal patches may have the added advantage of providing controlled delivery of pharmaceutical compositions described herein to the body. In general, transdermal patches may be prepared by dissolving and/or dispensing pharmaceutical compositions described herein in the proper medium. In some embodiments, rates of delivery may be controlled by dispersing pharmaceutical compositions in a polymer matrix and/or gel, providing rate controlling membranes, or any combination thereof.

In some embodiments, formulations suitable for topical administration may include liquid and/or semi liquid preparations (e.g., liniments and lotions), oil in water and/or water in oil emulsions (e.g., ointments, creams, and/or pastes), solutions and/or suspensions, and any combination thereof.

### Ophthalmic or Otic Administration

In some embodiments, pharmaceutical compositions described herein may be in formulations suitable for ophthalmic administration, otic administration, or both. In general, such formulations may be in the form of eye and/or ear drops including, but not limited to, a solution and/or suspension of the active ingredient in aqueous and/or oily liquid excipients. In some embodiments, such drops may comprise salts, buffering agents, one or more other of any additional ingredients described herein, and combinations thereof. In some embodiments, ophthalmically-administrable formulations include active ingredients in liposomal preparations and/or microcrystalline form. In some embodiments, pharmaceutical compositions may be administered via subretinal.

### Pulmonary Administration

In some embodiments, pharmaceutical compositions described herein may in formulations suitable for pulmonary administration. In some embodiments, pulmonary administration is via the buccal cavity. In some embodiments, pharmaceutical compositions may comprise dry particles comprising active ingredients. In some embodiments, dry particles for pulmonary administration may have a diameter in the range from about 0.5-7 nm or from about 1-6 nm.

In some embodiments, self-propelling solvent/powder dispensing containers may be used to administer the pharmaceutical composition. In general, the active ingredients may be dissolved and/or suspended in a low-boiling propellant in sealed containers. In some embodiments, pharmaceutical compositions may be in the form of dry powders for administration using devices comprising dry powder reservoirs to which streams of propellant may be directed to disperse such powder. In some embodiments utilizing dry powders, powders may comprise particles wherein at least 98% of the particles, by weight, have diameters greater than 0.5 nm and at least 95% of the particles, by number have diameters less than 7 nm. In some embodiments, at least 95% of the particles, by weight, have a diameter greater than 1 nm and at least 90% of the particles, by number, have a diameter less than 6 nm. In some embodiments, dry pharmaceutical compositions comprising powder may include a solid fine powder diluent (e.g., sugar) and may be provided in a unit dose form for convenience.

In some embodiments, low boiling propellants include liquid propellants having a boiling point of below 65° F. at atmospheric pressure. In some embodiments, propellants may constitute 50% to 99.9% (w/w) of the pharmaceutical composition, and active ingredient may constitute 0.1% to 20% (w/w) of the pharmaceutical composition. In some embodiments, propellants may comprise additional ingredients including, but not limited to, liquid non-ionic surfactants, solid anionic surfactants, solid diluents (including, for example, solid diluents which have particle sizes of the same order as particles comprising active ingredients), and any combination thereof.

In some embodiments, pharmaceutical compositions formulated for pulmonary delivery may be in the form of droplets of solution, suspension, and combinations thereof. Such formulations may be administered using any atomization and/or nebulization device when prepared, packaged, and/or sold as solutions, suspensions, or combinations thereof. In some embodiments, the solutions and/or suspensions may be sterile. Exemplary solutions and/or suspensions include aqueous and/or dilute alcoholic compositions. In some embodiments, pharmaceutical compositions formulated for pulmonary delivery may comprise a flavoring agent (e.g., saccharin sodium), a volatile oil, a surface-active agent, a buffering agent, a preservative (e.g., methylhydroxybenzoate), and any combination thereof. In some embodiments, droplets provided by this route of administration may have an average diameter in the range from about 0.1 nm to about 200 nm.

### Intranasal, Nasal, or Buccal Administration

In some embodiments, pharmaceutical compositions described herein may be administered intranasal, nasally, or both. In some embodiments, pharmaceutical compositions for intranasal delivery may include those described herein for pulmonary delivery. In some embodiments, pharmaceutical compositions for intranasal administration comprise a coarse powder, having an average particle diameter from about 0.2 μm to 500 μm, comprising the active ingredient. In some embodiments, the pharmaceutical composition may be administered by rapid inhalation through the nasal passage from a container of the powder held close to the nose, i.e., in the manner snuff is taken. Exemplary pharmaceutical formulations may comprise from about 0.1% (w/w) to 100% (w/w) of active ingredient and may comprise one or more of the additional ingredients described herein.

In some embodiments, a pharmaceutical composition may be in a formulation suitable for buccal administration including, but not limited to tablets, lozenges, and any combination thereof. In general, such tablets or lozenges may be made using conventional methods and may, include 0.1%-20% (w/w) active ingredient (given as a non-limiting example), any combination of orally dissolvable or orally degradable compositions, and, optionally, one or more of the additional ingredients described herein. In some embodiments, pharmaceutical compositions suitable for buccal administration may comprise any combination of powders, aerosolized solutions and/or suspensions, or atomized solutions and/or suspensions comprising active ingredients with a dispersed average particle and/or droplet size of about 0.1 nm-200 nm. In some embodiments, pharmaceutical compositions for buccal administration may further comprise one or more of any additional ingredients described herein.

### Depot Administration

In some embodiments, pharmaceutical compositions described herein are formulated in depots for extended release. In some embodiments, pharmaceutical compositions described herein are spatially retained within or proximal to target tissues.

Injectable depot forms are generally made by forming microencapsule matrices of the pharmaceutical composition in biodegradable polymers (e.g., polylactide-polyglycolide). In general, the rate of pharmaceutical composition release can be controlled by varying the ratio of pharmaceutical composition to polymer and the nature of the particular polymer used. Suitable biodegradable polymers include, but are not limited to, poly(orthoesters) and poly(anhydrides). Depot injectable formulations are prepared by entrapping the pharmaceutical composition in liposomes or microemulsions which are compatible with body tissues.

### Rectal and Vaginal Administration

In some embodiments, pharmaceutical compositions described herein may be administered rectally, vaginally, or any combination thereof. In general, compositions for rectal or vaginal administration are suppositories which can be prepared by mixing active ingredients with suitable non-irritating excipients (e.g., polyethylene glycol, cocoa butter, or a suppository wax) which are solid at ambient temperature but liquid at body temperature. The melting of the suppository in the rectum or vaginal cavity releases the active ingredient.

### Dose Amounts

The GIS and/or pharmaceutical compositions comprising the GIS may be administered at any amount (i.e., dose) that results in the desired effect in the subject (e.g., a desired therapeutic effect, research result, and so on). In some embodiments, the desired dose may be determined based subject parameters (e.g., subject size, state, or nature), effect parameters (e.g., degree of response required, therapeutically effective threshold, longevity of effect, or side effects present), or any combination thereof. In some embodiments, appropriate dose may be determined prior to initial administration, optionally based on at least one assay testing at least one subject parameter. In some embodiments, appropriate dose may be determined after an initial dose, optionally based on at least one assay testing at least one effect parameter. In some embodiments, the dose amount may remain unaltered throughout the course of administration. In some embodiments, the dose amount may be altered once, twice, or many times over the course of administration.

In some embodiments, the dose amount may be described as a ratio of mass of active ingredient to the mass of the subject (e.g., in mg/kg). For example, the dose amount may be between 0.1 to 100, 1 to 100, 2 to 100, 3 to 100, 4 to 100, 5 to 100, 6 to 100, 7 to 100, 8 to 100, 9 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, 0.1 to 95, 1 to 95, 2 to 95, 3 to 95, 4 to 95, 5 to 95, 6 to 95, 7 to 95, 8 to 95, 9 to 95, 10 to 95, 15 to 95, 20 to 95, 25 to 95, 30 to 95, 35 to 95, 40 to 95, 45 to 95, 50 to 95, 55 to 95, 60 to 95, 65 to 95, 70 to 95, 75 to 95, 80 to 95, 85 to 95, 90 to 95, 0.1 to 90, 1 to 90, 2 to 90, 3 to 90, 4 to 90, 5 to 90, 6 to 90, 7 to 90, 8 to 90, 9 to 90, 10 to 90, 15 to 90, 20 to 90, 25 to 90, 30 to 90, 35 to 90, 40 to 90, 45 to 90, 50 to 90, 55 to 90, 60 to 90, 65 to 90, 70 to 90, 75 to 90, 80 to 90, 85 to 90, 0.1 to 85, 1 to 85, 2 to 85, 3 to 85, 4 to 85, 5 to 85, 6 to 85, 7 to 85, 8 to 85, 9 to 85, 10 to 85, 15 to 85, 20 to 85, 25 to 85, 30 to 85, 35 to 85, 40 to 85, 45 to 85, 50 to 85, 55 to 85, 60 to 85, 65 to 85, 70 to 85, 75 to 85, 80 to 85, 0.1 to 80, 1 to 80, 2 to 80, 3 to 80, 4 to 80, 5 to 80, 6 to 80, 7 to 80, 8 to 80, 9 to 80, 10 to 80, 15 to 80, 20 to 80, 25 to 80, 30 to 80, 35 to 80, 40 to 80, 45 to 80, 50 to 80, 55 to 80, 60 to 80, 65 to 80, 70 to 80, 75 to 80, 0.1 to 75, 1 to 75, 2 to 75, 3 to 75, 4 to 75, 5 to 75, 6 to 75, 7 to 75, 8 to 75, 9 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 0.1 to 70, 1 to 70, 2 to 70, 3 to 70, 4 to 70, 5 to 70, 6 to 70, 7 to 70, 8 to 70, 9 to 70, 10 to 70, 15 to 70, 20 to 70, 25 to 70, 30 to 70, 35 to 70, 40 to 70, 45 to 70, 50 to 70, 55 to 70, 60 to 70, 65 to 70, 0.1 to 65, 1 to 65, 2 to 65, 3 to 65, 4 to 65, 5 to 65, 6 to 65, 7 to 65, 8 to 65, 9 to 65, 10 to 65, 15 to 65, 20 to 65, 25 to 65, 30 to 65, 35 to 65, 40 to 65, 45 to 65, 50 to 65, 55 to 65, 60 to 65, 0.1 to 60, 1 to 60, 2 to 60, 3 to 60, 4 to 60, 5 to 60, 6 to 60, 7 to 60, 8 to 60, 9 to 60, 10 to 60, 15 to 60, 20 to 60, 25 to 60, 30 to 60, 35 to 60, 40 to 60, 45 to 60, 50 to 60, 55 to 60, 0.1 to 55, 1 to 55, 2 to 55, 3 to 55, 4 to 55, 5 to 55, 6 to 55, 7 to 55, 8 to 55, 9 to 55, 10 to 55, 15 to 55, 20 to 55, 25 to 55, 30 to 55, 35 to 55, 40 to 55, 45 to 55, 50 to 55, 0.1 to 50, 1 to 50, 2 to 50, 3 to 50, 4 to 50, 5 to 50, 6 to 50, 7 to 50, 8 to 50, 9 to 50, 10 to 50, 15 to 50, 20 to 50, 25 to 50, 30 to 50, 35 to 50, 40 to 50, 45 to 50, 0.1 to 45, 1 to 45, 2 to 45, 3 to 45, 4 to 45, 5 to 45, 6 to 45, 7 to 45, 8 to 45, 9 to 45, 10 to 45, 15 to 45, 20 to 45, 25 to 45, 30 to 45, 35 to 45, 40 to 45, 0.1 to 40, 1 to 40, 2 to 40, 3 to 40, 4 to 40, 5 to 40, 6 to 40, 7 to 40, 8 to 40, 9 to 40, 10 to 40, 15 to 40, 20 to 40, 25 to 40, 30 to 40, 35 to 40, 0.1 to 35, 1 to 35, 2 to 35, 3 to 35, 4 to 35, 5 to 35, 6 to 35, 7 to 35, 8 to 35, 9 to 35, 10 to 35, 15 to 35, 20 to 35, 25 to 35, 30 to 35, 0.1 to 30, 1 to 30, 2 to 30, 3 to 30, 4 to 30, 5 to 30, 6 to 30, 7 to 30, 8 to 30, 9 to 30, 10 to 30, 15 to 30, 20 to 30, 25 to 30, 0.1 to 25, 1 to 25, 2 to 25, 3 to 25, 4 to 25, 5 to 25, 6 to 25, 7 to 25, 8 to 25, 9 to 25, 10 to 25, 15 to 25, 20 to 25, 0.1 to 20, 1 to 20, 2 to 20, 3 to 20, 4 to 20, 5 to 20, 6 to 20, 7 to 20, 8 to 20, 9 to 20, 10 to 20, 15 to 20, 0.1 to 15, 1 to 15, 2 to 15, 3 to 15, 4 to 15, 5 to 15, 6 to 15, 7 to 15, 8 to 15, 9 to 15, 10 to 15, 0.1 to 10, 1 to 10, 2 to 10, 3 to 10, 4 to 10, 5 to 10, 6 to 10, 7 to 10, 8 to 10, 9 to 10, 0.1 to 9, 1 to 9, 2 to 9, 3 to 9, 4 to 9, 5 to 9, 6 to 9, 7 to 9, 8 to 9, 0.1 to 8, 1 to 8, 2 to 8, 3 to 8, 4 to 8, 5 to 8, 6 to 8, 7 to 8, 0.1 to 7, 1 to 7, 2 to 7, 3 to 7, 4 to 7, 5 to 7, 6 to 7, 0.1 to 6, 1 to 6, 2 to 6, 3 to 6, 4 to 6, 5 to 6, 0.1 to 5, 1 to 5, 2 to 5, 3 to 5, 4 to 5, 0.1 to 4, 1 to 4, 2 to 4, 3 to 4, 0.1 to 3, 1 to 3, 2 to 3, 0.1 to 2, 1 to 2, or 0.1 to 1 mg/kg.

### Dose Schedules

The GIS and/or pharmaceutical compositions comprising the GIS may be administered at any frequency (i.e., dose schedule) that results in the desired effect in the subject (e.g., a desired therapeutic effect, research result, and so on). In some embodiments, dose schedule may be determined by any of the methods used to determine dose amount described herein. In some embodiments, the GIS may be administered only once.

In some embodiments, the GIS may be administered more than once. For example, the GIS may be administered 2, 3, 4, 5, 6, 7, 8, 9, 10, or more times. In some embodiments, the GIS may be administered intermittently and/or continuously over the course of treating a therapeutic indication in a subject. In some embodiments, the GIS may be administered repeatedly over the life of the subject.

### V. METHODS OF USE

### Target Area, Tissue, or Cell for Delivery of GIS Formulations

Provided herein are methods for delivering pharmaceutical compositions and/or formulations as described herein to at least one target location of a subject, by contacting at least one target (comprising one or more target cells), such as a physiological system, anatomical location, organ, tissue, cell type, cell population or the like with at least one of the pharmaceutical compositions and/or formulations described herein.

Pharmaceutical compositions and/or formulations described herein comprise enough active ingredient (e.g., a GIS of the invention) such that the effect of interest (e.g., insertion of at least one transgene into the subject genome) is produced in at least one cell located at the target.

In some embodiments, pharmaceutical compositions and/or formulations described herein generally comprise one or more cell penetration agents, although “naked” formulations (such as without cell penetration agents or other agents) are also contemplated, with or without pharmaceutically acceptable carriers.

### Physiological Systems

In some embodiments, pharmaceutical compositions and/or formulations described herein target a physiological system.

In some embodiments, physiological systems may include the auditory, cardiovascular, central nervous system, chemo-receptor system, circulatory, digestive, endocrine, excretory, exocrine, genital, integumentary, lymphatic, muscular, musculoskeletal, nervous, peripheral nervous system, renal, reproductive, respiratory, urinary, and visual systems.

In some embodiments, pharmaceutical compositions and/or formulations described herein target the Amine Precursor Uptake and Decarboxylation (APUD) System (a series of cells which have endocrine functions and secrete a variety of small amine or polypeptide hormones) such as, but not limited to, pituitary tissue, parathyroid tissue, thyroid tissue, bronchial tissue, adrenalmedulla tissue, pancreas tissue, stomach and intestines, carotid body, and chemo-receptor system tissue.

### Organs

In some embodiments, the pharmaceutical compositions and/or formulations described herein target an organ. Organs include the anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, and vulva.

In some embodiments, the pharmaceutical compositions and/or formulations described herein target the eye or eyes.

In some embodiments, the pharmaceutical compositions and/or formulations described herein target the liver.

In some embodiments, the pharmaceutical compositions and/or formulations described herein target the brain.

### Cells

In some embodiments, the pharmaceutical compositions and/or formulations described herein target a particular cell and/or cell type.

Cells include adipocytes, adrenergic neural cells, alpha cell, amacrine cells, ameloblast, anterior lens epithelial cell, anterior/intermediate pituitary cells, apocrine sweat gland cell, astrocytes, auditory inner hair cells of organ of corti, auditory outer hair cells of organ of corti, b cell, bartholin's gland cell, basal cell (stem cell) of cornea, tongue, mouth, nasal cavity, distal anal canal, distal urethra, and distal vagina, basal cells of olfactory epithelium, basket cells, basophil granulocyte and precursors, beta cell, betz cells, bone marrow reticular tissue fibroblasts, border cells of organ of corti, boundary cells, bowman's gland cell, brown fat cell, brunner's gland cell, bulbourethral gland cell, bushy cells, c cells, cajal-retzius cells, cardiac muscle cell, cardiac muscle cells, cartwheel cells, cells of the zona fasciculata produce glucocorticoids, cells of the zona glomerulosa produce mineralocorticoids, cells of the zona reticularis produce androgens, cells of the adrenal cortex, cementoblast, centroacinar cell, ceruminous gland cell in ear, chandelier cells, chemoreceptor glomus cells of carotid body cell, chief cell, cholinergic neurons, chromaffin cells, club cell, cold-sensitive primary sensory neurons, connective tissue macrophage (all types), corneal fibroblasts (corneal keratocytes), corpus luteum cell of ruptured ovarian follicle secreting progesterone, cortical hair shaft cell, corticotropes, crystallin-containing lens fiber cell, cuticular hair shaft cell, cytotoxic t cell, d cell, delta cell, dendritic cell, double-bouquet cells, duct cell, eccrine sweat gland clear cell, eccrine sweat gland dark cell, efferent ducts cell, elastic cartilage chondrocyte, endothelial cells, enteric glial cells, enterochromaffin cell, enterochromaffin-like cell, enteroendocrine cell, eosinophil granulocyte and precursors, ependymal cells, epidermal basal cell, epidermal langerhans cell, epididymal basal cell, epididymal principal cell, epithelial reticular cell, epsilon cell, erythrocyte, fibrocartilage chondrocyte, fork neurons, foveolar cell, g cell, gall bladder epithelial cell, germ cells, gland of litter cell, gland of moll cell in eyelid, glial cells, golgi cells, gonadal stromal cells, gonadotropes, granule cells, granulosa cell, granulosa lutein cells, grid cells, and head direction cells.

In some embodiments, cells may be cancerous cells. In some embodiments, cells may be non-cancerous cells.

In some embodiments, the eukaryotic cells may be stem cells. A variety of stem cell types are known in the art, any, or all of which may be used in the practice of this disclosure. Example stem cells include, but are not limited to, embryonic stem cells, hematopoietic stem cells, neural stem cells, epidermal neural crest stem cells, inducible pluripotent stem cells, mammary stem cells, intestinal stem cells, mesenchymal stem cells, olfactory adult stem cells, testicular cells, and progenitor cells (e.g., neural, angioblast, osteoblast, chondroblast, pancreatic, epidermal, etc.). In some embodiments, the stem cells may be stem cell lines derived from cells taken from the subject.

In some embodiments, the eukaryotic cell is a cell found in the circulatory system of a human, non-human primate, and/or other mammal, including mice and/or rats. Exemplary circulatory system cells include, but are not limited to, platelets, plasma cells, red blood cells, B-cells, T-cells, natural killer cells, macrophages, neutrophils, precursor cells of the same, or so on. In some embodiments, at least one eukaryotic cell may be derived from any of these circulating eukaryotic cells.

In some embodiments, at least one eukaryotic cell is a natural killer cell, or a precursor or progenitor cell to the natural killer cell.

In some embodiments, at least one eukaryotic cell is a B-cell, or a B-cell precursor or progenitor cell.

In some embodiments the eukaryotic cells may be plant cells. In some embodiments the plant cells are cells of monocotyledonous or dicotyledonous plants, including, but not limited to, zucchini, woody plants such as coniferous and deciduous trees, wheat, turnip, tomato, tobacco, sunflower, sugarcane, sugar beet, strawberry, spinach, soybean, sorghum, rye, rice, raspberry, rapeseed, radish, pumpkin, potato (including sweet potatoes), plum, pineapple, peanut, pea, papaya, oat, melon, mango, maize, lettuce, lentil, herbs, hemp, grass, flowers, eucalyptus, cucumber, cotton, coffee, citrus, chicory, cherry, celery, cauliflower, carrot, canola, cabbage, broccoli, brassicas, blackberry, bean, barley, banana, avocado, asparagus, Arabidopsis, and other fruiting, an ornamental plant, almonds, alfalfa, a perennial grass, a forage crop, other vegetables, other stone fruit (e.g., peach, nectarine, apricot, pears, plums etc.), other pome fruit (e.g. apples, pears etc.), other fruits, other bulb vegetables (e.g., garlic, onion, leek etc.), other agricultural crops, perennial plant parts (e.g., bulbs; tubers; roots; crowns; stems; stolons; tillers; shoots; cuttings, including un-rooted cuttings, rooted cuttings, and callus cuttings or callus-generated plantlets; apical meristems etc.), and any combinations or hybrids thereof. As used herein, the term “plants” refers to all physical parts of a plant, including seeds, seedlings, saplings, roots, tubers, stems, stalks, foliage, and fruits.

### Tumors

In some embodiments, pharmaceutical compositions and/or formulations described herein target a tumor. The tumor may be a benign tumor, a premalignant tumor, or a malignant tumor.

### Insertion of Transgenes

The invention provides methods for introducing a transgene to a subject, e.g., a human subject. In some embodiments, the method comprises introducing an effective amount of at least one GIS described herein to the subject. In some embodiments, the method comprises introducing an effective amount of at least one GIS which comprises a transgene to the subject.

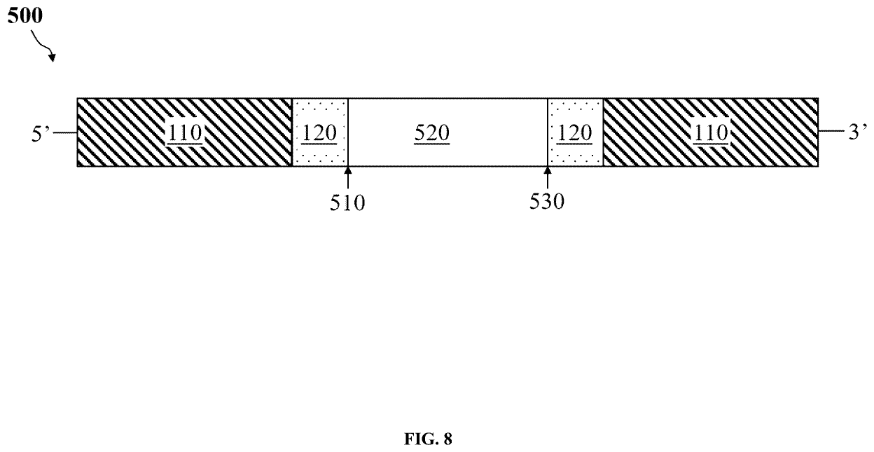

In some embodiments, the method may comprise inserting the transgene at a one or more target insertion sites. Turning now to FIG. 8 where a region of a subject genome with an inserted transgene is illustrated 500. The subject genome DNA includes, in this example, a target insertion site 120 and surrounding genomic DNA 110. For clarity, it should be understood that the target insertion site is part of the subject DNA. The 5′ junction 510 marks the point of transition between the subject DNA and the inserted transgene 520, on the transgenes 5′ end; this junction 510 may have a duplication of part or all of any upstream target site sequence present both in the subject genome and at the template RNA 5′ end. Conversely, the 3′ junction 530 marks the point of transition between the 3′ end of the transgene and the subject DNA; this junction 530 may have a duplication of part or all of any downstream target site sequence present both in the subject genome and in the template RNA 3′ module. Junctions 510 and/or 530 may also contain additional nucleotide(s) such as can result from non-templated nucleotide addition by the RT to an as-yet un-extended primer or to the cDNA 3′ end prior to enzyme dissociation from template-product duplex.

### Target Insertion Sites

In some embodiments, one or more target insertion sites comprise a safe harbor site. As used herein, the term “safe harbor site” refers to a location in the subject genome where insertion of a transgene does not result in unintended disruption of cellular functions. In general, a site in a subject genome may be identified as a safe harbor site if either (a) insertion of genetic material at that site does not alter expression of subject genes, or (b) insertion of genetic material at the that site alters the expression of a gene, but that alteration does not alter normal subject cell function (for example, due to a large number of repeats of the disrupted gene in the subject genome). As a non-limiting example of case (b), the genes coding for ribosomal RNA (rRNA) are repeated with such abundance in the genome that disruption of some rRNA genes does not perturb normal cell function.

In some embodiments, at least one safe harbor site and/or target insertion site comprises at least one ribosomal DNA (rDNA) sequence. As used herein, the term “ribosomal DNA” refers to any gene which encodes for rRNA. In some embodiments, at least one safe harbor site and/or target insertion site comprises at least one 28 S rDNA sequence.

### Transgenes

The methods and compositions of the invention may be used to insert any payload sequence (i.e., transgene) without limitation to the length or source of the payload sequence.

In some embodiments, the transgene comprises a therapeutically active gene. As used herein, the term “therapeutically active gene” refers to any gene with an expression product that is useful in the treatment, amelioration, or prevention of at least one therapeutic indication.

In some embodiments, at least one transgene may comprise at least one telomerase reverse transcriptase (TERT) gene. In some embodiments, at least one transgene may comprise at least one Factor VIII short form gene. In some embodiments, at least one transgene may comprise at least one phenylalanine hydroxylase (PAH) gene.

In some embodiments, at least one transgene is a reporter gene. As used herein, the term “reporter gene” refers to any gene with an expression product that may be detected by any assay.

In some embodiments, at least one reporter gene may include or encode, but is not limited to at least one green florescent protein (GFP), at least one red florescent protein (RFP), luciferase enzyme (LUC), β-galactosidase (LacZ), chloramphenicol acetyltransferase (cat), and the like.

### Non-Wild Type Transgenes

It will be understood by those skilled in the art that while many of the primary examples of transgenes given reference native or wild-type sequences, the GIS disclosed herein are in no way limited to inserting wild-type or naturally occurring genes or portions of gene sequences. The GIS of the invention may be used to insert, for example, genes that are derived from wild-type genes, comprise only portions of wild-type genes, are assemblies of portions from different wild-type genes, and/or are genes whose sequence is not known to exist in nature. Further, a GIS of the invention may be used to insert a transgene whose expression product is not normally present in a subject cell and/or is not normally the result of gene expression.

### Transgene Regulatory Elements

In some embodiments, the GIS of the invention may be used to insert at least one transgene which comprises or encodes at least one regulatory element. For example, a transgene may be designed and/or engineered to include any number of miRNA and/or siRNA binding regions in the transgene expression products. Generally, inclusion of miRNA and/or siRNA may allow for de-targeting of transgene expression from cell types that include the complimentary miRNA or siRNA in their transcriptome.

In some embodiments, a transgene may include or encode both a first expression product comprising or encoding at least one miRNA and/or siRNA and a second expression product (or more) which includes or encodes at least one miRNA and/or siRNA binding site which is complimentary to the first expression product. Without wishing to be bound by theory, this may prevent long term expression of the second expression product.

### Antibodies

As used herein, the term “antibody” is referred to in the broadest sense and specifically covers various embodiments including, but not limited to monoclonal antibodies, polyclonal antibodies, multispecific antibodies (e.g., bispecific antibodies formed from at least two intact antibodies), and antibody fragments (e.g., diabodies) so long as they exhibit a desired biological activity (e.g., “functional”). Antibodies are primarily amino acid-based molecules which are monomeric or multimeric polypeptides which comprise at least one amino acid region derived from a known or parental antibody sequence. The antibodies may comprise amino acid motifs that recruit one or more endogenous or non-native modifications (including, but not limited to the addition of sugar moieties, fluorescent moieties, chemical tags, etc.). For the purposes herein, an “antibody” may comprise a heavy and light variable domain as well as an Fc region.

The GIS of the invention may be used to insert a transgene which comprises or encodes at least one or more functional antibodies.

### Treatment of Therapeutic Indications

The invention provides methods for treating or preventing at least one therapeutic indication in a subject in need thereof. In some embodiments, the method comprises introducing an effective amount of at least one GIS described herein to the subject. In some embodiments, the method comprises introducing an effective amount of at least one GIS which comprises at least one therapeutically active transgene to the subject.

In some embodiments, the at least one therapeutic indication comprises at least one loss of function genetic condition. In some embodiments, at least one method for treatment of at least one therapeutic indication comprises administering at least one transgene which rescues the subject from a loss of function genetic condition. As used herein the term “rescue” refers to providing at least one composition to the subject which allows the subject to perform a native function it was otherwise lacking.

In some embodiments, at least one method comprises rescuing insufficient telomerase activity in a subject by administering an effective amount of GIS comprising at least one TERT transgene to the subject.

In some embodiments, the methods and compositions of the invention may be used to treat or prevent conditions caused by insufficient telomerase function in a subject. In some embodiments, at least one method comprises administering a therapeutically effective amount of at least one GIS comprising at least one TERT gene to a subject displaying insufficient telomerase activity. In some embodiments, at least one method comprises administering a therapeutically effective amount of at least one GIS, comprising at least one TERT gene of a subject suspected of developing a disease due to insufficient telomerase activity.

### Regulation of Heterologous Genes

The GIS of the invention, including the formulations and pharmaceutical compositions described herein, may be used in methods for regulating expression of heterologous genes. For the sake of clarity, the term “heterologous gene” when used in reference to regulate gene expression herein, refers to any gene in the subject genome other than the gene being inserted by the GIS.

In general, a method for regulating heterologous gene expression may include using a GIS of the invention to insert a sequence whose expression product acts on the expression pathway of another gene. For example, the expression product of an inserted gene may affect the transcription of the heterologous gene into mRNA, the translation of the heterologous gene mRNA into a polypeptide, the rate of degradation or inactivation of a heterologous gene's mRNA in the cytoplasm, or the like in any combination.

In some embodiments, at least one GIS of the invention may be used to insert a transgene which comprises or encodes at least one micro-RNA (miRNA). In some embodiments, a miRNA suitable for practicing this disclosure may include any miRNA known or yet to be discovered in the art. In some embodiments, at least one GIS may be used to insert a transgene which comprises or encodes at least one artificial miRNA, wherein said artificial miRNA is designed to bind to at least one gene expression product present in the subject. As used herein, the term “artificial miRNA” is used to refer to a miRNA whose sequence has been altered or designed to bind to a desired target sequence. Artificial miRNA may be designed through various methods known in the art.

In some embodiments, at least one GIS of the invention may be used to insert a transgene which comprises or encodes at least one small interfering RNA (siRNA). As used herein the term “small interfering RNA” refers to a double-stranded ribonucleic acid (dsRNA) having a nucleotide sequence that is substantially identical to at least a part of a target gene. Generally, siRNAs are usually 21-25 nt in length but may be less or more and interferes with (inhibits) target gene expression by promoting degradation of the target gene's mRNA. Any siRNA known or yet to be discovered may be suitable for use in the invention.

In some embodiments, at least one GIS of the invention may be used to insert a transgene which comprises or encodes at least one artificial siRNA. As used herein the term “artificial siRNA” refers to a siRNA whose sequence has been designed to complement at least one gene of interest.

In some embodiments, at least one GIS of the invention may be used to insert a transgene which comprises or encodes at least one transcription factor (TF). As used herein the term “transcription factor” refers to any polypeptide that binds to DNA and alters or affects transcription of at least one gene. Any TF known or yet to be discovered may be suitable for use in the invention.

A GIS of the invention may be used to insert a transgene which comprises or encodes any combination of miRNA, siRNA, and/or TF. For example, at least one GIS may be used to insert a transgene comprising or encoding any of: at least one miRNA and at least one siRNA; at least one miRNA and at least one TF; at least one siRNA and at least one TF; or at least one miRNA, at least one siRNA, and at least one TF.

### Preventative Applications

In some embodiments pharmaceutical compositions and/or formulations described herein may be used to prevent disease or stabilize the progression of a therapeutic indication.

In some embodiments pharmaceutical compositions and/or formulations described herein may be used as a prophylactic to prevent a therapeutic indication in the future.

In some embodiments pharmaceutical compositions and/or formulations described herein may be used to halt further progression of a therapeutic indication.

### Vaccine

In some embodiments pharmaceutical compositions and/or formulations described herein may be used as, and/or in a manner similar to that of a vaccine. As used herein, a “vaccine” is a biological preparation that improves immunity to a particular therapeutic indication or infectious agent.

In some embodiments pharmaceutical compositions and/or formulations described herein may be used as, and/or in a manner similar to that of a vaccine for a therapeutic area such as, but not limited to, dermatology, CNS, cardiovascular, oncology, endocrinology, immunology, respiratory, and anti-infective.

### Antigens

The GIS of the invention may be used to insert a transgene which comprises or encodes at least one antigen, which may be optionally excited by or presented on the surface of at least one subject cell. As used herein, the term “antigen” refers to a composition which causes an immune response in an organism. For example, a composition which causes a subject organism to produce antibodies against the composition in particular, which, in turn, provokes an adaptive immune response in the subject organism. Antigens can be any immunogenic substance including, for example, polypeptides, proteins, polysaccharides, nucleic acids, lipids, and the like. In some embodiments, antigens may be derived from infectious agents including but not limited to bacteria, viruses, protozoa, fungi, prions, and so forth.

In some embodiments, antigens may include parts or subunits of infectious agents, for example, coats, coat components, coat proteins, coat polypeptides, surface components, surface proteins, surface polypeptides, capsule components, cell wall components, flagella, fimbriae, toxins, or toxoids.

In some embodiments, at least one GIS of the invention may be used to insert a transgene which comprises or encodes at least one antigen to vaccinate a subject against at least one therapeutic indication.

### Research

In some embodiments pharmaceutical compositions and/or formulations described herein may be used for diagnostic purposes or as research tools for any of the therapeutic indications disclosed herein.

In some embodiments pharmaceutical compositions and/or formulations described herein may be used in any research experiment, e.g., in vivo, or in vitro experiments.

In some embodiments pharmaceutical compositions and/or formulations described herein may be used to detect a biomarker for research.

In some embodiments pharmaceutical compositions and/or formulations described herein may be used in cultured cells. The cultured cells may be derived from any origin known to one with skill in the art, and may be as non-limiting examples, derived from a stable cell line, an animal model or a human patient or control subject.

In some embodiments pharmaceutical compositions and/or formulations described herein may be used in in vivo experiments in animal models (i.e., mouse, rat, rabbit, cat, dog, non-human primate, guinea pig, drosophila, ferret, C. elegans, zebrafish, or any other animal used for research purposes, known in the art).

In some embodiments pharmaceutical compositions and/or formulations described herein may be used in stem cells and/or cell differentiation

In some embodiments pharmaceutical compositions and/or formulations described herein may be used in human research experiments or human clinical trials.

The invention provides methods for scientific and/or medical research on a subject. In some embodiments, the method comprises introducing an effective amount of at least one GIS described herein to the subject. In some embodiments, the method comprises introducing an effective amount of at least one GIS which comprises at least one reporter transgene to the subject.

### Solo and Combination Therapy

In some embodiments pharmaceutical compositions and/or formulations described herein may be used as a solo therapeutic or combination therapeutics for the treatment of diseases.

In some embodiments pharmaceutical compositions and/or formulations described herein may be used as a solo therapy. In some embodiments pharmaceutical compositions and/or formulations described herein may be used in combination therapy. The combination therapy may be in combination with one or more neuroprotective agents such as small molecule compounds, growth factors and hormones which have been tested for their neuroprotective effect on neuron degeneration.

In some embodiments pharmaceutical compositions and/or formulations described herein may be used in combination with one or more other therapeutic agents. By “in combination with,” it is not intended to imply that the agents must be administered at the same time and/or formulated for delivery together, although these methods of delivery are within the scope of the invention. The pharmaceutical compositions and/or formulations described herein, and other therapeutic agents can be administered concurrently with, prior to, or subsequent to, one or more other desired therapeutics or medical procedures. In general, each agent will be administered at a dose and/or on a time schedule determined for that agent.

Therapeutic agents that may be used in combination with the pharmaceutical compositions and/or formulations described herein can be small molecule compounds which are antioxidants, anti-inflammatory agents, anti-apoptosis agents, calcium regulators, anti-glutamatergic agents, structural protein inhibitors, compounds involved in muscle function, and compounds involved in metal ion regulation.

### In Vivo GIC Synthesis

The invention provides methods for the synthesis of GIS biopolymers, for example GIC biopolymers. In some embodiments, the method comprises administering at least one GIC synthesis constructs to a subject population of cells, maintaining the population of cells for sufficient time for the at least one GIS synthesis construct to be expressed by the subject cells, and collecting and purifying the GIS synthesis construct expression product by such methods as are known in the art.

In some embodiments, at least one GIC synthesis construct comprises or encodes the GIC of the invention. In some embodiments, at least one GIC synthesis construct comprises or encodes the GIC and the means for in vivo synthesis of at least one recombinant RNA. Such means may include providing or encoding an RNA polymerase promoter, sequences for selection and purification of the recombinant RNA, the complimentary GIC sequence, and post recombinant RNA production processing signals. In some embodiments, at least one GIC synthesis construct is administered in the form of a DNA plasmid which allows for the production of the encoded RNA by endogenous cellular machinery.

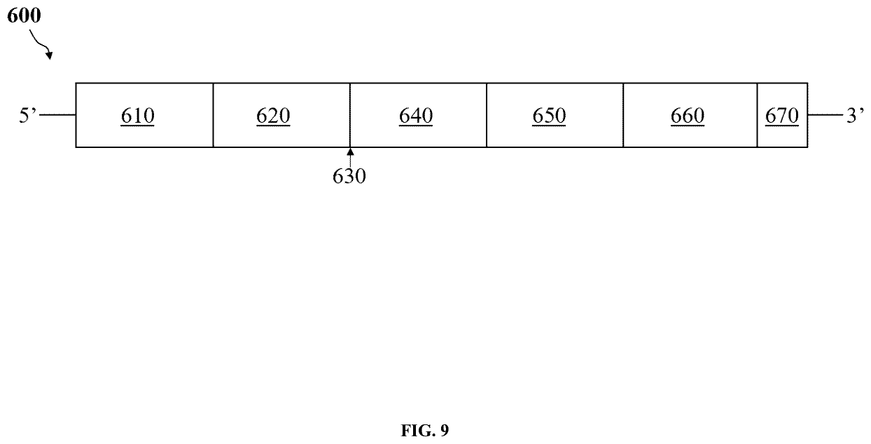

An exemplary GIC synthesis construct 600 is illustrated in FIG. 9. At the 5′ end of the construct, the RNAP module 610 may include any suitable RNA polymerase promoter (for example a T7 RNAP promoter). When present, the optional 5′ leader module 620 is located 3′ to the RNAP module and may include components which improve template 5′ module folding and self-cleavage and/or allow for expeditious removal of GIC transcripts with an immunogenic and/or transcript-destabilizing 5′ end (for example as would result from failure of RZ self-cleavage). Before use as a GIC, any expressed 5′ leader module RNA is cleaved at the RZ self-cleavage site 630. The 5′ module compliment 640 template module compliment 650 and 3′ module compliment 660 respectively encode the GIC 5′ module, template module, and 3′ module. Finally, on the 3′ end may be a linearization restriction enzyme site 670 that is the point of cleavage by a restriction enzyme providing for linearization of the GIC RNA and ensuring that all superfluous vector components remain on the vector.

### VII. ENUMERATED EMBODIMENTS

Embodiment 1. A system for genome editing comprising (i) at least one reverse transcriptase construct (RTC), said RTC comprising a polynucleotide encoding a polypeptide having enzymatic activity for reverse transcription of a polynucleotide template, and (ii) at least one gene insertion construct (GIC), said GIC comprising at least one polynucleotide template suitable for reverse transcription by a polypeptide encoded by the at least one RTC.

Embodiment 2. The system of embodiment 1, wherein the at least one reverse transcriptase construct comprises at least one biopolymer, said biopolymer comprising at least one nucleic acid, at least one amino acid, and any combination thereof.

Embodiment 3. The system of any one of embodiments 1 or 2, wherein the at least one reverse transcriptase construct comprises at least one reverse transcriptase module (RTC: RT-module), optionally at least one reverse transcriptase construct 5′ module (RTC: 5′ module), optionally at least one reverse transcriptase construct 3′ module (RTC: 3′ module), and any combination thereof.

Embodiment 4. The system of embodiment 3, wherein the at least one reverse transcriptase module comprises or encodes at least one reverse transcriptase.

Embodiment 5. The system of any one of embodiments 3 or 4, wherein the at least one reverse transcriptase module comprises or encodes at least one reverse transcriptase derived from a non-long terminal repeat (non-LTR) retroelement.

Embodiment 6. The system of any one of embodiments 4 or 5, wherein the at least one reverse transcriptase comprises or encodes a non-native translation start codon.

Embodiment 7. The system of any one of embodiments 4-6, wherein the at least one reverse transcriptase comprises at least one DNA binding domain, at least one RNA binding domain, at least one cDNA synthesis domain, at least one endonuclease domain, and any combination thereof.

Embodiment 8. The system of embodiment 7, wherein at least one of the at least one reverse transcriptase domain, at least one subject DNA binding domain, at least one template RNA binding domain, and at least one endonuclease domain, and any combination thereof, are derived from a species of reverse transcriptase which is different than at least one of the other at least one reverse transcriptase domain, at least one subject DNA binding domain, at least one template RNA binding domain, and at least one endonuclease domain.

Embodiment 9. The system of embodiment 3, wherein the optional at least one reverse transcriptase construct 5′ module comprises or encodes at least one RNA polymerase promoter, at least one 5′ untranslated region (5′-UTR), at least one Kozak sequence, at least one 5′ cap and any combination thereof.

Embodiment 10. The system of embodiment 3, wherein the optional at least one reverse transcriptase construct 3′ module comprises or encodes at least one reverse transcriptase translation stop codon, at least one 3′ untranslated region (3′ UTR), at least one poly-A tail, and any combination thereof.

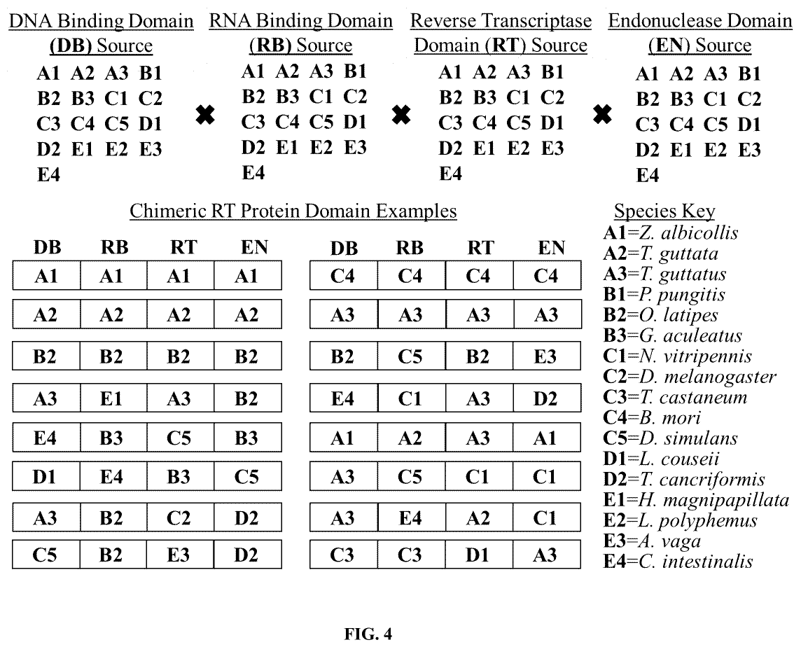

Embodiment 11. The system of any one of embodiments 1-10, wherein the at least one reverse transcription module comprises or encodes at least one structure illustrated in FIGS. 2-5 or any combination thereof.

Embodiment 12. The system of any of embodiments 1-11, wherein the at least one reverse transcriptase construct comprises, encodes, or is encoded by at least one of SEQ ID NOS 1-57 and any combination thereof.

Embodiment 13. The system of embodiment 1, wherein the at least one gene insertion construct comprises or encodes at least one nucleic acid biopolymer.

Embodiment 14. The system of any one of embodiments 1 or 13, wherein the at least one gene insertion construct comprises or encodes at least one optional GIC: 5′ module, at least one GIC: payload module, at least one optional GIC: 3′ module, and any combination thereof.

Embodiment 15. The system of embodiment 14, wherein the at least one GIC: 5′ module comprises or encodes at least one sequence derived from a native retroelement 5′ region, optionally at least one GIC: 5′ module rRNA sequence, optionally at least one GIC: 5′ module ribozyme sequence, optionally at least one GIC: 5′ module folding motif sequence, or any combination thereof.

Embodiment 16. The system of embodiment 15, wherein the optional at least one GIC: 5′ module rRNA sequence comprises or encodes between 1 and 30 nt of subject rRNA.

Embodiment 17. The system of embodiment 15, wherein the optional at least one GIC: 5′ module ribozyme sequence comprises or encodes at least one self-cleaving ribozyme, optionally wherein said self-cleaving ribozyme comprises a hepatitis delta virus ribozyme.

Embodiment 18. The system of embodiment 17, wherein the optional at least one GIC: 5′ module ribozyme sequence comprises or encodes a ribozyme derived from the 5′ region of at least one non-long terminal repeat retroelement.

Embodiment 19. The system of embodiment 15, wherein the optional at least one GIC: 5′ module folding motif sequence comprises or encodes at least one autonomous folding RNA sequence motif, optionally wherein said autonomous folding RNA sequence motif comprises at least one hairpin motif, at least one stem-loop motif, at least one paired stem 4 motif or any combination thereof.

Embodiment 20. The system of any one of embodiments 14-19, wherein the GIC: 5′ module comprises or encodes least one of SEQ ID NOS 60-153, 179-205, or 206-207 or any combination thereof.

Embodiment 21. The system of embodiment 14, wherein the at least one GIC: 3′ module comprises or encodes at least one GIC: 3′ module reverse transcriptase recognition sequence, optionally at least one GIC: 3′ module rRNA sequence, optionally at least one GIC: 3′ module A-Tract sequence, or any combination thereof.

Embodiment 22. The system of embodiment 21, wherein the at least one GIC: 3′ module reverse transcriptase recognition sequence comprises or encodes at least one sequence which interacts with at least one reverse transcriptase.

Embodiment 23. The system of any one of embodiments 21 or 22, wherein the at least one GIC: 3′ module reverse transcriptase recognition sequence is derived from the 3′ region of a native retroelement.

Embodiment 24. The system of embodiment 21, wherein the optional at least one GIC: 3′ module rRNA sequence comprises or encodes between 1 and 30 nt of rRNA.

Embodiment 25. The system of embodiment 21, wherein the optional at least one GIC: 3′ module A-Tract sequence comprises or encodes a sequence of between 1 and 50 adenine bases.

Embodiment 26. The system of any one of embodiment 14 or embodiments 21-25, wherein the at least one GIC: 3′ module comprises or encodes at least one of SEQ ID NOS 225-253, or any combination thereof.

Embodiment 27. The system of embodiment 14, wherein the at least one GIC: payload module comprises or encodes at least one transgene sequence, optionally at least one transgene promoter sequence, optionally at least one transgene 5′ untranslated sequence, optionally at least one transgene 3′ untranslated sequence, optionally at least one transgene polyadenylation signal sequence, optionally at least one transgene non-coding RNA (ncRNA) processing sequence, or any combination thereof.

Embodiment 28. The system of embodiment 27, wherein the at least one transgene sequence comprises or encodes at least one sequence of interest for insertion into a subject genome.

Embodiment 29. The system of embodiment 27, wherein at least one transgene promoter sequence comprises or encodes at least one sequence which promotes expression of a transgene in a subject genome.

Embodiment 30. The system of embodiment 27, comprising at least one transgene 5′ untranslated sequence that comprises or encodes at least one transgene mRNA 5′ untranslated region.

Embodiment 31. The system of embodiment 27, wherein at least one transgene 3′ untranslated sequence comprises or encodes at least one transgene mRNA 3′ untranslated region.

Embodiment 32. The system of embodiment 27, wherein at least one transgene polyadenylation signal sequence comprises or encodes at least one transgene polyadenylation signal.

Embodiment 33. The system of embodiment 27, wherein at least one transgene non-coding RNA (ncRNA) processing sequence comprises or encodes at least one termination signal, at least one 3′ processing signals, and any combination thereof for at least one transgene expressed ncRNA.

Embodiment 34. The system of any one of embodiment 14 or embodiments 27-33, wherein the at least one GIC: payload module comprises or encodes at least one of SEQ ID NOS 296-321, or any combination thereof.

Embodiment 35. The system of any one of embodiments 13-34, wherein at least one of the at least one GIC: 5′ module and at least one GIC: 3′ module comprise or encode at least one sequence derived from a species of non-long terminal repeat retroelement different from at least one of the other at least one GIC: 5′ module and at least one GIC: 3′ module.

Embodiment 36. The system of any one of embodiment 1 or embodiments 13-35, wherein the at least one gene insertion construct comprises or encodes at least one structure illustrated in FIGS. 6-9 and any combination thereof.

Embodiment 37. The system of any one of embodiment 1 or embodiments 13-36, wherein the system comprises: (i) at least one reverse transcriptase construct, wherein the at least one reverse transcriptase construct is comprised or encoded by at least one of SEQ ID NOS 1-57 and, (ii) at least one gene insertion construct, wherein at least one gene insertion construct is comprised or encoded by at least one sequence of SEQ ID NOS 60-153, 179-205, 206-207, 208-217, 225-253, 275-278, 279-281, 284-295, or 296-332.

Embodiment 38. The system of any one of embodiment 1 or embodiments 13-37, comprising a gene insertion construct synthesis construct (GIC: synthesis construct) which comprises or encodes at least one of the gene insertion constructs described in embodiments 13-37.

Embodiment 39. The system of any of embodiments 1-38, wherein at least one of the at least one reverse transcriptase construct and at least one gene insertion construct comprise or encode at least one sequence derived from a different species of retroelement than at least one of the other at least one reverse transcriptase construct and at least one gene insertion construct.

Embodiment 40. The system of any of embodiments 1-39, wherein the system for genome editing comprises at least one combination of, (i) at least one reverse transcriptase construct described in embodiments 2-12, and (ii) at least one gene insertion construct described in embodiments 13-37.

Embodiment 41. A method for inserting at least one transgene into a subject genome comprising administering an effective amount of at least one of the gene insertion systems (GIS) of embodiments 1-40.

Embodiment 42. The method of embodiment 41, wherein the transgene is inserted at one or more target sites in the subject genome, optionally wherein the one or more target sites comprise at least one safe harbor site.

Embodiment 43. The method of embodiment 42, wherein the optional at least one safe harbor site comprises at least one ribosomal DNA (rDNA) sequence, optionally wherein the at least one ribosomal DNA sequence comprises at least one 28 S rDNA sequence.

Embodiment 44. The method of any one of embodiments 40-43, comprising administering at least one of the gene insertion systems formulated with at least one delivery agent.

Embodiment 45. The method of embodiment 44, wherein the at least one delivery agent is at least one nanoparticle, optionally wherein the at least one nanoparticle comprises at least one lipid nanoparticle.

Embodiment 46. A pharmaceutical composition comprising at least one of the gene insertion system of embodiments 1-40 and, optionally at least one of at least one excipient, at least one delivery agent, at least one adjuvant, and any combination thereof.

Embodiment 47. A method of treating a therapeutic indication in a subject in need thereof comprising administering an effective amount of at least one of the gene insertion systems of embodiments 1-40 or at least one of the pharmaceutical compositions of embodiment 46, optionally comprising at least one of the methods of embodiment 41-45.

Embodiment 48. The method of embodiment 47, wherein the therapeutic indication is caused by loss of telomerase activity.

Embodiment 49. The method of any one of embodiments 46 or 47, wherein the at least one gene insertion system comprises at least one TERT transgene.

Embodiment 50. A kit for making a gene insertion system, comprising the methods of the gene insertion systems of embodiments 1-40, optionally the pharmaceutical composition of embodiment 46, and optionally further comprises buffers, DNA plasmids, or protocols to make said gene insertion systems or pharmaceutical composition.

### VIII. DEFINITIONS

28 S rDNA: As used herein, the term “28 S rDNA” refers to the portion of a subject genome which encodes for the large structural ribosomal RNA (rRNA) of the large subunit (LSU) of eukaryotic cytoplasmic ribosomes.

3′ Junction: As used herein, the term “3′ junction” refers to the location where the 3′ end of the inserted sequence connects to the 5′ end of the subject genome.

3′ Region: As used herein, the term “3′ region” refers to the portion of a retroelement gene that is located 3′ to the open reading frame.

5′ Junction: As used herein, the term “5′ junction” refers to the location where the 3′ end of the subject genome connects to the 3′ end of the inserted sequence.

5′ Region: As used herein, the term “5′ region” refers to the portion of a retroelement gene that is located 5′ to the open reading frame.

Activity: As used herein, the term “activity” refers to the condition in which things are happening or being done. Proteins and nucleic acids of the disclosure may have activity and this activity may involve one or more biological events.

Adapted: As used herein, the term “adapted” refers to the alteration of a protein or amino acid sequence in order to alter, add, or remove a property and/or activity

Assay: When used as a verb herein, the term “assay” is used in its broadest sense and refers to the act of testing via any suitable method known in the art. When used as a noun herein, the term “assay” refers to a test used to determine a property, state, and/or activity of the subject of the assay.

Biological Property: As used herein, the terms “biological property” and “property” refer to any characteristic or activity of an organism, physiological system, organ, tissue, cell, or molecule which may be measured or observed.

Cargo: In the context of delivery vehicles, the terms “cargo” and “payload” generally refer to any compounds or structures (e.g., the GIS of the invention) intended for deliver to, on, or near a subject cell, tissue, organ, or physiological system.

Cell: As used herein, the term “cell” is given its broadest possible meaning and refers to any living membrane-bound structure.

Cellular Process: As used herein, the term “cellular process” and its grammatical equivalents, refers to any process that is carried out at a cellular level, which may or may not be restricted to a single cell.

Characteristic: As used herein, the term “characteristic” refers to a feature or quality belonging typically to a person, place, or thing, and serving to identify it. The terms “characteristic” and property” have the same meaning and may be used interchangeably.

Confer: As used herein, the term “confer,” and its grammatical equivalents, refers to the process of adding features to a subject.

Construct: As used herein, the noun “construct” refers to an artificially designed biopolymer. Example biopolymers include DNA, RNA, and polypeptides. In general, constructs described herein are designed for use in an GIS.

Degradation: As used herein, “degradation” refers to the loss of function of a composition over time.

Delivery: As used herein, the term “delivery” refers to the act or manner of delivering a compound, substance, entity, moiety, cargo, or payload in a living cell or organism. The terms “delivery” and “biological delivery” may be used interchangeably unless specified otherwise.

Delivery System: As used herein, the term “delivery system” refers to any composition, method, or combination thereof which, when formulated with a GIS of the present invention, delivers the components of the GIS into the cytoplasm of the target cell. Non-limiting examples of delivery systems include systems comprised of delivery vehicles and systems for direct transfection.

Derived from: As used herein, the term “derived from” refers to a nucleic acid or protein sequence that is isolated from or obtained from a specific source, such as a non-long terminal repeat (non-LTR) retrotransposon. The term includes native sequences isolated from or obtained from a specific source. The term also includes man-made variants of sequences from the original source that have the same or similar functional properties, e.g., the variant can comprise a nucleic or amino acid sequence that has been modified from the original source to have improved functional properties compared to the original source molecule.

Designed: As used herein, the term “designed” refers to compositions that have been altered from their natural or current state to have new and desired properties and or activities.

DNA and RNA: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single stranded (i.e., ssRNA or ssDNA, respectively) or multi-stranded (e.g., double stranded, i.e., dsRNA and dsDNA, respectively). The term “mRNA” or “messenger RNA,” as used herein, refers to a single stranded RNA that encodes the amino acid sequence of one or more polypeptide chains. If an RNA sequence is recited using deoxyribonucleotides, any thymidines (“T”s) can be replaced with uridines (“U”s) or uridine analogs to convert the DNA sequence to an RNA sequence.

DNA Repair: As used herein, the term “DNA repair” refers to any of the endogenous processes carried out in a cell to correct damage to the cell's genome.

Efficient: As used herein, in reference to transgene insertion, the term “efficient,” and its grammatical equivalents, refers to the effectiveness of a given combination of RT protein, GIC: 5′ module, and GIC: 3′ module to effect insertion of the full length of a payload module at the desired target site.

Element: As used herein, the term “element” refers to any discrete component of a molecule, or system, or a single step of a method.

Expression Product: As used herein, the term “expression product” refers to either an RNA transcribed from a sequence of interest (e.g., an mRNA) or a polypeptide translated from an mRNA transcribed from a sequence of interest.

Encapsulate: As used herein, the term “encapsulate” means to enclose, surround, or encase.

Encode: As used herein, the term “encode” refers broadly to any process whereby the information in a polymeric macromolecule is used to direct the production of a second molecule that is different from the first. The second molecule may have a chemical structure that is different from the chemical nature of the first molecule.

Endonuclease: As used herein, the term “endonuclease” refers to any protein, or portion of a protein, which cleaves a polynucleotide chain by separating nucleotides other than the two end ones

Exosomes: As used herein, “exosome” is a vesicle secreted by mammalian cells or a complex involved in RNA degradation.

Ex vivo: The term “ex vivo” refers to removing cells from a donor subject, modifying the cells using the methods described herein, and adding the cells back to a recipient subject. The term includes autologous cells that are obtained from the same individual subject (i.e., the same subject is both the donor of unmodified cells and recipient of the ex vivo modified cells), and allogenic cells that are obtained from a donor subject that is a different individual than the recipient subject. The allogenic donor and recipient may be HLA-matched.

Facilitate: As used herein, the term “facilitate” is used in its broadest sense and refers to making an action or process more likely to occur by the addition of the specified element.

Fidelity: As used herein, the term “fidelity” refers to the accuracy with which a gene of interest is inserted into a subject genome. The term “high fidelity” corresponds to the gene of interest being inserted with a relatively small number of errors in nucleotide identity, sequence length, and target site location. For example, if a template RNA contains approximately 5,000 nucleotides and can be copied by the RT protein to produce cDNA without generating a base-pair mismatch, the gene insertion has high fidelity. Depending on the purpose of the transgene insertion, a limited number of mismatches could occur and still be high enough fidelity to create a functional transgene.

Flanking: As used herein, the term “flanking” refers to the positioning of one element either 5′ (5′ flanking) or 3′ (3′ flanking) to another element. Elements that are said to be flanking may be directly connected to each other or may have other elements interspaced between them.

Formulation: As used herein, a “formulation” includes at least one component of a GIS as described herein, and at least one delivery agent, pharmaceutically acceptable excipient, or both.

Functional/Active: As used herein, in reference to a biological molecule, the term “functional” refers to a biological molecule in a form in which it exhibits a property and/or activity by which it is characterized.

Gene: As used herein, the term “gene” is used in its broadest sense to refer to a distinct sequence of nucleotides which form, or may form, part of a chromosome, and the order of which determines the order of monomers in a polypeptide or nucleic acid molecule.

Gene Insertion Construct: As used herein, the term “Gene Insertion Construct”, or GIC, refers to an RNA construct which comprises the RNA template for an RT protein.

Gene Insertion System: As used herein, the term “Gene Insertion System” or “GIS,” is a system of components (modules) which may be used to insert a genetic sequence (transgene) into a specific location of a subject genome via reverse transcription, including TPRT.

GIC: 3′ Module: As used herein, the term “3′ module” refers to the portion of a GIC which comprises at least one element derived from or functionally substituting for the 3′ region of a retroelement gene.

GIC: 5′ Module: As used herein, the term “GIC: 5′ module” refers to the portion of a GIC which promotes full-length transgene insertion and may or may not derive from the 5′ region of a retroelement gene.

Generates: As used herein, the verb “generate,” and its conjugates is used in its broadest sense to refer to any process that causes the specified product to be present.

Genome: As used herein, the term “genome” is used in its broadest sense to refer to all the genetic material present in a cell.

HDV RZ Fold: As used herein, the term “HDV RZ fold” refers to any RNA sequence that can adopt the fold of the hepatitis delta virus (HDV) ribozyme and which retains ribozyme function.

Heterologous: As used herein, the term “heterologous” refers to any genetic or protein sequence or structure that is put into a cell that does not normally make that genetic or protein sequence or structure. The term also includes individual elements, modules, or portions of an RTC or GIC of the disclosure that comprise nucleic acid (DNA or RNA) sequences or amino acid sequences that are from different species. For example, a 5′ module of an RTC or GIC may comprise a sequence from one (or a first) species of bird, and a 3′ module of the same RTC or GIC may comprise a sequence from a different (or second) species of bird.

Homologous Recombination: As used herein, the term “homologous recombination” refers to any process of transgene insertion which relies on sequence homology between the transgene and the subject genome.

In Vitro: As used herein, the term “in vitro” is used to refer to reactions or processes being carried out outside of a living cell or organisms.

In Vivo: As used herein, the term “in vivo” is used to refer to reactions or processes being carried out inside or on the surface of a living cell or organisms.

Inactive: As used herein, in reference to a biological molecule, the term “inactive” refers to a biological molecule in a form in which it does not exhibit a property and/or activity by which it is characterized.

Inactive Ingredient: As used herein, the term “inactive ingredient” refers to one or more agents that do not contribute to the activity of the active ingredient of the pharmaceutical composition included in formulations. In some embodiments, all, none, or some of the inactive ingredients which may be used in the formulations of the invention may be approved by the US Food and Drug Administration (FDA).

Induce: As used herein, the term “induce,” and its grammatical equivalents, refers to a process which results in a stated outcome without any specific limitation on steps of the process.

Introduce: As used herein, the term “introduce” refers to adding genetic material, often DNA, to a cell.

Insert: As used herein, the term “insert” refers to adding nucleotides to a DNA sequence.

Junction: As used herein, the term “junction” refers to the location in a subject genome where the insertion site DNA of the subject is connected to the cDNA of the inserted transgene.

At least one: As used herein, the term “at least one” refers to one, two, three, four, five or more of the modified object, e.g., a construct, module or sequence of the disclosure.

Lipid Nanoparticle: As used herein, “lipid nanoparticle” or “LNP” refers to a delivery vehicle comprising one or more lipids (e.g., cationic lipids, non-cationic lipids, PEG-modified lipids).

Liposome: As used herein, “liposome” generally refers to a vesicle composed of lipids (e.g., amphiphilic lipids) arranged in one or more spherical bilayers or bilayers.

Loss Of Function: As used herein, the term “loss of function” refers to any change in a subject gene that results the altered gene product lacking a function of the wild-type gene.

Modified: As used herein, “modified” refers to a changed state or structure of a molecule. Molecules may be modified in many ways including chemically, structurally, and functionally.

Modular System: As used herein, “modular system” refers to a system that can be divided into multiple sets of strongly interacting parts that are relatively autonomous with respect to each other.

Motif: As used herein, the term “motif” refers to any sequence of a biopolymer with a recognizable structure that may or may not be defined by a unique chemical or biological function.

Native: As used herein, the term “native” refers to a wild-type or naturally occurring compound, biomolecule (e.g., protein or nucleic acid) or composition.

Non-LTR Retroelement Reverse Transcriptase: As used herein, the term “non-LTR Retroelement Reverse Transcriptase (RT)” refers to a protein with reverse transcription activity derived from a non-LTR Retroelement.

Non-LTR Retroelements: As used herein, the term “non-LTR retroelement” refers to a class of retroelement genes (aka retrotransposons) which do not contain long terminal repeats.

Outside: As used herein, in relation to an insertion site, the term “outside” refers to any part of the genome more than about 60 bp 5′ or 3′ to the insertion site.

Paired RT: As used herein, the term “paired RT” refers to the combination of a reverse transcriptase (RT) with at least one of the modules comprising the insertion payload module. A module may be homologous to its paired RT, meaning the RT and all elements in the module are derived from the same retroelement gene. A module may be heterologous to its paired RT, meaning at least one element of the module is not derived from the same retroelement gene as the RT.

Payload: With the exception of when used in the context of delivery vehicles, the term “payload” can refer to any sequence of nucleic acids (e.g., a gene of interest) included in a gene insertion system (GIS) intended for insertion into a subject genome.

Percent Homology: The terms “percent homology” or “% homology” refer to the amount of sequence that is identical or the same between two nucleic acid or amino acid sequences. The term percent homology” can be used interchangeably with the term “percent identity” or “percentage of sequence identity” as defined herein.

As used herein, “percent identity” or “percentage of sequence identity” or “percent homology” is determined by comparing two optimally aligned sequences over a comparison window, wherein the portion of the sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical,” “identity,” or “homology” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95% identity, at least 96% identity, at least 97% identity, at least 98% identity, at least 99% identity, at least 99.5% identity, or at least 99.9% identity over a specified region), when compared and aligned for maximum correspondence over a comparison window, or designated region as measured using one of the following sequence comparison algorithms or by manual alignment and visual inspection. These definitions also refer to the complement of a test sequence. Thus, unless otherwise indicated, all nucleic acid and amino acid sequences provided herein include sequences that are substantially identical to a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters.

Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natd. Acad. Sci. USA 89:10915, 1989) alignments (B) of 50, expectation (E) of 10, M=5, N=−4.

The BLAST algorithm also performs a statistical analysis of the similarity between two sequences (see, e.g., Karlin and Altschul, Proc. Natd. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

Peptide: As used herein, “peptide” refers to a chain or strand of amino acids which is less than or equal to 50 amino acids long, e.g., about 5, 10, 15, 20, 25, 30, 35, 40, 45, or 50 amino acids long.

Pharmaceutical Composition: As used herein, the term “pharmaceutical composition” refers to compositions comprising at least one active ingredient and optionally one or more pharmaceutically acceptable excipients.

Polyadenosine: As used herein, the term “polyadenosine” refers to a sequence of adenosine nucleotides of any length.

Polyadenosine Tail: As used herein, the term “polyadenosine tail”, or “poly-A tail”, is used to refer to a sequence of adenosine nucleotides of about 80 or more nucleotides in length.

Polyadenosine Tract: As used herein, the terms “polyadenosine tract,” “poly A-Tract,” and “A-Tract,” (all abbreviated PA) are equivalent and used interchangeably to refer to a sequence of adenosine nucleotides from about 1-50 nucleotides in length.

Promoter: As used herein, the term “promoter” refers to any sequence of DNA to which proteins bind that initiate transcription.

Pro-Protein: As used herein, the terms “protein precursor,” “pro-protein,” and “pro-peptide” refer to an inactive protein that can be turned into an active form by post-translational modification.

Protect: As used herein, the term “protect,” and its grammatical equivalents, refers to any composition or process that prevents degradation of all or a portion of a biopolymer.

Protein: As used herein, “protein” is used to refer to an amino acid biopolymer more than 50 amino acids long. non-limiting examples of proteins described herein are enzymes, reverse transcriptases, and endonucleases.

Region: As used herein, the term “region” refers to a portion of a sequence of nucleotides or amino acids. A region may be of unknown or undefined length, in which case it is specified by the function it refers to or its position relative to other elements in the sequence.

Retroelement/Retrotransposon: As used herein, the terms “retroelement” and “retrotransposon” interchangeably refer to a class of eucaryotic genes capable of replicating to new locations within their own genome through an RNA intermediate.

Reverse Transcriptase: As used herein, the term “reverse transcriptase” refers to any protein capable of synthesizing cDNA from an RNA template sequence.

Reverse Transcriptase Construct: As used herein, the term “reverse transcriptase construct” (RTC), as previously mentioned, refers to a biopolymer construct which includes or encodes at least one RT.

RTC: RT Module: As used herein, the term “RTC: RT Module” or “Reverse Transcriptase Module” refers to a biopolymer construct which includes or encodes at least one RT.

Ribosomal DNA: As used herein, the term “ribosomal DNA (rDNA)” refers to the portion of a subject genome which codes for the precursor ribosomal RNA synthesized by RNAP I.

Ribosomal RNA: As used herein, the term “ribosomal RNA (rRNA)” refers to the non-coding RNA components of ribosomes.

Segments: As used herein, the term “segment” refers to a portion of a sequence. For example, segments of a nucleotide sequence may comprise any portions of a gene less than its full length.

Selective: As used herein, the terms “selective” and “selectivity” refers to the molecules, including but not limited to enzymes, enzyme proteins and genes, which tend to bind to very limited kinds, structures, protein, or genetic sequences of other molecules.

Self-Cleaving Ribozyme: As used herein, the term “self-cleaving ribozyme” is used to refer to a class of RNA which catalyzes sequence-specific intramolecular (or intermolecular) cleavage.

Selectivity: As used herein, “selectivity” refers to how likely an RT is to efficiently utilize a heterologous-paired GIC 5′ or 3′ module.

Sequence: As used herein, the term “sequence” refers to either the order of amino acids given from N-terminus to C-terminus, or the order of nucleotides given 5′ to 3′ of a biopolymer.

Site-specific: As used herein, the phrase “site-specific” refers to a locus, for example of about a 60 bp sequence.

Stability: As used herein, the term “stability” refers to the ability of a composition to retain its properties over time.

Successful TPRT: As used herein, the phrase “successful TPRT” refers to synthesis of cDNA and/or insertion of a transgene using a primer made by target site nicking.

Suitable: As used herein, the term “suitable” refers to anything that is effective, workable, or fitting for a particular purpose or use,

Synthetic: As used herein, the term “synthetic” refers to anything produced, prepared, and/or manufactured by the hand of man. Synthesis of polynucleotides or polypeptides or other molecules of the invention may be chemical or enzymatic.

Target Cell: As used herein, the phrase “targeted cells” refers to any one or more cells of interest. The cells may be found in vitro, in vivo, in situ or in the tissue or organ of an organism. The organism may be an animal, preferably a mammal, more preferably a human and most preferably a patient.

Target Primed Reverse Transcription: As used herein, the term “target primed reverse transcription” refers to any process where a reverse transcriptase uses a genome-embedded nicked DNA 3′ end at the target site as the primer to initiate cDNA synthesis.

Template: As used herein, the terms “template” and “RNA template” refer to a sequence of RNA which is transcribed into cDNA by an RT.

Template Terminus: As used herein, the term “template terminus” refers to either the 5′ or 3′ end of an RNA template.

Therapeutically Active: As used herein, the term “therapeutically active” refers to a gene or gene product which is treats or alleviates a therapeutic indication in a subject.

Transcription: As used herein, the term “transcription” refers to the formation or synthesis of an RNA molecule by an RNA polymerase using a DNA molecule as a template.

Transfection: As used herein, the term “transfection” refers to methods to introduce exogenous nucleic acids into a cell. Methods of transfection include, but are not limited to, chemical methods, physical treatments and cationic lipids or mixtures.

Transgene: As used herein, the term “transgene” refers to any gene inserted into a subject genome.

Translation: As used herein, the term “translation” refers to the formation of a polypeptide molecule by a ribosome based upon an RNA template.

Treat and prevent: As used herein, the terms “treat” or “prevent” as well as words stemming therefrom do not necessarily require 100% or complete treatment or prevention. Rather there are varying degrees of treatment or prevention of which one of ordinary skill in the art recognizes as having a potential benefit or therapeutic effect. Also, “prevention” can encompass delaying the onset of the disease, symptom, or condition thereof.

Unmodified: As used herein, the term “unmodified” refers to any substance, compound, or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, the term “vector” is any molecule or moiety which transpo7, transduces, or otherwise acts as a carrier of a heterologous molecule.

### IX. EQUIVALENTS AND SCOPE

Those skilled in the art will recognize or be able to ascertain using no more than routine experimentation, many equivalents to the specific embodiments in accordance with the disclosure described herein. The scope of the invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits, but does not require, the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The invention is further illustrated by the following non-limiting examples.

### X. EXAMPLES

### EXAMPLE 1. Gene Insertion Construct (GIC) In Vitro Transcription (IVT)—GIC with No Payload

GIC RNA biopolymers of less than approximately 1000 nt, such as RNAs used for TPRT assays with purified RT in vitro, are generally prepared via an in vitro RNA transcription (IVT) reaction as follows.

GIC DNA templates for RNA transcription are generated by PCR using Q5 DNA polymerase (NEB) and purified by column clean-up (Bio Basic).

IVT reactions are performed using T7 RNA Polymerase (RNAP) by one of two protocols that generate equivalent purified RNA. By the first method, which uses purified reaction components, 1 μg of DNA template is transcribed in 25 μL of reaction solution containing 40 mM Tris pH 7.9, 2.5 mM spermidine, 26 mM MgCl₂, 0.01% Triton X-100, approximately 30 mM DTT, 8 mM GTP, 4 mM all other rNTPs, 0.5 uL RiboLock (Thermo Scientific), 0.5 uL inorganic pyrophosphatase (NEB), 0.5 uL T7 RNAP (purified after over-expression in bacteria and stored as 50 mg/mL in 20 mM KPO₄ pH 7.5, 100 mM NaCl, 50% glycerol, 10 mM DTT, 0.1 mM EDTA, 0.2% NaN₃). The reaction is incubated at 370 Celsius for 3-4 hours, followed by addition of 1 uL DNase RQ1 (Promega), 1.5 uL 20 mM CaCl₂, and 2 uL H₂O. By the second method, the NEB HiScribe T7 Kit is used according to manufacturer's instructions, with 1 μg of digested plasmid per 20 ul of reaction solution. The reaction is incubated at 37° C. for 2 hours, followed by addition of 1 uL DNase RQ1 (Promega), 1.5 uL 20 mM CaCl₂, and 2 uL H₂O.

Product RNA is then purified by desalting (Roche mini quick spin column), organic extraction, and precipitation following common procedures known in the art.

### EXAMPLE 2 Gene Insertion Construct (GIC) In Vitro Transcription (IVT)—GIC with Transgene Payload

GIC RNA biopolymers containing a transgene expression cassette payload are prepared via in vitro RNA transcription (IVT) reaction as follows.

GIC DNA transcription template sequences are cloned into pUC57-mini backbone (SEQ ID NO 269) with a T7 RNAP promoter upstream and a BbsI site downstream of the intended GIC RNA template. Purified plasmid DNA is linearized by digestion with BbsI-HF (NEB) at 37° Celsius for 4 hours. Then, the digested plasmid is purified by Qiagen PCR purification column and eluted in nuclease-free water.

IVT reaction is carried out utilizing the NEB HiScribe T7 Kit with 1 μg of digested plasmid per 20 ul of reaction solution. Specifically, each IVT reaction has 2 ul of each rNTP, 2 ul of 10× buffer, 2 ul of T7 polymerase mix, 1 μg of digested plasmid and ddH2O, and is incubated at 37″ C for 2 hours.

After IVT, the DNA template is removed by Rnase-free Dnase I treatment at 37° Celsius for 30 minutes. Next, synthesized RNA is purified by adding equal volume of 25:24:1 phenol:chloroform:isoamyl alcohol, pH 6.7 (PCI), vortexing vigorously, centrifuging and taking the aqueous layer to precipitate with 10% volume of 3 M sodium acetate (pH 5) and 3 volumes of 100% ethanol. After three washes in 70% ethanol, the RNA pellet is air dried and dissolved in 1 mM sodium citrate, pH 6.5.

### EXAMPLE 3. Reverse Transcriptase (RT) Protein Preparation for TPRT assays

RT proteins are produced by transient expression in human cells and purified as follows.

A codon-optimized ORF encoding the indicated RT (GenScript) is cloned between Kpn I and XbaI sites of pcDNA3.1 N-DYK plasmid (GenScript) to be in fusion with the vector-encoded N-terminal FLAG tag (SEQ ID NO. 270) The KpnI site adds a glycine-threonine linker between FLAG tag and RT amino acid sequence. The XbaI site follows translation stop codon(s) near the start of the 3′ UTR. 12 μg of plasmid DNA is reverse transfected using Lipofectamine 3000 (Invitrogen). First, DNA is mixed gently with 500 μL of OPTI-MEM and 24 μL of P3000. Then 500 μL of OPTI-MEM and 24 μL of Lipofectamine are mixed together and added to the DNA mixture. Lipofectamine/DNA complexes are incubated for 10 min at RT and added to cells prepared as below. Briefly, for each transfection, 1 10 cm dish of 80% confluent HEK 293T cells (hereafter 293T) are split onto Lipofectamine/DNA complexes and replated at 80% confluency. After 18-24 hours, cells are trypsinized to remove them from the plate, resuspended in 5 mL media and spun down at −2000 g for 3 minutes in 15 mL conical tubes. The pellet is washed with PBS containing 1 mM PMSF, transferred to a 1.5 mL tube, and re-pelleted at 2000 g for 1 minute at 4° Celsius.

Cell pellets are suspended in 4× pellet volume of 1× hypotonic lysis buffer [HLB; 20 mM HEPES (pH 8), 2 mM MgCl₂, 200 uM EGTA, 10% glycerol, 1 mM DTT, 0.2% serine protease inhibitor cocktail (SPIC, Sigma), 1 mM PMSF]and set on ice for 5 minutes to swell the cells. Cells will then be lysed by 3 cycles of snap freezing the sample in liquid nitrogen and thawing in room temperature water bath. Samples will then be brought to 400 mM NaCl, gently vortexed, and placed on ice for an additional 5 min. Samples will then be then spun at 17000 g for 5 minutes at 4° C. The supernatant is collected and the concentration of NaCl lowered to 200 mM and NP-40 raised to 0.1% through the addition of an equal volume of 1× HLB containing 0.2% NP-40. Samples are vortexed gently and spun at 17000 g for 10 minutes at 4° Celsius.

Clarified supernatant is collected in a new tube and 20 uL blocked and equilibrated FLAG antibody resin added (Sigma). Samples are rotated for 2 hours at 4° Celsius to immunoprecipitate the protein. FLAG resin will then be washed 4× total (2 quick, 2 with 5 minutes rotation at 4° Celsius) with IP buffer (1× HLB, 200 mM NaCl, 0.1% NP-40). Following the final wash, all buffer is removed with a 30G needle and resin resuspended in 40 uL IP buffer. Protein is partially eluted by adding 50 ng/uL triple-FLAG peptide (Sigma) and incubating at room temperature for 1 hr. The eluted protein is flash frozen in liquid nitrogen and stored at −80° Celsius for subsequent use.

### EXAMPLE 4. RTC mRNA Production

RNA (mRNA) RTC biopolymers are prepared as follows.

A codon-optimized ORF encoding the RT (GenScript) is amplified by PCR to append a BamHI site prior to the ORF and a XhoI site after stop codons that terminate the ORF. The BamHI site is in frame between an N-terminal FLAG tag and the RT ORF, and it adds a glycine-serine linker at that junction.

RT ORF is cloned between a 5′ UTR (SEQ ID NO 58) and 3′ UTR and template-encoded polyadenosine tail (SEQ ID NO 59) in pUC57-mini (SEQ ID NO 269) with T7 RNAP promoter sequence upstream and a BbsI site downstream. The mRNA transcription template plasmid is then linearized with BbsI and repurified as described in Example 2. AG Clean cap mRNA synthesis and purification using silica membrane is carried out by a commercial vendor (TriLink), or with TriLink reagents and protocols, typically using 5-methoxy-uridine ribonucleotide triphosphate (5moU) in 100% replacement of uridine ribonucleotide triphosphate (U). Comparison of 100% uridine replacement by 5moU versus N1-methyl pseudouridine demonstrated comparable function of mRNAs with either modified nucleotide.

### EXAMPLE 5. In Vitro RT Activity Screening

Candidate proteins are tested for reverse transcriptase activity in vitro as follows, using a DNA primer annealed to an RNA template, which is the field-standard RT assay.

RT proteins are prepared as in Example 3. Primer DNA oligo (SEQ ID NO 271 is purchased from IDT), and template RNA (SEQ ID NO 272) is generated by the first protocol of Example 1.

For each screening reaction, 2 μL of 8 uM DNA oligo and 2 μL of 4 uM template RNA are annealed by heating the sample to 65” Celsius for 3 minutes and placing the sample on ice for at least 5 minutes.

A non-radioactive master mix is created containing the following: 2 μL of 10× RT buffer (50 mM MgCl₂, 250 mM Tris (pH 7.5), and 750 mM KCl), 2 μL of 100 mM DTT, 2 μL of 20% PEG-6K, and 5 μL of nuclease-free H2O.

A radioactive master mix is also created, containing the following: 1 μL of 10 mM dA, dC, and dTTP; 1 μL of 2 mM dGTP; 4 μL of annealed DNA-RNA described above, and 1 μL of ³²P alpha-dGTP (Perkin Elmer).

For each reaction, 11 μL of the non-radioactive master mix, 2 μL of candidate RT protein, and 7 μL of the radioactive master mix is mixed, brining each reaction volume up to 20 μL. The reaction is allowed to proceed at 37° Celsius for 30 minutes, followed by heat inactivation at 70° Celsius for 5 minutes. 80 μL of stopping solution (50 mM Tris (pH 7.5), 20 mM EDTA, and 0.2% SDS) containing a 100 nt oligonucleotide (SEQ ID NO 218) previously 5′-end radiolabeled using gamma³²P ATP and T4 polynucleotide kinase (NEB) are added to the reaction, then the DNA is purified and concentrated by PCI extraction followed by ethanol precipitation (dry ice ethanol bath). DNA is pelleted at 14,000 g for 20 minutes in a table-top centrifuge, washed once with 75% ethanol, air dried, and resuspended in 5 uL H2O+5 uL 2× formamide loading buffer.

Samples are run on a 9% Urea-PAGE denaturing gel, dried, exposed on phosphoimager screens and imaged the following day on the Typhoon Trio Imager System.

### EXAMPLE 6. In Vitro TPRT Activity Assay

RT proteins are prepared as in Example 3. Template RNA for TPRT is prepared via IVT reaction as described in Example 1. RT protein and template RNA are combined with a target site oligonucleotide duplex either 64 or 84 bp in length duplex DNA (SEQ ID NO. 219 and SEQ ID NO. 220 respectively) with the bottom strand 5′-end-radiolabeled using gamma³²P ATP and T4 polynucleotide kinase (NEB) in magnesium reaction buffer for 30 minutes at 37° Celsius. Products are resolved by denaturing PAGE and the gel imaged with a Typhoon Trio Imager System.

### EXAMPLE 7. Cell Culture and Co-Transfection of RNA Based RTC and GIC

Indicated mammalian cell lines are plated immediately before transfection on 6-well plates at densities of 1.25-2.5 million cells per well.

5 ul of Messenger Max is diluted in 125 ul of Opti-MEM and incubated for 10 minutes.

RTC mRNA and GIC RNA (prepared as in Examples 4 and 2, respectively) are mixed at specified molar ratios then diluted in 125 ul of Opti-MEM. Then the Messenger Max in Opti-MEM solution and GIS RNAs in Opti-MEM solution are mixed well and incubated for 5 minutes at room temperature.

The resulting mixture is added dropwise to one well of cells in a 6-well plate, plates are returned to the cell incubator, and sufficient time is allowed to pass before cells are analyzed.

### EXAMPLE 8. FACS Analysis

### Flow Cytometry Analysis

One day after transfection (unless indicated otherwise), cells are harvested by trypsinization into DMEM media with 5% FBS and then analyzed on Attune N×T Flow Cytometer (Thermo), or equivalent. Live single cells are gated by forward and side scatter. The mCherry channel on Attune is YL2, excited at 561 nm, emission filter is 620/15 nm. The eGFP channel on Attune is BL1, excited at 488 nm, emission filter is 530/30 nm. The flow cytometry results are analyzed using FlowJo 10.8.1. Transfection with GIC RNA alone, without RT mRNA, is used as a background control; background is subtracted from signal when quantifying.

### Cell Sorting

One day after transfection (unless indicated otherwise), cells are harvested by trypsinization into DMEM media with 5% FBS and sorted on Sony SH800 sorter with 130 um chip under the ultra-purity mode, or equivalent. The sorted cells are collected by centrifugation and washed with PBS.

### EXAMPLE 9. RNA based RTC and GIC Composition

RTC mRNA for transfection is produced as in Example 4 and described in Table 1.

TABLE 1
2-RNA Component GIS RTCs

| | RTC: RT-Module Source | SEQ ID |
|---|---|---|
| RTC Identifier | Organism | NO. |
| F-ZoAl RT mRNA | Z. albicollis | 19 |
| F-TaGu RT mRNA | T. guttata | 28 |
| F-TriCasB RT mRNA | T. castaneum | 3 |
| OrLa-3F RT mRNA | O. latipes | 10 |
| ZoAl RT mRNA (untagged) | Z. albicollis | 21 |
| ZoAl_catdead RT mRNA | Z. albicollis | 23 |
| TriCasB RT mRNA | T. castaneum | 5 |
| (untagged) | | |

GIC RNA for transfection is produced as in Examples 1 and 2 and described in Table 2.

TABLE 2
2-RNA Component GIS GICs

| | | Transgene | | Transgene 3′ UTR & | | GIC |
|---|---|---|---|---|---|---|
| | 5′ Module | Promoter | | Poly-A | 3′ Module | SEQ |
| | Source | Region & | | Signal | Source | ID |
| GIC Identifier | Organism | 5′ UTR | Transgene | Regions | Organism | NO. |
| TriCas_ZoAl | T. | CBh | NLSeGFP | SV40LPA | Z. albicollis | |
| | castaneum | | | | | |
| TriCas_GeFo | T. | CBh | NLSeGFP | SV40LPA | G. fortis | |
| | castaneum | | | | | |
| TriCas_TaGu | T. | CBh | NLSeGFP | SV40LPA | T. guttata | |
| | castaneum | | | | | |
| TriCasFlipZoAl | T. | CBh_Flip | GFP | SV40LPA | Z. albicollis | |
| | castaneum | | | | | |
| TriCasBsiZoAl | T. | CBh_Bsi | GFP | SV40LPA | Z. albicollis | |
| | castaneum | | | | | |
| TCA5_ZoAl | T. | CBh | GFP | SV40LPA | Z. albicollis | |
| | castaneum | | | | | |
| TCA5_GeFo | T. | mPGK | GFP | SV40LPA | G. fortis | |
| | castaneum | | | | | |
| TCA5_TaGu | T. | CBh_Bsi | GFP | SV40LPA | T. guttata | |
| | castaneum | | | | | |
| TCA5_TiGu | T. | CBh_Bsi | GFP | SV40LPA | T. guttatus | |
| | castaneum | | | | | |
| TCARZ_GeFo | T. | CBh_Bsi | GFP | SV40LPA | G. fortis | |
| | castaneum | | | | | |
| TCARZ_Cher_Ge | T. | CBh_Bsi | mCherry | SV40LPA | G. fortis | |
| Fo | castaneum | | | | | |
| HDVgu5_GeFo | none | CBh_Bsi | GFP | SV40LPA | G. fortis | |
| HDVgu5b_GeFo | none | CBh_Bsi | GFP | SV40LPA | G. fortis | |
| HDVgu5c_GeFo | none | CBh_Bsi | GFP | SV40LPA | G. fortis | |
| HDVgu5d_GeFo | none | CBh_Bsi | GFP | SV40LPA | G. fortis | |
| HDVac11_GeFo | none | CBh_Bsi | GFP | SV40LPA | G. fortis | |
| HDVac11b_GeFo | none | CBh_Bsi | GFP | SV40LPA | G. fortis | |
| HDVac12_GeFo | none | CBh_Bsi | GFP | SV40LPA | G. fortis | |
| HDVac12b_GeFo | none | CBh_Bsi | GFP | SV40LPA | G. fortis | |
| etc. | | | | | | |

### EXAMPLE 10. Candidate Protein Screening for Reverse Transcription Activity

Candidate R2-family retroelement proteins screened for reverse transcription (See Table 3) were prepared as in Example 3 and tested for reverse transcription activity as in Example 5. Some TPRT or RT proteins were detected as active in only a subset of assays (indicated as Low/None).

TABLE 3
Candidate Proteins for Reverse Transcriptase Activity

| | | | FIG. | |
|---|---|---|---|---|
| SEQ | | Species | 10 | |
| ID | | Reference | Lane | |
| NO. | Species Derived From | Code | # | RT Activity |
| 47 | Drosophila mercatorum | DrMerc | 15 | None |
| 57 | Lepidurus couesii | LeCoB | 11 | None |
| 55 | Triops cancriformis | TriCan | 12 | None |
| 43 | Ciona intestinalis | Ciln | 3 | None |
| 51 | Gasterosteus aculeatus | GaAc | 19 | None |
| 49 | Drosophila melanogaster | DrMe | 14 | Low/None |
| 45 | Limulus polyphemus | LiPo | 13 | Low/None |
| 53 | Pungitis pungitis | PuPu | 16 | Low |
| 7 | Nasonia vitripennis | NaviB | 9 | High |
| | (lineage B) | | | |
| 9 | Oryzias latipes | OrLa | 8 | Low |
| 18 | Zonotrichia albicollis | ZoAl | 10 | Low/Moderate |
| 27 | Taeniopygia guttata | TaGu | 18 | High |
| 2 | Tribolium castaneum | TriCasB | 5 | High |
| | (lineage B) | | | |
| 25 | Tinamus guttatus | TiGu | 17 | Low |
| 33 | Drosophila simulans | DroSi | 4 | High |
| 36 | Bombyx mori | BoMo | 2 | High |
| 39 | Adineta vaga | AdVa | 7 | Moderate |
| 41 | Hydra magnipapillata | HyMa | 6 | None |
| 31 | Geospiza fortis | GeFo | NS | Low/None |

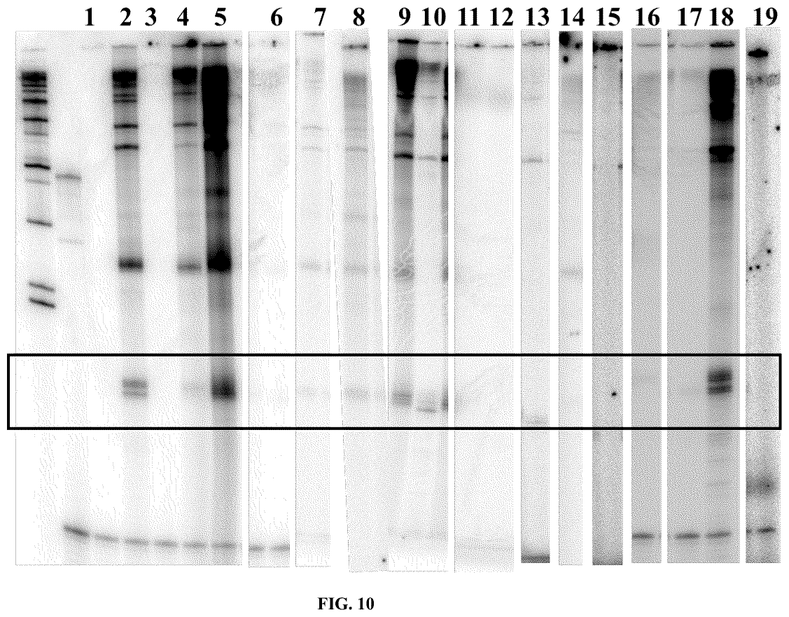

RT activity varied dramatically among species. As seen from the PAGE image results in FIG. 10, initial reverse transcription products of the expected lengths are observed in the dark solid box for candidate RT proteins TriCasB, DroSi, TaGu, NaViB, BoMo, OrLa, AdVa (when normalized to protein expression), ZoAl, LiPo (variably detectable product), PuPu, and TiGu, and GeFo (variably detected product). No reproducible RT products were detected for Ciln, LeCoB, TriCan, DroMer, DroMe, HyMa, and GaAc. Very low activity was sometimes detected for DrMe and GeFo. The opacity of the band at the expected product length, combined with the amount of purified protein detected by immunoblot using antibody against the RT protein FLAG epitope tag, allowed for a comparative estimate of reverse transcription activity levels and sorting the candidate proteins into those with a high, moderate, low, or no (not detectable with assay used) reverse transcription activity as seen in Table 3. In general, candidate proteins TriCasB, DroSi, TaGu, NaViB, and BoMo showed the highest levels of reverse transcriptase ability and are therefore strong candidates for inclusion in an RTC of the invention.

### EXAMPLE 11. In vivo RT assay for 3′ Module specificity

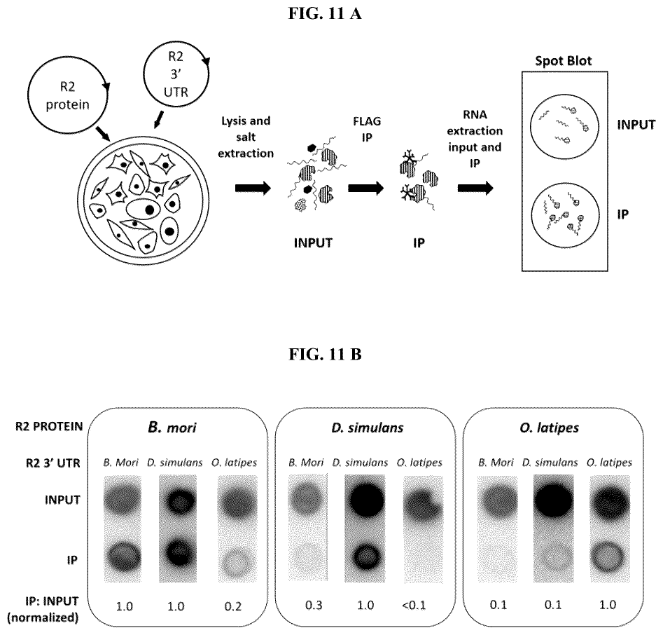

9 populations of HEK293T cells were transfected with different combinations of plasmids comprised of one of the pcDNA3.1 backbone plasmids expressing RT protein ORFs modified from B. mori (SEQ ID NO. 35, D. simulans (SEQ ID NO. 32), and O. latipes (SEQ ID NO. 8), and an additional plasmid expressing the 3′ UTR RNA from B. mori (SEQ ID 163), D. simulans (SEQ ID NO. 164), or O. latipes (SEQ ID NO. 154) R2 elements (see FIG. 11 A). Each RT protein was co-expressed with each 3′ UTR RNA.

After allowing sufficient time for the RT protein plasmids to be transcribed and translated and to associate with the transcribed 3′ UTR RNAs, cells were lysed and any RT protein+RNA template complexes were purified by FLAG immunopurification (Sigma FLAG antibody resin). RNA present in each input cell lysate and RNA associated with each immunopurified sample was purified. Equivalent aliquots of each input RNA sample and each RT-bound RNA sample were affixed to Hybond N+membrane (Cytiva) in a grid of spots. Membranes containing spots for each type of 3′ UTR RNA were probed together for the presence of the 3′ UTR RNA, as detected by hybridization to complementary oligonucleotide probes that were ³²P 5′-end-radiolabeled using T4 polynucleotide kinase (NEB). In other words, samples from cells expressing B. mori R2 3′ UTR were probed for the B. mori 3′ UTR sequence (B. mori 3UTR probes were CATCATGGATTAGGATCGGAAGACCCCCG, (SEQ ID NO. 335); GTACGCCGGCGAAATTGGATCAGTAGATG (SEQ ID NO. 336), and GAGAAACAGACGGGCCTGATCTACACCC) (SEQ ID NO. 337). Samples expressing D. simulans R2 3′ UTR RNA were probed for the D. simulans 3′ UTR sequence (D. simulans 3′UTR probes were CTATCTGAACCGAAGTTCCGCAACGCCTACGTAC (SEQ ID NO. 338), CACTGCGTGTGGTCAGTTTTCCTAGCATGCACG (SEQ ID NO. 339), and GATGTTATGCCAAGACAGCAAGCAAATGTTTTGAACCAAACG) (SEQ ID NO. 340). Samples expressing O. latipes R2 3′ UTR RNA were probed for the O. latipes 3′ UTR sequence (O. latipes 3′UTR probes were TTGAGGCGAGTCACCACTCGCTTTCCGG (SEQ ID NO. 341), and GTGTCCGTCACGGGGACGACATCCGAGTG) (SEQ ID NO. 342).

As can be seen in FIG. 11 B, modified B. mori RT protein binds its cognate 3′ UTR but also the 3′ UTR sequences of D. simulans and O. latipes R2 elements, whereas modified D. simulans and O. latipes proteins have more selectivity. B. mori RT has what findings described here show to be relatively indiscriminate RNA interaction in human cells.

### EXAMPLE 12. In Vitro TPRT Specificity of B. mori, D. simulans and O. latipes RTs

RT proteins from B. mori (SEQ ID NO. 36), D. simulans (SEQ ID NO. 33), and O. latipes (SEQ ID NO. 9) were prepared as in Example 3. GICs comprising a GIC: RT recognition sequence derived from O. latipes 3′UTR (SEQ ID NO. 154) with or without a 3′-appended 4 nt sequence of rRNA (SEQ ID 208) “R4” and GIC: RT recognition sequence derived from D. simulans 3′UTR (SEQ ID NO. 164) with or without a 3′-appended 4 nt sequence of rRNA (SEQ ID 208) “R4” were prepared as in Example 1.

An in vitro TPRT assay was performed as in Example 6 to test each RTs ability to utilize each GIC.

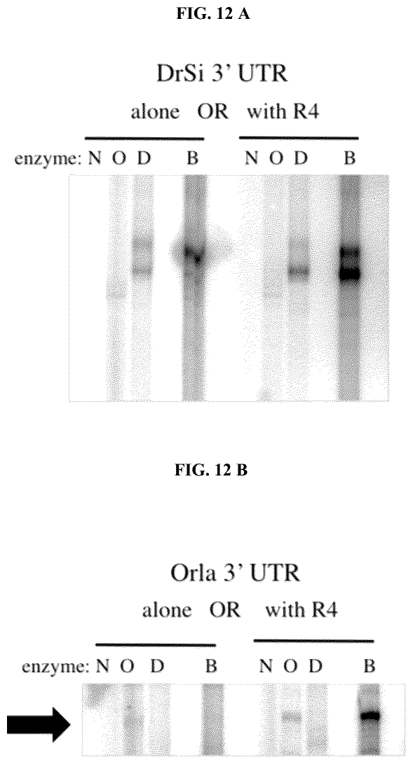

RT proteins derived from D. simulans did not use a GIC comprising the GIC: RT recognition sequence derived from O. latipes 3′ UTR and RT proteins derived from O. latipes RT did not use a GIC comprising the GIC: RT recognition sequence derived from D. simulans 3′UTR for TPRT. RT proteins derived from B. mori, however could use both for TPRT (FIG. 12).

B. mori RT protein had indiscriminate template copying during TPRT (i.e., it was not selective for its homologous GIC), in contrast to other modified R2 RT proteins. For example, the RTs derived from O. latipes or D. simulans were selective for their homologous GIC: RT recognition sequence, and therefore may be preferable when designing a more selective GIS.

### EXAMPLE 13. Phylogenetic Screening for RT Specificity

RT proteins derived from various species retroelements and GICs including GIC: RT recognition sequences derived from various species native retroelement 3′ UTR as outlined in Table 4 were prepared as in Examples 3 and 1 respectively. For this in vitro TRPT comparison all GIC: RT recognition sequences had 3′-appended “R4” 4 nt sequence of rRNA (SEQ ID 208) and if necessary had 5′-appended guanosine(s) for T7 RNAP transcription initiation

An in vitro TPRT assay was performed as in Example 6 to test the ability of each RT to recognize a given GIC: RT recognition sequence. The opacity of the band on the denaturing PAGE gel at the expected product length allowed for a comparative estimate of target primed reverse transcription activity levels and sorting the candidate proteins into those with a high, moderate, low, or no (nondetectable with assay) target primed reverse transcription activity

The results of the TPRT assays were summarized in Table 4 as follows. Each data row was labeled with the RT protein used including the source organism from which the RT sequence was derived. Each data column was labeled with the GIC used including the source organism from which the GIC: RT recognition sequence was derived. Cells with a minus sign (−) indicate that no product of the expected length was observed for the combination of a given RT and GIC. Cells with a plus and minus sign (+/−) signify that a barely detectable amount of product of the expected length was observed in at least some assays. Cells with a single plus sign (+) signify that a low amount product of the expected length was observed, two plus signs (++) indicate that a moderate amount of product of the expected length was observed, and three plus signs (+++) indicate that a high amount of product of the expected length was observed.

RT proteins derived from Taeniopygia guttata, Oryzias latipes, Zonotrichia albicollis, Tinamus guttatus, Tribolium castaneum (R2 lineage B), and Drosophila simulans were more selective for GICs including their homologous GIC: RT recognition sequence than RT protein derived from Bombyx mori. Therefore, RT proteins derived from T. guttata, O. latipes, Z. albicollis, T. guttatus, T. castaneum and/or D. simulans may be preferable for inclusion in a GIS of the invention over B. mori derived RT proteins in order to minimize or prevent insertion of unintended template sequences into a subject genome.

Further, RT protein derived from Z. albicollis, T. guttata and/or T. guttatus were highly specific for GIC: RT recognition sequences derived from among species of birds. Therefore, RT proteins derived from Z. albicollis, T. guttata and/or T. guttatus may be preferential for inclusion in a GIS of the invention, as they may prevent insertion of unintended template sequences into a subject genome while allowing flexibility to engineer the 3′ module.

TABLE 4
RT Specificity

| | | GIC: 3′ Module RT Recognition Sequence (Derived from Indicated Source) | | | | | | | | | |
|---|---|---|---|---|---|---|---|---|---|---|---|
| SEQ | | 171 | 166 | 167 | 168 | 165 | 169 | 162 | 161 | 160 | 158 |
| ID | Soure | DrMerc- | LeCoB- | TriCan- | Ciln- | GaAc- | DrMe- | LiPo- | PuPu- | NaviB- | GeFo- |
| NO. | Code | GIC | GIC | GIC | GIC | GIC | GIC | GIC | GIC | GIC | GIC |
| 7 | NaviB- | − | − | − | − | − | − | + | + | ++ | + |
| | RT | | | | | | | | | | |
| 9 | OrLa- | − | + | − | − | − | − | +/− | + | +/− | + |
| | RT | | | | | | | | | | |
| 18 | ZoAl- | − | − | − | − | − | − | − | − | − | ++ |
| | RT | | | | | | | | | | |
| 27 | TaGu- | − | − | − | − | − | − | − | − | − | +++ |
| | RT | | | | | | | | | | |
| 2 | TriCasB- | +/− | − | − | − | − | +/− | +/− | ++ | ++ | ++ |
| | RT | | | | | | | | | | |
| 25 | TiGu- | ND | ND | ND | ND | ND | ND | ND | ND | ND | + |
| | RT | | | | | | | | | | |
| 33 | DroSi- | +/− | ND | ND | +/− | ND | +/− | ND | ND | ND | ND |
| | RT | | | | | | | | | | |
| 36 | BoMo- | + | + | + | − | − | ++ | ++ | ++ | ++ | ++ |
| | RT | | | | | | | | | | |

RT Specificity

| | | GIC: 3′ Module RT Recognition Sequence (Derived from Indicated Source) | | | | | | | | |
|---|---|---|---|---|---|---|---|---|---|---|
| SEQ | | 154 | 156 | 157 | 155 | 159 | 164 | 163 | 172 | 173 |
| ID | Soure | OrLa- | ZoAl- | TaGu- | TriCasB- | TiGu- | DroSi- | BoMo- | AdVa- | HyMa- |
| NO. | Code | GIC | GIC | GIC | GIC | GIC | GIC | GIC | GIC | GIC |
| 7 | NaviB- | +/− | ++ | + | +++ | ++ | +/− | +/− | +/− | − |
| | RT | | | | | | | | | |
| 9 | OrLa- | ++ | + | + | +/− | ++ | − | + | +/− | − |
| | RT | | | | | | | | | |
| 18 | ZoAl- | − | + | ++ | − | ++ | − | − | − | − |
| | RT | | | | | | | | | |
| 27 | TaGu- | − | +++ | +++ | − | +++ | − | − | − | − |
| | RT | | | | | | | | | |
| 2 | TriCasB- | ++ | ++ | ++ | ++ | ++ | + | + | +/− | − |
| | RT | | | | | | | | | |
| 25 | TiGu- | − | +/− | +/− | − | + | ND | ND | ND | ND |
| | RT | | | | | | | | | |
| 33 | DroSi- | +/− | ND | ND | + | ND | ++ | + | + | ND |
| | RT | | | | | | | | | |
| 36 | BoMo- | ++ | ++ | ++ | ++ | ++ | +++ | +++ | ++ | +/− |
| | RT | | | | | | | | | |

### EXAMPLE 14. Effect of 3′ Module Engineering on B. mori Derived RT TPRT

RT protein derived from B. mori (SEQ ID NO 36) were prepared as in Example 3. GICs containing the sequence of BoMo 3′ UTR (SEQ ID 163) with 5′ and/or 3′ flanking sequences described in Table 5 were prepared as in Example 1.

TABLE 5
B. mori Derived GICs

| | | RE | 3′ | |
|---|---|---|---|---|
| | GIC: 5′ | Derived | Subject | A |
| Template | rRNA | Sequences | rRNA | Tract |
| Reference | Length | Source | Length | Length |
| GG*-BM3UTR-R3 | 0 nt | B. Mori | 3 nt (SEQ | 0 nt |
| | | | ID 214) | |
| R26_ BM3UTR | 26 nt (SEQ | B. Mori | 0 nt | 0 nt |
| | ID 183) | | | |
| GG*_BM3UTR_R4 | 0 nt | B. Mori | 4 nt (SEQ | 0 nt |
| | | | ID 208) | |
| GGG*- | 4 nt (SEQ | B. Mori | 4 nt (SEQ | 0 nt |
| R4_BM3UTR_R4 | ID 204) | | ID 208) | |
| R26_BM3UTR_R4 | 26 nt (SEQ | B. Mori | 4 nt (SEQ | 0 nt |
| | ID 183) | | ID 208) | |
| R26_BM3UTR_R4_PA | 26 nt (SEQ | B. Mori | 4 nt (SEQ | 22 nt |
| | ID 183) | | ID 208) | |
| R26_BM3UTR_R20 | 26 nt (SEQ | B. Mori | 20 nt (SEQ | 0 nt |
| | ID 183) | | ID 213) | |

*indicates 5′ guanosines added for T7 RNAP transcription initiation

In vitro TPRT assay was performed as described in Example 6, with B. mori derived RT protein combined separately with each template and a 64 or 84 bp target site DNA duplex (SEQ IDs 219 and 220 respectively). Arrow marks region of expected TPRT product length for expected 3′ junction formation.

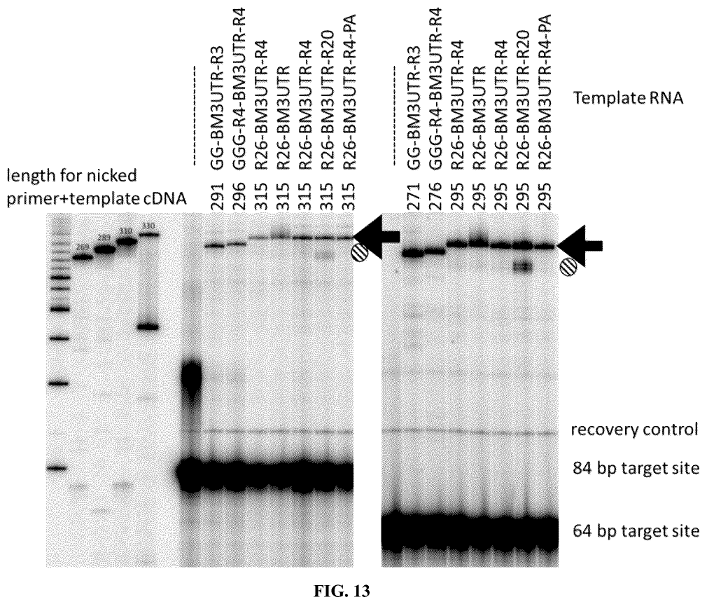

As seen FIG. 13, sequence extension from the 3′ end of B. mori 3′UTR RNA does not greatly influence efficiency of target primed reverse transcription (TPRT) by B. mori RT. In particular, no 3′-flanking rRNA was necessary on the template for TPRT. 3′ addition of 4 nt of rRNA increased the homogeneity of TPRT product length but did not increase the actual TPRT product length as would be expected if the entire template RNA was copied into cDNA. Instead, the extra 4 nt of template length may base-pair with nicked target-site primer in order to initiate cDNA synthesis.

Increase in length of 3′ rRNA to 20 nt reduces 3′ junction fidelity by enabling internal initiation (circle marked position) compared to the higher precision of intended TPRT synthesis using template RNA with only 4 nt of 3′ rRNA (arrow marks region of high-fidelity 3′ junction formation). Therefore a 20 nt 3′-flanking rRNA sequence was unfavorable relative to a 4 nt 3′-flanking rRNA sequence. Of note, 3′-flanking rRNA could be extended by an at least 22 nt tract of adenosine (PA) without loss of efficiency or precision of correct product synthesis.

### EXAMPLE 15. Effect of 3′ Module Engineering on TPRT Efficiency of O. latipes Derived RT

RT protein derived from O. latipes (SEQ ID NO 9) were prepared as in Example 3. GICs containing the sequence of OrLa 3′ UTR (SEQ ID 154) with 5′ and/or 3′ flanking sequences described in Table 6 were prepared as in Example 16.

TABLE 6
O. latipes Derived GICs

| | | | GIC: 3′ | GIC: 3′ |
|---|---|---|---|---|
| | | | Module | Module |
| | GIC: 5′ | RE | rRNA | A-Tract |
| Template | rRNA | Derived | Sequence | Sequence |
| Reference | Length | Regions | Length | Length |
| R26_OL | 26 nt (SEQ | O. latipes | 0 nt | 0 nt |
| | ID 183) | | | |
| R4_OL_R4 | 4 nt (SEQ | O. latipes | 4 nt (SEQ | 0 nt |
| | ID 204) | | ID 208) | |
| R26_OL_R4 | 26 nt (SEQ | O. latipes | 4 nt (SEQ | 0 nt |
| | ID 183) | | ID 208) | |
| R26_OL_R20 | 26 nt (SEQ | O. latipes | 20 nt (SEQ | 0 nt |
| | ID 183) | | ID 213) | |
| R26_OL_R4_PA | 26 nt (SEQ | O. latipes | 4 nt (SEQ | 22 nt |
| | ID 183) | | ID 208) | |
| GG*-R0-OL3-R0 | 0 nt | O. latipes | 0 nt | 0 nt |
| GG*-R0-OL3-R4 | 0 nt | O. latipes | 4 nt (SEQ | 0 nt |
| | | | ID 208) | |
| GG*-R0-OL3-R8 | 0 nt | O. latipes | 8 nt (SEQ | 0 nt |
| | | | ID 215) | |
| GG*-R0-OL3-R12 | 0 nt | O. latipes | 12 nt (SEQ | 0 nt |
| | | | ID 216) | |
| GG*-R0-OL3-R16 | 0 nt | O. latipes | 16 nt (SEQ | 0 nt |
| | | | ID 217) | |
| GG*-R0-OL3-R20 | 0 nt | O. latipes | 20 nt (SEQ | 0 nt |
| | | | ID 213) | |

*indicates 5′ guanosine(s) added for T7 RNAP transcription initiation

In vitro TPRT assay was performed as described in Example 6, with O. latipes derived RT protein combined separately with each template. Product formation indicates that O. latipes derived RT is biochemically active for TPRT.

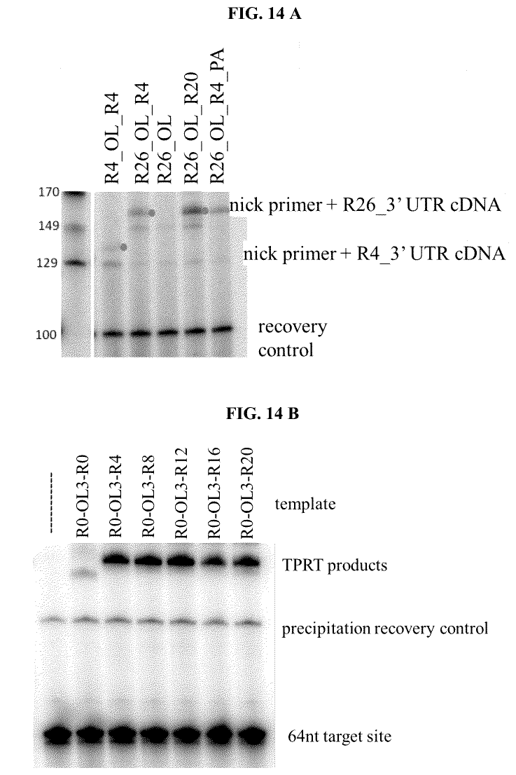

As seen in FIG. 14(A), O. latipes 3′ UTR lacking a 3′ extension of rRNA was not efficiently used for TPRT by O. latipes RT, unlike results in FIG. 13 demonstrating B. mori RT use of B. mori 3′ UTR RNA for efficient TPRT without 3′-flanking rRNA. In common with B. mori components, 3′-flanking rRNA could be extended by an at least 22 nt tract of polyadenosine (PA) without inhibition of O. latipes RT TPRT and with increased homogeneity of product length.

A second set of TPRT assays were conducted to systematically examine the effect of different 3′ subject rRNA lengths.

As seen in FIG. 14(B), these results confirm those observed above. The lack of a 3′ rRNA extension resulted in both poor activity and improper internal initiation by the O. latipes RT, and the presence of 4 nt of rRNA was sufficient to stimulate TPRT and improve 3′ junction precision. Therefore, it may be preferential to include only 4 nt of 3′ subject rRNA in the GIC 3′ module rRNA sequence in GICs of the invention. The increasing length of GIC 3′ rRNA sequence does not correspondingly increase the length of TPRT product, indicating that the GIC 3′ rRNA sequence is not copied; instead it can base-pair with nicked target-site primer DNA in order to initiate cDNA synthesis.

### EXAMPLE 16. Effect of 3′ Module Engineering on TPRT Efficiency of T. castaneum Derived RT

RT protein from T. castaneum prepared as in Example 3 (SEQ ID NO. 2). GICs containing the sequence of TriCasB 3′ UTR (SEQ ID 155) with 5′ and/or 3′ flanking sequences described in Table 7 were prepared as in Example 1.

TABLE 7
T. castaneum Derived GICs

| | | | GIC: 3′ | GIC: 3′ |
|---|---|---|---|---|
| | GIC: 5′ | | Module | Module |
| | rRNA | RE | rRNA | A-Tract |
| Template | Sequence | Derived | Sequence | Sequence |
| Reference | Length | Regions | Length | Length |
| R25-TC_UTR- | 25 nt (SEQ | T. castaneum | 4 nt (SEQ | 0 nt |
| R4 | ID 205) | | ID 208) | |
| R25-TC_UTR- | 25 nt (SEQ | T. castaneum | 4 nt (SEQ | 22 nt |
| R4_PA | ID 205) | | ID 208) | |
| R25-TC_UTR- | 25 nt (SEQ | T. castaneum | 10 nt (SEQ | 0 nt |
| R10 | ID 205) | | ID 208) | |

In vitro TPRT assay was performed as described in Example 6, with T. castaneum derived RT protein combined separately with each template. Arrow indicates the position of the intended TPRT products. Target site DNA is detected as the dark band at the bottom of the image. Product formation indicates that T. castaneum derived RT is biochemically active for TPRT.

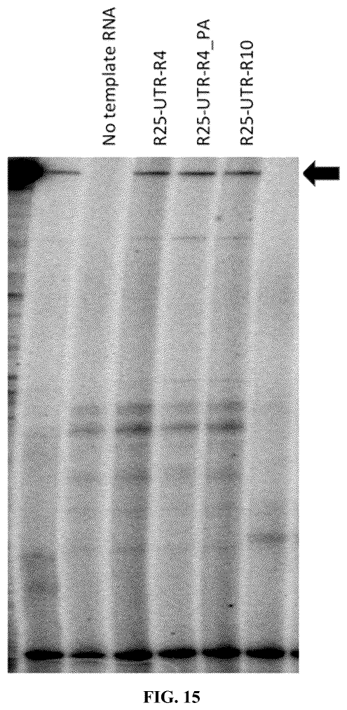

As can be seen in FIG. 15, no improvement in product synthesis was discernable by addition of more than 4 nt of the GIC: 3′ module rRNA sequence, and 3′-flanking rRNA could be extended by an at least 22 nt tract of polyadenosine (PA) without inhibition of correct product synthesis.

### EXAMPLE 17. Effect of 3′ Module Engineering on TPRT Efficiency of Z. albicollis and T. guttata Derived RTs

RT protein derived from Z. albicollis (SEQ ID NO 18) was prepared as in Example 3. GICs containing the 3′ module RT recognition sequence of Z. albicollis (ZoAl) 3′ UTR (SEQ ID 156) or T. guttatus (TiGu) 3′ UTR (SEQ ID 159) or T. guttata (TaGu) 3′ UTR (SEQ ID 157) with 5′ and/or 3′ flanking sequences described in Table 8 were prepared as in Example 1.

TABLE 8
Bird R2 GICs

| | | | GIC: 3′ | GIC: 3′ | |
|---|---|---|---|---|---|
| | FIG. | GIC: 5′ | Module RT | Module | |
| | 16 | IRNA | Recognition | rRNA | GIC: 3′ |
| Template | Lane | Sequence | Sequence | Sequence | Module A- |
| Reference | # | Length | Source | Length | TractLength |
| R26(-28)- | 1 | 26 nt (SEQ | Z. albicollis | 0 nt | 0 nt |
| ZA3-R0 | | ID 183) | | | |
| R26(-28)- | 2 | 26 nt (SEQ | Z. albicollis | 4 nt (SEQ | 0 nt |
| ZA3-R4 | | ID 183) | | ID 208) | |
| R26(-28)- | 3 | 26 nt (SEQ | Z. albicollis | 20 nt (SEQ | 0 nt |
| ZA3-R20 | | ID 183) | | ID 213) | |
| R26(-28)- | 4 | 26 nt (SEQ | Z. albicollis | 4 nt (SEQ | 22 nt |
| ZA3-R4PA | | ID 183) | | ID 208) | |
| R26(-28)- | 5 | 26 nt (SEQ | T. guttatus | 0 nt | 0 nt |
| TiG3-R0 | | ID 183) | | | |
| Product | 6 | | | | |
| Lost | | | | | |
| R26(-28)- | 7 | 26 nt (SEQ | T. guttatus | 20 nt (SEQ | 0 nt |
| TiG3-R20 | | ID 183) | | ID 213) | |
| R26(-28)- | 8 | 26 nt (SEQ | T. guttatus | 4 nt (SEQ | 22 nt |
| TiG3-R4PA | | ID 183) | | ID 208) | |
| R28(-28)- | 9 | 28 nt (SEQ | T. guttata | 0 nt | 0 nt |
| TaG3-R0 | | ID 181) | | | |
| R28(-28)- | 10 | 28 nt (SEQ | T. guttata | 4 nt (SEQ | 0 nt |
| TaG3-R4 | | ID 181) | | ID 208) | |
| R28(-28)- | 11 | 28 nt (SEQ | T. guttata | 20 nt (SEQ | 0 nt |
| TaG3-R20 | | ID 181) | | ID 213) | |
| R28(-28)- | 12 | 28 nt (SEQ | T. guttata | 4 nt (SEQ | 22 nt |
| TaG3-R4PA | | ID 181) | | ID 208) | |

In vitro TPRT assay was performed as described in Example 6, with Z. albicollis derived RT protein combined separately with each template. Box with solid line encloses TPRT products, box with dashed line encloses the precipitation recovery control, and box with mixed dash and dot outline encloses the 64 bp target site DNA. These results demonstrate that Z. albicollis derived RT is biochemically active for target primed reverse transcription.

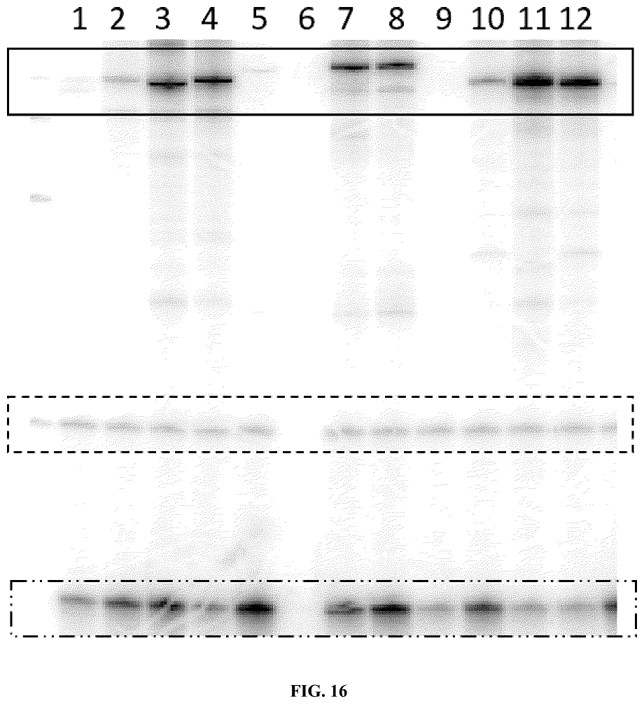

As can be seen in FIG. 16, Z. albicollis derived RT proteins do not efficiently utilize a GIC with a 3′ module design lacking a GIC: 3′ module rRNA sequence, therefore showing increased efficiency of cDNA synthesis at a target site with which GIC 3′ rRNA sequence can base-pair. The increase in length of GIC 3′ rRNA sequence does not increase the length of TPRT product, indicating that the GIC 3′ rRNA sequence is not copied; it must base-pair with nicked target-site primer in order to initiate cDNA synthesis. The highest amount of TPRT product synthesis was produced with a GIC including either 4 nt 3′ rRNA sequence with A-tract 22 nt tail or with 20 nt rRNA sequence. Finally, Z. albicollis derived RT proteins were able to utilize GICs containing GIC: 3′ module RT recognition sequence derived from several bird species tested. Parallel experiments were performed with RT protein derived from T. guttata (SEQ ID 27), with the result that the T. guttata derived bird RT protein could utilize GICs containing GIC: 3′ module RT recognition sequence derived from several bird species and was selective in its utilization of GICs containing GIC: 3′ rRNA sequences.

These results further support that a GIS may include RT proteins derived from Z. albicollis or T. guttata combined with GIC: 3′ module RT recognition sequences derived from various bird species, with GIC: 3′ module rRNA sequence with or without GIC: 3′ module A-Tract sequence, to alter the TPRT reaction efficiency. Without the capability of GIC: 3′ module rRNA sequence to base-pair to the nicked target-site primer, no cDNA synthesis was observed. If the target site sequence downstream of the nick that can base-pair with GIC: 3′ module rRNA was altered to a different sequence (mutant target site; SEQ ID 224), cleavage was still observed but TPRT was blocked by the failure of base-pairing of the GIC: 3′ module rRNA to the primer strand 3′ end. Therefore, only with a nick at the correct sequence of target site, generating a primer 3′ end matched to the GIC: 3′ module rRNA sequence, is TPRT productive for cDNA synthesis. Using the mutant target site, if the GIC: 3′ module rRNA sequence was changed to the sequence that would base-pair with the primer 3′ end created by the nick, cDNA synthesis by TRPT was rescued. This demonstrates that the mechanism of function of GIC: 3′ module RNA sequence is to base-pair with the 3′ terminus region of the primer strand.

### EXAMPLE 18. Effect of GIC: 3′ Module Tail Engineering on Insertion of a Transgene into the Human Genome in Vivo

Part A: T. guttata Derived RTC: RT-Module

RTC mRNA derived from T. guttata (SEQ ID NO 28) was produced as in Example 4. GIC RNAs that include a GFP transgene expression cassette payload and have the same GIC: 5′ module and GIC: 3′ module RT recognition sequence (TCA5_CBhBsi_GFP_GeFo3) were produced as in Example 2 and are enumerated in Table 9.

hTERT RPE-1 cells were co-transfected with an RTC and the indicated GIC (1:1 molar ratio) using Lipofectamine Messenger Max then harvested after 24 hours. The percent of GFP positive cells in each treatment was determined by FACS analysis with results reported in Table 9.

TABLE 9
3′ module tail Engineering Effects in Vivo

| GIC: 3′module | GIC: 3′ module | | Percent GFP |
|---|---|---|---|
| rRNA Length | A-Tract Length | GIC SEQ | Positive |
| (nt) | (nt) | ID NO | Cells |
| 0 | 0 | 297 | 0.12 |
| 0 | 22 | 298 | 0.17 |
| 4 | 0 | 299 | 4.05 |
| 4 | 22 | 300 | 15.67 |
| 20 | 0 | 301 | 6.84 |
| 20 | 22 | 302 | 4.23 |

These results showed that utilizing a GIC: 3′ module comprising 4 nt of GIC: 3′ module rRNA sequence and a 22 nt A-Tract sequence resulted in significantly greater rates of transgene insertion than other combinations tested. It is worth noting that other combinations that included at least 4 nt of GIC: 3′ module rRNA sequence did result in successful insertion and expression of a transgene in a mammalian cell line. However, with 20 nt of GIC: 3′ module rRNA sequence, a 22 nt length of A-Tract sequence was inhibitory.

### Part B: Comparison of T. Guttata and Z. Albicollis Derived RTC: RT-Modules

RTC mRNA derived from T. guttata (SEQ ID NO 28) or Z. albicollis (SEQ ID NO 19) was produced as in Example 4. GIC RNAs that include a GFP transgene expression cassette payload and the same GIC: 5′ module and GIC: 3′ module RT recognition sequence (TCA5_CBhBsi_GFP_GeFo3) were produced as in Example 2 as enumerated in Table 10.

hTERT RPE-1 cells were co-transfected with an RTC and the indicated GIC (molar ratio 1:3) using Lipofectamine Messenger Max then harvested after 24 hours. The percent of GFP positive cells and median intensity of GFP expression in GFP-positive cells was determined for each treatment by FACS analysis as shown in Table 10.

TABLE 10
Additional 3′ module tail Engineering Effects in Vivo

| | | | | | GFP |
|---|---|---|---|---|---|
| | | | | | Intensity |
| | | | | | (relative |
| | GIC: | GIC: 3′ | | Percent | units of |
| RTC: RT- | 3′module | module A- | | GFP | fluorescence |
| Module | rRNA | Tract | | Positive | above |
| Source | Length | Length | GIC SEQ | Cells | GIC-alone |
| Organism | (nt) | (nt) | ID NO | (%) | background) |
| T. guttata | 0 | 0 | 297 | 0.093 | 1705 |
| T. guttata | 0 | 22 | 298 | 0.17 | 2098 |
| T. guttata | 4 | 0 | 299 | 2.84 | 4570 |
| T. guttata | 4 | 22 | 300 | 14.708 | 9011 |
| T. guttata | 20 | 0 | 301 | 5.342 | 5003 |
| T. guttata | 20 | 22 | 302 | 2.235 | 3835 |
| Z. albicollis | 0 | 0 | 297 | 0 | 0 |
| Z. albicollis | 0 | 22 | 298 | 0.25 | 2183 |
| Z. albicollis | 4 | 0 | 299 | 3.83 | 4260 |
| Z. albicollis | 4 | 22 | 300 | 13.608 | 7364 |
| Z. albicollis | 20 | 0 | 301 | 4.972 | 4315 |
| Z. albicollis | 20 | 22 | 302 | 2.075 | 3145 |

These results corroborated those seen in Part A for an RTC mRNA derived from T. guttata. Further, they showed that an RTC mRNA derived from Z. albicollis showed the same pattern of efficiency regarding GIC: 3′ module rRNA sequence and A-Tract length as an RTC mRNA derived from T. guttata. The Z. albicollis derived RTC: RT-module was only slightly less efficient at transgene insertion than the T. guttata derived RTC: RT-module using the optimal R4A22 template.

Both T. guttata and Z. albicollis derived RTC: RT-modules were viable components of a GIS of the invention. Both showed the ability to utilize a GIC with variable lengths of GIC: 3′ module rRNA and/or GIC: 3′ module A-Tract, with a potentially optimal GIC composition including a GIC: 3′module rRNA sequence length of about 4 nt and a GIC: 3′ module A-Tract sequence length of about 22 nt.

### EXAMPLE 19. Effect of 3′ Module Engineering on TPRT Efficiency of T. guttata Derived RT

RT protein derived from T. guttata (SEQ ID NO 27) was prepared as in Example 3. GICs containing different GIC: 3′ module RT recognition sequence with or without 5′ guanosine(s) added for T7 RNAP transcription initiation and with GIC: 3′ module rRNA sequence R4 (SEQ ID 208) were prepared as in Example 1 as described in Table 11.

TABLE 11
T. guttata RT Specificity for GIC:
3′ module RT recognition equence

| | | | GIC: 3′ | |
|---|---|---|---|---|
| | FIG. | | Module | |
| | 17 | GIC: 3′ Module RT | rRNA | SEQ |
| | Lane | Recognition Sequence | Sequence | ID |
| Template Reference | # | Source and SEQ ID (#) | Length | NO. |
| No template control | 2 | NA | NA | NA |
| GGG*-HM3-R4 | 3 | H. magnipapillata | 4 nt | |
| | | (219) | | |
| GGG*-AV3-R4 | 4 | A. vaga (218) | 4 nt | |
| G*-LP3-R4 | 5 | L. polyphemus (208) | 4 nt | |
| G*-ZA3-R4 | 6 | Z. albicollis (202) | 4 nt | |
| G*-TiG3-R4 | 7 | T. guttatus (205) | 4 nt | |
| G*-TaG3-R4 | 8 | T. guttata (203) | 4 nt | |
| G*-GF3-R4 | 9 | G. fortis (204) | 4 nt | |
| GA3-R4 | 10 | G. aculeatus (211) | 4 nt | |
| OL3-R4 | 11 | O. latipes (200) | 4 nt | |
| G*-PP3-R4 | 12 | P. pungitis (207) | 4 nt | |
| GGG*-TCasB3-R4 | 13 | T. castaneum (201) | 4 nt | |
| G*-NVB3-R4 | 14 | N. vitripennis (206) | 4 nt | |
| GGG*-CI3-R4 | 15 | C. intestinalis (214) | 4 nt | |
| BM3-R4 | 16 | B. mori (209) | 4 nt | |
| G*-LCB3-R4 | 17 | L. couesii (212) | 4 nt | |
| G*-TCan3-R4 | 18 | T. cancriformis (213) | 4 nt | |
| G*-DS3-5iA-R4 | 19 | D. simulans (210) | 4 nt | |
| GG*-DMer3-R4 | 20 | D. mercatorum (217) | 4 nt | |
| G*-DMel3-5iA-R4 | 21 | D. melanogaster (215) | 4 nt | |
| GG*-DN3-R4 | 22 | D. nasuta (216) | 4 nt | |

*indicates 5′ guanosine(s) added for T7 RNAP transcription initiation

In vitro TPRT assay was performed as described in Example 6, with T. guttata derived RT protein combined separately with each template. Template sequences were comprised of retroelement 3′ UTR sequences with 5′ guanosine(s) added if necessary to support T7 RNAP transcription, and with GIC: 3′ module rRNA sequence length of 4 nt and no GIC: 3′ module A-Tract rRNA sequence. Box with solid line encloses the expected TPRT products, box with dashed line encloses the precipitation recovery control, and box with mixed dash and dot outline encloses the remaining intact 64 bp target site DNA.

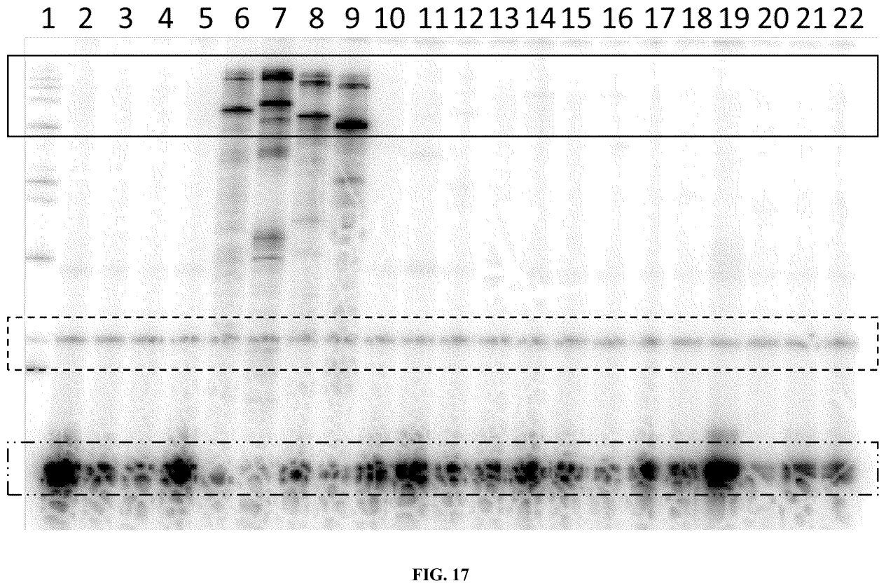

As shown in FIG. 17 RT protein derived from T. guttata was able to recognize GIC's with GIC: 3′ module RT recognition sequences derived from various bird species with very little to no TPRT activity observed in the presence of GICs that included GIC: 3′ module RT recognition sequences from non-bird species. Further, high TPRT activity was observed with the combination of a T. guttata derived RT protein and a G. fortis derived GIC with the shortest tested bird GIC: 3′ module RT recognition sequence.

Therefore, it may be preferential to design at least one GIS of the invention to include at least one RTC: RT-module comprising or encoding at least one T. guttata derived RT protein and at least one GIC comprising or encoding at least one G. fortis derived GIC: 3′ module RT recognition sequence, particularly to be administered to a non-bird subject. This combination may allow for a GIS that is both highly efficient at inserting its payload sequence into a subject genome and highly specific for its GIC.

### EXAMPLE 20. Effect of 5′ and 3′ Module Engineering on Efficiency of T. castaneum RT Insertion of a Transgene into the Human Genome in Vivo

293T cells were transfected with plasmid as in Example 3 to express a protein modified from one of the three lineages of T. castaneum R2, with a synthetic-sequence ORF presenting a single AUG start codon for translation (SEQ ID NO. 1). Some cells were not transfected with plasmid in parallel as a negative control. After 48 hours, these cells were transfected using lipofectamine3000 with a purified GIC RNA prepared as in Example 1 in the combinations described in Table 12. Genomic DNA was purified from transfected cells 1 day after the second transfection.

TABLE 12
T. castaneum Derived GICs

| | GIC: 5′ | GIC: 5′ | GIC: 3′ | GIC: 3′ | GIC: 3′ | |
|---|---|---|---|---|---|---|
| | Module | Module | Module RT | Module | Module | |
| | rRNA | RE | Recognition | rRNA | A-Tract | |
| | Sequence | Sequence | Sequence | Sequence | Sequence | SEQ ID |
| Template Reference | Length* | Source** | Source | Length | Length | NO. |
| R25-TCB3-R4 | 25 nt | NA | T. | 4 nt | 0 nt | 254 |
| | | | castaneum | | | |
| R25-TCB3-R10 | 25 nt | NA | T. | 10 nt | 0 nt | 255 |
| | | | castaneum | | | |
| R25-TCB3-R4-PA | 25 nt | NA | T. | 4 nt | 22 nt | 256 |
| | | | castaneum | | | |
| R25*-TCB5_TCB3-R4 | 25 nt* | T. | T. | 4 nt | 0 nt | 257 |
| | | castaneum | castaneum | | | |
| R25*-TCB5_TCB3-R10 | 25 nt* | T. | T. | 10 nt | 0 nt | 258 |
| | | castaneum | castaneum | | | |
| R25*-TCB5_TCB3-R4- | 25 nt* | T. | T. | 4 nt | 22 nt | 259 |
| PA | | castaneum | castaneum | | | |
| R25*-TCB5_TCB3-PA | 25 nt* | T. | T. | 0 nt | 22 nt | 260 |
| | | castaneum | castaneum | | | |
| R25*-TCB5_TCB3- | 25 nt* | T. | T. | 10 nt | 22 nt | 261 |
| R10PA | | castaneum | castaneum | | | |

The 3′ 13 of 25 nt of rRNA are contained within the GIC: 5′ Module and will remain after self-cleavage. The 5′ 12 nt will be removed.
**TriCasB 5′ module sequences are modified from the native to include 13 nt of rRNA upstream of the target-site first nick that match the human genome, rather than the shorter native length of rRNA and the evolutionarily altered rRNA sequence.

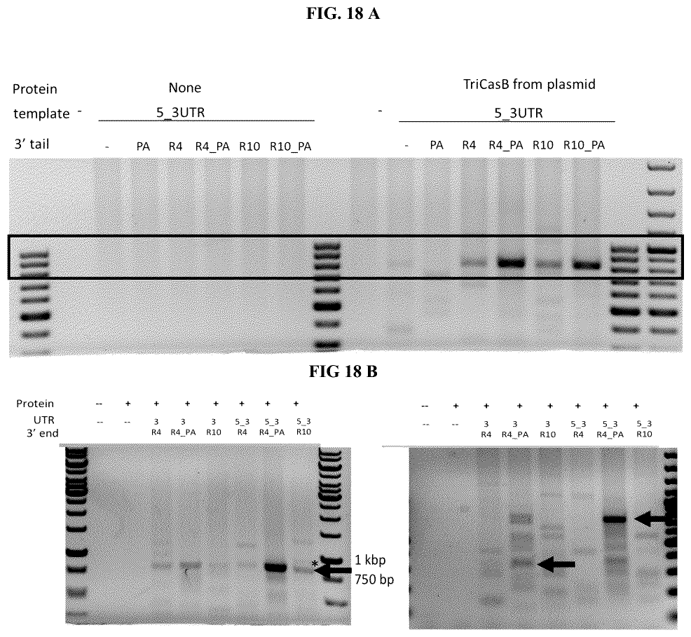

In one experiment evidenced by FIG. 18A, GICs had both T. castaneum R2 lineage B 5′ module and T. castaneum R2 lineage B 3′ module (“5_3UTR”) and differed in the GIC: 3′ module rRNA length (0, R4 or R10) and presence or absence of GIC: 3′ module 22 nt A-Tract (PA). PCR was performed to detect transgene insertion 3′ junctions using a consistent amount of genomic DNA from different cell populations (Forward Primer: CTCCTGACCAACTAGCTCACTGACTAATTTTAAAC (SEQ ID NO: 343)) and Reverse Primer: CCACTTATTCTACACCTCTCATGTCTCTTCACCG (SEQ ID NO: 344)). PCR product DNA was resolved on a non-denaturing agarose gel and detected with ethidium bromide. Junction PCR products of the size expected for the intended 3′ junction were most abundant in cells transfected with GIC: 3′ module 22 nt A-Tract (PA), especially with GIC: 3′ module rRNA length of 4 nt. A GIC: 3′ module A-Tract without GIC: 3′ module rRNA was not sufficient for detectable transgene insertion, which is favorable in excluding adenosine-tailed human host cell mRNAs as potential templates for transgene synthesis.

In a separate experiment evidenced by FIG. 18B, GICs had T. castaneum R2 lineage B 3′ module with or without T. castaneum R2 lineage B 5′ module (“53” or “3”, respectively). GICs also differed in the GIC: 3′ module rRNA length (R4 or R10) and/or presence or absence of GIC: 3′ module A-Tract (PA). PCR was performed to detect transgene insertion 3′ and 5′ junctions using a consistent amount of genomic DNA from different cell populations using 3′ insertion junction primers (Forward Primer: CTCCTGACCAACTAGCTCACTGACTAATTTTAAAC (SEQ ID NO: 343) and Reverse Primer: CCACTTATTCTACACCTCTCATGTCTCTTCACCG (SEQ ID NO: 344) or 5′ insertion junction primers (Forward Primer: CTAGCAGCCGACTTAGAACTGGTGCGG (SEQ ID NO: 345) and Reverse Primer: CTTCGTCTTCGGAATCCATGTCCATAGC (SEQ ID NO: 346)). PCR product DNA was resolved on a non-denaturing agarose gel run in 1× TAE and detected with ethidium bromide and imaged on the BioRad molecular imager ChemiDoc XRS+.

In the left panel, PCR products of the size expected for the perfect 3′ junction, indicated with an arrow, were most abundant in cells transfected with GIC: 3′ module rRNA length of 4 nt and GIC: 3′ module A-Tract (PA). Also, the presence of the T. castaneum R2 lineage B 5′ module had increased 3′ junction product indicative of more inserted transgene. Minimal if any incorrectly sized PCR products were detected for R4_PA GICs, indicating high fidelity of 3′ junction formation. However, cells transfected with other GICs had additional 3′ junction PCR products.

In the right panel, PCR products of the size expected for the 5′ junction of a full-length transgene were different size for GICs with or without the 5′ module, in each case are indicated with an arrow. The PCR product for 5′ junction of a full-length transgene insertion was most abundant in cells transfected with GIC: 3′ module rRNA length of 4 nt and GIC: 3′ module A-Tract (PA). Also, the presence of the T. castaneum R2 lineage B 5′ module increased 5′ junction product amount and homogeneity despite the longer 5′ junction PCR product length (which would bias towards less efficient PCR), indicative of more inserted transgene and higher insertion fidelity.

Both 5′ and 3′ junction formation were detectable only when both RT protein expression and RNA template transfection occurred. Cells that expressed RT protein without template RNA or were transfected with template RNA without RT protein expression showed no or minor non-specific PCR products.

These results showed that shorter lengths of GIC: 3′ module rRNA sequence, such as 4 nt long sequences, may provide a GIS of the invention with superior TPRT activity, including higher reaction yields and more specific transgene junction formation (both 5′ and 3′ junctions).

### EXAMPLE 21. Effect of 5′ Module RZ Engineering on Efficiency of T. castaneum RT Insertion of a Transgene into the Human Genome in Vivo

293T cells were transfected to express a T. castaneum derived RT protein (SEQ ID 1) as in Example 3. Subsequently, these cells were transfected using Lipofectamine3000 with a GIC RNA prepared as in Example 1 in the combinations described in Table 13. All GIC constructs included a GIC: 3′ module RT recognition sequence derived from T. castaneum, a GIC: 3′ module rRNA sequence length of 4 nt, and a GIC: 3′ module A-Tract sequence length of 22 nt (SEQ ID 262). GIC constructs differed in the GIC: 5′ module.

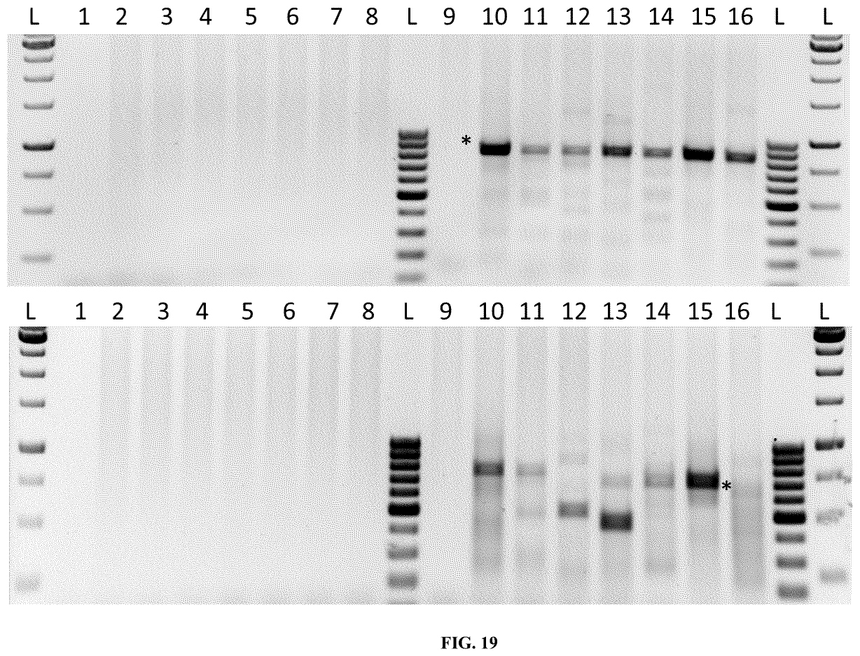

TABLE 13
T. castaneum Derived GICs with Alternate RZs

| | GIC: 5′ Module | | GIC: 5′ Module | GIC: 5′ | |
|---|---|---|---|---|---|
| | rRNA | | RZ Sequence | Module RE | |
| | Sequence | FIG. 19 | Source / | Sequence | SEQ |
| Template Reference | Length** | Lane #s | Modification | Source | ID NO. |
| TriCasB_5 (SEQ ID 62) | 13 (SEQ ID | 2 & 10 | T. castaneum/ | T. | |
| | 195) | | None extra* | castaneum | |
| TriCasB_5rzdead (SEQ | 13 (SEQ ID | 3 & 11 | T. castaneum/ | T. | |
| ID 63) | 195) | | Inactivated | castaneum | |
| TriCasB_5RZ (SEQ ID | 13 (SEQ ID | 4 & 12 | T. castaneum/ | T. | |
| 64) | 195) | | None extra* | castaneum | |
| TriCasB_5RZmin (SEQ | 13 (SEQ ID | 5 & 13 | T. castaneum/ | T. | |
| ID 65) | 195) | | Shortened 5RZ | castaneum | |
| TriCasB_5RZmin + down | 13 (SEQ ID | 6 & 14 | T. castaneum/ | T. | |
| (SEQ ID 144) | 195) | | Shortened 5RZ | castaneum | |
| | | | replaced for | | |
| | | | native RZ region | | |
| | | | of TriCasB 5 | | |
| OrLa_5L (SEQ ID 60) | 26 (SEQ ID | 7 & 15 | O. latipes/ | O. latipes | |
| | 183) | | None | | |
| DroSi_5 (SEQ ID 70) | 0 | 8 & 16 | D. simulans/ | D. simulans | |
| | | | None | | |

*TriCasB 5′ module sequences are modified from the native to include 13 nt of rRNA upstream of the target-site first nick that match the human genome, rather than the shorter native length of rRNA and the evolutionarily altered rRNA sequence.
**5′ rRNA length after self-cleavage

2 separate PCR amplifications of genomic DNA from the transfected cell pool were used to detect a 3′ insertion junction (top panel) and a 5′ insertion junction (bottom panel) as in Example 20. PCR PRIMERS: 3′ junction:

Forward Primer:
(SEQ ID NO: 343)
CTCCTGACCAACTAGCTCACTGACTAATTTTAAAC,
Reverse Primer:
(SEQ ID NO: 344)
CCACTTATTCTACACCTCTCATGTCTCTTCACCG;
5′ junction:
Forward Primer:
(SEQ ID NO: 347)
CCAGGGGAATCCGACTGTTTAATTAAAACAAAGC,
Reverse Primer:
(SEQ ID NO: 348)
GCGACTCGCATCACTGACTTTAATTGGTTG.

As observed in FIG. 19 GIC with 5′ module components derived from T. castaneum lineage B or O. latipes R2 retroelements supported the most transgene insertion and junction fidelity, evidenced by a predominant single PCR product of the expected length for full-length transgene insertion with precise 3′ and 5′ junction formation. A single nt change in the T. castaneum lineage B 5′ module RZ active site that killed RZ activity (TriCasB_5rzdead) severely reduced transgene insertion efficiency and compromised insertion fidelity. Also, GIC including the full length of the T. castaneum GIC: 5′ module RE sequence (TriCasB_5) produced superior transgene insertion relative to a GIC that contained only the T. castaneum derived RZ region of the full 5′ module sequence (TriCasB_5RZ). However, a GIC with a length-minimized version of the T. castaneum RZ alone (TriCasB_5RZmin) performed comparably to GIC “TriCasB_5,” better than “TriCasB_5RZ,” and better than “TriCasB_5RZmin+down” that has added-back sequence from the T. castaneum 5′UTR downstream of the RZ that was removed from “TriCasB_5” to make “TriCasB_5RZ.”

Finally, although a GIC including O. latipes 5′ module components (OrLa_5L) performed as well as “TriCasB_5” when combined with a T. castaneum derived RT protein, with GIC: 3′ module components derived from T. castaneum, this was not the case for D. simulans 5′ module components (DroSi_5). The D. simulans 5′ module RZ self-cleavage activity removes all sequence in the initial GIC transcript that is 5′ of the 5′ UTR, including any 5′ rRNA. Without 5′ rRNA protected within the self-cleaving RZ, initial first-strand cDNA synthesis could still occur but second-strand synthesis necessary for 5′ junction formation and stable transgene insertion had reduced efficiency and precision relative to GIC with “TriCasB_5” or “OrLa_5L”. This was evident from the smeared distribution of lengths of 5′ PCR junction products (FIG. 19, bottom panel land 16).

### EXAMPLE 22: GIC: 5′ Module rRNA Lengths

RTC mRNA for F-ZoAl RT (SEQ ID NO 19) was produced as in Example 4. GIC RNAs including a GFP transgene expression cassette (SEQ ID 303, CBhBsi_GPF_GeFo_R4A22), differing only in the sequence of the 5′ module, were produced as in Example 2. De novo designed GIC: 5′ module sequences optimized to adopt a self-cleaving HDV RZ fold were developed that enforced a self-cleaved GIC 5′ end to be at a specific position of rRNA sequence upstream of the target-site nick, for example at position −28 (HDV-28) or at position −13 (HDV-13) or at another position permissive for the +1 guanosine requirement and empirically validated to result in T7 RNAP transcript self-cleavage.

Further, de novo designed GIC: 5′ module sequences optimized to adopt a self-cleaving HDV RZ fold were tailored by amount of rRNA sequence present in the GIC: 5′ module given each position of self-cleavage. For example, a GIC: 5′ module that induced self-cleavage at position −28 relative to the TPRT nick could contain 28 nt of 5′ rRNA or, by trimming the rRNA sequence from its 3′ boundary, could contain another length of rRNA such as 25, 26, or 27 nt.

hTERT RPE-1 cells were co-transfected with an RTC mRNA and the indicated GIC RNA, mixed at 1:3 molar ratio, using Lipofectamine Messenger Max. Transfected cell pools were analyzed by flow cytometry to detect % GFP+cells after 24 hours. The percent of GFP positive cells was determined by FACS analysis as reported in Table 14.

TABLE 14
Effects of GIC: 5′ Module rRNA Sequence Length

| | GIC: 5′ | | | | Normalized |
|---|---|---|---|---|---|
| | Module | GIC: 5′ | | | GFP+ % |
| | rRNA | Module | | Percent | cells |
| | Starting | rRNA | RZ self- | GFP | per self- |
| | Sequence | Sequence | cleavage | Positive | cleaved |
| GIC: 5′ Module RZ Sequence ID | Position | Length | efficiency | Cells | GIC |
| HDV-28(26)gu1 (SEQ ID 106) | −28 | 26 | 76 | 12.6 | 17 |
| HDV-28(26)ac2 (SEQ ID 108) | −28 | 26 | 58 | 10.3 | 18 |
| HDV-28(28)ac2b (SEQ ID 112) | −28 | 28 | 57 | 9.5 | 17 |
| HDV-28(27)ac2c (SEQ ID 113) | −28 | 27 | 59 | 9.2 | 16 |
| HDV-28(25)ac2d (SEQ ID 114) | −28 | 25 | 56 | 10.9 | 19 |
| HDV-13(13)ac11 (SEQ ID 115) | −13 | 13 | ~100 | 2.7 | 2.7 |
| HDV-13(11)ac11b (SEQ ID 117) | −13 | 11 | ~100 | 4.9 | 4.9 |

Results reveal several themes for successful transgene insertion. First, designed RZ are highly efficient relative to native RZ for the purpose of transgene insertion. Second, for any given RZ cleavage site in 5′-flanking rRNA sequence (e.g., −28 or −13), the length of GIC: 5′ rRNA sequence has an influence that can improve transgene insertion by including less than maximal rather than maximal rRNA sequence (for example, compare within the “ac2” series of RZ backbone sequence ac2 with 26 or 25 nt rRNA (normalized % 18 or 19 GFP+for ac2 and ac2d respectively) to ac2 with 28 or 27 nt of rRNA sequence (normalized % 17 or 16 GFP+for ac2b and ac2c respectively). Third, the upstream site of RZ cleavage influences transgene insertion efficiency (for example, 5′ modules of HDV-13 RZ are inferior to 5′ modules of HDV-28 RZ in transgene insertion efficiency when matched for rRNA sequence extending to the bottom-strand nick, in HDV-28(28) or HDV-13(13), or when improved in efficiency by leaving a gap between 5′ module rRNA and the bottom-strand nick site, in HDV-28(26) or HDV-13(11).

### EXAMPLE 23. GIC: 5′ Module Engineering

RTC mRNA for F-ZoAl RT (SEQ ID NO 19) was produced as in Example 4. GIC RNAs including a GFP transgene expression cassette (SEQ ID 303, CBhBsi_GPF_GeFo_R4A22), differing only in the sequence of the 5′ module, were produced as in Example 2 as enumerated in Table 20.

hTERT RPE-1 cells were co-transfected with an RTC mRNA and the indicated GIC RNA, mixed at 1:3 molar ratio, using Lipofectamine Messenger Max. Transfected cell pools were analyzed by FACS to detect % GFP+cells after 24 hours. The percent of GFP+cells in each treatment was determined by FACS analysis as shown in Table 15.

TABLE 15
Engineered GIC: 5′ Module Components

| | T7 RNAP | GIC: 5′ | GIC: 5′ RZ | |
|---|---|---|---|---|
| | transcript | Module | self- | Percent |
| | 5′ leader | rRNA | cleavage | GFP |
| | before | Sequence | efficiency | Positive |
| 5′ Module | RZ* | Length | (%) | Cells |
| HDV-28(26)gu1 | PP7hp | 26 nt | 60 | 3.2 |
| (SEQ ID 106) | | | | |
| HDV-28(28)gu5b | PP7hp | 28 nt | 80 | 4.4 |
| (SEQ ID 120) | | | | |
| HDV-28(28)NL | none | 28 nt | 0 | 1.8 |
| (SEQ ID 120) | | | | |
| HDV-28(28)_rzdead | PP7hp | 28 nt | 0 | 0.44 |
| (SEQ ID 125) | | | | |
| -28(28) (No RZ) | none | 28 nt | 0 | 0.022 |
| (SEQ ID 181) | | | | |
| TCARZ-28(28) | PP7hp | 28 nt | 87 | 3.9 |
| (SEQ ID 67) | | | | |
| TCA5-28(28) | PP7hp | 28 nt | 89 | 3.2 |
| (SEQ ID 62) | | | | |
| TCA5_rzdead | PP7hp | 28 nt | 0 | 0.29 |
| (SEQ ID 63) | | | | |

*PP7hp indicates the presence of a hairpin stem-loop of the consensus sequence for binding to phage PP7 coat proteins

Results supported several conclusions. First, presence of upstream rRNA in the template RNA did not support efficient transgene insertion without its inclusion in an efficiently folding RZ (compare 5′ module “−28(28) (No RZ)” to any RZ-active 5′ module such as TCA5 or TCARZ or de novo designed HDV-28 variant). Second, at least some of the self-cleaving 5′ module RZ-fold sequences support higher transgene insertion efficiency if the T7 RNAP transcript has a 5′ leader sequence to promote RZ self-cleavage (compare transgene insertion efficiency for HDV-28(28)NL (no leader) to the same sequence of RZ-cleaved template RNA produced with the presence of PP7 phage hairpin leader sequence in HDV-28(28)gu5b). Third, optimal transgene insertion efficiency by a 5′ module with RZ and leader sequence requires a catalytically active RZ (compare rzdead to RZ-active 5′ module versions).

### EXAMPLE 24. 2-RNA Component GIS: 5′ Junction Fidelity

RTC mRNA RTCs were prepared as in Example 4. GIC RNA was prepared as in Example 2 as described in Table 16.

TABLE 16
GICs for 2-RNA component 5′ Junction Assays

| | | Lane | | | GIC |
|---|---|---|---|---|---|
| | | Symbol | 5′ Module | 3′ Module | SEQ |
| | | in FIG. | Source | Source | ID |
| RTC mRNA | GIC Identifier | 20 | Organism | Organism | NO. |
| None | | A | | | |
| O. latipes (SEQ ID 10) | TCA5_OrLa3 | B | T. castaneum | O. latipes | 263 |
| Z. albicollis (SEQ ID 19) | TCA5_ZoAl3 | C | T. castaneum | Z. albicollis | 264 |
| T. castaneum (SEQ ID 3) | TCA5_TCB3 | D | T. castaneum | T. castaneum | 265 |
| T. castaneum untag (SEQ ID 5) | TCA5_TCB3 | E | T. castaneum | T. castaneum | 265 |
| None | | F | | | |
| O. latipes (SEQ ID 10) | OrLa5L_OrLa3 | G | O. latipes | O. latipes | 266 |
| Z. albicollis (SEQ ID 19) | OrLa5L_ZoAl3 | H | O. latipes | Z. albicollis | 267 |
| T. castaneum (SEQ ID 3) | OrLa5L_TCB3 | I | O. latipes | T. castaneum | 268 |
| T. castaneum untag (SEQ ID 5) | OrLa5L_TCB3 | J | O. latipes | T. castaneum | 268 |

All RNAs were prepared in a final buffer of 1 mM sodium citrate, pH 6.5. Per well of a 6-well plate, total RNA amount was fixed at 2.5 ug. If spike-in mRNA for a fluorescent protein was included as a transfection efficiency control (mCherry mRNA from Trilink with 100% 5moU instead of U), 50 ng of this mRNA was added to the mixed RTC mRNA and GIC RNA.

293T cells were transfected with RTC mRNA and GIC RNA largely as described in Example 7 except using Lipofectamine3000 rather than MessengerMax and using a 1:1 molar ratio of RTC:GIC. Each RTC mRNA was transfected with either the GIC RNA construct comprising (i) a 5′ module derived from T. castaneum lineage A or O. latipes and, (ii) a 3′ module derived from the same species as the RT protein and if relevant the same retroelement lineage of species (e.g., T. castaneum R2 lineage B components TriCasB RT is paired with TriCasB 3′UTR “TCB”, distinct from the T. castaneum R2 lineage A 5′ module “TCA5”).

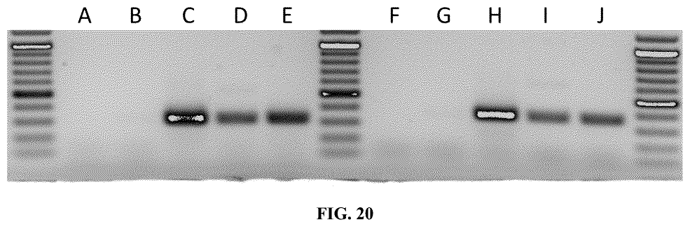

After 24 hours, to extract genomic DNA cell pellets were lysed using 200 ul denaturing RIPA buffer (150 mM NaCl, 50 mM Tris pH 7.5, 1 mM EDTA, 1% TX-100, 0.5% Na Deoxycholate, 0.1% SDS, and 1 mM DTT). 10 ul RNase A was added and the sample was incubated at 37° C. for 30 min. Then 5 ul Proteinase K was added and the sample was incubated at 50° C. overnight. An equal volume of PCI solution (phenol:chloroform:isoamyl alcohol 25:24:1) was added. After vertexing and a 5-min spin, the aqueous layer was extracted. One ul of glycogen (20 ug/ul), 10% volume of 5 M sodium chloride, and 3 volumes of 100% ethanol were added. After mixing and 30 min incubation at −20° C., the sample was centrifuged at 4° C. for 30 min. The genomic DNA pellet was washed in 70% ethanol three times. After air drying, the pellet was dissolved in TE buffer. 500 ng genomic DNA was used for PCR assays of insertion junctions. After PCR, 6 ul of loading dye was mixed with 25 ul of PCR reaction and half of the mixture was loaded into wells of 1.2% agarose gel in 1× TAE buffer with ethidium bromide. After electrophoresis the gel was imaged on the BioRad molecular imager ChemiDoc XRS+ as seen in FIG. 20.

The analysis above indicates that two RNA component GIS systems can insert a full-length transgene at the intended target site of the human genome.

Utilizing an expressed RT protein derived from Z. albicollis and corresponding GIC: 3′ module RT recognition sequence produced more PCR product of the expected size than systems utilizing expressed RT protein and GIC: 3′ module RT recognition sequence derived from O. latipes or T. castaneum lineage B, and Those using an expressed RT protein and corresponding GIC: 3′ module RT recognition sequence derived from T. castaneum lineage B produced more PCR product of the expected size than systems utilizing expressed RT proteins and GIC: 3′ module RT recognition sequence derived from O. latipes.

The comparison of each RTC using GIC with each of two GIC: 5′ module components indicates that both “OrLa5L” from the O. latipes R2 5′ region and “TCA5” from the T. castaneum R2 lineage A 5′ region enable full-length transgene insertions. This outcome was unchanged these GIC: 5′ modules were paired with any GIC: 3′ module RT recognition sequence tested. This Example demonstrates RNA-only delivery of a GIS.

### EXAMPLE 25. 2-RNA GIS Delivery in Multiple Cell Lines

RTC mRNA for F-ZoAl RT (SEQ ID NO 19) was produced as in Example 4. GIC RNAs including a GFP transgene expression cassette (TCA5_CBh_NLSGPF_ZoA13_R4A22 or TCA5_CBh_NLSGPF_GeFo3_R4A22, SEQ IDs 304 and 305 respectively) were produced as in Example 2 as described in Table 17.

SK-HEP1, 293T, HCT116, hTERT RPE-1, HeLa, Huh7, IMR-90, and HaCaT human cell lines, as well as Cos7 and Vero monkey cell lines and C2C12 mouse cell line, were cultured and co-transfected as in Example 7 with RTC mRNA mixed with GIC RNA at a 1:3 molar ratio of mRNA: template RNA. After 24 hours, transfected cell pools were analyzed by flow cytometry to detect % GFP+cells with results given in Table 17A&B.

TABLE 17
Cell type panel of transgene insertion via 2-RNA delivery GIS

| | | | hTERT | | HCT | |
|---|---|---|---|---|---|---|
| | SK-HEP1 | 293T | RPE-1 | HeLa | 116 | Huh7 |
| GIC: 3′ ZoAl | 1.15% | 0.19% | 2.12% | 0.26% | 0.36% | 1.02% |

TABLE 17B
Additional cell type panel of transgene insertion via 2-RNA
delivery GIS

| | | | hTERT | | | |
|---|---|---|---|---|---|---|
| | IMR-90 | HaCaT | RPE-1 | C2C12 | Cos7 | Vero |
| GIC: 3′ GeFo | 0.52% | 3.26% | 2.59% | 2.77% | 1.08% | 0.52% |

All populations showed at least some percent of cells expressing GFP, indicating that both combinations of RTC and GIC were at least minimally effective at inserting an GFP expression transgene into the subject genomes. Further, relatively high percentage of GFP+cells were observed in the hTERT RPE-1 primary human cell line compared to human cancer-derived cell lines such as HeLa or 293T.

Additional experiments were performed that demonstrate 2-RNA GIS Delivery in Multiple Cell Lines. RTC mRNA encoding F-ZoAl RT (made with N1methylpseudouridine) was separately co-transfected with two different GIS RNA templates: i) 5′ TCA5_RNAPJterml_sylacO_CBh promoter_eGFP_SV40LPA_sylacO_GeFo3_R4A22, comprised of regular uridine nucleotides, or ii) 5′ TCARZ_CMV*promoter_eGFP_minpA_GeFo3_R4A22, comprising a modified CMV promoter for expression of the transgene RNA and comprising pseudoU nucleotides. Expression of the transgene was determined by flow cytometry at day 1 (or day 1 and day 3) following 2-RNA delivery. mRNA encoding mCherry (TniLink) was co-transfected as a way to compare overall transfection efficiency relative to % cells GFP+. The results are shown in Tables 17C and 17D below.

TABLE 17C
Additional cell type panel of transgene insertion via 2-RNA delivery
GIS using RNA template 5′ TCA5_RNAPIterm1_sylacO_CBhpro-
moter_eGFP_SV40LPA_sylacO_GeFo3_R4A22 comprising
regular uridine nucleotides.

| | Day 1 | | Day 3 |
|---|---|---|---|
| Cell lines | GFP % | mCherry % | GFP % |
| RPEhTERT | 20.71 | 92.657 | 18.89 |
| ARPE19 | 19.64 | 91.32 | 17.1 |
| 293T | 0.21 | 90.866 | 2.34 |
| HaCat | 3.74 | 84.801 | 2.47 |
| Hela | 0.92 | 62.78 | 0.77 |
| Huh7 | 0 | 98.12 | 11.68 |
| IMR90 | 6.07 | 75.12 | 8.51 |
| MRC5 | 5.42 | 82.99 | 5.7 |
| Cos7 | 3.41 | 95.18 | 4.66 |
| Vero | 1.91 | 91.938 | 2.38 |
| C2C12 | 9.26 | 96.98 | 5.69 |
| G8 | 1.9 | 84.338 | 1.04 |
| C26 | 1.03 | 77.744 | 1.26 |

TABLE 17D
Additional cell type panel of transgene insertion
via 2-RNA delivery GIS using RNA template 5′
TCARZ_CMV*promoter_eGFP_minpA_GeFo3_R4A22,
comprising a modified CMV promoter for expression
of the transgene RNA and comprising pseudoU nucleotides.

| | | | GFP | | |
|---|---|---|---|---|---|
| Cell | GFP % | S.D. | median | mCherry % | S.D. |
| lines | Mean | GFP | intensity | Mean | mCherry |
| RPE | 61.31 | 0.8386 | 44537 | 90.61333 | 0.1528 |
| ARPE-19 | 53.57 | 0.3512 | 61436 | 90.57333 | 0.2517 |
| 293T | 9.567 | 0.2003 | 3511 | 74.125 | 0.1732 |
| Hela | 11.52 | 0.1 | 5827 | 51.47667 | 0.6506 |
| IMR90 | 38.01 | 0.0577 | 27271 | 66.22 | 0.755 |
| MRC5 | 40.52 | 0.1155 | 28822 | 71.65333 | 0.5859 |
| Vero | 10.06 | 0.3 | 4071 | 83.20333 | 0.6429 |
| C2C12 | 30.53 | 1.701 | 5560 | 78.00667 | 1.5044 |

SD = standard deviation.

The data above demonstrates that 2-RNA delivery works in multiple cell types from humans, monkeys, and mice. The data also demonstrates that the combination of modified CMV promoter and pseudoU nucleotides increases the percentage of cells that express the transgene.

### EXAMPLE 26. RTC and GIC Combinations

[0756]hTERT RPE-1 cell lines were cultured and transfected with one of either ZoAl RT mRNA, ZoAl RT-dead mRNA, or TaGu RT mRNA RTC (SEQ IDs 19, 24 and 28 respectively) and one of TCA5_ZoAl3, TCA5_GeFo3, or TCA5_TaGu3 GICs RNA (SEQ IDs 306, 300, 307 respectively) as described in Example 9 at an RTC to GIC ratio of 1:3.

After 5 days populations were harvested and counted as previously described and the percent of GFP positive cells and median intensity for GFP positive populations was determined and reported in Table 18.

TABLE 18
RTC and GIC Combinations

| | | Percent GFP |
|---|---|---|
| RTC | GIC | Positive Cells (%) |
| F-ZoAl RT mRNA | TCA5_TaGu3 | 2.38 |
| F-TaGu RT mRNA | TCA5_TaGu3 | 3.56 |
| F-ZoAl RT mRNA | TCA5_ZoAl3 | 11.75 |
| F-TaGu RT mRNA | TCA5_ZoAl3 | 13.28 |
| F-ZoAl RT mRNA | TCA5_GeFo3 | 11.71 |
| F-TaGu RT mRNA | TCA5_GeFo3 | 13.87 |

Any combination of the administered RTCs (ZoAl RT mRNA or TaGu RT mRNA) with GICs TCA5_ZoA13 or TCA5_GeFo3 resulted in a significantly higher percent of cells expressing GFP. This indicated that a GIC with 3′ module RT recognition sequence derived from either Z. albicollis or G. fortis is preferable to pair with an RTC: RT-module derived from Z. albicollis or T. guttata in order to achieve a higher percentage of transgene insertion. Further, all combinations did result in a stable insertion (as determined by PCR to detect 5′ and 3′ junction insertion sites) and transgene expression. ZoAl RT-dead mRNA in combination with any GIC construct did not result in GFP flourescence above background.

### EXAMPLE 27. RTC to GIC Ratios by Cell Line and 3′ Module

### Part A

hTERT RPE-1, SK-HEP1, and HeLa human cell lines were cultured and transfected with ZoAl RT mRNA RTC and either TCA5_ZoA13 or TCA5_GeFo3 GICs RNA as described above.

After 5 days populations were harvested and counted as previously described. Table 19 shows the percent (%) of cells that expressed eGFP.

TABLE 19
RTC to GIC Ratios

| Cell | | Ratio RTC to GIC | | | | | |
|---|---|---|---|---|---|---|---|
| Line | GIC | No RTC | 1:1 | 1:3 | 1:5 | 1:8 | 1:10 |
| hTERT | TCA5_ | 0.01% | 2.47% | 2.8% | 2.63% | N.A. | 2.3% |
| RPE-1 | ZoAl3 | | | | | | |
| hTERT | TCA5_ | 0.04% | 1.96% | 2.48% | 2.57% | 2.34% | 2.28% |
| RPE-1 | GeFo3 | | | | | | |
| SK- | TCA5_ | 0.04% | 0.38% | 0.58% | 0.62% | 0.64% | 0.7% |
| HEP1 | ZoAl3 | | | | | | |

### Part B

SK-HEP 1 and HeLa cells lines were cultured, transfected, harvested, and analyzed as above and described in Table 20. Ratios of RTC to GIC were varied as indicated in Table 20.

TABLE 20
RTC to GIC Ratios

| | | Ratio RTC to GIC | | | | | |
|---|---|---|---|---|---|---|---|
| Cell Line | GIC | No RTC | 3:1 | 2:1 | 1:1 | 1:2 | 1:3 |
| SK-HEP 1 | TCA5_ZoAl3 | 0.09 | 1.07 | 1.58 | 2.44 | 3.23 | 3.60 |
| HeLa | TCA5_ZoAl3 | 0.04 | 0.15 | 0.20 | 0.26 | 0.27 | 0.32 |

Table 20B shows the results of similar experiments using hTERT RPE-1 human cells cultured and transfected with F-TaGu mRNA RTC and F-ZoAl mRNA RTC (both made with 5moU) and either TCA5_ZoAl3 or TCA5_GeFo3 GICs RNA as described above.

TABLE 20B
RTC to GIC Ratios

| RTC mRNA/ | | | GFP |
|---|---|---|---|
| GIC RNA | Molar ratio: | GFP % | intensity |
| TaGu/ZoAl3 | 10:1 | 6.27 | 3846 |
| | 3:1 | 11.26 | 5562 |
| | 1:1 | 14.36 | 6412 |
| | 1:3 | 14.36 | 6347 |
| | 1:6 | 14.76 | 6746 |
| | 1:12 | 13.36 | 6156 |
| | 1:20 | 11.26 | 5773 |
| TaGu/GeFo3 | 10:1 | 4.5 | 3405 |
| | 3:1 | 9.1 | 4330 |
| | 1:1 | 12.21 | 4841 |
| | 1:3 | 13.91 | 5146 |
| | 1:6 | 13.31 | 5413 |
| | 1:12 | 13.51 | 5600 |
| | 1:20 | 11.61 | 5323 |
| ZoAl/ZoAl3 | 10:1 | 1.89 | 3014 |
| | 3:1 | 5.35 | 4540 |
| | 1:1 | 9.96 | 5676 |
| | 1:3 | 11.46 | 6347 |
| | 1:6 | 11.06 | 6521 |
| | 1:12 | 9.96 | 5911 |
| | 1:20 | 7.82 | 5487 |
| ZoAl/GeFo3 | 10:1 | 0.77 | 3014 |
| | 3:1 | 4.25 | 3393 |
| | 1:1 | 8.12 | 4330 |
| | 1:3 | 10.31 | 5233 |
| | 1:6 | 10.71 | 5146 |
| | 1:12 | 9.91 | 5146 |
| | 1:20 | 7.84 | 4744 |

The ratio of RTC to GIC that yielded the most effective transgene insertion varied somewhat but was optimal with a molar ratio that had more GIC RNA than RTC RNA.

These results indicated that the ideal ratio for insertion of a transgene by a 2-component GIS to a particular subject may need to be determined through experimentation rather than being predictable from the component or subject identity. For a GIS intended to be administered to hTERT RPE-1 cells that comprises an RTC including a Z. albicollis derived RT-module and a GIC including a Z. albicollis derived GIC: 3′ module RT recognition sequence, a ratio of 1:3 (RTC:GIC) may be preferable. For a GIS intended to be administered to hTERT RPE-1 cells that comprises an RTC including a Z. albicollis derived RTC: RT-module and a GIC including a G. fortis derived GIC: 3′ module RT recognition sequence, a ratio of 1:5 (RTC:GIC) may be preferable. For a GIS intended to be administered to SK-HEP1 or HeLa cells that comprises an RTC including a Z. albicollis derived RTC: RT-module and a GIC including a Z. albicollis derived GIC: RT recognition sequence, a ratio of 1:3 (RTC:GIC) may be preferable.

### EXAMPLE 29: Durability of Transgene Expression

RTC mRNA encoding F-ZoAl RT (SEQ ID NO 19) was produced as in Example 4. GIC RNA including a GFP transgene expression cassette TCA5_CBh_NLSGFP_ZoA13 (SEQ ID NO 304) was produced as in Example 2.

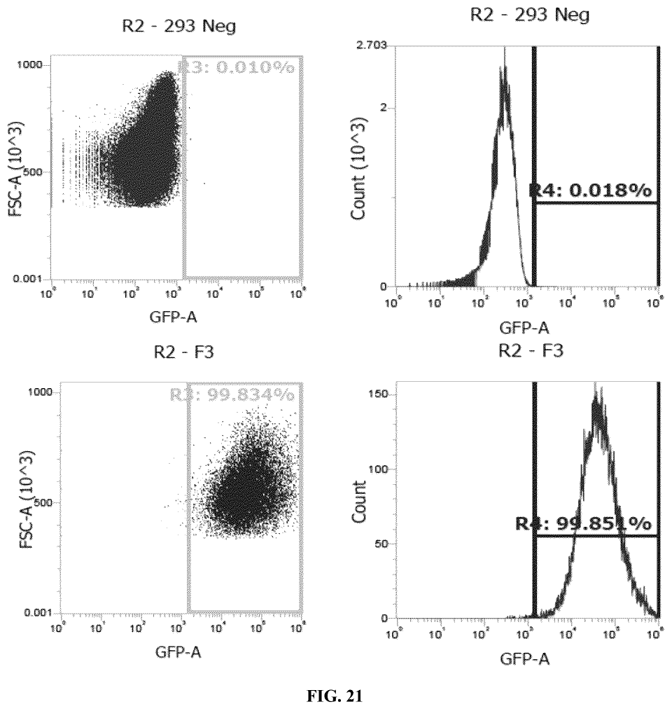

RTC and GIC constructs were co-transfected into 293T cell cultures described in Example 7 and sorted to enrich GFP+cells at day 3 post-transfection, which 1 day later were sorted to separate individual GFP-positive cells into individual wells of 96-well plates using Fusion Aria sorter plate holder. After about 3 weeks of proliferation, the individual wells were screened for viable GFP-positive cell lines, which were then transferred to master 24-well plates and split twice per week. 37 cell lines were considered clonal by having a single peak distribution of GFP fluorescence intensity (FIG. 21); each cell line had different absolute GFP intensity clearly distinguishable from GFP-negative clonal cell lines (FIG. 21). Aliquots of cells were screened using an Attune N×T Acoustic Focusing Cytometer approximately weekly during in continuous culture. Over 2 months of passaging as clonal cell lines, almost 3 months since initial transfection, only one of the 37 showed any decrease in GFP intensity and that was only of −50%.

These results showed that a transgene inserted into a mammalian cell genome by a GIS of the invention could be stably expressed for 3 months or more.

### EXAMPLE 30: Insertion and Expression of Multiple Transgenes

RTC mRNA for F-ZoAl RT (SEQ ID NO 19) was produced as in Example 4. GIC RNAs with a GFP transgene expression cassette (TCA5_CBhBsi_GFP_GeFo3, SEQ ID NO 300) and an mCherry transgene expression cassette (TCA5_CBhBsi_mCherry_GeFo3, SEQ ID NO 308) were produced as in Example 2.

hTERT RPE-1 cells were co-transfected with an RTC mRNA and one of the 2 GIC constructs or an equal mixture of both, with molar ratio of RTC mRNA to total GIC template RNA of 1:3. For controls, some cells were not transfected (negative control), transfected with RTC alone (RTC control), or transfected with GFP or mCherry GIC alone (GFP and mCherry template only controls). Cells were also transfected with RTC and one of three GIC: GFP, mCherry, or an equal mixture of both. After 24 hours, cells were assayed by flow cytometry for GFP and mCherry expression. The percent of cells expressing the intended transgene product was recorded in Table 21.

TABLE 21
Insertion and Expression of 2 Transgenes

| | Percent of | Percent of | Percent of |
|---|---|---|---|
| Components | Cells GFP | Cells mCherry | Cells GFP & |
| Transfected | Positive only | Positive only | mCherry Positive |
| None | 0.0041 | 0.0055 | 0.0014 |
| RTC Only | 0.026 | 0.024 | 0 |
| GFP GIC Only | 0.043 | 0.0020 | 0.0061 |
| mCherry GIC Only | 0.024 | 0.010 | 0.017 |
| RTC + GFP GIC | 21.7 | 0.29 | 0.044 |
| RTC + mCherry GIC | 0.3 | 15.3 | 0.038 |
| RTC + GFP & | 5.43 | 3.54 | 8.94 |
| mCherry GIC | | | |

These results showed that a GIS of the invention may insert more than one transgene comprised in a single GIC into a subject genome such that both transgenes may be expressed by the subject cell. As a corollary, multiple transgenes may be inserted into the genome using a single GIC resulting in a higher level of payload expression by the subject cells. If multiple transgene copies are not desirable, the transgene payload may contain a negative feedback mechanism halting additional transgene insertions after the first, using strategies known to those versed in the art.

Additional experiments were performed where two different GIC template RNAs were mixed together and transfected into cells or a single GIC template RNA encoding two different transgenes was used (referred to as a tandem template). Cells were co-transfected with RTC mRNA encoding F-ZoAl RT (SEQ ID 19, comprising 5moU) or RT catalytic dead ZoAl (“ZoAl RTD” SEQ ID 23), comprising N1methylpseudouridine) and the single transgene templates TCARZ_SV40*_GFP_GeFo3 (SEQ ID NO: 325) and TCARZ_CMV*_mCherry_GeFo3 (SEQ ID NO: 327), or the tandem template TCARZ_SV40*_GFP_minPA_CMV*_mCherry_SV40LPA_GeFo3 (SEQ ID NO: 329). The results are shown in Table 21B below. eGFP+mCherry mRNA is the positive control.

TABLE 21B
Percent of cells positive for mCherry,
eGFP, or both mCherry and eGFP.

| | Percent of | Percent of | Percent of |
|---|---|---|---|
| Components | Cells mCherry | Cells GFP | Cells GFP & |
| Transfected | Positive only | Positive only | mCherry Positive |
| ZoAl RTD + | 0.01 ± 0.004 | 0.01 ± 0.002 | 0.0004 ± 0.0004 |
| CMV-mCherry | | | |
| ZoAl RTD + | 0.06 ± 0.006 | 0.005 ± 0.003 | 0.005 ± 0.002 |
| SV40-eGFP | | | |
| ZoAl RTD + | 0.005 ± 0.002 | 0.03 ± 0.003 | 0.008 ± 0.004 |
| Tandem SV40- | | | |
| eGFP_CMV- | | | |
| mCherry | | | |
| ZoAl + | 69.4 ± 0.88 | 0.02 ± 0.0009 | 0.06 ± 0.006 |
| CMV-mCherry | | | |
| ZoAl + | 0.002 ± 0.0009 | 68.0 ± 0.38 | 0.04 ± 0.005 |
| SV40-eGFP | | | |
| ZoAl + | 7.77 ± 0.54 | 3.3 ± 0.23 | 52.2 ± 2.8 |
| SV40-eGFP + | | | |
| CMV-mCherry | | | |
| ZoAl + | 12.5 ± 0.20 | 0.6 ± 0.02 | 45.6 ± 0.73 |
| Tandem SV40- | | | |
| eGFP_CMV- | | | |
| mCherry | | | |
| eGFP + | 0.4 ± 0.05 | 0.3 ± 0.2 | 97.4 ± 0.2 |
| mCherry mRNA | | | |

Mean ± SEM, n = 3.

The data demonstrates that two different transgene RNAs can be successfully inserted into the same cell, and that two different transgene RNAs can be successfully delivered on the same GIC template RNA.

### EXAMPLE 31: Recruitment of Endogenous Repair Mechanism by GIS

### Part A—MUS81 Knockdown by RNA Interference

RTC mRNA for F-ZoAl (SEQ ID NO 19) was produced as in Example 4. GIC RNA including a GFP transgene expression cassette TCA5_CBhBsi_GFP_GeFo3 (SEQ ID NO 300), was produced as in Examples 2. Validated anti-MUS81 siRNA and anti-MSH2 siRNA as described in Table 22 were purchased from ThermoFisher Scientific. Silencer Select Negative Control No. 1 siRNA was purchased from Invitrogen.

TABLE 2
iRNA Duplex Design

| SIRNA | Target | Sense | Antisense |
|---|---|---|---|
| ID | Gene | Sequence | Sequence |
| s37038 | MUS81 | CGCGCUU | UUCUGAA |
| | | CGUAUUU | AUACGAA |
| | | CAGAAtt | GCGCGtg |
| | | (SEQ ID | (SEQ ID |
| | | NO: 349) | NO: 350) |
| s37039 | MUS81 | UGACCUC | AGAGGGU |
| | | UCCAAAC | UUGGAGA |
| | | CCUCUtt | GGUCAtg |
| | | (SEQ ID | (SEQ ID |
| | | NO: 351) | NO: 352) |
| s37040 | MUS81 | GGGAGCA | UUAGGAU |
| | | CCUGAAU | UCAGGUG |
| | | CCUAAtt | CUCCCgg |
| | | (SEQ ID | (SEQ ID |
| | | NO: 353) | NO: 354) |
| s8966 | MSH2 | GGAUAUU | UUACACG |
| | | ACUUUCG | AAAGUAA |
| | | UGUAAtt | UAUCCaa |
| | | (SEQ ID | (SEQ ID |
| | | NO: 355) | NO: 356) |
| s8967 | MSH2 | CGUCGAU | UAAGAUC |
| | | UCCCAGA | UGGGAAU |
| | | UCUUAtt | CGACGaa |
| | | (SEQ ID | (SEQ ID |
| | | NO: 357) | NO: 358) |
| s8968 | MSH2 | GAAUCGC | UAUCAUA |
| | | AAGGAUA | UCCUUGC |
| | | UGAUAtt | GAUUCtc |
| | | (SEQ ID | (SEQ ID |
| | | NO: 359) | NO: 360) |

Each siRN duplex a sense an antisense annealed, with ower case indicating overhang. Three siRNA duplexes were mixed for each siRNA treatment.

siRNA mix for transfection was prepared by combining two tubes: one tube with 625 μl of OptiMEM (Gibco) mixed with 37.5 μl Lipofectamine 3000 and one tube containing 625 μl OptiMEM mixed with 375 pmol siRNA. Three different siRNA for any target were pooled and 375 pmol of Silencer Select Negative Control No. 1 siRNA (Invitrogen) was used as a negative control.

Following 10-mmn incubation, 1.25 ml of the siRNA-lipid complex mixture was added to plates, followed by approximately 4.5 million hTERT RPE-1 cells (equating to about 75% confluency when attached), bringing the total volume of media in the wells to 10 ml (final concentration of 37.5 nM siRNA). 24 hours later, the cells were split 1:3 to be around 60% confluent 2 days after siRNA introduction, when they were then transfected with 2-RNA combination. qRT-PCR was performed to measure target mRNA knockdown efficiency 72 hours post-transfection.

hTERT RPE-1 cells were first transfected with anti-MUS81, anti-MSH2 siRNA, or a scrambled siRNA to serve as a control. One (1) or two (2) days later cells were either not transfected with a GIS (negative control), transfected only with a GIC, or co-transfected with the RTC and GIC as described above.

Twenty-four hours after the final transfection, cells were harvested and percent of cells expressing GFP determined by FACS analysis as described in Example 8 and reported in Table 23.

TABLE 23
Effect of Endogenous Repair Knockdown on GIS Function

| siRNA | GIS | Days Between | Percent GFP |
|---|---|---|---|
| Transfected | Transfected | Transfections | Positive Cells |
| Scrambled | None | 1 | 0.0016 |
| Scrambled | None | 2 | 0.073 |
| Scrambled | GIC Only | 1 | 0.029 |
| Scrambled | GIC Only | 2 | 0.028 |
| Scrambled | RTC + GIC | 1 | 4.57 |
| Scrambled | RTC + GIC | 2 | 1.48 |
| siMSH2 | None | 1 | 0.024 |
| siMSH2 | None | 2 | not tested |
| siMSH2 | GIC Only | 1 | 0.01 |
| siMSH2 | GIC Only | 2 | 0.017 |
| siMSH2 | RTC + GIC | 1 | 4.36 |
| siMSH2 | RTC + GIC | 2 | 1.72 |
| siMUS81 | None | 1 | 0.043 |
| siMUS81 | None | 2 | 0.034 |
| siMUS81 | GIC Only | 1 | 0.021 |
| siMUS81 | GIC Only | 2 | 0.06 |
| siMUS81 | RTC + GIC | 1 | 3.06 |
| siMUS81 | RTC + GIC | 2 | 0.22 |

### Part B GIS Activity in MUS8G Deficient Cell Lines

RTC mRNA for F-ZoAl RT (SEQ ID NO 19) was produced as in Example 4. GIC RNA including a GFP transgene expression cassette TCA5_CBhBsi_GFP_GeFo3 (SEQ ID NO 300, was produced as in Examples 2.

Either wild-type or MUS81-negative mutant HTC 116 cell lines were co-transfected as described previously. Cells were harvested 24 hours post transfection and percent of cells expressing GFP determined by FACS analysis as reported in Table 24.

TABLE 24
GIS Activity in MUS81 Negative Cell Lines

| | GIS | Percent GFP |
|---|---|---|
| Cell Line | Transfected | Positive Cells |
| HCT116 MUS81+ | None | 0.00677 |
| HCT116 MUS81+ | None | 0.028 |
| HCT116 MUS81+ | GIC Only | 0.26 |
| HCT116 MUS81− | GIC Only | 0.01 |
| HCT116 MUS81− | RTC + GIC | 0.00721 |
| HCT116 MUS81− | RTC + GIC | 0.036 |

These results show that MUS81 activity was required for maximum efficiency of transgene insertion by a GIS of the invention. Therefore GIS as described herein may recruit endogenous genomic repair mechanism (e.g., MUS81) to accomplish successful transgene insertion.

Given that loss of MSH2 activity, another enzyme known to function in genomic repair, did not significantly hamper the rate of transgene insertion by a GIS, the GIS of the invention may have selectively recruited MUS81 for transgene insertion. It should be noted that MUS81 was not known to function in any native retroelement or transgene insertion mechanisms.

Part C: RNA Interference Knockdown with Reporter Co-Transfection

RTC mRNA (SEQ ID NO F-ZoAl RT 19) was produced as in Example 4. GIC RNA including a GFP transgene expression cassette TCA5_CBhBsi_GFP_GeFo3 (SEQ ID NO 300), was produced as in Examples 2. mRNA encoding mCherry (TriLink #L-7203), anti-MUS81 siRNA (equal mixture of ThermoFisher Silencer Select ID number s37038, s37039, s37040), and Silencer Select Negative Control No. 1 siRNA (Invitrogen) were purchased.

hTERT RPE-1 cells were first transfected with anti-MUS81 or negative control siRNA. Two (2) days later cells were either not transfected with a GIS (negative control), transfected only with a GIC, or co-transfected with the RTC, GIC and the mCherry mRNA. All transfections were carried out using Lipofectamine MessengerMax. The mCherry mRNA was designed to translate mCherry via classic cap-dependent mRNA translation (i.e., without the need for GIS activity) and served as a control for transfection efficiency when GFP insertion efficiency is reduced.

Twenty-four hours after the final transfection, cells were harvested and percent of cells expressing GFP and mCherry determined by FACS analysis as reported in Table 25 (percent of GFP positive cells relative to the background included in parenthesis where applicable).

TABLE 25
siRNA Knockdown of MUS81

| | | Percent | | Percent | |
|---|---|---|---|---|---|
| | | GFP | GFP | mCherry | mCherry |
| siRNA | Constructs | Positive | Median | Positive | Median |
| Transfected | Transfected | Cells | Intensity | Cells | Intensity |
| Scrambled | None | 0.00279 | 1968 | 0.00558 | 187 |
| Scrambled | GIC Only | 0.025 | 2299 | 0.034 | 321 |
| Scrambled | RTC + GIC + | 3.8 | 8126 | 90.666 | 2307 |
| | mCherry(mRNA) | | | | |
| siMus81 | None | 0.069 | 2163 | 0.05 | 291 |
| siMus81 | GIC Only | 0.17 | 2331 | 0.22 | 337 |
| siMus81 | RTC + GIC + | 0.39 | 2705 | 88.78 | 2460 |
| | mCherry(mRNA) | | | | |

These results confirmed drastic decrease in GFP transgene expression in cells depleted for or lacking MUS81, observed reproducibly in Parts A and B and C, was not due to any effect of MUS81 knockdown on the ability to transfect cells with RNA or the ability of the GIS-containing hTERT RPE-1 cells to translate transfected mRNA.

### EXAMPLE 32. Template Modules with Different Promoters

[0798]hTERT RPE-1 cells were cultured and transfected with F-ZoAl RT mRNA RTC 19) with GIC containing a GFP ORF+/−N-terminal nuclear localization sequence (NLS) with different expression contexts (SEQ ID 309-313). Transcription promoters tested included CBh, EFS, and mPGK (SEQ IDs 275-402 or 282-283). Direction of payload cassette transcription was either codirectional with RNAPI or the reverse “flip” orientation convergent with RNAPI transcription; the “flip” orientation also removed the positioning of an RNAPI transcription termination signal cassette from upstream of the RNAPII promoter.

GFP synthesis was monitored by FACS at 1 day and 5 days post-transfection (Table 26A)_Several comparisons are of special interest. First, the codirectionally oriented CBh_GFP or CBh_NLSGFP and convergently oriented [CBh_NLSGFP]flip had similar % GFP cells on day 1 post-transfection, but 4 days later the convergently oriented [CBh_NLSGFP]flip GFP % cells decreased while codirectionally oriented transgenes' GFP signal remained high. This suggests that codirectional transcription and/or RNAPI transcription termination signal ahead of the RNAPII expression cassette is favorable for sustained transgene expression, while the flip context is favorable when transient expression is desired. Second, detectable GFP transgene expression with mPGK and EFS promoters indicates that different promoters can be used for productive transgene expression.

TABLE 26A
Transgene Promoters and Contexts for OptimalExpression

| | | Percent GFP | Percent GFP |
|---|---|---|---|
| | GIC SEQ | Positive Cells | Positive Cells |
| Promoter and ORF | ID | day1 | day5 |
| CBh_GFP | 309 | 4.041 | 4.46 |
| CBh_NLSGFP | 310 | 3.192 | 2.2 |
| [CBh_NLSGFP]flip | 311 | 3.934 | 0.6 |
| mPGK_GFP | 312 | 0.614 | 0.16 |
| EFS_GFP | 313 | 0.963 | 0.57 |

Additional experiments were performed with GICs containing other transgene transcription promoters. A modified cytomegalovirus promoter with CpG mutation and neo3 5′UTR (CMV*, SEQ ID NO 282) was tested, and a modified simian virus 40 promoter with improved TATA box (SV40*, SEQ ID NO 283) was tested. These were used in GIC to insert a GFP expression transgene. hTERT RPE-1 cells were co-transfected with ZoAl RTC mRNA and one of the GIC constructs, with molar ratio of RTC mRNA to total GIC template RNA of 1:3. After 24 hours, cells were assayed by flow cytometry for GFP expression. The percent of cells expressing the intended transgene product is shown in Table 26B.

TABLE 26B
Transgene Promoters for Optimal Expression

| | | | Percent | Percent |
|---|---|---|---|---|
| Promoter_ Reporter | GIC SEQ | Regular U | GFP+ | mCherry+ |
| protein | ID | (U)) | Cells day1 | Cells day1 |
| CBh_GFP | 309 | U | 20.7 | n.a. |
| CMV*_GFP | 324 | U | 50.7 | n.a. |
| SV40*_GFP | 325 | U | 44.8 | n.a. |
| CBh_mCherry | 308 | U | n.a. | 19.9 |
| CMV*_mCherry | 327 | U | n.a. | 33.5 |
| SV40*_mCherry | 328 | U | n.a. | 16.6 |

### EXAMPLE 33. Inserted Transgene Sequencing from Genomic DNA to Determine Insertion Site-Specificity

RTC mRNA for F-ZoAl RT (SEQ ID NO 19) or F-TaGu RT (SEQ ID NO 28) was produced as in Example 4. GIC RNA with a GFP transgene expression cassette containing 5′ module TCA5 (TCA5_CBhBsi_GFP_GeFo3, SEQ ID NO 300) or 5′ module TCARZ (TCARZ_CBhBsi_GFP_GeFo3, SEQ ID NO 322) was produced as in Example 2.

hTERT RPE-1 cells were co-transfected with an RTC mRNA and GIC RNA, with molar ratio of RTC mRNA to GIC template RNA of 1:3. After 24 hours, cells were sorted to enrich GFP+population as described in Example 8. Enriched GFP+cells were harvested for genomic DNA purification as described in Example 24. One ug of DNA was submitted for standard library preparation and Illumina whole genome shotgun (WGS) sequencing by the University of California, Berkeley Functional Genomics Laboratory and Vincent J. Coates Genomics Sequencing Laboratory, respectively. Human WGS preps are performed with Kapa Hyper Prep reagents and Unique Dual Indexed Y-Adapters with 1 cycle of PCR. Sequencing is performed at 30× coverage on a NovaSeq 6000 S4 with 150 bp paired-end reads.

After adaptor trimming, reads were mapped to a custom contig that contained transgene sequence. Any read with a region that mapped uniquely to the transgene sequence region of the custom contig (SEQ ID NO 273) that also had an unmapped portion of the read (a “clipped” portion) was evaluated as a candidate junction sequence of transgene and genome. Candidate transgene 3′ junction reads were first mapped to transgene sequence flanked by the precise expected downstream target site (SEQ ID NO 274) to count the “at target site” insertions (the vast majority). The clipped region of any candidate 3′ junction that didn't match the precise target site was then mapped to an entire human rDNA consensus scaffold to count imprecisely joined but still rDNA-targeted insertions (“rDNA but not precise target site”).

Any clipped region not mapping to rDNA was mapped to human genome assembly GRCh39. Candidate off-target insertion junction reads (“uncertain”) from ZoAl RTC transfections did not have the transgene 3′ end hallmark of an insertion, suggesting that they were artifactual rearrangements of sequence during extensive sequencing library amplification. No off-target insertion site was evident. Seven candidate off-target insertion junction reads from TaGu RTC transfection joined the expected transgene 3′ end to human genome sequence other than rDNA, giving a maximum off-target insertion frequency of less than 1%.

TABLE 27
Insertion Site Specificity based on Genomic Sequencing

| | | | | Uncertain (library |
|---|---|---|---|---|
| | | | rDNA but not | production |
| RTC mRNA | GIC SEQ | At target | precise target | artifact or |
| (seq ID) | ID | site | site | off-target) |
| ZoAl (19) | 300 | 531 | 1 | 1 |
| ZoAl (19) | 322 | 1033 | 3 | 1 |
| TaGu (28) | 322 | 964 | 5 | 7 |

### EXAMPLE 34. RTC mRNA and GIC RNA with Uridine Analogs

RTC mRNA for F-ZoAl RT (SEQ ID NO 19) was produced as in Example 4 using uridine or modified uridine nucleotides. GIC template RNA with a GFP transgene expression cassette was produced as in Example 2 using uridine or modified uridine nucleotides. The RNAs for each experiment contained either 100% of the uridine analog listed or if two uridines are listed a mix of 50% each. The Tables below show the results of transfection with 2 separate RNAs, one an mRNA for ZoAl RT and the other a GIC template RNA with a GFP transgene expression cassette. The cells were harvested 1 day after transfection and the percentage of GFP positive cells determined by flow cytometry.

Table 28 shows the data for F-ZoA1 mRNA comprising the indicated uridine analogs and a GIC template RNA TCA5_CBhBsi_GFP_GeFo3_R4A22 (SEQ ID 300) with unmodified uridine (uridine ribonucleotide triphosphate “regU”).

TABLE 28
RTC mRNA for ZoAl RT with Uridine Analogs

| ZoAl mRNA | | | GFP median | |
|---|---|---|---|---|
| Uridine | GFP % | | intensity | |
| nucleotide | average | S.D. | average | S.D. |
| regU | 7.17633333 | 0.79286401 | 5645.33333 | 133.881789 |
| regU:N1mpsU | 13.2296667 | 0.56862407 | 9568.66667 | 520.66528 |
| 50:50 mixture | | | | |
| N1mpsU | 13.2296667 | 0.66583281 | 9354 | 733.130957 |
| N1mpsU:psU | 12.2963333 | 0.45092498 | 9160.33333 | 580.542275 |
| 50:50 mixture | | | | |
| psU | 12.463 | 0.43588989 | 9086.33333 | 933.837423 |
| 5mU | 11.163 | 0.7 | 7338.33333 | 354.425357 |
| 5moU | 12.9296667 | 0.37859389 | 8715.66667 | 177.902595 |

Abbrevations: uridine ribonucleotide triphosphate (regU), 5-methoxy-uridine ribonucleotide triphosphate (5moU), 5-methyl-uridine ribonucleotide triphosphate (5mU), pseudouridine ribonucleotide triphosphate (psU), N1-methyl-pseudouridine ribonucleotide triphosphate (N1mpsU).

Table 29 shows the data for F-ZoA1 mRNA comprising 5moU and the GIC template RNA TCA5_CBhBsi_GFP_GeFo3_R4A22 (SEQ ID 300) comprising the indicated uridine analogs.

TABLE 29
GIC template RNA with Uridine Analogs

| GIC template | | | GFP median | |
|---|---|---|---|---|
| RNA uridine | GFP % | | intensity | |
| nucleotide | average | SD | average | SD |
| regU | 12.9296667 | 0.37859389 | 8715.66667 | 177.902595 |
| 5moU | 1.04333333 | 0.0321455 | 1171.66667 | 41.0528115 |
| 5mU | 17.81 | 0.26457513 | 5845.66667 | 113.160653 |
| psU | 41.44 | 0.45825757 | 6311.33333 | 86.3153134 |
| N1mpsU | 30.1433333 | 0.75055535 | 3959.66667 | 307.034743 |

Table 30 shows the data for ZoAl mRNA (SEQ ID 21) made with N1methylpseudouridine and six different GIC template RNAs comprising psU (transgenes expressing GFP or mCherry, each with CBh, CMV* or SV40* promoter), with SEQ ID as indicated in the Table. These results were determined in parallel with results in Table 26B. Comparing the two Tables indicates that transgene delivery efficiency was better using psU template than regular U template.

TABLE 30
GIC template RNAs encoding different
promoters benefit from pseudouridine.

| | | | Percent | Percent |
|---|---|---|---|---|
| Promoter_ Reporter | GIC SEQ | pseudouridine | GFP+ | mCherry+ |
| protein | ID | (psU) | Cells day1 | Cells day1 |
| CBh_GFP | 309 | psU | 38 | n.a. |
| CMV*_GFP | 324 | psU | 81.9 | n.a. |
| SV40*_GFP | 325 | psU | 65 | n.a. |
| CBh_mCherry | 308 | psU | n.a. | 42.2 |
| CMV*_mCherry | 327 | psU | n.a. | 70.1 |
| SV40*_mCherry | 328 | psU | n.a. | 51.9 |

The results show that when RTC mRNA encoding the RT protein comprises modified uridine nucleotides, an increase in trangene expression is observed. Likewise, when the GIC template RNA comprises modified uridine nucleotides, an increase in trangene expression is observed when the uridine is psU or N2mpsU.

### Example 35. GIC 3′ module RNA with truncated GeFo 3′UTR comprising Uridine Analogs Increase Frequency of cells that express transgene

This example shows that a GIC 3′ module with truncated GeFo 3′UTR and template RNA comprising a uridine analog increases the frequency of transgene expression. F-ZoAl mRNA (SEQ ID 19) was synthesized with 5moU and GIC template RNAs (TCARZ_CBh_GFP_GeFo3_R4A22, SEQ ID 322) were synthesized with regular U or pseudoU. The GIC template RNAs comprised a full length 3′UTR (GeFo3, SEQ ID NO 158) or three different truncated 3′UTRs (GeFo217, SEQ ID NO 176; GeFo98, SEQ ID NO 177; and GeFo68, SEQ ID NO 178). The results are shown in Table 31 below.

TABLE 31
Transgene Expression using GIC template RNA
comprising Truncated GeFo 3′UTR and pseudoU.

| GIC 3′ | | GFP median |
|---|---|---|
| UTR | GFP % | intensity |
| GeFo3 regU | 8.54 | 2855 |
| GeFo217 regU | 9.7 | 3129 |
| GeFo98 regU | 10.4 | 3846 |
| GeFo68 regU | 9.33 | 3487 |
| GeFo3 psU | 25.86 | 2642 |
| GeFo217 psU | 28.16 | 2875 |
| GeFo98 psU | 29.26 | 3558 |
| GeFo68 psU | 17.96 | 2001 |

The data demonstrates that a GIC RNA template comprising a truncated 3′ UTR increased the frequency of cells that express functional transgene protein compared to a full length 3′ UTR. The data also demonstrates that a GIC RNA template comprising pseudoU increased the frequency of cells that express functional transgene protein compared to templates that are synthesized with regU.

1. A system for genome editing, comprising
(i) at least one reverse transcriptase construct (RTC), said RTC comprising at least one reverse transcriptase module (RTC: RT-module) comprising an mRNA encoding a reverse transcriptase (RT), at least one reverse transcriptase construct 5′ module (RTC: 5′ module), and/or at least one reverse transcriptase construct 3′ module (RTC: 3′ module), and
(ii) at least one gene insertion construct (GIC), said GIC comprising at least one RNA template suitable for reverse transcription by a polypeptide encoded by the at least one RTC, wherein the at least one gene insertion construct comprises at least one optional GIC: 5′ module, at least one GIC: payload module, and at least one GIC: 3′ module.

2. The system of claim 1, wherein:
(i) the RTC 5′ module comprises a 5′ untranslated region (5′-UTR), a Kozak sequence or an internal ribosome entry site, a non-native translation start codon, and/or a 5′ cap;
(ii) the RT-module comprises an mRNA encoding a RT from an organism selected from the group consisting of Zonotrichia albicollis (ZoAl), Taeniopygia guttata (TaGu), Tinamus guttatus (TiGu), Oryzias latipes (OrLa), and Tribolium castaneum (lineage B) (TriCasB);
(iii) the RTC 3′ module comprises a reverse transcriptase translation stop codon, a 3′ untranslated region (3′ UTR), and a poly-A tail;
(iv) the GIC: 5′ module comprises a sequence derived from a native retroelement 5′ region, an rRNA sequence, a ribozyme sequence, a folding motif sequence, and/or an RNA polymerase terminator sequence;
(v) the GIC: payload module comprises at least one transgene ORF or non-coding RNA (ncRNA) sequence, a transgene promoter sequence, a transgene 5′ untranslated sequence, a transgene 3′ untranslated sequence, a transgene polyadenylation signal sequence, and/or a transgene ncRNA processing sequence; and/or
(vi) the GIC: 3′ module comprises a reverse transcriptase recognition sequence, a rRNA sequence, and/or an A-Tract sequence.

3. The system of claim 1, wherein
(i) the at least one reverse transcriptase is from a non-long terminal repeat (non-LTR) retroelement, or a modified variant thereof; and/or
(ii) the at least one reverse transcriptase comprises at least one DNA binding domain, at least one RNA binding domain, at least one cDNA synthesis domain, at least one endonuclease domain, and any combination thereof; and/or
(iii) the at least one reverse transcription module comprises or encodes at least one structure illustrated in FIGS. 2-5 or any combination thereof; and/or
(iv) the at least one reverse transcriptase construct comprises, encodes, or is encoded by at least one sequence selected from the group consisting of SEQ ID NOS 1-57 and any combination thereof; and/or
(v) the reverse transcriptase is from a bird species,
wherein optionally the reverse transcriptase is from Zonotrichia albicollis (ZoA1), Taeniopygia guttata (TaGu) or Tinamus guttatus (TiGU),
wherein further optionally the reverse transcriptase comprises an amino acid sequence having at least 90% identity to SEQ ID NO:18, SEQ ID NO:20, SEQ ID NO:27, SEQ ID NO:29, or SEQ ID NO:25.

4. The system of claim 2, wherein the optional at least one GIC: 5′ module rRNA sequence comprises or encodes between 1 and 30 nt of subject rRNA,
wherein optionally the rRNA sequence comprises a sequence selected from the group consisting of SEQ ID NOs: 250-276, or a sequence having one, two or three nucleotide changes relative to a sequence selected from the group consisting of SEQ ID NOs: 250-276,
wherein further optionally the GIC: 5′ module does not comprise a rRNA sequence.

5. The system of claim 2, wherein
(i) the GIC: 5′ module ribozyme sequence comprises at least one self-cleaving ribozyme, optionally wherein said self-cleaving ribozyme comprises a hepatitis delta virus (HDV) ribozyme fold,
wherein optionally the HDV ribozyme comprises a sequence having at least 90% identity to a sequence selected from the group consisting of SEQ ID NOs: 102-127, and 129-154; or
(ii) the GIC: 5′ module ribozyme sequence comprises a ribozyme from the 5′ region of at least one non-long terminal repeat retroelement,
wherein optionally the ribozyme comprises a sequence having at least 90% identity to a sequence selected from the group consisting of SEQ ID NOs: 64-65, 67, 75-76, 86, 89-101, and 128.

6. The system of claim 2, wherein the GIC: 5′ module folding motif sequence comprises at least one autonomous folding RNA sequence motif, optionally wherein said autonomous folding RNA sequence motif comprises at least one hairpin motif, at least one stem-loop motif, at least one paired stem 4 motif or any combination thereof; wherein further optionally
(i) the folding motif sequence comprises SEQ ID NOS 278 or 279, or a sequence having at least 90% identity to SEQ ID NOS 278 or 279,
(ii) the GIC: 5′ module comprises a sequence having at least 90% identity to a sequence selected from the group consisting of SEQ ID NOS 60-154;
(iii) the GIC: 3′ module reverse transcriptase recognition sequence comprises at least one sequence which interacts with at least one reverse transcriptase,
optionally wherein the GIC: 3′ module reverse transcriptase recognition sequence is from the 3′ region of a native retroelement and/or comprises a sequence having at least 90% identity to a sequence selected from the group consisting of SEQ ID NOS 200-224;
(iv) the GIC: 3′ module rRNA sequence comprises between 1 and 30 nt of rRNA, wherein optionally the rRNA sequence is selected from the group consisting of SEQ ID NOs 280-289, or a sequence comprising one or two nucleotide substitutions thereof;
(v) the GIC: 3′ module A-Tract sequence comprises between 1 and 50 adenine bases; and/or
(vi) the GIC: 3′ module comprises a sequence having at least 90% identity to a sequence selected from the group consisting of SEQ ID NOS 300-329, or any combination thereof, or comprises a 3′ UTR sequence from ZoAl, TaGu, GeFo, or TiGu,
wherein optionally the 3′ UTR sequence comprises a sequence having at least 90% identity to a sequence selected from the group consisting of SEQ ID NOS 202-205, or SEQ ID NOS 222-224;
(vii) the at least one transgene sequence comprises or encodes at least one sequence of interest for insertion into a subject genome,
wherein optionally the transgene sequence comprises or encodes at least one mRNA, microRNA, siRNA, rRNA, tRNA, long non-coding RNA, small cytoplasmic RNA, small nuclear RNA, small nucleolar RNA, small Cajal body RNA, circular RNA, regulatory RNA, peptide, polypeptide, protein, inhibitory protein, and/or sequences which control expression of at least one transgene,
wherein further optionally the transgene encodes a protein selected from hTERT, hPAH, hFactor VIII, a mutant hFactor VIII having variable size B domains, or Factor IX;
(viii) the transgene promoter sequence comprises at least one sequence which promotes expression of a transgene in a subject genome;
(ix) the transgene 5′ untranslated sequence comprises at least one transgene mRNA 5′ untranslated region;
(x) the transgene 3′ untranslated sequence comprises at least one transgene mRNA 3′ untranslated region;
(xi) the transgene polyadenylation signal sequence comprises at least one transgene polyadenylation signal;
(xii) the transgene non-coding RNA (ncRNA) processing sequence comprises at least one termination signal, at least one 3′ processing signal, and any combination thereof for at least one transgene expressed ncRNA;
(xiii) the at least one GIC: payload module comprises or encodes at least one sequence having at least 90% identity to a sequence selected from the group consisting of SEQ ID NOS 411-422 or SEQ ID NOS 499-536, or any combination thereof;
(xiv) at least one of the at least one GIC: 5′ module and at least one GIC: 3′ module comprise or encode at least one sequence derived from a species of non-long terminal repeat retroelement different from at least one of the other at least one GIC: 5′ module and at least one GIC: 3′ module;
(xv) the at least one gene insertion construct comprises or encodes at least one structure illustrated in FIGS. 6-9 and any combination thereof;
(xvi) the system comprises two different gene insertion constructs comprising GIC: payload modules comprising different transgene ORFs,
wherein optionally the two different GICs are present on the same RNA template or on different RNA templates; and/or
(xvii) the system comprises:
(a) at least one reverse transcriptase construct, wherein the at least one reverse transcriptase construct comprises or is encoded by at least one sequence having at least 90% identity to a sequence selected from the group consisting of SEQ ID NOS 1-57;
(b) at least one gene insertion construct, wherein the at least one gene insertion construct comprises:
a GIC: 5′ module comprising a sequence having at least 90% identity to a sequence selected from the group consisting of SEQ ID NOs: 60-154;
a rRNA sequence comprising a sequence selected from the group consisting of SEQ ID NOs: 250-276, or a sequence having one, two or three nucleotide changes relative to a sequence selected from the group consisting of SEQ ID NOs: 250-276; or does not comprise a rRNA sequence;
a GIC: payload module comprising at least one transgene sequence; and
a GIC: 3′ module comprising a sequence having at least 90% identity to a sequence selected from the group consisting of SEQ ID NOS 300-329;
a GIC: 3′ module reverse transcriptase recognition sequence comprising a sequence having at least 90% identity to a sequence selected from the group consisting of SEQ ID NOS 200-224;
a GIC: 3′ module rRNA sequence selected from the group consisting of SEQ ID NOS 280-289, or a sequence comprising one or two nucleotide substitutions thereof; and/or
a GIC: 3′ module A-Tract sequence comprising 1 to 100 adenine bases;
wherein optionally the GIC: payload module comprises at least one sequence having at least 90% identity to a sequence selected from the group consisting of SEQ ID NOS 411-422 or 499-536.

7. The system of claim 1, wherein
(i) at least one of the at least one reverse transcriptase construct and at least one gene insertion construct comprise or encode at least one sequence derived from a different species of retroelement than at least one of the other at least one reverse transcriptase construct and at least one gene insertion construct; and/or
(ii) the RTC and/or the GIC RNA comprises at least one modified uracil, or the RTC and/or the GIC RNA comprises 100% modified uracils,
wherein optionally the modified uracil is selected from the group consisting of 5-methyl-uridine, 5-methoxy-uridine, pseudouridine, N1-methyl-pseudouridine, and/or 2-thiouridine.

8. A method for inserting at least one transgene into a subject genome comprising administering an effective amount of at least one of the gene insertion systems (GIS) of claim 1 to the subject, wherein optionally
(i) the transgene is inserted at one or more target sites in the subject genome, optionally wherein the one or more target sites comprise at least one safe harbor site,
wherein optionally the optional at least one safe harbor site comprises at least one ribosomal DNA (rDNA) sequence, optionally wherein the at least one ribosomal DNA sequence comprises at least one 28 S rDNA sequence; and/or
(ii) the method comprises administering at least one of the gene insertion systems formulated with at least one delivery agent,
wherein optionally the at least one delivery agent is at least one nanoparticle, optionally wherein the at least one nanoparticle comprises at least one lipid nanoparticle.

9. The method of claim 8, wherein
(i) the transgene is inserted with a target site-specificity of greater than 90%,
wherein optionally the RTC RNA encodes a RT from Zonotrichia albicollis (ZoA1), Taeniopygia guttata (TaGu) or Tinamus guttatus (TiGU), or comprises an amino acid sequence having at least 90% identity to SEQ ID NO:18, SEQ ID NO:20, SEQ ID NO:27, SEQ ID NO:29, or SEQ ID NO:25; and/or
(ii) the transgene is expressed at the target site for 3 months or more.

10. A pharmaceutical composition comprising at least one of the gene insertion system of claim 1 and at least one of at least one excipient, at least one delivery agent, at least one adjuvant, and any combination thereof.

11. A method of treating a therapeutic indication in a subject in need thereof comprising administering an effective amount of at least one of the pharmaceutical composition of claim 10, optionally comprising a method for inserting at least one transgene into a subject genome comprising administering an effective amount of at least one of the gene insertion systems (GIS) to the subject, wherein optionally
(i) the transgene is inserted at one or more target sites in the subject genome, optionally wherein the one or more target sites comprise at least one safe harbor site,
wherein optionally the optional at least one safe harbor site comprises at least one ribosomal DNA (rDNA) sequence, optionally wherein the at least one ribosomal DNA sequence comprises at least one 28 S rDNA sequence; and/or
(ii) the method comprises administering at least one of the gene insertion systems formulated with at least one delivery agent,
wherein optionally the at least one delivery agent is at least one nanoparticle, optionally wherein the at least one nanoparticle comprises at least one lipid nanoparticle;
wherein optionally:
(a) the therapeutic indication is caused by loss of telomerase activity; and/or
(b) the at least one gene insertion system comprises at least one TERT transgene.

12. A kit for making a gene insertion system, comprising the gene insertion system of claim 1, optionally a pharmaceutical composition comprising at least one of the gene insertion system of claim 1 and at least one of at least one excipient, at least one delivery agent, at least one adjuvant, and any combination thereof, and optionally further comprises buffers, DNA plasmids, or protocols to make said gene insertion systems or pharmaceutical composition.

13. A method comprising de novo design of a 5′ module that recruits host machinery for second strand nicking and thus second strand synthesis, the method optionally providing efficiency of insertion gain by de novo design of the 5′ module to (a) include a predetermined length and position of rRNA, (b) have enhanced RZ folding, and/or (c) recruit host cell machinery.

14. A method for inserting at least one transgene into a genome of a cell comprising contacting the cell with at least one of the gene insertion systems (GIS) of claim 1, wherein optionally
(i) the transgene is inserted at one or more target sites in the subject genome, optionally
wherein the one or more target sites comprise at least one safe harbor site, optionally wherein the optional at least one safe harbor site comprises at least one ribosomal DNA (rDNA) sequence, optionally wherein the at least one ribosomal DNA sequence comprises at least one 28 S rDNA sequence; and/or
(ii) the method comprises administering at least one of the gene insertion systems formulated with at least one delivery agent,
wherein optionally the at least one delivery agent is at least one nanoparticle, optionally wherein the at least one nanoparticle comprises at least one lipid nanoparticle and/or
(iii) wherein the transgene is inserted with a target site-specificity of greater than 90%,
wherein optionally the RTC RNA encodes an RT from Zonotrichia albicollis (ZoA1), Taeniopygia guttata (TaGu) or Tinamus guttatus (TiGU), or comprises an amino acid sequence having at least 90% identity to SEQ ID NO:18, SEQ ID NO:20, SEQ ID NO:27, SEQ ID NO:29, or SEQ ID NO:25; and/or
(iv) the transgene is expressed at the target site for 3 months or more; and/or
(v) the molar ratio of the RTC to GIC is from about 10:1 to 1:20 and/or
(vi) the method is an in vitro method, an ex vivo method, or an in vivo method; and/or
(vii) the cell is selected from the group consisting of a primary cell, a transformed cell, an epithelial cell, a fibroblast, a human cell, a monkey cell and a mouse cell; and/or
(viii) the cell is an allogenic cell or autologous cell,
wherein optionally the autologous cell is an HLA-matched cell.


SEQUENCE LISTING (360 sequences)

SEQ ID NO: 1 - DNA, 3519 bp, synthetic construct
  source 1..3519: mol_type = other DNA
atggattacaaggatgacgacgataagggtaccctgccttttcagagcagaagctgcggcatctgtctgaatgccggtaaaggtaacttccgcgcgctgagcctggacgacgaagaacggcacctgcgtgaacggcatccactgtccctgatcctctataaatgcagcgattgcaagggccagtacagatccaagagggccgccctgtgccacgcccccaagtgcaccggaccgacccctgatcctcaaggcaatgccctgcgttgtcatctgtgtggtcttgtttgtaaaagccagagtggtgttacccagcatttacgtcatagacaccctctggtcagaaacacccagcgggcagctgaagaaagcggtagagccgaacgtgctgcactgcctcggcctctgcgtcgtaacacccgttcggttttcagcgaagaggatgaagcaaaaatgctggagctggaagtgcggttccagaacgagcgttgtgttgcaaaatgcatgctgccgttttttccgaatagaacttgcaagcagatccgtgataagcgtaataccgatgcatataaacggagaagagaactgtacttcgagggcgtccgggtgcaggaccctgcaggcgccgaggacagcgttctccctgttgtggaaaccgacgaacccgccgaggaaaatattccgctggagtaccccgagctgcctggcgatgaagagggtgctcctgcctgcagccagactattctgaacacagaaggtccggatggactgggcagcccaccggtgcccgttgaagaagaaatggcaagttcgggtagcacatctaataacgtggataccggttggagagaaagcattatcacagctgcactcggcgttgaaattccgaaagcaatcagccaagagcccgccgccgttattcaggagctgcaggatgctctgcgcgaggccgtgatcggcgtgtttccgcaggatcgcctggacgagatgtacgagcgggtactcaaagtggtcaacccggatgatacacaggaacgcccgaaacgtcaaagaaagaaaggcaagtctcgtaatgccttccgccgttatgtgtacagccagacccaggacctgttcaagaaaaatcctggacagctggcacgttatgttagggaagatgtgagatggctggaacagggccgggtgcagttgcagagagatgatattgaaagaatgtacaacaagctgtggggcaccaagccggatgtgctgcctccccactgggattatccactgccactcgacaccgctgatgttctgaccccgattgagctgaaagaagtccggaaaagaatatctcagacgaaactcaaatctgcagcagggcccgacggtctgcagaaaagacatctggtgcgtcgcgttgtgcaagaaattctgcgcctgctgtataacctgctgatgtgttgtgcaatgcagcctacacagtggcggatgaaccgtacccaactgttactgaagcagggtaaagatcctctggatgtcgctagttatcgtcctataaccatctccagcatcctttgtcgtctgtactggggtataatcgaccagaagctgcgtgagcatgttcgtttccacccacgtcagaaaggcttcgtgagcgaggcaggttgttttaataatgtgcaaattctgaatgaactgctgcgtcacagcaagggccagcacaaaaatctggttgccgtgtgcctggatgtttctaaagcatttgataccgttcctcatagcatcctcggccccgccctgcgcatgaagggcctgccggaacaggtcgttcgtctggttgaagatagctacaaagatctgcatactgtcgttaaacaggggaccgcagaagtgacgctgagcctgcagcgtggagtgaagcagggcgaccccctgagccccttcctgttcaacgccgtgctggagccgctgctgctgcagctggaaagccatcctggttataaagtgggcggtgaactggcctctgttagctgtatggcctttgcagatgatatctttctgattgcagctaatgttccgcaggcctgtaccctgctgagggtcacggaagattatctggaaagactgggcatgcgtatcagcgcccctaaatgtaccagctttgaaatccgtccgaccaaagatagctggtatgttgcagatccggggcttacactgaccaaaggagaacgtatccctgtcgctgctgtggatgccgtttttagctacctgggtgttgaaattagcccttgggcaggtatcaccagcgagggcatcgaacgggattggcggggtacactgcatcgtgtgcaacgcctgccgctgaagccccaccagaaactggaactgatcagcagatacctggttcctcattttctgtataaactggtggtgaccatccctagcataaccctgattagacagctggatcaggaactgcgggttgtggtgaagcagatctgtcatctgcctcagagcaccgccgacggcatgatctattgtcggagagtggacggcggtctgggtattccgaagctggaaattgttaccgtgaccagcatactgaaagcaggcctgaaatttagagatagccaggacaaaatcatgcaggcactctggctggcatcaggtatgagcagccgtctgaacagcctggccaaggcgaccagagtacaaccttggcccccgaacaatattaaagatctggacagacataaagttgctcgtaagaaagaagaactggcccgatgggccagtttgaccagccagggtaaaagcgtgaaaagcttcgccggcagccgtaccgccaatgcatggctgattaacaagaagttactgaagccctctacctttatcagcgccttaagactgagaggcaatgtcgctggagaccgtgtggccctgaatagagcaatcccgcaggccaacctgatgtgcagacgttgcggtagccagagggaaactctgggccacatcctgggtatctgtaccagcaccaaagccctacgtatttcacgccatgatgagatcaagaatctgatcgtggacgaagcagcaaagaaggacgacgaagtggctgttacactggagccaaccattcgtcaccctgttcgtggtaacctgaaaccggacctggtggttcaaaacagagaaggcgtgtacgttgttgacgtgacagtgagacacgaggatggcaacctgcttgcacagggacgtcaggataaactggacaagtacgaagtgctgctgccgattctgcaagaaagactgggtgctcctaccggtgaggttctgccgattgttgttggcacccgtggcgccatgcctaaagagacagtggaagccttgaagaaactgcgcattaccgaccggcagaccctgctcacgatcagcctgattgccctgagaatgtctgtgaaaatttatcataccttcatggactatgcaaacgccagaccgcgtccgggcggcggtgcaaactacccccacagatgataa

SEQ ID NO: 2 - protein, 1171 aa, synthetic construct
MDYKDDDDKGTLPFQSRSCGICLNAGKGNFRALSLDDEERHLRERHPLSLILYKCSDCKGQYRSKRAALCHAPKCTGPTPDPQGNALRCHLCGLVCKSQSGVTQHLRHRHPLVRNTQRAAEESGRAERAALPRPLRRNTRSVFSEEDEAKMLELEVRFQNERCVAKCMLPFFPNRTCKQIRDKRNTDAYKRRRELYFEGVRVQDPAGAEDSVLPVVETDEPAEENIPLEYPELPGDEEGAPACSQTILNTEGPDGLGSPPVPVEEEMASSGSTSNNVDTGWRESIITAALGVEIPKAISQEPAAVIQELQDALREAVIGVFPQDRLDEMYERVLKVVNPDDTQERPKRQRKKGKSRNAFRRYVYSQTQDLFKKNPGQLARYVREDVRWLEQGRVQLQRDDIERMYNKLWGTKPDVLPPHWDYPLPLDTADVLTPIELKEVRKRISQTKLKSAAGPDGLQKRHLVRRVVQEILRLLYNLLMCCAMQPTQWRMNRTQLLLKQGKDPLDVASYRPITISSILCRLYWGIIDQKLREHVRFHPRQKGFVSEAGCFNNVQILNELLRHSKGQHKNLVAVCLDVSKAFDTVPHSILGPALRMKGLPEQVVRLVEDSYKDLHTVVKQGTAEVTLSLQRGVKQGDPLSPFLFNAVLEPLLLQLESHPGYKVGGELASVSCMAFADDIFLIAANVPQACTLLRVTEDYLERLGMRISAPKCTSFEIRPTKDSWYVADPGLTLTKGERIPVAAVDAVFSYLGVEISPWAGITSEGIERDWRGTLHRVQRLPLKPHQKLELISRYLVPHFLYKLVVTIPSITLIRQLDQELRVVVKQICHLPQSTADGMIYCRRVDGGLGIPKLEIVTVTSILKAGLKFRDSQDKIMQALWLASGMSSRLNSLAKATRVQPWPPNNIKDLDRHKVARKKEELARWASLTSQGKSVKSFAGSRTANAWLINKKLLKPSTFISALRLRGNVAGDRVALNRAIPQANLMCRRCGSQRETLGHILGICTSTKALRISRHDEIKNLIVDEAAKKDDEVAVTLEPTIRHPVRGNLKPDLVVQNREGVYVVDVTVRHEDGNLLAQGRQDKLDKYEVLLPILQERLGAPTGEVLPIVVGTRGAMPKETVEALKKLRITDRQTLLTISLIALRMSVKIYHTFMDYANARPRPGGGANYPHR

SEQ ID NO: 3 - RNA, 3979 nt, synthetic construct
  source 1..3979: mol_type = other RNA
aggaataaactagtattcttctggtccccacagactcagagagaacccgccaccatggattacaaggatgacgacgataagggatccctgccttttcagagcagaagctgcggcatctgtctgaatgccggtaaaggtaacttccgcgcgctgagcctggacgacgaagaacggcacctgcgtgaacggcatccactgtccctgatcctctataaatgcagcgattgcaagggccagtacagatccaagagggccgccctgtgccacgcccccaagtgcaccggaccgacccctgatcctcaaggcaatgccctgcgttgtcatctgtgtggtcttgtttgtaaaagccagagtggtgttacccagcatttacgtcatagacaccctctggtcagaaacacccagcgggcagctgaagaaagcggtagagccgaacgtgctgcactgcctcggcctctgcgtcgtaacacccgttcggttttcagcgaagaggatgaagcaaaaatgctggagctggaagtgcggttccagaacgagcgttgtgttgcaaaatgcatgctgccgttttttccgaatagaacttgcaagcagatccgtgataagcgtaataccgatgcatataaacggagaagagaactgtacttcgagggcgtccgggtgcaggaccctgcaggcgccgaggacagcgttctccctgttgtggaaaccgacgaacccgccgaggaaaatattccgctggagtaccccgagctgcctggcgatgaagagggtgctcctgcctgcagccagactattctgaacacagaaggtccggatggactgggcagcccaccggtgcccgttgaagaagaaatggcaagttcgggtagcacatctaataacgtggataccggttggagagaaagcattatcacagctgcactcggcgttgaaattccgaaagcaatcagccaagagcccgccgccgttattcaggagctgcaggatgctctgcgcgaggccgtgatcggcgtgtttccgcaggatcgcctggacgagatgtacgagcgggtactcaaagtggtcaacccggatgatacacaggaacgcccgaaacgtcaaagaaagaaaggcaagtctcgtaatgccttccgccgttatgtgtacagccagacccaggacctgttcaagaaaaatcctggacagctggcacgttatgttagggaagatgtgagatggctggaacagggccgggtgcagttgcagagagatgatattgaaagaatgtacaacaagctgtggggcaccaagccggatgtgctgcctccccactgggattatccactgccactcgacaccgctgatgttctgaccccgattgagctgaaagaagtccggaaaagaatatctcagacgaaactcaaatctgcagcagggcccgacggtctgcagaaaagacatctggtgcgtcgcgttgtgcaagaaattctgcgcctgctgtataacctgctgatgtgttgtgcaatgcagcctacacagtggcggatgaaccgtacccaactgttactgaagcagggtaaagatcctctggatgtcgctagttatcgtcctataaccatctccagcatcctttgtcgtctgtactggggtataatcgaccagaagctgcgtgagcatgttcgtttccacccacgtcagaaaggcttcgtgagcgaggcaggttgttttaataatgtgcaaattctgaatgaactgctgcgtcacagcaagggccagcacaaaaatctggttgccgtgtgcctggatgtttctaaagcatttgataccgttcctcatagcatcctcggccccgccctgcgcatgaagggcctgccggaacaggtcgttcgtctggttgaagatagctacaaagatctgcatactgtcgttaaacaggggaccgcagaagtgacgctgagcctgcagcgtggagtgaagcagggcgaccccctgagccccttcctgttcaacgccgtgctggagccgctgctgctgcagctggaaagccatcctggttataaagtgggcggtgaactggcctctgttagctgtatggcctttgcagatgatatctttctgattgcagctaatgttccgcaggcctgtaccctgctgagggtcacggaagattatctggaaagactgggcatgcgtatcagcgcccctaaatgtaccagctttgaaatccgtccgaccaaagatagctggtatgttgcagatccggggcttacactgaccaaaggagaacgtatccctgtcgctgctgtggatgccgtttttagctacctgggtgttgaaattagcccttgggcaggtatcaccagcgagggcatcgaacgggattggcggggtacactgcatcgtgtgcaacgcctgccgctgaagccccaccagaaactggaactgatcagcagatacctggttcctcattttctgtataaactggtggtgaccatccctagcataaccctgattagacagctggatcaggaactgcgggttgtggtgaagcagatctgtcatctgcctcagagcaccgccgacggcatgatctattgtcggagagtggacggcggtctgggtattccgaagctggaaattgttaccgtgaccagcatactgaaagcaggcctgaaatttagagatagccaggacaaaatcatgcaggcactctggctggcatcaggtatgagcagccgtctgaacagcctggccaaggcgaccagagtacaaccttggcccccgaacaatattaaagatctggacagacataaagttgctcgtaagaaagaagaactggcccgatgggccagtttgaccagccagggtaaaagcgtgaaaagcttcgccggcagccgtaccgccaatgcatggctgattaacaagaagttactgaagccctctacctttatcagcgccttaagactgagaggcaatgtcgctggagaccgtgtggccctgaatagagcaatcccgcaggccaacctgatgtgcagacgttgcggtagccagagggaaactctgggccacatcctgggtatctgtaccagcaccaaagccctacgtatttcacgccatgatgagatcaagaatctgatcgtggacgaagcagcaaagaaggacgacgaagtggctgttacactggagccaaccattcgtcaccctgttcgtggtaacctgaaaccggacctggtggttcaaaacagagaaggcgtgtacgttgttgacgtgacagtgagacacgaggatggcaacctgcttgcacagggacgtcaggataaactggacaagtacgaagtgctgctgccgattctgcaagaaagactgggtgctcctaccggtgaggttctgccgattgttgttggcacccgtggcgccatgcctaaagagacagtggaagccttgaagaaactgcgcattaccgaccggcagaccctgctcacgatcagcctgattgccctgagaatgtctgtgaaaatttatcataccttcatggactatgcaaacgccagaccgcgtccgggcggcggtgcaaactacccccacagatgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 4 - protein, 1161 aa, synthetic construct
MLPFQSRSCGICLNAGKGNFRALSLDDEERHLRERHPLSLILYKCSDCKGQYRSKRAALCHAPKCTGPTPDPQGNALRCHLCGLVCKSQSGVTQHLRHRHPLVRNTQRAAEESGRAERAALPRPLRRNTRSVFSEEDEAKMLELEVRFQNERCVAKCMLPFFPNRTCKQIRDKRNTDAYKRRRELYFEGVRVQDPAGAEDSVLPVVETDEPAEENIPLEYPELPGDEEGAPACSQTILNTEGPDGLGSPPVPVEEEMASSGSTSNNVDTGWRESIITAALGVEIPKAISQEPAAVIQELQDALREAVIGVFPQDRLDEMYERVLKVVNPDDTQERPKRQRKKGKSRNAFRRYVYSQTQDLFKKNPGQLARYVREDVRWLEQGRVQLQRDDIERMYNKLWGTKPDVLPPHWDYPLPLDTADVLTPIELKEVRKRISQTKLKSAAGPDGLQKRHLVRRVVQEILRLLYNLLMCCAMQPTQWRMNRTQLLLKQGKDPLDVASYRPITISSILCRLYWGIIDQKLREHVRFHPRQKGFVSEAGCFNNVQILNELLRHSKGQHKNLVAVCLDVSKAFDTVPHSILGPALRMKGLPEQVVRLVEDSYKDLHTVVKQGTAEVTLSLQRGVKQGDPLSPFLFNAVLEPLLLQLESHPGYKVGGELASVSCMAFADDIFLIAANVPQACTLLRVTEDYLERLGMRISAPKCTSFEIRPTKDSWYVADPGLTLTKGERIPVAAVDAVFSYLGVEISPWAGITSEGIERDWRGTLHRVQRLPLKPHQKLELISRYLVPHFLYKLVVTIPSITLIRQLDQELRVVVKQICHLPQSTADGMIYCRRVDGGLGIPKLEIVTVTSILKAGLKFRDSQDKIMQALWLASGMSSRLNSLAKATRVQPWPPNNIKDLDRHKVARKKEELARWASLTSQGKSVKSFAGSRTANAWLINKKLLKPSTFISALRLRGNVAGDRVALNRAIPQANLMCRRCGSQRETLGHILGICTSTKALRISRHDEIKNLIVDEAAKKDDEVAVTLEPTIRHPVRGNLKPDLVVQNREGVYVVDVTVRHEDGNLLAQGRQDKLDKYEVLLPILQERLGAPTGEVLPIVVGTRGAMPKETVEALKKLRITDRQTLLTISLIALRMSVKIYHTFMDYANARPRPGGGANYPHR

SEQ ID NO: 5 - RNA, 3948 nt, synthetic construct
  source 1..3948: mol_type = other RNA
aggaataaactagtattcttctggtccccacagactcagagagaacccgccaccatgctgccttttcagagcagaagctgcggcatctgtctgaatgccggtaaaggtaacttccgcgcgctgagcctggacgacgaagaacggcacctgcgtgaacggcatccactgtccctgatcctctataaatgcagcgattgcaagggccagtacagatccaagagggccgccctgtgccacgcccccaagtgcaccggaccgacccctgatcctcaaggcaatgccctgcgttgtcatctgtgtggtcttgtttgtaaaagccagagtggtgttacccagcatttacgtcatagacaccctctggtcagaaacacccagcgggcagctgaagaaagcggtagagccgaacgtgctgcactgcctcggcctctgcgtcgtaacacccgttcggttttcagcgaagaggatgaagcaaaaatgctggagctggaagtgcggttccagaacgagcgttgtgttgcaaaatgcatgctgccgttttttccgaatagaacttgcaagcagatccgtgataagcgtaataccgatgcatataaacggagaagagaactgtacttcgagggcgtccgggtgcaggaccctgcaggcgccgaggacagcgttctccctgttgtggaaaccgacgaacccgccgaggaaaatattccgctggagtaccccgagctgcctggcgatgaagagggtgctcctgcctgcagccagactattctgaacacagaaggtccggatggactgggcagcccaccggtgcccgttgaagaagaaatggcaagttcgggtagcacatctaataacgtggataccggttggagagaaagcattatcacagctgcactcggcgttgaaattccgaaagcaatcagccaagagcccgccgccgttattcaggagctgcaggatgctctgcgcgaggccgtgatcggcgtgtttccgcaggatcgcctggacgagatgtacgagcgggtactcaaagtggtcaacccggatgatacacaggaacgcccgaaacgtcaaagaaagaaaggcaagtctcgtaatgccttccgccgttatgtgtacagccagacccaggacctgttcaagaaaaatcctggacagctggcacgttatgttagggaagatgtgagatggctggaacagggccgggtgcagttgcagagagatgatattgaaagaatgtacaacaagctgtggggcaccaagccggatgtgctgcctccccactgggattatccactgccactcgacaccgctgatgttctgaccccgattgagctgaaagaagtccggaaaagaatatctcagacgaaactcaaatctgcagcagggcccgacggtctgcagaaaagacatctggtgcgtcgcgttgtgcaagaaattctgcgcctgctgtataacctgctgatgtgttgtgcaatgcagcctacacagtggcggatgaaccgtacccaactgttactgaagcagggtaaagatcctctggatgtcgctagttatcgtcctataaccatctccagcatcctttgtcgtctgtactggggtataatcgaccagaagctgcgtgagcatgttcgtttccacccacgtcagaaaggcttcgtgagcgaggcaggttgttttaataatgtgcaaattctgaatgaactgctgcgtcacagcaagggccagcacaaaaatctggttgccgtgtgcctggatgtttctaaagcatttgataccgttcctcatagcatcctcggccccgccctgcgcatgaagggcctgccggaacaggtcgttcgtctggttgaagatagctacaaagatctgcatactgtcgttaaacaggggaccgcagaagtgacgctgagcctgcagcgtggagtgaagcagggcgaccccctgagccccttcctgttcaacgccgtgctggagccgctgctgctgcagctggaaagccatcctggttataaagtgggcggtgaactggcctctgttagctgtatggcctttgcagatgatatctttctgattgcagctaatgttccgcaggcctgtaccctgctgagggtcacggaagattatctggaaagactgggcatgcgtatcagcgcccctaaatgtaccagctttgaaatccgtccgaccaaagatagctggtatgttgcagatccggggcttacactgaccaaaggagaacgtatccctgtcgctgctgtggatgccgtttttagctacctgggtgttgaaattagcccttgggcaggtatcaccagcgagggcatcgaacgggattggcggggtacactgcatcgtgtgcaacgcctgccgctgaagccccaccagaaactggaactgatcagcagatacctggttcctcattttctgtataaactggtggtgaccatccctagcataaccctgattagacagctggatcaggaactgcgggttgtggtgaagcagatctgtcatctgcctcagagcaccgccgacggcatgatctattgtcggagagtggacggcggtctgggtattccgaagctggaaattgttaccgtgaccagcatactgaaagcaggcctgaaatttagagatagccaggacaaaatcatgcaggcactctggctggcatcaggtatgagcagccgtctgaacagcctggccaaggcgaccagagtacaaccttggcccccgaacaatattaaagatctggacagacataaagttgctcgtaagaaagaagaactggcccgatgggccagtttgaccagccagggtaaaagcgtgaaaagcttcgccggcagccgtaccgccaatgcatggctgattaacaagaagttactgaagccctctacctttatcagcgccttaagactgagaggcaatgtcgctggagaccgtgtggccctgaatagagcaatcccgcaggccaacctgatgtgcagacgttgcggtagccagagggaaactctgggccacatcctgggtatctgtaccagcaccaaagccctacgtatttcacgccatgatgagatcaagaatctgatcgtggacgaagcagcaaagaaggacgacgaagtggctgttacactggagccaaccattcgtcaccctgttcgtggtaacctgaaaccggacctggtggttcaaaacagagaaggcgtgtacgttgttgacgtgacagtgagacacgaggatggcaacctgcttgcacagggacgtcaggataaactggacaagtacgaagtgctgctgccgattctgcaagaaagactgggtgctcctaccggtgaggttctgccgattgttgttggcacccgtggcgccatgcctaaagagacagtggaagccttgaagaaactgcgcattaccgaccggcagaccctgctcacgatcagcctgattgccctgagaatgtctgtgaaaatttatcataccttcatggactatgcaaacgccagaccgcgtccgggcggcggtgcaaactacccccacagatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 6 - DNA, 3534 bp, synthetic construct
  source 1..3534: mol_type = other DNA
atggattacaaggatgacgacgataagggtaccatggagatcgtggccatcgccaagaatccacaggcatgcccaaagtgtctgcagggaggaacccagctgctgtgcatgggcagctgggagctgtccagacacatcaacaaggagcaccccagcgtggacgtgacatgggtgtgcggcgcctgtcagcggagatgtaccacactgcgcagctggtcctgccacgtgcctcactgtaagggccggcaggagcctaaggatctgccattcaagtgcgagcactgttctctgagctttgacagccagatcggcctgtcccagcacgagaggcacgtgcacccagaggtgcggaatgataagagagccgccgaggccaacaagccaaagggcaagagcggcaggaggccttccatctggtctgatgaggacctgctgctgatcagggagctggagtccgagtaccacggagcccggaatatcaacgagaagatcgcagagcacttccctgacaggaccggaaggcaggtgtccgatgcacggagaaggaaggactatgccgccctgaggggccgcggaggaccacagggacctgcagagggagtggaggcaatcgaggaggtggatgagggcgagatcccagagggcgaggagctggtggccacagacggagcagcactggagtctggaccacctgagaatggaggcagcgcacctgcagagcaggtgaacgcaccagcactggagagctcctctcagcaggatagggagtgctctccagcagtgggcagcgatgagcagatcgaggacagctccgacgatgacgagttttctgacgcactgggagagatcagcctgccagagcctctgtccgtggagaggaccacaatcagcccaccacctagggatgactggaagggacctatgcggtgggagatctgtaatgcctccgaggaggccggctcttacgccaactgggtgaccggactgcaggagctggtgcgcaacaatgccctgtctgagatcggcctggatagcctgtatgaccagctgatccagatcatgcggcaccccagcgatgacaatgagcaggacaggctgcagctgaacgcaagaggaccaccaaggaggggacaccgcaagaatagaaggaggaggagactgacagcagcagataggaagagattcgcctttgcccggtgccaggacctgtggaacaataaccctaagaagctggccgagctggtcatcgccaacgatctgtccatcctgcagaggaggcaggcaccaggaagaaccgagacacagaccctgtacaatgagctgtggggaagggtgggaccaaacatcgaggcaccaaggagaaccgaggacccaatccccgtgtcccgcatcttcacaccaatcaccccccaggagatcatgggccggatcaggcgcatcaagaacgattctgcagcaggacctgacggagtgacaaaggatgacctgaggggccgcggcgtgtctatcgcactgagcaagctgtttaatagcatcctgctggccggctactatccaaaggcctggcgggagaacagaaccacactgctgcctaagccagagaaggaccccgccgacgtgaagaattggagacccatcaccatctctagcatggtgtccagggtgtattctggcctgctggatcagcgggtgagagccgtgatcaagcagtgcgacaggcagaagggcttcacagaggagaatggctgtttttccaacatccagctgctggatgacgccgtgtctaacgccaagaaggccggcggcgtgatcaccatcctggatgtgagcaaggccttcgacacagtgccacacgccgtgatccagggctgcctggagaagaagggcatccccgagaccgtggccgcctacatctcctctatgtatcgcgattgtagcacagcaatcaggaccaggtccggcgacgtgaagatcggcatgaagaggggagtgaagcagggcgaccctctgtccccactgatctttaatctggtgctggagccactgctggagaggctgcaggagaccagcggcgtggagatcgagggcatgaatctgtcctgcgccgccttcgccgatgacatcgtgtgttttgcaaacacagcacctgaggcaggaaggcagctgcgcatggtggccgattacctgggcagactggacatgtccctgtctgtgagcaagtgcatcgccgtggagtacgtgcctcacaggaagacatggtataccaagaacccaggcctggaagtgaatggcaacgccgtgccctccatctctcctagcgagaccttcaagtacctgggcgccaaggtgagcccatggaagggcctgctggagggcttcgagtccgatgcctttcgcgaagtgatctctcgggtgcagagactgcccctgaagcctatgcagaaggtggacctgctgcagatgtatatcttccccagatacacctacggcctgatcaccagccctccagccaaggccgtgctgaagacaatcgataggatcatccggaccagaatcaaggagatcctgcacctgccagagtccgtgagctcctcttttctgtacaccccaaggaagcagggaggactgggactgctggaggtggagaagatggtgctgatcgcagcactgagaaatggactgagggcaaggcagtctcacgacccagtgaccagggccgccatgaattctaacgccgccgatgacagactgaagagctacgccgatgcactgaggctgcactggccactgaccacaaaggagctggacacatacaagtatcagctgagactgtcctatgcccagaagtgggcagagcagaagtggcagggacagggagtggaggagttcgcacaggaccccgtgggcaattcttggctgcagagatacgatctgctgcctgccagcaggtatatcgacgccatcaagctgcggacaaacacctaccccaccagagccctgatgaagatcatcgatggcagggtggacagctcctgccgcaagtgtcagggctctagcgagaccctgggacacatcctgggaaggtgtagatatacaaaggataagaggatcagccgccacaatgagatcaaggacctgctgaaggcccgcctggccaagaaccaccaggtcatggatgagcctcagatcaccgtgcggggccagagattcaagccagacctggtggtgaagacaaatgagggccgggtgcacgtgatcgatgtgacagtgaggtacgagcaccgcacctatctggacgagggccgcacagagaagatcggcaagtaccggcagatcctgagcaccctgcggagagatctgcactccaacgccgaggaagtgatccccatcgtgatcggctccagaggagcaatccctagggagaccaggaaggcactgtctaagctgggcatcggcaagtccgactggctgacaatctctctgatcgccctgaggtcctctctggagatcgtgaacgcctttatggatgactag

SEQ ID NO: 7 - protein, 1177 aa, synthetic construct
MDYKDDDDKGTMEIVAIAKNPQACPKCLQGGTQLLCMGSWELSRHINKEHPSVDVTWVCGACQRRCTTLRSWSCHVPHCKGRQEPKDLPFKCEHCSLSFDSQIGLSQHERHVHPEVRNDKRAAEANKPKGKSGRRPSIWSDEDLLLIRELESEYHGARNINEKIAEHFPDRTGRQVSDARRRKDYAALRGRGGPQGPAEGVEAIEEVDEGEIPEGEELVATDGAALESGPPENGGSAPAEQVNAPALESSSQQDRECSPAVGSDEQIEDSSDDDEFSDALGEISLPEPLSVERTTISPPPRDDWKGPMRWEICNASEEAGSYANWVTGLQELVRNNALSEIGLDSLYDQLIQIMRHPSDDNEQDRLQLNARGPPRRGHRKNRRRRRLTAADRKRFAFARCQDLWNNNPKKLAELVIANDLSILQRRQAPGRTETQTLYNELWGRVGPNIEAPRRTEDPIPVSRIFTPITPQEIMGRIRRIKNDSAAGPDGVTKDDLRGRGVSIALSKLFNSILLAGYYPKAWRENRTTLLPKPEKDPADVKNWRPITISSMVSRVYSGLLDQRVRAVIKQCDRQKGFTEENGCFSNIQLLDDAVSNAKKAGGVITILDVSKAFDTVPHAVIQGCLEKKGIPETVAAYISSMYRDCSTAIRTRSGDVKIGMKRGVKQGDPLSPLIFNLVLEPLLERLQETSGVEIEGMNLSCAAFADDIVCFANTAPEAGRQLRMVADYLGRLDMSLSVSKCIAVEYVPHRKTWYTKNPGLEVNGNAVPSISPSETFKYLGAKVSPWKGLLEGFESDAFREVISRVQRLPLKPMQKVDLLQMYIFPRYTYGLITSPPAKAVLKTIDRIIRTRIKEILHLPESVSSSFLYTPRKQGGLGLLEVEKMVLIAALRNGLRARQSHDPVTRAAMNSNAADDRLKSYADALRLHWPLTTKELDTYKYQLRLSYAQKWAEQKWQGQGVEEFAQDPVGNSWLQRYDLLPASRYIDAIKLRTNTYPTRALMKIIDGRVDSSCRKCQGSSETLGHILGRCRYTKDKRISRHNEIKDLLKARLAKNHQVMDEPQITVRGQRFKPDLVVKTNEGRVHVIDVTVRYEHRTYLDEGRTEKIGKYRQILSTLRRDLHSNAEEVIPIVIGSRGAIPRETRKALSKLGIGKSDWLTISLIALRSSLEIVNAFMDD

SEQ ID NO: 8 - DNA, 3912 bp, synthetic construct
  source 1..3912: mol_type = other DNA
atgggcaccgataccgtttatgtgggtcaagattatccaagcggcctgtccaagcgcgttccggctcgtctggttgctggtccaatgctgcgcgagcgcagctgccatgcccacgtgttccgtgctggtcacatgtggaattggcgcaccagcctgccgagcggtcgttgggaccagccggccctggagaagagccgcgtgctgacccgtagcgtggctaccgccaccgatccggagatcaccagctacccgggcaagagcgtgagcaccagcacccaggtgcaggaagaggactggtgcagccgtgaaagcggctggatcagcccgggtctggctccagaggaaccgagcgtggtgagcgagattaccgctagcatggtggctaccatgcgtgtggctaccgaggaagtggtgctggaaccgcagccggagcaggtggtgaccattctgccagaacatggtcgtaacgtgccgccgggtctggctgaacaggacaccgcgagcccgattgaggtgagcgtgctgctgccggatctggcggagaactgcccactgtgcggtgtgccgagcggcggtctgcgtctgctgggcaagcacttcgcggtgcgtcatgctggtgtgccagtgacctacgagtgccgcaaatgcgcgtggcgtagcccgaacagccacagcatcagctgccatgtgccgaagtgccgcggtcgtgctcgtatgccgagcggcgacccgggtattgcttgcgatctgtgcgaagcgcgctttgctaccgaggtgggtgtggcccagcacaagcgtcacgtgcacccggtggaatggaacaaagtgcgcctggagcgtcgcggtgcccgtggcggtggcatcaaggcgaccaaactgtggagcgtggctgaagtggaaaccctgatccgtctgattcgtgagcacggcgacagcggtgcgacctatcagctgatcgctgatgaactgggtcgtggcaagaccgccgagcaggtgcgtagcaagaaacgcctgctgcgtattgacaccgccagcaacagcccggacgatgcggaagtggaggaagagcgcctggagagcctggccgtgcgtagcagcagccgtagcccgccgagcctggtggccacccgcgtgcgtgaagcggtggctcgtggtgaaagcgagggtggcgaagagatccgcgccattgccgcgctgatccgtgacgtggatcagaacccgtgcctgattgaaaccagcgccagcgacatcattagcaagctgggccgtcgcgtggacggcccgaaacgcccgcgtccggtggtgcgtgaacagacccaggagaagggttgggtgcgtcgcctggcccgtcgcaaacgcgaataccgtgaggctcagtacctgtatagccgcgaccaggctcgtctggctgcgcagatcctggacggcgccgctagccaggagtgcgccctgccagtggaccaggtgtatggtgctttccgtgagaagtgggaaaccgtgggccagttccacggtctgggcgaatttcgcaccggtgcgcgtgctgataactgggagttctacagcccgatcctggccgcggaagtgaaggagaacctgatgcgcatggctaatggtaccgccccaggtccagaccgtattagcaagaaagccctgctggattgggacccgcgtggtgaacagctggcccgtctgtataccacctggctgatcggtggcgtgattccgcgcgtgttcaaggaatgccgtaccaagctgctgccgaagagcagcgacccggtggagctgcaggatatcggtggctggcgtccggtgaccattggtagcatggtgacccgcctgtttagccgtatcctgaccatgcgtctgacccgcgcgtgcccgattaacccgcgccagcgtggcttcctggccagcagcagcggttgcgccgagaacctgctgatctttgacgagattgtgcgtcgcagccgtcgcgatggtggcccactggccgtggtgttcgtggactttgcccgtgccttcgacagcatcagccacgaacacattctgtgcgtgctggaagagggtggcctggaccgtcacgtgatcggtctgattcgtaacagctacgtggattgcgtgacccgtgtgggctgcgtggagggtatgaccccgccgatccagatgaaagtgggcgtgaaacagggtgacccgatgagcccgctgctgtttaacctggcgatggacccgctgatccacaagctggaaaccgctggtaccggcctgaaatggggcgacctgagcattgccaccctggctttcgccgacgatctggtgctggtgagcgatagcgaagagggtatgggccgtagcctgggcattctggagaagttctgccagctgaccggtctgcgtgtgcagccgcgtaagtgccacggcttctttatggataaaggtgtggtgaacggttgcggcacctgggaaatctgcggcagcccgatccacatgattccgccgggtgagagcgtgcgctacctgggtgtgcaagtgggtccgggtcgtggtgtgatggagccagacctgattccgaccgtgcacacctggatcgaacgcattagcgaagccccgctgaaaccgagccagcgcatgcgtgtgctgaacagcttcgctctgccgcgtatcatctaccaggccgacctgggcaaggtgaccgtgaccaaactggcccagatcgatggtattgtgcgtaaggcggtgaagaaatggctgcacctgagcccgagcacctgcaacggcctgctgtacagccgcaaccgtgatggtggcctgggtctgctgaagctggagcgtctgatcccgagcgtgcgcaccaaacgtatctaccgcatgagccgtagcccggacatctggacccgtcgcatgaccagccacagcgtgagcaagagcgattgggaaatgctgtgggtgcaggcgggtggcgagcgtggcagcgccccggttatgggtgcggtggaagcggctccaaccgacgtggagcgtagcccggactacccagattggcgtcgcgaagagaacctggcctggagcgcgctgcgtgtgcagggcgtgggtgccgaccagttccgcggcgaccgtaccagcagcagctggatcgctgaaccagccagcgtgggcttcgcccagcgtcactggctggcggctctggcgctgcgtgctggcgtgtatccaacccgcgaattcctggcccgtggcaaggagaagagcggtgctgcctgccgtcgctgcccagctcgtctggagagctgcagccacattctgggccagtgcccgtttgtgcaggctaaccgcattgcccgtcacaacaaagtgtgcgtgctgctggctaccgaagccgagcgtttcggctggaccgtgatccgcgaatttcgtctggaggacgctgccggcggcctgaagatcccggatctggtgtgcaagaaagccgacaccgtgctgattgtggacgtgaccgtgcgctacgaaatggacggtgaaaccctgaagcgtgcggctagcgagaaggtgaagcactatctgccggtgggtcagcagatcaccgataaagtgggtggccgctgcttcaaagtgatgggctttccggtgggtgctcgtggcaagtggccggccagcaacaataccgtgctggccgaactgggcgtgccggctggtcgcatgcgtaccttcgcgcgtctggtgagccgtcgcaccctgctgtatagcctggacattctgcgcgacttcatgcgtgaaccagcaggtcgtggcacccgtgtggcactgattccagcagcaaccggcgcagcaaatatggggagcgactacaaagaccatgacggtgattataaagatcatgacatcgattacaaggatgacgatgacaagaagtgataa

SEQ ID NO: 9 - protein, 1302 aa, synthetic construct
MGTDTVYVGQDYPSGLSKRVPARLVAGPMLRERSCHAHVFRAGHMWNWRTSLPSGRWDQPALEKSRVLTRSVATATDPEITSYPGKSVSTSTQVQEEDWCSRESGWISPGLAPEEPSVVSEITASMVATMRVATEEVVLEPQPEQVVTILPEHGRNVPPGLAEQDTASPIEVSVLLPDLAENCPLCGVPSGGLRLLGKHFAVRHAGVPVTYECRKCAWRSPNSHSISCHVPKCRGRARMPSGDPGIACDLCEARFATEVGVAQHKRHVHPVEWNKVRLERRGARGGGIKATKLWSVAEVETLIRLIREHGDSGATYQLIADELGRGKTAEQVRSKKRLLRIDTASNSPDDAEVEEERLESLAVRSSSRSPPSLVATRVREAVARGESEGGEEIRAIAALIRDVDQNPCLIETSASDIISKLGRRVDGPKRPRPVVREQTQEKGWVRRLARRKREYREAQYLYSRDQARLAAQILDGAASQECALPVDQVYGAFREKWETVGQFHGLGEFRTGARADNWEFYSPILAAEVKENLMRMANGTAPGPDRISKKALLDWDPRGEQLARLYTTWLIGGVIPRVFKECRTKLLPKSSDPVELQDIGGWRPVTIGSMVTRLFSRILTMRLTRACPINPRQRGFLASSSGCAENLLIFDEIVRRSRRDGGPLAVVFVDFARAFDSISHEHILCVLEEGGLDRHVIGLIRNSYVDCVTRVGCVEGMTPPIQMKVGVKQGDPMSPLLFNLAMDPLIHKLETAGTGLKWGDLSIATLAFADDLVLVSDSEEGMGRSLGILEKFCQLTGLRVQPRKCHGFFMDKGVVNGCGTWEICGSPIHMIPPGESVRYLGVQVGPGRGVMEPDLIPTVHTWIERISEAPLKPSQRMRVLNSFALPRIIYQADLGKVTVTKLAQIDGIVRKAVKKWLHLSPSTCNGLLYSRNRDGGLGLLKLERLIPSVRTKRIYRMSRSPDIWTRRMTSHSVSKSDWEMLWVQAGGERGSAPVMGAVEAAPTDVERSPDYPDWRREENLAWSALRVQGVGADQFRGDRTSSSWIAEPASVGFAQRHWLAALALRAGVYPTREFLARGKEKSGAACRRCPARLESCSHILGQCPFVQANRIARHNKVCVLLATEAERFGWTVIREFRLEDAAGGLKIPDLVCKKADTVLIVDVTVRYEMDGETLKRAASEKVKHYLPVGQQITDKVGGRCFKVMGFPVGARGKWPASNNTVLAELGVPAGRMRTFARLVSRRTLLYSLDILRDFMREPAGRGTRVALIPAATGAANMGSDYKDHDGDYKDHDIDYKDDDDKK

SEQ ID NO: 10 - RNA, 4371 nt, synthetic construct
  source 1..4371: mol_type = other RNA
aggaataaactagtattcttctggtccccacagactcagagagaacccgccaccatgggcaccgataccgtttatgtgggtcaagattatccaagcggcctgtccaagcgcgttccggctcgtctggttgctggtccaatgctgcgcgagcgcagctgccatgcccacgtgttccgtgctggtcacatgtggaattggcgcaccagcctgccgagcggtcgttgggaccagccggccctggagaagagccgcgtgctgacccgtagcgtggctaccgccaccgatccggagatcaccagctacccgggcaagagcgtgagcaccagcacccaggtgcaggaagaggactggtgcagccgtgaaagcggctggatcagcccgggtctggctccagaggaaccgagcgtggtgagcgagattaccgctagcatggtggctaccatgcgtgtggctaccgaggaagtggtgctggaaccgcagccggagcaggtggtgaccattctgccagaacatggtcgtaacgtgccgccgggtctggctgaacaggacaccgcgagcccgattgaggtgagcgtgctgctgccggatctggcggagaactgcccactgtgcggtgtgccgagcggcggtctgcgtctgctgggcaagcacttcgcggtgcgtcatgctggtgtgccagtgacctacgagtgccgcaaatgcgcgtggcgtagcccgaacagccacagcatcagctgccatgtgccgaagtgccgcggtcgtgctcgtatgccgagcggcgacccgggtattgcttgcgatctgtgcgaagcgcgctttgctaccgaggtgggtgtggcccagcacaagcgtcacgtgcacccggtggaatggaacaaagtgcgcctggagcgtcgcggtgcccgtggcggtggcatcaaggcgaccaaactgtggagcgtggctgaagtggaaaccctgatccgtctgattcgtgagcacggcgacagcggtgcgacctatcagctgatcgctgatgaactgggtcgtggcaagaccgccgagcaggtgcgtagcaagaaacgcctgctgcgtattgacaccgccagcaacagcccggacgatgcggaagtggaggaagagcgcctggagagcctggccgtgcgtagcagcagccgtagcccgccgagcctggtggccacccgcgtgcgtgaagcggtggctcgtggtgaaagcgagggtggcgaagagatccgcgccattgccgcgctgatccgtgacgtggatcagaacccgtgcctgattgaaaccagcgccagcgacatcattagcaagctgggccgtcgcgtggacggcccgaaacgcccgcgtccggtggtgcgtgaacagacccaggagaagggttgggtgcgtcgcctggcccgtcgcaaacgcgaataccgtgaggctcagtacctgtatagccgcgaccaggctcgtctggctgcgcagatcctggacggcgccgctagccaggagtgcgccctgccagtggaccaggtgtatggtgctttccgtgagaagtgggaaaccgtgggccagttccacggtctgggcgaatttcgcaccggtgcgcgtgctgataactgggagttctacagcccgatcctggccgcggaagtgaaggagaacctgatgcgcatggctaatggtaccgccccaggtccagaccgtattagcaagaaagccctgctggattgggacccgcgtggtgaacagctggcccgtctgtataccacctggctgatcggtggcgtgattccgcgcgtgttcaaggaatgccgtaccaagctgctgccgaagagcagcgacccggtggagctgcaggatatcggtggctggcgtccggtgaccattggtagcatggtgacccgcctgtttagccgtatcctgaccatgcgtctgacccgcgcgtgcccgattaacccgcgccagcgtggcttcctggccagcagcagcggttgcgccgagaacctgctgatctttgacgagattgtgcgtcgcagccgtcgcgatggtggcccactggccgtggtgttcgtggactttgcccgtgccttcgacagcatcagccacgaacacattctgtgcgtgctggaagagggtggcctggaccgtcacgtgatcggtctgattcgtaacagctacgtggattgcgtgacccgtgtgggctgcgtggagggtatgaccccgccgatccagatgaaagtgggcgtgaaacagggtgacccgatgagcccgctgctgtttaacctggcgatggacccgctgatccacaagctggaaaccgctggtaccggcctgaaatggggcgacctgagcattgccaccctggctttcgccgacgatctggtgctggtgagcgatagcgaagagggtatgggccgtagcctgggcattctggagaagttctgccagctgaccggtctgcgtgtgcagccgcgtaagtgccacggcttctttatggataaaggtgtggtgaacggttgcggcacctgggaaatctgcggcagcccgatccacatgattccgccgggtgagagcgtgcgctacctgggtgtgcaagtgggtccgggtcgtggtgtgatggagccagacctgattccgaccgtgcacacctggatcgaacgcattagcgaagccccgctgaaaccgagccagcgcatgcgtgtgctgaacagcttcgctctgccgcgtatcatctaccaggccgacctgggcaaggtgaccgtgaccaaactggcccagatcgatggtattgtgcgtaaggcggtgaagaaatggctgcacctgagcccgagcacctgcaacggcctgctgtacagccgcaaccgtgatggtggcctgggtctgctgaagctggagcgtctgatcccgagcgtgcgcaccaaacgtatctaccgcatgagccgtagcccggacatctggacccgtcgcatgaccagccacagcgtgagcaagagcgattgggaaatgctgtgggtgcaggcgggtggcgagcgtggcagcgccccggttatgggtgcggtggaagcggctccaaccgacgtggagcgtagcccggactacccagattggcgtcgcgaagagaacctggcctggagcgcgctgcgtgtgcagggcgtgggtgccgaccagttccgcggcgaccgtaccagcagcagctggatcgctgaaccagccagcgtgggcttcgcccagcgtcactggctggcggctctggcgctgcgtgctggcgtgtatccaacccgcgaattcctggcccgtggcaaggagaagagcggtgctgcctgccgtcgctgcccagctcgtctggagagctgcagccacattctgggccagtgcccgtttgtgcaggctaaccgcattgcccgtcacaacaaagtgtgcgtgctgctggctaccgaagccgagcgtttcggctggaccgtgatccgcgaatttcgtctggaggacgctgccggcggcctgaagatcccggatctggtgtgcaagaaagccgacaccgtgctgattgtggacgtgaccgtgcgctacgaaatggacggtgaaaccctgaagcgtgcggctagcgagaaggtgaagcactatctgccggtgggtcagcagatcaccgataaagtgggtggccgctgcttcaaagtgatgggctttccggtgggtgctcgtggcaagtggccggccagcaacaataccgtgctggccgaactgggcgtgccggctggtcgcatgcgtaccttcgcgcgtctggtgagccgtcgcaccctgctgtatagcctggacattctgcgcgacttcatgcgtgaaccagcaggtcgtggcacccgtgtggcactgattccagcagcaaccggcgcagcaaatatggggagcgactacaaagaccatgacggtgattataaagatcatgacatcgattacaaggatgacgatgacaagaagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 11 - DNA, 3909 bp, synthetic construct
  source 1..3909: mol_type = other DNA
atgggaacagatacagtgtatgtcggccaggactacccttctggcttatcaaaacgggtaccagcacggttagtggcgggaccgatgctgcgagagcgaagctgtcacgcccatgtgtttagggctggacacatgtggaactggcgaaccagccttccgagcgggcgctgggaccagcccgctttggagaagtctcgggtcctaacccggtcggtggcgacggccaccgaccccgaaattacctcttacccaggaaagtccgtatcgacaagtacgcaggttcaggaggaggactggtgtagccgggagagcgggtggatctcgccaggacttgctcctgaagaaccctcggtggtgtccgaaattacagcctccatggtagcgacaatgagggtagcaaccgaggaggtcgtgctggaaccacagcctgaacaggtcgtcacaatactgccggagcatggtcgaaacgttcctccggggctggcagaacaggacaccgccagccccatagaagtctcggtgctcctcccagacctcgctgagaactgcccattgtgtggcgtgccgagcgggggcctacgcttgctcgggaagcattttgctgtccgacatgcgggggtgcctgtaacgtatgagtgccgtaagtgtgcgtggcggagccccaacagccactcaatctcgtgtcacgtccccaaatgccgggggcgtgcgcggatgcccagtggcgatccagggatcgcctgcgatctctgtgaagcccggtttgccacggaggttggggtcgcccaacacaagcggcacgttcatccggtggagtggaacaaggtgaggctggaaaggagaggtgcgcgcggagggggaattaaggcgacgaagctctggagtgtagcggaggtagagacgctaatccggctcatccgtgagcacggagattcaggtgccacttaccagctcattgccgatgagctgggaaggggcaagacggccgaacaggtgaggagtaaaaagaggctcctgcgcatagatacggcaagcaatagcccagatgatgcagaggttgaggaggagaggttggaatctctggcggttcggtcctcgtcacggtcacccccgagcctggtggcgaccagggtcagggaggcagttgccaggggtgaatcagaaggtggcgaggagatcagggctattgctgctctcattagggacgtagatcagaatccttgtctgattgaaacctcggcgtcggacatcatctcgaagctgggaaggagggtggatgggcccaagagacccaggcccgttgtcagagaacagacccaagagaagggatgggtaaggcggcttgcccggcggaaaagggagtacagagaagcgcagtacctgtactcaagggatcaagcaaggctggcggcccagatcctcgatggtgccgccagccaggaatgcgccctcccggtggaccaggtctacggagcgttccgtgagaaatgggaaaccgtagggcagttccacggacttggtgagttccggacgggtgcacgcgcagacaactgggagttctactctccaattctggcggctgaggtgaaagaaaacctaatgagaatggctaacggcacggccccgggaccagacaggataagcaaaaaggctctgcttgactgggacccccggggtgagcaactggcacggctgtacacgacgtggctgatcggtggggtcataccaagggtcttcaaggagtgcaggactaagctgctaccgaaatccagcgacccggtcgagttgcaggacatcggtggatggaggccggtgacgattgggtcgatggtgactaggctgttcagtcggattctaacgatgaggctaacccgagcctgtccgatcaatccgaggcagcgcggtttcttggcctcctcgagtggatgcgcggaaaacctgttgatctttgacgagatcgtcaggcgctcgaggcgggacggggggccgctggcagtggtgtttgtggactttgcgagggcctttgactccatctcacatgaacatatcctgtgtgttctcgaagaaggcgggcttgacaggcacgttatcgggttgatccgaaactcgtacgtggattgcgtgaccagggtgggttgtgtcgagggcatgacaccaccaatacaaatgaaggttggagtgaagcagggagaccccatgtcccccttgctcttcaacctggctatggatcccctcatccataaactcgagacggccggaactggactgaaatggggcgatctttcaatcgccacgctggcctttgccgacgatctggtgctggtgagtgactctgaggaaggcatggggaggagtctcgggattttggagaagttttgccaactgactgggctgagggttcagcccaggaagtgtcacggtttctttatggacaagggcgtggtgaacggctgtggaacctgggaaatctgtgggtcaccgatccacatgattcccccgggggaatcagttcgttatttgggagtccaggtaggcccggggcgcggcgtgatggaaccggatcttatccctacggtccacacgtggatcgaaaggatctcggaggctcctctaaagccctcacaacgcatgagggttttgaactcattcgctctcccccggataatttaccaggccgatctagggaaggttacggtaaccaaattggcccagatagatgggattgtccggaaggctgtgaagaagtggctccatttgtcaccatccacgtgcaatggactgctgtattcacggaaccgcgacggtggtttgggcctcctaaagctggaaagactaatcccatccgtgcgcacgaagcgtatctatcggatgtccaggtctccggatatctggacacggcgaatgaccagccattctgtgtcaaaatctgactgggagatgttgtgggtccaagcgggaggtgagaggggcagtgcacctgtaatgggtgccgtggaggctgccccgaccgatgtggagagatcgccagactacccagactggcggcgtgaggaaaacctggcatggtcggccctgcgggtgcagggtgtgggtgcagaccagtttcgaggcgacaggaccagcagctcttggatcgccgagcccgcttcggttgggttcgcgcagcgccactggttggctgccctggcgctgagggctggggtgtatccgactcgggagtttctggctcggggtaaggaaaagtcaggagcagcttgcagacgctgcccggccaggttggaatcatgttcacacatacttgggcaatgtccgttcgttcaggcgaacagaattgcgaggcacaacaaggtgtgtgtgctcttggccacggaggcggagaggttcggctggacggtaataagggagttccgtcttgaggacgccgctggcggtctcaagatacccgacctggtttgcaagaaggccgacacagttctcattgtcgacgtgaccgtccggtacgagatggatggagagacgctaaaaagggccgcatcggagaaggtgaaacactatctcccagtagggcaacagataacggacaaggtcggagggcgttgctttaaagtcatggggttccctgtaggtgctaggggaaagtggccggcgagcaacaacacagttttggctgagttaggcgtccctgcaggtcggatgaggacctttgccaggctggtgagccggaggactcttctttattctttggatatattgagggacttcatgcgtgagccggccggcaggggaactcgggttgctctcatccctgcggcaacgggtgccgcgaatatggggagcgactacaaagaccatgacggtgattataaagatcatgacatcgattacaaggatgacgatgacaagaagtga

SEQ ID NO: 12 - protein, 1302 aa, synthetic construct; identical to SEQ ID NO: 9

SEQ ID NO: 13 - DNA, 3858 bp, synthetic construct
  source 1..3858: mol_type = other DNA
atggattacaaggatgacgacgataagggtaccgataccgtttatgtgggtcaagattatccaagcggcctgtccaagcgcgttccggctcgtctggttgctggtccaatgctgcgcgagcgcagctgccatgcccacgtgttccgtgctggtcacatgtggaattggcgcaccagcctgccgagcggtcgttgggaccagccggccctggagaagagccgcgtgctgacccgtagcgtggctaccgccaccgatccggagatcaccagctacccgggcaagagcgtgagcaccagcacccaggtgcaggaagaggactggtgcagccgtgaaagcggctggatcagcccgggtctggctccagaggaaccgagcgtggtgagcgagattaccgctagcatggtggctaccatgcgtgtggctaccgaggaagtggtgctggaaccgcagccggagcaggtggtgaccattctgccagaacatggtcgtaacgtgccgccgggtctggctgaacaggacaccgcgagcccgattgaggtgagcgtgctgctgccggatctggcggagaactgcccactgtgcggtgtgccgagcggcggtctgcgtctgctgggcaagcacttcgcggtgcgtcatgctggtgtgccagtgacctacgagtgccgcaaatgcgcgtggcgtagcccgaacagccacagcatcagctgccatgtgccgaagtgccgcggtcgtgctcgtatgccgagcggcgacccgggtattgcttgcgatctgtgcgaagcgcgctttgctaccgaggtgggtgtggcccagcacaagcgtcacgtgcacccggtggaatggaacaaagtgcgcctggagcgtcgcggtgcccgtggcggtggcatcaaggcgaccaaactgtggagcgtggctgaagtggaaaccctgatccgtctgattcgtgagcacggcgacagcggtgcgacctatcagctgatcgctgatgaactgggtcgtggcaagaccgccgagcaggtgcgtagcaagaaacgcctgctgcgtattgacaccgccagcaacagcccggacgatgcggaagtggaggaagagcgcctggagagcctggccgtgcgtagcagcagccgtagcccgccgagcctggtggccacccgcgtgcgtgaagcggtggctcgtggtgaaagcgagggtggcgaagagatccgcgccattgccgcgctgatccgtgacgtggatcagaacccgtgcctgattgaaaccagcgccagcgacatcattagcaagctgggccgtcgcgtggacggcccgaaacgcccgcgtccggtggtgcgtgaacagacccaggagaagggttgggtgcgtcgcctggcccgtcgcaaacgcgaataccgtgaggctcagtacctgtatagccgcgaccaggctcgtctggctgcgcagatcctggacggcgccgctagccaggagtgcgccctgccagtggaccaggtgtatggtgctttccgtgagaagtgggaaaccgtgggccagttccacggtctgggcgaatttcgcaccggtgcgcgtgctgataactgggagttctacagcccgatcctggccgcggaagtgaaggagaacctgatgcgcatggctaatggtaccgccccaggtccagaccgtattagcaagaaagccctgctggattgggacccgcgtggtgaacagctggcccgtctgtataccacctggctgatcggtggcgtgattccgcgcgtgttcaaggaatgccgtaccaagctgctgccgaagagcagcgacccggtggagctgcaggatatcggtggctggcgtccggtgaccattggtagcatggtgacccgcctgtttagccgtatcctgaccatgcgtctgacccgcgcgtgcccgattaacccgcgccagcgtggcttcctggccagcagcagcggttgcgccgagaacctgctgatctttgacgagattgtgcgtcgcagccgtcgcgatggtggcccactggccgtggtgttcgtggactttgcccgtgccttcgacagcatcagccacgaacacattctgtgcgtgctggaagagggtggcctggaccgtcacgtgatcggtctgattcgtaacagctacgtggattgcgtgacccgtgtgggctgcgtggagggtatgaccccgccgatccagatgaaagtgggcgtgaaacagggtgacccgatgagcccgctgctgtttaacctggcgatggacccgctgatccacaagctggaaaccgctggtaccggcctgaaatggggcgacctgagcattgccaccctggctttcgccgacgatctggtgctggtgagcgatagcgaagagggtatgggccgtagcctgggcattctggagaagttctgccagctgaccggtctgcgtgtgcagccgcgtaagtgccacggcttctttatggataaaggtgtggtgaacggttgcggcacctgggaaatctgcggcagcccgatccacatgattccgccgggtgagagcgtgcgctacctgggtgtgcaagtgggtccgggtcgtggtgtgatggagccagacctgattccgaccgtgcacacctggatcgaacgcattagcgaagccccgctgaaaccgagccagcgcatgcgtgtgctgaacagcttcgctctgccgcgtatcatctaccaggccgacctgggcaaggtgaccgtgaccaaactggcccagatcgatggtattgtgcgtaaggcggtgaagaaatggctgcacctgagcccgagcacctgcaacggcctgctgtacagccgcaaccgtgatggtggcctgggtctgctgaagctggagcgtctgatcccgagcgtgcgcaccaaacgtatctaccgcatgagccgtagcccggacatctggacccgtcgcatgaccagccacagcgtgagcaagagcgattgggaaatgctgtgggtgcaggcgggtggcgagcgtggcagcgccccggttatgggtgcggtggaagcggctccaaccgacgtggagcgtagcccggactacccagattggcgtcgcgaagagaacctggcctggagcgcgctgcgtgtgcagggcgtgggtgccgaccagttccgcggcgaccgtaccagcagcagctggatcgctgaaccagccagcgtgggcttcgcccagcgtcactggctggcggctctggcgctgcgtgctggcgtgtatccaacccgcgaattcctggcccgtggcaaggagaagagcggtgctgcctgccgtcgctgcccagctcgtctggagagctgcagccacattctgggccagtgcccgtttgtgcaggctaaccgcattgcccgtcacaacaaagtgtgcgtgctgctggctaccgaagccgagcgtttcggctggaccgtgatccgcgaatttcgtctggaggacgctgccggcggcctgaagatcccggatctggtgtgcaagaaagccgacaccgtgctgattgtggacgtgaccgtgcgctacgaaatggacggtgaaaccctgaagcgtgcggctagcgagaaggtgaagcactatctgccggtgggtcagcagatcaccgataaagtgggtggccgctgcttcaaagtgatgggctttccggtgggtgctcgtggcaagtggccggccagcaacaataccgtgctggccgaactgggcgtgccggctggtcgcatgcgtaccttcgcgcgtctggtgagccgtcgcaccctgctgtatagcctggacattctgcgcgacttcatgcgtgaaccagcaggtcgtggcacccgtgtggcactgattccagcagcaaccggcgcagcaaattgataa

SEQ ID NO: 14 - protein, 1284 aa, synthetic construct
MDYKDDDDKGTDTVYVGQDYPSGLSKRVPARLVAGPMLRERSCHAHVFRAGHMWNWRTSLPSGRWDQPALEKSRVLTRSVATATDPEITSYPGKSVSTSTQVQEEDWCSRESGWISPGLAPEEPSVVSEITASMVATMRVATEEVVLEPQPEQVVTILPEHGRNVPPGLAEQDTASPIEVSVLLPDLAENCPLCGVPSGGLRLLGKHFAVRHAGVPVTYECRKCAWRSPNSHSISCHVPKCRGRARMPSGDPGIACDLCEARFATEVGVAQHKRHVHPVEWNKVRLERRGARGGGIKATKLWSVAEVETLIRLIREHGDSGATYQLIADELGRGKTAEQVRSKKRLLRIDTASNSPDDAEVEEERLESLAVRSSSRSPPSLVATRVREAVARGESEGGEEIRAIAALIRDVDQNPCLIETSASDIISKLGRRVDGPKRPRPVVREQTQEKGWVRRLARRKREYREAQYLYSRDQARLAAQILDGAASQECALPVDQVYGAFREKWETVGQFHGLGEFRTGARADNWEFYSPILAAEVKENLMRMANGTAPGPDRISKKALLDWDPRGEQLARLYTTWLIGGVIPRVFKECRTKLLPKSSDPVELQDIGGWRPVTIGSMVTRLFSRILTMRLTRACPINPRQRGFLASSSGCAENLLIFDEIVRRSRRDGGPLAVVFVDFARAFDSISHEHILCVLEEGGLDRHVIGLIRNSYVDCVTRVGCVEGMTPPIQMKVGVKQGDPMSPLLFNLAMDPLIHKLETAGTGLKWGDLSIATLAFADDLVLVSDSEEGMGRSLGILEKFCQLTGLRVQPRKCHGFFMDKGVVNGCGTWEICGSPIHMIPPGESVRYLGVQVGPGRGVMEPDLIPTVHTWIERISEAPLKPSQRMRVLNSFALPRIIYQADLGKVTVTKLAQIDGIVRKAVKKWLHLSPSTCNGLLYSRNRDGGLGLLKLERLIPSVRTKRIYRMSRSPDIWTRRMTSHSVSKSDWEMLWVQAGGERGSAPVMGAVEAAPTDVERSPDYPDWRREENLAWSALRVQGVGADQFRGDRTSSSWIAEPASVGFAQRHWLAALALRAGVYPTREFLARGKEKSGAACRRCPARLESCSHILGQCPFVQANRIARHNKVCVLLATEAERFGWTVIREFRLEDAAGGLKIPDLVCKKADTVLIVDVTVRYEMDGETLKRAASEKVKHYLPVGQQITDKVGGRCFKVMGFPVGARGKWPASNNTVLAELGVPAGRMRTFARLVSRRTLLYSLDILRDFMREPAGRGTRVALIPAATGAAN

SEQ ID NO: 15 - DNA, 3336 bp, synthetic construct
  source 1..3336: mol_type = other DNA
atggattacaaggatgacgacgataagggtaccgatctggcggagaactgcccactgtgcggtgtgccgagcggcggtctgcgtctgctgggcaagcacttcgcggtgcgtcatgctggtgtgccagtgacctacgagtgccgcaaatgcgcgtggcgtagcccgaacagccacagcatcagctgccatgtgccgaagtgccgcggtcgtgctcgtatgccgagcggcgacccgggtattgcttgcgatctgtgcgaagcgcgctttgctaccgaggtgggtgtggcccagcacaagcgtcacgtgcacccggtggaatggaacaaagtgcgcctggagcgtcgcggtgcccgtggcggtggcatcaaggcgaccaaactgtggagcgtggctgaagtggaaaccctgatccgtctgattcgtgagcacggcgacagcggtgcgacctatcagctgatcgctgatgaactgggtcgtggcaagaccgccgagcaggtgcgtagcaagaaacgcctgctgcgtattgacaccgccagcaacagcccggacgatgcggaagtggaggaagagcgcctggagagcctggccgtgcgtagcagcagccgtagcccgccgagcctggtggccacccgcgtgcgtgaagcggtggctcgtggtgaaagcgagggtggcgaagagatccgcgccattgccgcgctgatccgtgacgtggatcagaacccgtgcctgattgaaaccagcgccagcgacatcattagcaagctgggccgtcgcgtggacggcccgaaacgcccgcgtccggtggtgcgtgaacagacccaggagaagggttgggtgcgtcgcctggcccgtcgcaaacgcgaataccgtgaggctcagtacctgtatagccgcgaccaggctcgtctggctgcgcagatcctggacggcgccgctagccaggagtgcgccctgccagtggaccaggtgtatggtgctttccgtgagaagtgggaaaccgtgggccagttccacggtctgggcgaatttcgcaccggtgcgcgtgctgataactgggagttctacagcccgatcctggccgcggaagtgaaggagaacctgatgcgcatggctaatggtaccgccccaggtccagaccgtattagcaagaaagccctgctggattgggacccgcgtggtgaacagctggcccgtctgtataccacctggctgatcggtggcgtgattccgcgcgtgttcaaggaatgccgtaccaagctgctgccgaagagcagcgacccggtggagctgcaggatatcggtggctggcgtccggtgaccattggtagcatggtgacccgcctgtttagccgtatcctgaccatgcgtctgacccgcgcgtgcccgattaacccgcgccagcgtggcttcctggccagcagcagcggttgcgccgagaacctgctgatctttgacgagattgtgcgtcgcagccgtcgcgatggtggcccactggccgtggtgttcgtggactttgcccgtgccttcgacagcatcagccacgaacacattctgtgcgtgctggaagagggtggcctggaccgtcacgtgatcggtctgattcgtaacagctacgtggattgcgtgacccgtgtgggctgcgtggagggtatgaccccgccgatccagatgaaagtgggcgtgaaacagggtgacccgatgagcccgctgctgtttaacctggcgatggacccgctgatccacaagctggaaaccgctggtaccggcctgaaatggggcgacctgagcattgccaccctggctttcgccgacgatctggtgctggtgagcgatagcgaagagggtatgggccgtagcctgggcattctggagaagttctgccagctgaccggtctgcgtgtgcagccgcgtaagtgccacggcttctttatggataaaggtgtggtgaacggttgcggcacctgggaaatctgcggcagcccgatccacatgattccgccgggtgagagcgtgcgctacctgggtgtgcaagtgggtccgggtcgtggtgtgatggagccagacctgattccgaccgtgcacacctggatcgaacgcattagcgaagccccgctgaaaccgagccagcgcatgcgtgtgctgaacagcttcgctctgccgcgtatcatctaccaggccgacctgggcaaggtgaccgtgaccaaactggcccagatcgatggtattgtgcgtaaggcggtgaagaaatggctgcacctgagcccgagcacctgcaacggcctgctgtacagccgcaaccgtgatggtggcctgggtctgctgaagctggagcgtctgatcccgagcgtgcgcaccaaacgtatctaccgcatgagccgtagcccggacatctggacccgtcgcatgaccagccacagcgtgagcaagagcgattgggaaatgctgtgggtgcaggcgggtggcgagcgtggcagcgccccggttatgggtgcggtggaagcggctccaaccgacgtggagcgtagcccggactacccagattggcgtcgcgaagagaacctggcctggagcgcgctgcgtgtgcagggcgtgggtgccgaccagttccgcggcgaccgtaccagcagcagctggatcgctgaaccagccagcgtgggcttcgcccagcgtcactggctggcggctctggcgctgcgtgctggcgtgtatccaacccgcgaattcctggcccgtggcaaggagaagagcggtgctgcctgccgtcgctgcccagctcgtctggagagctgcagccacattctgggccagtgcccgtttgtgcaggctaaccgcattgcccgtcacaacaaagtgtgcgtgctgctggctaccgaagccgagcgtttcggctggaccgtgatccgcgaatttcgtctggaggacgctgccggcggcctgaagatcccggatctggtgtgcaagaaagccgacaccgtgctgattgtggacgtgaccgtgcgctacgaaatggacggtgaaaccctgaagcgtgcggctagcgagaaggtgaagcactatctgccggtgggtcagcagatcaccgataaagtgggtggccgctgcttcaaagtgatgggctttccggtgggtgctcgtggcaagtggccggccagcaacaataccgtgctggccgaactgggcgtgccggctggtcgcatgcgtaccttcgcgcgtctggtgagccgtcgcaccctgctgtatagcctggacattctgcgcgacttcatgcgtgaaccagcaggtcgtggcacccgtgtggcactgattccagcagcaaccggcgcagcaaattgataa

SEQ ID NO: 16 - protein, 1110 aa, synthetic construct
MDYKDDDDKGTDLAENCPLCGVPSGGLRLLGKHFAVRHAGVPVTYECRKCAWRSPNSHSISCHVPKCRGRARMPSGDPGIACDLCEARFATEVGVAQHKRHVHPVEWNKVRLERRGARGGGIKATKLWSVAEVETLIRLIREHGDSGATYQLIADELGRGKTAEQVRSKKRLLRIDTASNSPDDAEVEEERLESLAVRSSSRSPPSLVATRVREAVARGESEGGEEIRAIAALIRDVDQNPCLIETSASDIISKLGRRVDGPKRPRPVVREQTQEKGWVRRLARRKREYREAQYLYSRDQARLAAQILDGAASQECALPVDQVYGAFREKWETVGQFHGLGEFRTGARADNWEFYSPILAAEVKENLMRMANGTAPGPDRISKKALLDWDPRGEQLARLYTTWLIGGVIPRVFKECRTKLLPKSSDPVELQDIGGWRPVTIGSMVTRLFSRILTMRLTRACPINPRQRGFLASSSGCAENLLIFDEIVRRSRRDGGPLAVVFVDFARAFDSISHEHILCVLEEGGLDRHVIGLIRNSYVDCVTRVGCVEGMTPPIQMKVGVKQGDPMSPLLFNLAMDPLIHKLETAGTGLKWGDLSIATLAFADDLVLVSDSEEGMGRSLGILEKFCQLTGLRVQPRKCHGFFMDKGVVNGCGTWEICGSPIHMIPPGESVRYLGVQVGPGRGVMEPDLIPTVHTWIERISEAPLKPSQRMRVLNSFALPRIIYQADLGKVTVTKLAQIDGIVRKAVKKWLHLSPSTCNGLLYSRNRDGGLGLLKLERLIPSVRTKRIYRMSRSPDIWTRRMTSHSVSKSDWEMLWVQAGGERGSAPVMGAVEAAPTDVERSPDYPDWRREENLAWSALRVQGVGADQFRGDRTSSSWIAEPASVGFAQRHWLAALALRAGVYPTREFLARGKEKSGAACRRCPARLESCSHILGQCPFVQANRIARHNKVCVLLATEAERFGWTVIREFRLEDAAGGLKIPDLVCKKADTVLIVDVTVRYEMDGETLKRAASEKVKHYLPVGQQITDKVGGRCFKVMGFPVGARGKWPASNNTVLAELGVPAGRMRTFARLVSRRTLLYSLDILRDFMREPAGRGTRVALIPAATGAAN

SEQ ID NO: 17 - DNA, 3504 bp, synthetic construct
  source 1..3504: mol_type = other DNA
atggattacaaggatgacgacgataagggtaccatgaacatcgtgaaggtgaccgtgcccgacaagaatcccccttgtccttgctgtggcgtgcgcctgaacagcgtgctggcactgatcgagcacctgaagggctcccacggaaggagaagggtgtgcttccgctgtgccaagtgcggccgggagaactttaatcaccacagcaccgtgtgtcactacgccaagtgcaagggcccacagatcgagagaccacccgtgggcgagtggatctgtgaggtgtgcggcagggatttcaccacaaagatcggcctgggccagcacaagcgccacatgcacgccatggtgcgcaaccaggagcggatcgacgccagccagcccaaggagacctccaatcgcggcgcccacaagcggtgctggacaaaggaggaggaggagctgctgatgaagctggaggtgcagtttgagaaccacaagaacatcaataagctgatcgccgagcagctgaccacaaagaccgccaagcagatctccgataagcgccggatgctgctgaagaagggcagaggcaccacaggcaacctggagacagagcctggcatgagccaccagtcccaggccaaggtgaaggacaatggactgggaggcgatcacctgccaggaggacctgtggtggacaagggcaccatcggcaagccaggccagcacctggacaccgataattctcaccagatcacagcaggcaagaagaagggaggaggactgcaggccagatacagaaggcgcatcatgaagaggctggccgccggcaccatcaacatcttcccaaaggtgtttaaggagctgatcaacgaccaggaggccaggcccctgatcaatcagaccacagaggactgtttcggcctgctggattccgcctgccagatcagaaccgccctgagggagaagggcaagtctcaggaggagcggcccagaaagcagtaccagaagtggatgaagaagcgcgccatcaagcggggcgactatctgcggttccagagactgtttcacctggatagaggcaagctggccaggatcatcctggacaacaccgagtccctgtcttgtgatatcagcccttccgagatctacagcgtgttcaaggcccgctgggagacaccaggccacttcaatggcctgggcgattttgagatcaagggcaaggccaacaataaggccttccgggactttatcaccgccaaggagatcgagaagaacgtgagagagatgtctaagggcagcgcaccaggacctgatggaatcgccctgggcgacatcaagaagatggaccccggctattctcggaccgccgagctgtttaatctgtggctgacagccggcgacatcccagatatggtgaggggatgcaggaccgtgctgatcccaaagagcaccacacccgagcgcctgaaggacatcaacaattggcggcccatcacaatcggctccatcctgctgagactgttctctaggatcatcaccgccagaatgacaaaggcatgtccactgaacccaaggcagaggggctttatctccgcaccaggctgctctgagaacctgaagctgctgcagagcatcatcaggaccgccaagaatgagcacaagcccctgggcgtgatcttcgtggacatcgccaaggcctttgatacagtgtcccaccagcacatcatccacgtgctgcagcagcggagagtggacccccacatcgtgggcctggtgaacaatatgtacaaggacatctccacctatgtgaccacaaagaagaacacccacacagacaagatccagatcagagtgggcgtgaagcagggcgatcctctgtctccactgctgttcaatctggccatggacccactgctgtgtaagctggaggagagcggcaagggcttccacaggggccagagctccatcaccgccatggcctttgccgacgatctggtgctgctgtctgatagctgggagaacatgaaggagaatatcaagatcctggagacattctgtaatctgaccggcctgaagacacagggccagaagtgccacggcttttacatcaagcctaccaaggactcctatacaatcaacaattgcccagcctggaccatcaacggcacacctctgaacatgatcaatccaggcgagtctgagaagtacctgggactgcagatcgacccatggaccggcgtggccaagtatgatctgagcacaaagctgaagatctggctggagtccatcgacagggcccccctgaagcctctgcagaagctggatatcctgaagacctacacaatcccccgcctgacctatctggccgatcacagcgagatgaaggcaggagcactggaggcactggaccagcagatcaggaccgccgtgaaggattggctgcacctgccttcctgcacatgtgacgccatcctgtacgtgtctaccagggatggaggactgggagtgacaaagctggcaggactgatcccatctgtgcaggcaaggaggctgcacaggatcgcacagagccctgacgagaccatgaaggatttcctggagaaggcccagatggagaagatgtacgagaagctgtgggtgcaggccggcggcaagaagaagggcatgccatccatctgggaggccctgcccatgacagtgcctccaaccaacacaggcaatctgtctgagtgggaggccccaaatcccaagagcaagtatcctaagccatgcgactggcggagaaaggagctgaagaagtggaccaagctggagagccagggcagaggcgtgaagaactttaggaatgacacaatctccaacgattggattcagtactataggcgcatcccacaccgcaagctgctgaccgccatccagctgcgcgccaacgtgtacccaacaagggagttcctggcacggggcagaggcgataattatgtgaagttttgtcggcactgcgaggccgacctggagacctgtggccacatcatcggcttctgccccgtgacaaaggacgcccggatcaagagacacaacaggatctgtgatagactgtgcgaggaggcagcaaagagggagtgggtggtgtttaaggagccccacctgagggacgccaccacagagctgttcaagcctgatgtgatctttgtgaaggaggacagggcactggtggtggatgtgaccgtgcggtacgagtccgccaagaccacactggaggctgccgccatggagaaggtggataagtataagcacctggaggccgaggtgaaggagctgacaaatgccaaggacgtggtgttcatgggatttccactgggagcaaggggcaagttctacaagggcaactttaatctgctggagaccctgggcctgcctaagacaagacagctgtctgtggccaagaccctgagcacctacgccctgatgtctagcgtggacatcgtgcacatgttcgcctccaggtctcgcaagcctaacgtgtag

SEQ ID NO: 18 - protein, 1167 aa, synthetic construct
MDYKDDDDKGTMNIVKVTVPDKNPPCPCCGVRLNSVLALIEHLKGSHGRRRVCFRCAKCGRENFNHHSTVCHYAKCKGPQIERPPVGEWICEVCGRDFTTKIGLGQHKRHMHAMVRNQERIDASQPKETSNRGAHKRCWTKEEEELLMKLEVQFENHKNINKLIAEQLTTKTAKQISDKRRMLLKKGRGTTGNLETEPGMSHQSQAKVKDNGLGGDHLPGGPVVDKGTIGKPGQHLDTDNSHQITAGKKKGGGLQARYRRRIMKRLAAGTINIFPKVFKELINDQEARPLINQTTEDCFGLLDSACQIRTALREKGKSQEERPRKQYQKWMKKRAIKRGDYLRFQRLFHLDRGKLARIILDNTESLSCDISPSEIYSVFKARWETPGHFNGLGDFEIKGKANNKAFRDFITAKEIEKNVREMSKGSAPGPDGIALGDIKKMDPGYSRTAELFNLWLTAGDIPDMVRGCRTVLIPKSTTPERLKDINNWRPITIGSILLRLFSRIITARMTKACPLNPRQRGFISAPGCSENLKLLQSIIRTAKNEHKPLGVIFVDIAKAFDTVSHQHIIHVLQQRRVDPHIVGLVNNMYKDISTYVTTKKNTHTDKIQIRVGVKQGDPLSPLLFNLAMDPLLCKLEESGKGFHRGQSSITAMAFADDLVLLSDSWENMKENIKILETFCNLTGLKTQGQKCHGFYIKPTKDSYTINNCPAWTINGTPLNMINPGESEKYLGLQIDPWTGVAKYDLSTKLKIWLESIDRAPLKPLQKLDILKTYTIPRLTYLADHSEMKAGALEALDQQIRTAVKDWLHLPSCTCDAILYVSTRDGGLGVTKLAGLIPSVQARRLHRIAQSPDETMKDFLEKAQMEKMYEKLWVQAGGKKKGMPSIWEALPMTVPPTNTGNLSEWEAPNPKSKYPKPCDWRRKELKKWTKLESQGRGVKNFRNDTISNDWIQYYRRIPHRKLLTAIQLRANVYPTREFLARGRGDNYVKFCRHCEADLETCGHIIGFCPVTKDARIKRHNRICDRLCEEAAKREWVVFKEPHLRDATTELFKPDVIFVKEDRALVVDVTVRYESAKTTLEAAAMEKVDKYKHLEAEVKELTNAKDVVFMGFPLGARGKFYKGNFNLLETLGLPKTRQLSVAKTLSTYALMSSVDIVHMFASRSRKPNV

SEQ ID NO: 19 - RNA, 3964 nt, synthetic construct
  source 1..3964: mol_type = other RNA
aggaataaactagtattcttctggtccccacagactcagagagaacccgccaccatggattacaaggatgacgacgataagggatccaacatcgtgaaggtgaccgtgcccgacaagaatcccccttgtccttgctgtggcgtgcgcctgaacagcgtgctggcactgatcgagcacctgaagggctcccacggaaggagaagggtgtgcttccgctgtgccaagtgcggccgggagaactttaatcaccacagcaccgtgtgtcactacgccaagtgcaagggcccacagatcgagagaccacccgtgggcgagtggatctgtgaggtgtgcggcagggatttcaccacaaagatcggcctgggccagcacaagcgccacatgcacgccatggtgcgcaaccaggagcggatcgacgccagccagcccaaggagacctccaatcgcggcgcccacaagcggtgctggacaaaggaggaggaggagctgctgatgaagctggaggtgcagtttgagaaccacaagaacatcaataagctgatcgccgagcagctgaccacaaagaccgccaagcagatctccgataagcgccggatgctgctgaagaagggcagaggcaccacaggcaacctggagacagagcctggcatgagccaccagtcccaggccaaggtgaaggacaatggactgggaggcgatcacctgccaggaggacctgtggtggacaagggcaccatcggcaagccaggccagcacctggacaccgataattctcaccagatcacagcaggcaagaagaagggaggaggactgcaggccagatacagaaggcgcatcatgaagaggctggccgccggcaccatcaacatcttcccaaaggtgtttaaggagctgatcaacgaccaggaggccaggcccctgatcaatcagaccacagaggactgtttcggcctgctggattccgcctgccagatcagaaccgccctgagggagaagggcaagtctcaggaggagcggcccagaaagcagtaccagaagtggatgaagaagcgcgccatcaagcggggcgactatctgcggttccagagactgtttcacctggatagaggcaagctggccaggatcatcctggacaacaccgagtccctgtcttgtgatatcagcccttccgagatctacagcgtgttcaaggcccgctgggagacaccaggccacttcaatggcctgggcgattttgagatcaagggcaaggccaacaataaggccttccgggactttatcaccgccaaggagatcgagaagaacgtgagagagatgtctaagggcagcgcaccaggacctgatggaatcgccctgggcgacatcaagaagatggaccccggctattctcggaccgccgagctgtttaatctgtggctgacagccggcgacatcccagatatggtgaggggatgcaggaccgtgctgatcccaaagagcaccacacccgagcgcctgaaggacatcaacaattggcggcccatcacaatcggctccatcctgctgagactgttctctaggatcatcaccgccagaatgacaaaggcatgtccactgaacccaaggcagaggggctttatctccgcaccaggctgctctgagaacctgaagctgctgcagagcatcatcaggaccgccaagaatgagcacaagcccctgggcgtgatcttcgtggacatcgccaaggcctttgatacagtgtcccaccagcacatcatccacgtgctgcagcagcggagagtggacccccacatcgtgggcctggtgaacaatatgtacaaggacatctccacctatgtgaccacaaagaagaacacccacacagacaagatccagatcagagtgggcgtgaagcagggcgatcctctgtctccactgctgttcaatctggccatggacccactgctgtgtaagctggaggagagcggcaagggcttccacaggggccagagctccatcaccgccatggcctttgccgacgatctggtgctgctgtctgatagctgggagaacatgaaggagaatatcaagatcctggagacattctgtaatctgaccggcctaaagacacagggccagaagtgccacggcttttacatcaagcctaccaaggactcctatacaatcaacaattgcccagcctggaccatcaacggcacacctctgaacatgatcaatccaggcgagtctgagaagtacctgggactgcagatcgacccatggaccggcgtggccaagtatgatctgagcacaaagctgaagatctggctggagtccatcgacagggcccccctgaagcctctgcagaagctggatatcctgaaaacctacacaatcccccgcctgacctatctggccgatcacagcgagatgaaggcaggagcactggaggcactggaccagcagatcaggaccgccgtgaaggattggctgcacctgccttcctgcacatgtgacgccatcctgtacgtgtctaccagggatggaggactgggagtgacaaagctggcaggactgatcccatctgtgcaggcaaggaggctgcacaggatcgcacagagccctgacgagaccatgaaggatttcctggagaaggcccagatggagaagatgtacgagaagctgtgggtgcaggccggcggcaagaagaagggcatgccatccatctgggaggccctgcccatgacagtgcctccaaccaacacaggcaatctgtctgagtgggaggccccaaatcccaagagcaagtatcctaagccatgcgactggcggagaaaggagctgaagaagtggaccaagctggagagccagggcagaggcgtgaagaactttaggaatgacacaatctccaacgattggattcagtactataggcgcatcccacaccgcaagctgctgaccgccatccagctgcgcgccaacgtgtacccaacaagggagttcctggcacggggcagaggcgataattatgtgaagttttgtcggcactgcgaggccgacctggagacctgtggccacatcatcggcttctgccccgtgacaaaggacgcccggatcaagagacacaacaggatctgtgatagactgtgcgaggaggcagcaaagagggagtgggtggtgtttaaggagccccacctgagggacgccaccacagagctgttcaagcctgatgtgatctttgtgaaggaggacagggcactggtggtggatgtgaccgtgcggtacgagtccgccaagaccacactggaggctgccgccatggagaaggtggataagtataagcacctggaggccgaggtgaaggagctgacaaatgccaaggacgtggtgttcatgggatttccactgggagcaaggggcaagttctacaagggcaactttaatctgctggagaccctgggcctgcctaagacaagacagctgtctgtggccaagaccctgagcacctacgccctgatgtctagcgtggacatcgtgcacatgttcgcctccaggtctcgcaagcctaacgtgtgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 20 - protein, 1156 aa, synthetic construct
MNIVKVTVPDKNPPCPCCGVRLNSVLALIEHLKGSHGRRRVCFRCAKCGRENFNHHSTVCHYAKCKGPQIERPPVGEWICEVCGRDFTTKIGLGQHKRHMHAMVRNQERIDASQPKETSNRGAHKRCWTKEEEELLMKLEVQFENHKNINKLIAEQLTTKTAKQISDKRRMLLKKGRGTTGNLETEPGMSHQSQAKVKDNGLGGDHLPGGPVVDKGTIGKPGQHLDTDNSHQITAGKKKGGGLQARYRRRIMKRLAAGTINIFPKVFKELINDQEARPLINQTTEDCFGLLDSACQIRTALREKGKSQEERPRKQYQKWMKKRAIKRGDYLRFQRLFHLDRGKLARIILDNTESLSCDISPSEIYSVFKARWETPGHFNGLGDFEIKGKANNKAFRDFITAKEIEKNVREMSKGSAPGPDGIALGDIKKMDPGYSRTAELFNLWLTAGDIPDMVRGCRTVLIPKSTTPERLKDINNWRPITIGSILLRLFSRIITARMTKACPLNPRQRGFISAPGCSENLKLLQSIIRTAKNEHKPLGVIFVDIAKAFDTVSHQHIIHVLQQRRVDPHIVGLVNNMYKDISTYVTTKKNTHTDKIQIRVGVKQGDPLSPLLFNLAMDPLLCKLEESGKGFHRGQSSITAMAFADDLVLLSDSWENMKENIKILETFCNLTGLKTQGQKCHGFYIKPTKDSYTINNCPAWTINGTPLNMINPGESEKYLGLQIDPWTGVAKYDLSTKLKIWLESIDRAPLKPLQKLDILKTYTIPRLTYLADHSEMKAGALEALDQQIRTAVKDWLHLPSCTCDAILYVSTRDGGLGVTKLAGLIPSVQARRLHRIAQSPDETMKDFLEKAQMEKMYEKLWVQAGGKKKGMPSIWEALPMTVPPTNTGNLSEWEAPNPKSKYPKPCDWRRKELKKWTKLESQGRGVKNFRNDTISNDWIQYYRRIPHRKLLTAIQLRANVYPTREFLARGRGDNYVKFCRHCEADLETCGHIIGFCPVTKDARIKRHNRICDRLCEEAAKREWVVFKEPHLRDATTELFKPDVIFVKEDRALVVDVTVRYESAKTTLEAAAMEKVDKYKHLEAEVKELTNAKDVVFMGFPLGARGKFYKGNFNLLETLGLPKTRQLSVAKTLSTYALMSSVDIVHMFASRSRKPNV

SEQ ID NO: 21 - RNA, 3934 nt, synthetic construct
  source 1..3934: mol_type = other RNA
aggaataaactagtattcttctggtccccacagactcagagagaacccgccaccatgaacatcgtgaaggtgaccgtgcccgacaagaatcccccttgtccttgctgtggcgtgcgcctgaacagcgtgctggcactgatcgagcacctgaagggctcccacggaaggagaagggtgtgcttccgctgtgccaagtgcggccgggagaactttaatcaccacagcaccgtgtgtcactacgccaagtgcaagggcccacagatcgagagaccacccgtgggcgagtggatctgtgaggtgtgcggcagggatttcaccacaaagatcggcctgggccagcacaagcgccacatgcacgccatggtgcgcaaccaggagcggatcgacgccagccagcccaaggagacctccaatcgcggcgcccacaagcggtgctggacaaaggaggaggaggagctgctgatgaagctggaggtgcagtttgagaaccacaagaacatcaataagctgatcgccgagcagctgaccacaaagaccgccaagcagatctccgataagcgccggatgctgctgaagaagggcagaggcaccacaggcaacctggagacagagcctggcatgagccaccagtcccaggccaaggtgaaggacaatggactgggaggcgatcacctgccaggaggacctgtggtggacaagggcaccatcggcaagccaggccagcacctggacaccgataattctcaccagatcacagcaggcaagaagaagggaggaggactgcaggccagatacagaaggcgcatcatgaagaggctggccgccggcaccatcaacatcttcccaaaggtgtttaaggagctgatcaacgaccaggaggccaggcccctgatcaatcagaccacagaggactgtttcggcctgctggattccgcctgccagatcagaaccgccctgagggagaagggcaagtctcaggaggagcggcccagaaagcagtaccagaagtggatgaagaagcgcgccatcaagcggggcgactatctgcggttccagagactgtttcacctggatagaggcaagctggccaggatcatcctggacaacaccgagtccctgtcttgtgatatcagcccttccgagatctacagcgtgttcaaggcccgctgggagacaccaggccacttcaatggcctgggcgattttgagatcaagggcaaggccaacaataaggccttccgggactttatcaccgccaaggagatcgagaagaacgtgagagagatgtctaagggcagcgcaccaggacctgatggaatcgccctgggcgacatcaagaagatggaccccggctattctcggaccgccgagctgtttaatctgtggctgacagccggcgacatcccagatatggtgaggggatgcaggaccgtgctgatcccaaagagcaccacacccgagcgcctgaaggacatcaacaattggcggcccatcacaatcggctccatcctgctgagactgttctctaggatcatcaccgccagaatgacaaaggcatgtccactgaacccaaggcagaggggctttatctccgcaccaggctgctctgagaacctgaagctgctgcagagcatcatcaggaccgccaagaatgagcacaagcccctgggcgtgatcttcgtggacatcgccaaggcctttgatacagtgtcccaccagcacatcatccacgtgctgcagcagcggagagtggacccccacatcgtgggcctggtgaacaatatgtacaaggacatctccacctatgtgaccacaaagaagaacacccacacagacaagatccagatcagagtgggcgtgaagcagggcgatcctctgtctccactgctgttcaatctggccatggacccactgctgtgtaagctggaggagagcggcaagggcttccacaggggccagagctccatcaccgccatggcctttgccgacgatctggtgctgctgtctgatagctgggagaacatgaaggagaatatcaagatcctggagacattctgtaatctgaccggcctaaagacacagggccagaagtgccacggcttttacatcaagcctaccaaggactcctatacaatcaacaattgcccagcctggaccatcaacggcacacctctgaacatgatcaatccaggcgagtctgagaagtacctgggactgcagatcgacccatggaccggcgtggccaagtatgatctgagcacaaagctgaagatctggctggagtccatcgacagggcccccctgaagcctctgcagaagctggatatcctgaaaacctacacaatcccccgcctgacctatctggccgatcacagcgagatgaaggcaggagcactggaggcactggaccagcagatcaggaccgccgtgaaggattggctgcacctgccttcctgcacatgtgacgccatcctgtacgtgtctaccagggatggaggactgggagtgacaaagctggcaggactgatcccatctgtgcaggcaaggaggctgcacaggatcgcacagagccctgacgagaccatgaaggatttcctggagaaggcccagatggagaagatgtacgagaagctgtgggtgcaggccggcggcaagaagaagggcatgccatccatctgggaggccctgcccatgacagtgcctccaaccaacacaggcaatctgtctgagtgggaggccccaaatcccaagagcaagtatcctaagccatgcgactggcggagaaaggagctgaagaagtggaccaagctggagagccagggcagaggcgtgaagaactttaggaatgacacaatctccaacgattggattcagtactataggcgcatcccacaccgcaagctgctgaccgccatccagctgcgcgccaacgtgtacccaacaagggagttcctggcacggggcagaggcgataattatgtgaagttttgtcggcactgcgaggccgacctggagacctgtggccacatcatcggcttctgccccgtgacaaaggacgcccggatcaagagacacaacaggatctgtgatagactgtgcgaggaggcagcaaagagggagtgggtggtgtttaaggagccccacctgagggacgccaccacagagctgttcaagcctgatgtgatctttgtgaaggaggacagggcactggtggtggatgtgaccgtgcggtacgagtccgccaagaccacactggaggctgccgccatggagaaggtggataagtataagcacctggaggccgaggtgaaggagctgacaaatgccaaggacgtggtgttcatgggatttccactgggagcaaggggcaagttctacaagggcaactttaatctgctggagaccctgggcctgcctaagacaagacagctgtctgtggccaagaccctgagcacctacgccctgatgtctagcgtggacatcgtgcacatgttcgcctccaggtctcgcaagcctaacgtgtgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 22 - protein, 1156 aa, synthetic construct
MNIVKVTVPDKNPPCPCCGVRLNSVLALIEHLKGSHGRRRVCFRCAKCGRENFNHHSTVCHYAKCKGPQIERPPVGEWICEVCGRDFTTKIGLGQHKRHMHAMVRNQERIDASQPKETSNRGAHKRCWTKEEEELLMKLEVQFENHKNINKLIAEQLTTKTAKQISDKRRMLLKKGRGTTGNLETEPGMSHQSQAKVKDNGLGGDHLPGGPVVDKGTIGKPGQHLDTDNSHQITAGKKKGGGLQARYRRRIMKRLAAGTINIFPKVFKELINDQEARPLINQTTEDCFGLLDSACQIRTALREKGKSQEERPRKQYQKWMKKRAIKRGDYLRFQRLFHLDRGKLARIILDNTESLSCDISPSEIYSVFKARWETPGHFNGLGDFEIKGKANNKAFRDFITAKEIEKNVREMSKGSAPGPDGIALGDIKKMDPGYSRTAELFNLWLTAGDIPDMVRGCRTVLIPKSTTPERLKDINNWRPITIGSILLRLFSRIITARMTKACPLNPRQRGFISAPGCSENLKLLQSIIRTAKNEHKPLGVIFVDIAKAFDTVSHQHIIHVLQQRRVDPHIVGLVNNMYKDISTYVTTKKNTHTDKIQIRVGVKQGDPLSPLLFNLAMDPLLCKLEESGKGFHRGQSSITAMAFAAALVLLSDSWENMKENIKILETFCNLTGLKTQGQKCHGFYIKPTKDSYTINNCPAWTINGTPLNMINPGESEKYLGLQIDPWTGVAKYDLSTKLKIWLESIDRAPLKPLQKLDILKTYTIPRLTYLADHSEMKAGALEALDQQIRTAVKDWLHLPSCTCDAILYVSTRDGGLGVTKLAGLIPSVQARRLHRIAQSPDETMKDFLEKAQMEKMYEKLWVQAGGKKKGMPSIWEALPMTVPPTNTGNLSEWEAPNPKSKYPKPCDWRRKELKKWTKLESQGRGVKNFRNDTISNDWIQYYRRIPHRKLLTAIQLRANVYPTREFLARGRGDNYVKFCRHCEADLETCGHIIGFCPVTKDARIKRHNRICDRLCEEAAKREWVVFKEPHLRDATTELFKPDVIFVKEDRALVVDVTVRYESAKTTLEAAAMEKVDKYKHLEAEVKELTNAKDVVFMGFPLGARGKFYKGNFNLLETLGLPKTRQLSVAKTLSTYALMSSVDIVHMFASRSRKPNV

SEQ ID NO: 23 - RNA, 3934 nt, synthetic construct
  source 1..3934: mol_type = other RNA
aggaataaactagtattcttctggtccccacagactcagagagaacccgccaccatgaacatcgtgaaggtgaccgtgcccgacaagaatcccccttgtccttgctgtggcgtgcgcctgaacagcgtgctggcactgatcgagcacctgaagggctcccacggaaggagaagggtgtgcttccgctgtgccaagtgcggccgggagaactttaatcaccacagcaccgtgtgtcactacgccaagtgcaagggcccacagatcgagagaccacccgtgggcgagtggatctgtgaggtgtgcggcagggatttcaccacaaagatcggcctgggccagcacaagcgccacatgcacgccatggtgcgcaaccaggagcggatcgacgccagccagcccaaggagacctccaatcgcggcgcccacaagcggtgctggacaaaggaggaggaggagctgctgatgaagctggaggtgcagtttgagaaccacaagaacatcaataagctgatcgccgagcagctgaccacaaagaccgccaagcagatctccgataagcgccggatgctgctgaagaagggcagaggcaccacaggcaacctggagacagagcctggcatgagccaccagtcccaggccaaggtgaaggacaatggactgggaggcgatcacctgccaggaggacctgtggtggacaagggcaccatcggcaagccaggccagcacctggacaccgataattctcaccagatcacagcaggcaagaagaagggaggaggactgcaggccagatacagaaggcgcatcatgaagaggctggccgccggcaccatcaacatcttcccaaaggtgtttaaggagctgatcaacgaccaggaggccaggcccctgatcaatcagaccacagaggactgtttcggcctgctggattccgcctgccagatcagaaccgccctgagggagaagggcaagtctcaggaggagcggcccagaaagcagtaccagaagtggatgaagaagcgcgccatcaagcggggcgactatctgcggttccagagactgtttcacctggatagaggcaagctggccaggatcatcctggacaacaccgagtccctgtcttgtgatatcagcccttccgagatctacagcgtgttcaaggcccgctgggagacaccaggccacttcaatggcctgggcgattttgagatcaagggcaaggccaacaataaggccttccgggactttatcaccgccaaggagatcgagaagaacgtgagagagatgtctaagggcagcgcaccaggacctgatggaatcgccctgggcgacatcaagaagatggaccccggctattctcggaccgccgagctgtttaatctgtggctgacagccggcgacatcccagatatggtgaggggatgcaggaccgtgctgatcccaaagagcaccacacccgagcgcctgaaggacatcaacaattggcggcccatcacaatcggctccatcctgctgagactgttctctaggatcatcaccgccagaatgacaaaggcatgtccactgaacccaaggcagaggggctttatctccgcaccaggctgctctgagaacctgaagctgctgcagagcatcatcaggaccgccaagaatgagcacaagcccctgggcgtgatcttcgtggacatcgccaaggcctttgatacagtgtcccaccagcacatcatccacgtgctgcagcagcggagagtggacccccacatcgtgggcctggtgaacaatatgtacaaggacatctccacctatgtgaccacaaagaagaacacccacacagacaagatccagatcagagtgggcgtgaagcagggcgatcctctgtctccactgctgttcaatctggccatggacccactgctgtgtaagctggaggagagcggcaagggcttccacaggggccagagctccatcaccgccatggcctttgccgccgcactggtgctgctgtctgatagctgggagaacatgaaggagaatatcaagatcctggagacattctgtaatctgaccggcctaaagacacagggccagaagtgccacggcttttacatcaagcctaccaaggactcctatacaatcaacaattgcccagcctggaccatcaacggcacacctctgaacatgatcaatccaggcgagtctgagaagtacctgggactgcagatcgacccatggaccggcgtggccaagtatgatctgagcacaaagctgaagatctggctggagtccatcgacagggcccccctgaagcctctgcagaagctggatatcctgaaaacctacacaatcccccgcctgacctatctggccgatcacagcgagatgaaggcaggagcactggaggcactggaccagcagatcaggaccgccgtgaaggattggctgcacctgccttcctgcacatgtgacgccatcctgtacgtgtctaccagggatggaggactgggagtgacaaagctggcaggactgatcccatctgtgcaggcaaggaggctgcacaggatcgcacagagccctgacgagaccatgaaggatttcctggagaaggcccagatggagaagatgtacgagaagctgtgggtgcaggccggcggcaagaagaagggcatgccatccatctgggaggccctgcccatgacagtgcctccaaccaacacaggcaatctgtctgagtgggaggccccaaatcccaagagcaagtatcctaagccatgcgactggcggagaaaggagctgaagaagtggaccaagctggagagccagggcagaggcgtgaagaactttaggaatgacacaatctccaacgattggattcagtactataggcgcatcccacaccgcaagctgctgaccgccatccagctgcgcgccaacgtgtacccaacaagggagttcctggcacggggcagaggcgataattatgtgaagttttgtcggcactgcgaggccgacctggagacctgtggccacatcatcggcttctgccccgtgacaaaggacgcccggatcaagagacacaacaggatctgtgatagactgtgcgaggaggcagcaaagagggagtgggtggtgtttaaggagccccacctgagggacgccaccacagagctgttcaagcctgatgtgatctttgtgaaggaggacagggcactggtggtggatgtgaccgtgcggtacgagtccgccaagaccacactggaggctgccgccatggagaaggtggataagtataagcacctggaggccgaggtgaaggagctgacaaatgccaaggacgtggtgttcatgggatttccactgggagcaaggggcaagttctacaagggcaactttaatctgctggagaccctgggcctgcctaagacaagacagctgtctgtggccaagaccctgagcacctacgccctgatgtctagcgtggacatcgtgcacatgttcgcctccaggtctcgcaagcctaacgtgtgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 24 - DNA, 3516 bp, synthetic construct
  source 1..3516: mol_type = other DNA
atggattacaaggatgacgacgataagggtaccatggtgggcggaagccctcctggcgagggcgttagaagccctggaatcgcctctcctagcggtacagccgtgcagcacgacttcggctctccaatcctggtgcctggcgctgaagctgccgaagtttctacacctgtggtgaaagtgccccaggaccacccagcctgtccatgctgcggcacacgggtggtgaaggtgacagccctgtctgagcacctccggcgcgcccacgggagaaagcgggtgctgttccagtgctctcggtgcggcagaatgaacgagaagcaccattctatcgcctgccacttccccaagtgccggggacctcctgtggaggaaggccccctgggcgctcctgaatggtgctgtgaagagtgcggacagaagttcaacacaaagtccggcctgagccagcataagcgcagcgtgcaccccctgaccagaaatgtggaaagaattgaagccgcccgccccaagggtaagggcaaaagaggagcccataagggctgttggacagaggccgaggtggcacagctcatcgaactggagggcagattcaagaaccagagattcatcaacaagctgattgcggagcacctgccttctaaatccgccaagcagatcagcgacaagagacggcagctggccgctgctaccaagacctctagccccgagaagagggtgaccagcagcacaagcggagagagcagtcctgaggtggagaaagtggagggaataaaacgggagtaccgtagacgggtgggcgagtggctgtgcgccggtagtctgaacgaccagaccagttttcagaaaatcctggaagatgtggagagcggcagcgagatcgtgaccgggccactggaggaactggccagcttcgccaggggcaagctggccgccgctagagtgcggcaccaccggaagcatcccgccgaggccgtgcctgcgagagaggagcagagatggatgaagcgcagagtcggacgacggggactgtacctgagattccagagactgttcgccctggaccggagaaagctagccggcatcatcctggacgatgtggaatctatcaagtgccctctgcctatggaagaagtggccgacgtgttccggaggaggtgggaagaagtggcccctttcaccgggtcaggcagcttcaggtctctgggcaaggccgacaacggcgccttcaagcctatgatcagcgccaaggaagttatgaagaacgtgcgggagatgagccgacggagcgctcctggacctgatggcctgtccctgagggatctcatgaagatcgatcctcagggctccagaatggccgaactgttcaatctgtggctgctggctggaagagtgccagaccaggtgaaggccggcagaaccgtcctgattcctaagagcgccgatcctgggaagatcggcaatatcgacaactggcggcctatcaccatcggaagcgtgctgcttagaatgttcagcagaatcctgagtgccagactgcggagagcctgtcctatcaacagacggcagcggggctttatcgccgcccccggctgcagcgagaacctgaagttactgcaagccctcattaagagcgccaagcgggaccacagaacactgggcgtggtgttcgtggacctggcgaaggccttcgacagcgtgaaccaccagcacatcttccaagtgctggtacagaagggggtggatggccacatcatcgacatcctgagagatctttacaccaacgccggcacctacctggaatccggctctcagagaagcggctttatcaagatcctgagaggcgtcaagcagggcgacccactgagcccaatcctgtttaacctggccctggaccccctgctatgtagactggaggaccggggcctgggatataagtacggcgatcagcaaatcaccagcctggcattcgccgatgatctggccctactgagcgacagctgggagggcatgcagcaaagcatccgggtcgtcgaagagttttgccagaggacaggcctgcgagtgcaagctccaaagtgccacggcttcctgatcagacctaccaaagaatcttacaccattaacgactgcgacccttggacaatcgccgacatgcagctggatatgatcgacccaggctccagcgaaaaatacctgggcctgggcatcgacccttggatcggcctgagccgtcctgagctgtccgaagtgctgaccagatgggtgaagaacatcggaggcgcccccctgaagcccctgcaaaaggtggacatcctgagaagctacgccctgcctagactgctgtttatcgccgaccacgccggcctgtccgccacctgcctgcactctctggatctttccatcagaagcgctgtgaagggctggctgcacctgcctcccagcacctgtgatgccatcatctacgtgagctacaaggacggcggactgggcctgcccagactggcaagcctgatccctaacgtgcaggcaagacggctggtgagaatcgcccaaagcgaggatgacgtgatccgcagcgtggtgctgcaggagggaatccaggaggagatcagaaaagtgtggatctctgccggcggcagacctgagaaggtccctagcgtgaccggcgaattccccgtgatggaagcccaggccgccgacgaggcactgtccgagtgggagagacgggcccctcgcaccatctaccccatcccttgtaaatggagaaagcgcgagatggaaaactggacgaatctcaaatctcagggccacggcattagaaatttcgagaatgacagaatcagcaacgactggctgctgcactacgggagaatccctcacagaaagctgatcacagctatccagctgagagccaacgtgtatcctacaagagagttcctggctagaggccttggcgagggcgctcctagaggctgcagacactgtcccgccgaatgggaatcctgcagccacatcatcggctactgccctgccgtgcaggaggccagaatcaagagacacaacgacatctgcggcgtgcttgctgaagaggccagaaagctgggatgggtgatcttcatcgagcctcacttgagagataataccaacgagctgttcaaacccgacctggtgttggtgaaaggatcttgcgccaaggtggtggacgtgaccatcagatacgagagcgggctaaccaccctgtcagatgctgccgctgagaaagcacgaaaataccagcacctggcgggcgaggtgagagccctgactagcgccacaaccgtggactttctgggcttccccatcggtgcccggggcaaatggtacgttggaaacaacggcctcctgagcgacctgggcttcagcaccagtagagttgttcggatcgcccgggccctgagcaagaaggccctgctgagctctgtggacataatccacatctttgctagcagagccagacaggcccagaccagcgaatag

SEQ ID NO: 25 - protein, 1171 aa, synthetic construct
MDYKDDDDKGTMVGGSPPGEGVRSPGIASPSGTAVQHDFGSPILVPGAEAAEVSTPVVKVPQDHPACPCCGTRVVKVTALSEHLRRAHGRKRVLFQCSRCGRMNEKHHSIACHFPKCRGPPVEEGPLGAPEWCCEECGQKFNTKSGLSQHKRSVHPLTRNVERIEAARPKGKGKRGAHKGCWTEAEVAQLIELEGRFKNQRFINKLIAEHLPSKSAKQISDKRRQLAAATKTSSPEKRVTSSTSGESSPEVEKVEGIKREYRRRVGEWLCAGSLNDQTSFQKILEDVESGSEIVTGPLEELASFARGKLAAARVRHHRKHPAEAVPAREEQRWMKRRVGRRGLYLRFQRLFALDRRKLAGIILDDVESIKCPLPMEEVADVFRRRWEEVAPFTGSGSFRSLGKADNGAFKPMISAKEVMKNVREMSRRSAPGPDGLSLRDLMKIDPQGSRMAELFNLWLLAGRVPDQVKAGRTVLIPKSADPGKIGNIDNWRPITIGSVLLRMFSRILSARLRRACPINRRQRGFIAAPGCSENLKLLQALIKSAKRDHRTLGVVFVDLAKAFDSVNHQHIFQVLVQKGVDGHIIDILRDLYTNAGTYLESGSQRSGFIKILRGVKQGDPLSPILFNLALDPLLCRLEDRGLGYKYGDQQITSLAFADDLALLSDSWEGMQQSIRVVEEFCQRTGLRVQAPKCHGFLIRPTKESYTINDCDPWTIADMQLDMIDPGSSEKYLGLGIDPWIGLSRPELSEVLTRWVKNIGGAPLKPLQKVDILRSYALPRLLFIADHAGLSATCLHSLDLSIRSAVKGWLHLPPSTCDAIIYVSYKDGGLGLPRLASLIPNVQARRLVRIAQSEDDVIRSVVLQEGIQEEIRKVWISAGGRPEKVPSVTGEFPVMEAQAADEALSEWERRAPRTIYPIPCKWRKREMENWTNLKSQGHGIRNFENDRISNDWLLHYGRIPHRKLITAIQLRANVYPTREFLARGLGEGAPRGCRHCPAEWESCSHIIGYCPAVQEARIKRHNDICGVLAEEARKLGWVIFIEPHLRDNTNELFKPDLVLVKGSCAKVVDVTIRYESGLTTLSDAAAEKARKYQHLAGEVRALTSATTVDFLGFPIGARGKWYVGNNGLLSDLGFSTSRVVRIARALSKKALLSSVDIIHIFASRARQAQTSE

SEQ ID NO: 26 - DNA, 3552 bp, synthetic construct
  source 1..3552: mol_type = other DNA
atggattacaaggatgacgacgataagggtaccgagaaggtgatggtcaccgttccggacaagaaccctccatgtccatgctgcggcacccgggtgaacagcgtgctgaaccttatcgagcacctgaaggtcagccacggaaagcggggcgtgtgcttccggtgcgcgaagtgtgggaaggaaaatagcaactaccacagcgtggtgtgccatttcccaaaatgtagaggacctgagacagagaaagctccagccggcgagtggatttgtgaagtgtgcaacagagacttcaccaccaaaatcggcctgggccagcataagagattagctcaccccgctgttcggaatcaggagagaatcgtcgccagccagcctaaggaaactagcaacagaggcgctcacaaaagatgctggaccaaagaggaggaggaactgctgatcagactggaagcccagtttgagggcaacaagaacatcaacaagctgatcgcagaacacatcaccacaaagaccgccaagcagatctccgataagaggagactgctgagccgcaagcctgccgaggagcctagagaggaacctggcacatgccaccacaccagacgggcagctgccagcctgagaacagagcccgagatgtcccaccacgctcaggccgaagatcgggataatggccctggccggagacccctgcctggtagagccgccgccggcggcagaacaatggacgagatcaggagacaccctgacaagggcaatggacagcagcggcctaccaaacagaagtctgaggagcagctgcaagcctactacaaaaagacactggaggagcggctgagcgccggagccctgaataccttcccccgcgccttcaagcaagtgatggaaggcagagatatcaagctggtgatcaaccagacagcccaggactgcttcggctgtctggagagcatctctcagatcagaacagcgaccagagataagaaagataccgtgaccagagagaagcacccgaagaagccattccaaaagtggatgaaggaccgggccataaagaagggcaactacctgaggttccagagattgttctacctggaccgaggcaaactggccaagatcatcctggacgatatcgaatgcctgagctgcgacatccccctgtctgagatctacagcgtgtttaagacaagatgggaaaccacaggaagcttcaaatctctgggcgattttaaaacctacggcaaggccgacaacactgccttcagagaactgattacagctaaggaaatcgagaaaaacgtgcaggagatgagcaagggcagcgcccctggccctgacggtatcaccctgggagatgtggtgaaaatggaccccgagttcagccgcacaatggagatcttcaacctgtggctgaccactggcaaaatccctgacatggtgcggggatgccgcaccgtgctgatacctaagagcagcaagccagatagactgaaggatatcaacaactggcggcctatcacaatcggcagcatcctgctccggctgtttagcagaatcgtaaccgcccgtctgagtaaggcctgtcctctgaaccccagacagcggggcttcatccgggccgccggctgcagcgagaacctcaaactgctgcagaccatcatctggtcggccaaacgagagcacagacctctcggcgtggtgttcgtggacatcgccaaagcttttgataccgtctcccaccagcacatcatccacgccctgcagcagcgggaagtggaccctcacatcgtgggactggtgtccaacatgtacgagaacatcagcacttatatcaccaccaagagaaacacccacacagacaagatccagatcagagtgggcgttaagcagggcgatcctatgtctcctctgctgttcaacctggctatggaccctctgctctgtaaactggaggagtctggcaagggataccacagaggccaaagctccatcaccgctatggcctttgccgacgacctggtgctgctgtctgattcttgggagaacatgaataccaacataagcatcctggaaacattctgcaaccttacaggcctgaaaacccagggacaaaagtgccacggcttttacatcaagcctaccaaagacagctacaccatcaacgactgcgccgcctggaccatcaacggcacccctttaaacatgatcgaccctggagaaagcgagaagtatctgggcctgcagttcgacccctggatcggcatcgctagaagcggcctgagcacaaagctggacttttggctgcagagaattgaccaggcccctctgaagcctctgcagaaaaccgacatcctgaaaacctacacaatccctcggctgatctacattgccgatcacagcgaggtaaagaccgccctgctggaaaccctggaccaaaagatcagaaccgccgtgaaggaatggctccacctgcccccctgcacctgcgacgctatcctgtacagcagcaccagggacggcggcctgggcatcaccaagctggcgggcctgatcccctccgtccaggctagacggctgcatagaatcgcccagagcagcgatgacacaatgaagtgctttatggaaaaggaaaagatggaacagctgcacaagaagctgtggattcaggccggtggcgacagagagaacattcctagcatctgggaggcgccgcctagtagcgagcctcccaacaacgtgtctaccaattctgagtgggaagcccctacacagaaggacaagttccctaagccttgtaattggagaaagaacgagttcaagaagtggacaaagctggcctctcagggccggggaattgtgaatttcgagcgggacaagatcagcaatcactggattcagtactacagaagaatcccacacagaaagctgctgacggcccttcagctgagagccaacgtgtaccccacgcgggagttcctggccagaggtagacaggaccagtacatcaaggcctgcagacattgtgatgctgatatcgagtcttgcgcccacatcatcggcaactgccctgtgacacaggacgcgagaatcaaaagacacaactacatctgcgagctgctgctggaagaggccaagaagaaggactgggtggtgttcaaggaaccccacatcagagacagcaataaggaactctataaacctgacctgatcttcgtgaaggacgcccgggccctggtcgtggacgtgaccgtgagatacgaggccgccaagtctagcctggaggaggctgccgccgagaaagtgcggaagtacaagcaccttgaaacagaagtgcggcacctgaccaacgccaaggacgtcacattcgtgggcttccccctgggcgccagaggcaaatggcaccaggataacttcaagctgctgacagagctgggcctgagcaagtcccgccaagtgaagatggccgagaccttcagcaccgtggccctgttctcctctgtggacatcgtgcacatgttcgctagcagagccagaaagagcatggtgatgtag

SEQ ID NO: 27 - protein, 1183 aa, synthetic construct
MDYKDDDDKGTEKVMVTVPDKNPPCPCCGTRVNSVLNLIEHLKVSHGKRGVCFRCAKCGKENSNYHSVVCHFPKCRGPETEKAPAGEWICEVCNRDFTTKIGLGQHKRLAHPAVRNQERIVASQPKETSNRGAHKRCWTKEEEELLIRLEAQFEGNKNINKLIAEHITTKTAKQISDKRRLLSRKPAEEPREEPGTCHHTRRAAASLRTEPEMSHHAQAEDRDNGPGRRPLPGRAAAGGRTMDEIRRHPDKGNGQQRPTKQKSEEQLQAYYKKTLEERLSAGALNTFPRAFKQVMEGRDIKLVINQTAQDCFGCLESISQIRTATRDKKDTVTREKHPKKPFQKWMKDRAIKKGNYLRFQRLFYLDRGKLAKIILDDIECLSCDIPLSEIYSVFKTRWETTGSFKSLGDFKTYGKADNTAFRELITAKEIEKNVQEMSKGSAPGPDGITLGDVVKMDPEFSRTMEIFNLWLTTGKIPDMVRGCRTVLIPKSSKPDRLKDINNWRPITIGSILLRLFSRIVTARLSKACPLNPRQRGFIRAAGCSENLKLLQTIIWSAKREHRPLGVVFVDIAKAFDTVSHQHIIHALQQREVDPHIVGLVSNMYENISTYITTKRNTHTDKIQIRVGVKQGDPMSPLLFNLAMDPLLCKLEESGKGYHRGQSSITAMAFADDLVLLSDSWENMNTNISILETFCNLTGLKTQGQKCHGFYIKPTKDSYTINDCAAWTINGTPLNMIDPGESEKYLGLQFDPWIGIARSGLSTKLDFWLQRIDQAPLKPLQKTDILKTYTIPRLIYIADHSEVKTALLETLDQKIRTAVKEWLHLPPCTCDAILYSSTRDGGLGITKLAGLIPSVQARRLHRIAQSSDDTMKCFMEKEKMEQLHKKLWIQAGGDRENIPSIWEAPPSSEPPNNVSTNSEWEAPTQKDKFPKPCNWRKNEFKKWTKLASQGRGIVNFERDKISNHWIQYYRRIPHRKLLTALQLRANVYPTREFLARGRQDQYIKACRHCDADIESCAHIIGNCPVTQDARIKRHNYICELLLEEAKKKDWVVFKEPHIRDSNKELYKPDLIFVKDARALVVDVTVRYEAAKSSLEEAAAEKVRKYKHLETEVRHLTNAKDVTFVGFPLGARGKWHQDNFKLLTELGLSKSRQVKMAETFSTVALFSSVDIVHMFASRARKSMVM

SEQ ID NO: 28 - RNA, 4015 nt, synthetic construct
  source 1..4015: mol_type = other RNA
aggaataaactagtattcttctggtccccacagactcagagagaacccgccaccatggattacaaggatgacgacgataagggatccgagaaggtgatggtcaccgttccggacaagaaccctccatgtccatgctgcggcacccgggtgaacagcgtgctgaaccttatcgagcacctgaaggtcagccacggaaagcggggcgtgtgcttccggtgcgcgaagtgtgggaaggaaaatagcaactaccacagcgtggtgtgccatttcccaaaatgtagaggacctgagacagagaaagctccagccggcgagtggatttgtgaagtgtgcaacagagacttcaccaccaaaatcggcctgggccagcataagagattagctcaccccgctgttcggaatcaggagagaatcgtcgccagccagcctaaggaaactagcaacagaggcgctcacaaaagatgctggaccaaagaggaggaggaactgctgatcagactggaagcccagtttgagggcaacaagaacatcaacaagctgatcgcagaacacatcaccacaaagaccgccaagcagatctccgataagaggagactgctgagccgcaagcctgccgaggagcctagagaggaacctggcacatgccaccacaccagacgggcagctgccagcctgagaacagagcccgagatgtcccaccacgctcaggccgaagatcgggataatggccctggccggagacccctgcctggtagagccgccgccggcggcagaacaatggacgagatcaggagacaccctgacaagggcaatggacagcagcggcctaccaaacagaagtctgaggagcagctgcaagcctactacaaaaagacactggaggagcggctgagcgccggagccctgaataccttcccccgcgccttcaagcaagtgatggaaggcagagatatcaagctggtgatcaaccagacagcccaggactgcttcggctgtctggagagcatctctcagatcagaacagcgaccagagataagaaagataccgtgaccagagagaagcacccgaagaagccattccaaaagtggatgaaggaccgggccataaagaagggcaactacctgaggttccagagattgttctacctggaccgaggcaaactggccaagatcatcctggacgatatcgaatgcctgagctgcgacatccccctgtctgagatctacagcgtgtttaagacaagatgggaaaccacaggaagcttcaaatctctgggcgattttaaaacctacggcaaggccgacaacactgccttcagagaactgattacagctaaggaaatcgagaaaaacgtgcaggagatgagcaagggcagcgcccctggccctgacggtatcaccctgggagatgtggtgaaaatggaccccgagttcagccgcacaatggagatcttcaacctgtggctgaccactggcaaaatccctgacatggtgcggggatgccgcaccgtgctgatacctaagagcagcaagccagatagactgaaggatatcaacaactggcggcctatcacaatcggcagcatcctgctccggctgtttagcagaatcgtaaccgcccgtctgagtaaggcctgtcctctgaaccccagacagcggggcttcatccgggccgccggctgcagcgagaacctcaaactgctgcagaccatcatctggtcggccaaacgagagcacagacctctcggcgtggtgttcgtggacatcgccaaagcttttgataccgtctcccaccagcacatcatccacgccctgcagcagcgggaagtggaccctcacatcgtgggactggtgtccaacatgtacgagaacatcagcacttatatcaccaccaagagaaacacccacacagacaagatccagatcagagtgggcgttaagcagggcgatcctatgtctcctctgctgttcaacctggctatggaccctctgctctgtaaactggaggagtctggcaagggataccacagaggccaaagctccatcaccgctatggcctttgccgacgacctggtgctgctgtctgattcttgggagaacatgaataccaacataagcatcctggaaacattctgcaaccttacaggcctgaaaacccagggacaaaagtgccacggcttttacatcaagcctaccaaagacagctacaccatcaacgactgcgccgcctggaccatcaacggcacccctttaaacatgatcgaccctggagaaagcgagaagtatctgggcctgcagttcgacccctggatcggcatcgctagaagcggcctgagcacaaagctggacttttggctgcagagaattgaccaggcccctctgaagcctctgcagaaaaccgacatcctgaaaacctacacaatccctcggctgatctacattgccgatcacagcgaggtaaagaccgccctgctggaaaccctggaccaaaagatcagaaccgccgtgaaggaatggctccacctgcccccctgcacctgcgacgctatcctgtacagcagcaccagggacggcggcctgggcatcaccaagctggcgggcctgatcccctccgtccaggctagacggctgcatagaatcgcccagagcagcgatgacacaatgaagtgctttatggaaaaggaaaagatggaacagctgcacaagaagctgtggattcaggccggtggcgacagagagaacattcctagcatctgggaggcgccgcctagtagcgagcctcccaacaacgtgtctaccaattctgagtgggaagcccctacacagaaggacaagttccctaagccttgtaattggagaaagaacgagttcaagaagtggacaaagctggcctctcagggccggggaattgtgaatttcgagcgggacaagatcagcaatcactggattcagtactacagaagaatcccacacagaaagctgctgacggcccttcagctgagagccaacgtgtaccccacgcgggagttcctggccagaggtagacaggaccagtacatcaaggcctgcagacattgtgatgctgatatcgagtcttgcgcccacatcatcggcaactgccctgtgacacaggacgcgagaatcaaaagacacaactacatctgcgagctgctgctggaagaggccaagaagaaggactgggtggtgttcaaggaaccccacatcagagacagcaataaggaactctataaacctgacctgatcttcgtgaaggacgcccgggccctggtcgtggacgtgaccgtgagatacgaggccgccaagtctagcctggaggaggctgccgccgagaaagtgcggaagtacaagcaccttgaaacagaagtgcggcacctgaccaacgccaaggacgtcacattcgtgggcttccccctgggcgccagaggcaaatggcaccaggataacttcaagctgctgacagagctgggcctgagcaagtcccgccaagtgaagatggccgagaccttcagcaccgtggccctgttctcctctgtggacatcgtgcacatgttcgctagcagagccagaaagagcatggtgatgtagtgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 29 - protein, 1169 aa, synthetic construct
MVTVPDKNPPCPCCGTRVNSVLNLIEHLKVSHGKRGVCFRCAKCGKENSNYHSVVCHFPKCRGPETEKAPAGEWICEVCNRDFTTKIGLGQHKRLAHPAVRNQERIVASQPKETSNRGAHKRCWTKEEEELLIRLEAQFEGNKNINKLIAEHITTKTAKQISDKRRLLSRKPAEEPREEPGTCHHTRRAAASLRTEPEMSHHAQAEDRDNGPGRRPLPGRAAAGGRTMDEIRRHPDKGNGQQRPTKQKSEEQLQAYYKKTLEERLSAGALNTFPRAFKQVMEGRDIKLVINQTAQDCFGCLESISQIRTATRDKKDTVTREKHPKKPFQKWMKDRAIKKGNYLRFQRLFYLDRGKLAKIILDDIECLSCDIPLSEIYSVFKTRWETTGSFKSLGDFKTYGKADNTAFRELITAKEIEKNVQEMSKGSAPGPDGITLGDVVKMDPEFSRTMEIFNLWLTTGKIPDMVRGCRTVLIPKSSKPDRLKDINNWRPITIGSILLRLFSRIVTARLSKACPLNPRQRGFIRAAGCSENLKLLQTIIWSAKREHRPLGVVFVDIAKAFDTVSHQHIIHALQQREVDPHIVGLVSNMYENISTYITTKRNTHTDKIQIRVGVKQGDPMSPLLFNLAMDPLLCKLEESGKGYHRGQSSITAMAFADDLVLLSDSWENMNTNISILETFCNLTGLKTQGQKCHGFYIKPTKDSYTINDCAAWTINGTPLNMIDPGESEKYLGLQFDPWIGIARSGLSTKLDFWLQRIDQAPLKPLQKTDILKTYTIPRLIYIADHSEVKTALLETLDQKIRTAVKEWLHLPPCTCDAILYSSTRDGGLGITKLAGLIPSVQARRLHRIAQSSDDTMKCFMEKEKMEQLHKKLWIQAGGDRENIPSIWEAPPSSEPPNNVSTNSEWEAPTQKDKFPKPCNWRKNEFKKWTKLASQGRGIVNFERDKISNHWIQYYRRIPHRKLLTALQLRANVYPTREFLARGRQDQYIKACRHCDADIESCAHIIGNCPVTQDARIKRHNYICELLLEEAKKKDWVVFKEPHIRDSNKELYKPDLIFVKDARALVVDVTVRYEAAKSSLEEAAAEKVRKYKHLETEVRHLTNAKDVTFVGFPLGARGKWHQDNFKLLTELGLSKSRQVKMAETFSTVALFSSVDIVHMFASRARKSMVM

SEQ ID NO: 30 - DNA, 3531 bp, synthetic construct
  source 1..3531: mol_type = other DNA
atggattacaaggatgacgacgataagggtaccatgggcaacatcgtcatggtgaccttaccaaacaagaaccctccatgtccctgttgccgggtgcggctgcacagcgtgctggctctgatcgagcatctgaagggcagccacggaaaaaaacgggcatgcttccggtgcgtgaaatgcggacgtgagaacttcaactaccacagcaccgtgtgccacatcgccaagtgcaagggccccaaagtagaaaaagcccctgtgggcgagtggatctgtgaagtttgcggccgagatttcaccaccaagatcggcctgggacaacacaagagactggctcatcctcttgtgcggaaccaggagcggatcgacgcttctcagcctaaggaaaccagcaacagaggcgcccataagagatgttggacaaaagaggaagaagagatgctgattaagctggaggtgcagttcgaaggccacagaaacatcaacaagctcatcgctgagcacctgaccacaaagaccagcaagcagatctctgacaaaaggagactgctgcccaggaagcagctgacagacctgagcaagggggtggccggccagaaggtgctggaccctggcctgagccaccagcctcagctgggcgtggtggacaatggcctgggcggaggacacctacctggcggcccggccgccgaaggcagaacgatcgaacctcttggccaccaccttgataaggacaacggccaccgggagatcgccgaccagcacaaggccggcagactgcaggcccactacagaaagaagatccggaagcggctgagcgagggcatgatctccaacttccccgaggtgttcgagcaactgctggactgccaggaggcccagcctctgattaaccaggccgcacaggactgcttcggctgcctggattctgccagccagattagaaaagctctgcggaagcagaacacccagaaggatcagggcgaccagcctaagcggcccgcccagaagtggatgaagaagagagccgtcaagcggggccacttcctgagattccagaaactgttccacctggatcggggcaagctggctaagatcatcttggacgacgtggaatgcctgtcttgtgatatccctccaagtgagatctacagcgtgttcaaggccagatgggagacacccggacagttcgctggcctgggcgacttcgagatcaacagaaaggcaaacaataaagctttcagagatctgattaccgcgaaagagatcctgaagaacgtgagagaaatgacaaagggctctgcccctggtcctgatggcatcgctctgggcgacatcagaaagatggaccccgagtacaccagaactgccgagctgtttaatctgtggctgacatctggcgagatccccgacatggtgagaggctgtagaacagtgctgatccccaagagcagcaaacctgagcggctcaaggacatcaataattggagacccatcaccatcgggagcatcctgctgcggctgttcagcagaatcatcaccgctagactgacaaaagcctgccctctgaatccaagacagagaagctttatcagcgccgccggatgtagcgagaacctgaagctcctgcagaccatcatccggaccgccaagaacgagcaccggccactgggcgttgtgttcgtggacatcgccaaggccttcgacaccgtgagccaccagcacatcatccacgtgctgcagagaagaagagtggacccacacatcatcggcctcgtgaagaacatgtacaaagatatcagcaccgtcatcaccaccaagaagaacacctatacagacaagattcagatccaggtgggcgtgaagcaaggcgatcccctgagccctctcctttttaacctggctatggaccctctgctgtgcaagctggaggaacacggcaagggatttcacagaggccagagcaagatcacagctatggcctttgccgatgacctcgtgctgctgtccgacagctgggaggacatgaacgccaatatcaagatactggaaaccttctgcgacctgacaggcctgaaaactcagggacagaagtgtcacggtttctacatcaaacctaccaaggacagctacacagtgaacaactgcgccgcctggacgatcaacggtacacctctgaacatgattaaccctggagagagcgagaagtacctgggcctgcagttcgatccctgggtaggcatcgccaaaacaagcctgcctgagaagctggacttctggctggaacgcattgatcgggcccctctgaagcctttccaaaagctggacatcctgaaaacctacaccatccctagactcacctatgtggccgaccactctgagatgaaggccggcgccctggaagccctggatagaaccatccggagcgccgtgaaggactggctgcacctgcctagcagcacctgtgacgcgatcctgtacaccagtatgaaggacggtggactgggcgtgaccaaactggtgggcctgatcccatccgtgcaggccagacgactgcacagaatcgcccaaagccccgaggaaaccatgaaggacttcctggagaaggctcagatggaaaaaatgtacgagaagctgtgggtgcaggccggaggcaagagaaagagaatgccttctatctgggaggctctgccagaagtggtcccctccatcgataccgccacaactagcgagtgggaggcccctaaccccaagtccaagtaccctagaccttgtaattggcgccggaaagagttcaagaagtggaccaagctgattgctcaaggatggggcatccggtgctttaaaggggataagatctccaataactggatcaggcattatagatacattcctcacagaaagctgctgacagccattcaactgagagccagcgtctaccccacccgggagttcctggcccggggcagagaagataactgcgtgaaaagctgccggcactgcgaggccgctgaggaaagctgcgcccacatcatcggaatgtgccccgtggtgcgggacgccagaatcaagagacacaacagaatctgcgagaggctcatggaagaagccggcaagcgcgattggaccgtgtttcaggagcctcatatacgtgacgtgacaaaggaactgtacaagcccgacctgatcttcgtgaaagaaggcctggccctggtggtggacgtgaccatccggttcgaaagcaccaagaccaccctggaagaggcagccgctgagaaggtgaacaagtacaagcacctggaaacggaagtcagaaatctgacaaacgccaaggacgtgatcttcatgggctttcctctgggggccagaggccaatggtacaacaagaacttcgagctgctggacaccctgggcctgcctagaagcaggcaggacatcatcgccaaaaccctgtccaccgatgctctgatctcttccgtggatattatccacatgttcgccagcagaggcagacggcagcacgcctgatga

SEQ ID NO: 31 - protein, 1175 aa, synthetic construct
MDYKDDDDKGTMGNIVMVTLPNKNPPCPCCRVRLHSVLALIEHLKGSHGKKRACFRCVKCGRENFNYHSTVCHIAKCKGPKVEKAPVGEWICEVCGRDFTTKIGLGQHKRLAHPLVRNQERIDASQPKETSNRGAHKRCWTKEEEEMLIKLEVQFEGHRNINKLIAEHLTTKTSKQISDKRRLLPRKQLTDLSKGVAGQKVLDPGLSHQPQLGVVDNGLGGGHLPGGPAAEGRTIEPLGHHLDKDNGHREIADQHKAGRLQAHYRKKIRKRLSEGMISNFPEVFEQLLDCQEAQPLINQAAQDCFGCLDSASQIRKALRKQNTQKDQGDQPKRPAQKWMKKRAVKRGHFLRFQKLFHLDRGKLAKIILDDVECLSCDIPPSEIYSVFKARWETPGQFAGLGDFEINRKANNKAFRDLITAKEILKNVREMTKGSAPGPDGIALGDIRKMDPEYTRTAELFNLWLTSGEIPDMVRGCRTVLIPKSSKPERLKDINNWRPITIGSILLRLFSRIITARLTKACPLNPRQRSFISAAGCSENLKLLQTIIRTAKNEHRPLGVVFVDIAKAFDTVSHQHIIHVLQRRRVDPHIIGLVKNMYKDISTVITTKKNTYTDKIQIQVGVKQGDPLSPLLFNLAMDPLLCKLEEHGKGFHRGQSKITAMAFADDLVLLSDSWEDMNANIKILETFCDLTGLKTQGQKCHGFYIKPTKDSYTVNNCAAWTINGTPLNMINPGESEKYLGLQFDPWVGIAKTSLPEKLDFWLERIDRAPLKPFQKLDILKTYTIPRLTYVADHSEMKAGALEALDRTIRSAVKDWLHLPSSTCDAILYTSMKDGGLGVTKLVGLIPSVQARRLHRIAQSPEETMKDFLEKAQMEKMYEKLWVQAGGKRKRMPSIWEALPEVVPSIDTATTSEWEAPNPKSKYPRPCNWRRKEFKKWTKLIAQGWGIRCFKGDKISNNWIRHYRYIPHRKLLTAIQLRASVYPTREFLARGREDNCVKSCRHCEAAEESCAHIIGMCPVVRDARIKRHNRICERLMEEAGKRDWTVFQEPHIRDVTKELYKPDLIFVKEGLALVVDVTIRFESTKTTLEEAAAEKVNKYKHLETEVRNLTNAKDVIFMGFPLGARGQWYNKNFELLDTLGLPRSRQDIIAKTLSTDALISSVDIIHMFASRGRRQHA

SEQ ID NO: 32 - DNA, 3150 bp, synthetic construct
  source 1..3150: mol_type = other DNA
atgaccacccgtccgagcgtggatatcttcccggaagaccagtacgagccgaatgcggccgctaccctgagccgtgtgccatgcaccgtgtgcggtcgtagctttaacagcaaacgcggtctgggcgtgcacatgcgcagccgtcacccggacgagctggacgaggaacgtcgccgtgtggatatcaaggcgcgttggagcgaggaagagaaatggatgatggctcgcaaggaagtggagctgaccgccaacggccacaagcacatgaacaaacagctggctgtgtacttcgccaaccgtagcgtggaggccattaagaaactgcgccagcgtggtgactataaggaaaaaattgagcagatccgtggtcagagcgctctggtgccagaagtggctaacctgaccattcgtcgtcgtccgagccgtagcgagcagaaccaccaggtgaccaccagcgaaaccaccccgatcaccccgttcgaacagagcaaccgtgagattctgcgtaccctgcgtggttacagcccagtggagtgccacagcaaatggcgtgctcaggaactgcagaccatcattgaccgcgccgaactggagggcaaggagaccaccctgcagtgcctgagcctgtacctgctgggtatttttccggcgcagggcgtgcgtcataccctgacccgtccaccacgtcgtccgcgtaaccgtcgtgaaagccgtcgtcagcagtatgctgtggtgcagcgtaactgggataagcacaaaggtcgctgcatcaaaagcctgctgaacggcaccgacgagagcgtgatgccgagccaggaagtgatggtgccgtattggcgtgaggtgatgacccagccaagcccaagcagctgcagcggtgaagtgattcagatggatcacagcctggagcgtgtgtggagcgccatcaccgaacacgacctgcgtgctagccgcattagcctgagcagcagcccaggtccagatggtatcaccccaaagagcgcccgtgaggtgccgagcggtattatgctgcgcatcatgaacctgattctgtggtgcggcaacctgccgcacagcattcgtctggcccgcaccgtgttcattccgaagaccgtgaccgcgaaacgtccgcaggactttcgcccaatcagcgtgccgagcgtgctggtgcgtcagctgaacgctatcctggccacccgcctgaacagcagcattaactgggacccgcgtcagcgtggtttcctgccaaccgatggctgcgccgacaacgctaccatcgtggacctggtgctgcgtcacagccacaaacacttccgcagctgctacattgccaacctggacgtgagcaaggccttcgacagcctgagccacgctagcatctacgataccctgcgtgcctatggtgcgccgaagggctttgtggactacgtgcagaacacctatgagggcggtggcaccagcctgaacggcgacggctggagcagcgaagagttcgtgccggcccgtggtgtgaaacagggcgatccgctgagcccgatcctgttcaacctggttatggaccgtctgctgcgcaacctgccgagcgagatcggtgctaaggtgggtaacgccattaccaacgccgcggctttcgccgacgatctggtgctgtttgcggaaacccgtatgggtctgcaggtgctgctggacaagaccctggacttcctgagcctggtgggcctgaagctgaacgccgacaaatgctttaccgtgggtatcaagggccagccgaagcagaaatgcaccgtgctggaggcgcagagcttctacgtgggtagccgtgagatcccgagcctgaaacgcaccgatgaatggaagtatctgggtattaactttaccgctaccggtcgcgtgcgttgcaacccagcggaggacattggcccgaaactgcagcgtctgaccaaggcgccgctgaaaccgcagcagcgtatgttcgctctgcgcaccgtgctgatcccgcagctgtaccacaagctggcgctgggtagcgtggctatcggcgtgctgcgtaagaccgataaactgattcgctactatgtgcgtcgttggctgaacctgccactggatgtgccaattgcgttcattcatgctccgccgaaaagcggtggtctgggtattccaagcctgcgttgggtggccccaatgctgcgtctgcgtcgtctgagcaacatcaaatggccgcacctgacccagaacgaggtggctagcagctttctggaagcggagaaacagcgtgcccgtgatcgtctgctggctgaacagaacgagctgctgagccgtccggcgatcgagaagtactgggctaacaaactgtatctgagcgtggatggtagcggtctgcgtgaagccggtcactggggtccacagcatggttgggtgaaccagccaacccgtctgctgaccggtaaagagtacattgatggcatccgtctgcgcattaatgctctgccaaccaagagccgtaccacccgtggtcgtcatgaactggaacgccagtgccgtgctggttgcgatgccccggaaaccaccaaccacatcatgcagaaatgctatcgtagccatggtcgtcgtgtggctcgtcacaactgcgtggtgaaccgtatcaagcgcggtctggaagagcgtggctgcgtggtgattgtggaaccgagcctgcagtgcgagagcggtctgaacaaaccggatctggtggctctgcgtcaggaccacattgatgtgatcgacattcagatcgtgaccgacggccacagcatggacgatgcccaccagcgtaagatcaaccgttacgatcgcccggacattcgcaccgaactgcgtcgtcgttttgaggcggccggtgatatcgaatttcacagcgcgaccctgaactggcgtggtatctggagcggccagagcgtgaagcgcctgattgctaaaggcctgctgagcaagtatgacagccacatcattagcgtgcaggtgatgcgtggttccctgggctgtttcaagcaattcatgtatctgagcggtttctcccgtgactggaccatggggagcgactacaaagaccatgacggtgattataaagatcatgacatcgattacaaggatgacgatgacaagaagtagtaa

SEQ ID NO: 33 - protein, 1048 aa, synthetic construct
MTTRPSVDIFPEDQYEPNAAATLSRVPCTVCGRSFNSKRGLGVHMRSRHPDELDEERRRVDIKARWSEEEKWMMARKEVELTANGHKHMNKQLAVYFANRSVEAIKKLRQRGDYKEKIEQIRGQSALVPEVANLTIRRRPSRSEQNHQVTTSETTPITPFEQSNREILRTLRGYSPVECHSKWRAQELQTIIDRAELEGKETTLQCLSLYLLGIFPAQGVRHTLTRPPRRPRNRRESRRQQYAVVQRNWDKHKGRCIKSLLNGTDESVMPSQEVMVPYWREVMTQPSPSSCSGEVIQMDHSLERVWSAITEHDLRASRISLSSSPGPDGITPKSAREVPSGIMLRIMNLILWCGNLPHSIRLARTVFIPKTVTAKRPQDFRPISVPSVLVRQLNAILATRLNSSINWDPRQRGFLPTDGCADNATIVDLVLRHSHKHFRSCYIANLDVSKAFDSLSHASIYDTLRAYGAPKGFVDYVQNTYEGGGTSLNGDGWSSEEFVPARGVKQGDPLSPILFNLVMDRLLRNLPSEIGAKVGNAITNAAAFADDLVLFAETRMGLQVLLDKTLDFLSLVGLKLNADKCFTVGIKGQPKQKCTVLEAQSFYVGSREIPSLKRTDEWKYLGINFTATGRVRCNPAEDIGPKLQRLTKAPLKPQQRMFALRTVLIPQLYHKLALGSVAIGVLRKTDKLIRYYVRRWLNLPLDVPIAFIHAPPKSGGLGIPSLRWVAPMLRLRRLSNIKWPHLTQNEVASSFLEAEKQRARDRLLAEQNELLSRPAIEKYWANKLYLSVDGSGLREAGHWGPQHGWVNQPTRLLTGKEYIDGIRLRINALPTKSRTTRGRHELERQCRAGCDAPETTNHIMQKCYRSHGRRVARHNCVVNRIKRGLEERGCVVIVEPSLQCESGLNKPDLVALRQDHIDVIDIQIVTDGHSMDDAHQRKINRYDRPDIRTELRRRFEAAGDIEFHSATLNWRGIWSGQSVKRLIAKGLLSKYDSHIISVQVMRGSLGCFKQFMYLSGFSRDWTMGSDYKDHDGDYKDHDIDYKDDDDKK

SEQ ID NO: 34 - RNA, 3609 nt, synthetic construct
  source 1..3609: mol_type = other RNA
aggaataaactagtattcttctggtccccacagactcagagagaacccgccaccatgaccacccgtccgagcgtggatatcttcccggaggaccagtacgagccgaatgcggccgctaccctgagccgtgtgccatgcaccgtgtgcggtcgtagctttaacagcaaacgcggtctgggcgtgcacatgcgcagccgtcacccggacgagctggacgaggaacgtcgccgtgtggatatcaaggcgcgttggagcgaggaagagaaatggatgatggctcgcaaggaagtggagctgaccgccaacggccacaagcacatgaacaaacagctggctgtgtacttcgccaaccgtagcgtggaggccattaagaaactgcgccagcgtggtgactataaggaaaaaattgagcagatccgtggtcagagcgctctggtgccagaagtggctaacctgaccattcgtcgtcgtccgagccgtagcgagcagaaccaccaggtgaccaccagcgaaaccaccccgatcaccccgttcgaacagagcaaccgtgagattctgcgtaccctgcgtggttacagcccagtggagtgccacagcaaatggcgtgctcaggaactgcagaccatcattgaccgcgccgaactggagggcaaggagaccaccctgcagtgcctgagcctgtacctgctgggtatttttccggcgcagggcgtgcgtcataccctgacccgtccaccacgtcgtccgcgtaaccgtcgtgaaagccgtcgtcagcagtatgctgtggtgcagcgtaactgggataagcacaaaggtcgctgcatcaaaagcctgctgaacggcaccgacgagagcgtgatgccgagccaggaagtgatggtgccgtattggcgtgaggtgatgacccagccaagcccaagcagctgcagcggtgaagtgattcagatggatcacagcctggagcgtgtgtggagcgccatcaccgaacacgacctgcgtgctagccgcattagcctgagcagcagcccaggtccagatggtatcaccccaaagagcgcccgtgaggtgccgagcggtattatgctgcgcatcatgaacctgattctgtggtgcggcaacctgccgcacagcattcgtctggcccgcaccgtgttcattccgaaaaccgtgaccgcgaaacgtccgcaggactttcgcccaatcagcgtgccgagcgtgctggtgcgtcagctgaacgctatcctggccacccgcctgaacagcagcattaactgggacccgcgtcagcgtggtttcctgccaaccgatggctgcgccgacaacgctaccatcgtggacctggtgctgcgtcacagccacaaacacttccgcagctgctacattgccaacctggacgtgagcaaggccttcgacagcctgagccacgctagcatctacgataccctgcgtgcctatggtgcgccgaagggctttgtggactacgtgcagaacacctatgagggcggtggcaccagcctgaacggcgacggctggagcagcgaagagttcgtgccggcccgtggtgtgaaacagggcgatccgctgagcccgatcctgttcaacctggttatggaccgtctgctgcgcaacctgccgagcgagatcggtgctaaggtgggtaacgccattaccaacgccgcggctttcgccgacgatctggtgctgtttgcggaaacccgtatgggtctgcaggtgctgctggacaagaccctggacttcctgagcctggtgggcctgaagctgaacgccgacaaatgctttaccgtgggtatcaagggccagccgaagcagaaatgcaccgtgctggaggcgcagagcttctacgtgggtagccgtgagatcccgagcctgaaacgcaccgatgaatggaagtatctgggtattaactttaccgctaccggtcgcgtgcgttgcaacccagcggaggacattggcccgaaactgcagcgtctgaccaaggcgccgctgaaaccgcagcagcgtatgttcgctctgcgcaccgtgctgatcccgcagctgtaccacaagctggcgctgggtagcgtggctatcggcgtgctgcgtaagaccgataaactgattcgctactatgtgcgtcgttggctgaacctgccactggatgtgccaattgcgttcattcatgctccgccgaaaagcggtggtctgggtattccaagcctgcgttgggtggccccaatgctgcgtctgcgtcgtctgagcaacatcaaatggccgcacctgacccagaacgaggtggctagcagctttctggaagcggagaaacagcgtgcccgtgatcgtctgctggctgaacagaacgagctgctgagccgtccggcgatcgagaagtactgggctaacaaactgtatctgagcgtggatggtagcggtctgcgtgaagccggtcactggggtccacagcatggttgggtgaaccagccaacccgtctgctgaccggtaaagagtacattgatggcatccgtctgcgcattaatgctctgccaaccaagagccgtaccacccgtggtcgtcatgaactggaacgccagtgccgtgctggttgcgatgccccggaaaccaccaaccacatcatgcagaaatgctatcgtagccatggtcgtcgtgtggctcgtcacaactgcgtggtgaaccgtatcaagcgcggtctggaagagcgtggctgcgtggtgattgtggaaccgagcctgcagtgcgagagcggtctgaacaaaccggatctggtggctctgcgtcaggaccacattgatgtgatcgacattcagatcgtgaccgacggccacagcatggacgatgcccaccagcgtaagatcaaccgttacgatcgcccggacattcgcaccgaactgcgtcgtcgttttgaggcggccggtgatatcgaatttcacagcgcgaccctgaactggcgtggtatctggagcggccagagcgtgaagcgcctgattgctaaaggcctgctgagcaagtatgacagccacatcattagcgtgcaggtgatgcgtggttccctgggctgtttcaagcaattcatgtatctgagcggtttctcccgtgactggaccatggggagcgactacaaagaccatgacggtgattataaagatcatgacatcgattacaaggatgacgatgacaagaagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 35 - DNA, 3249 bp, synthetic construct
  source 1..3249: mol_type = other DNA
atgaagaaaagcaacaaggagaaccgtccggaagcgagcggtctgccgctggagagcgaacgtaccggcgataacccgaccgtgcgtggtagcgcgggtgcggacccggttggtcaggatgcgccgggttggacctgccaattctgcgagcgtacctttagcaccaaccgtggtctgggcgtgcacaagcgtcgtgcgcacccggttgaaaccaacaccgacgcggcgccgatgatggtgaaacgtcgttggcacggcgaggaaatcgatctgctggcgcgtaccgaggcgcgtctgctggcggaacgtggccagtgcagcggtggcgacctgttcggcgcgctgccgggttttggtcgtaccctggaggcgattaaaggtcaacgtcgtcgtgaaccgtatcgtgcgctggttcaggcgcatctggcgcgttttggtagccaaccgggtccgagcagcggtggctgcagcgcggagccggattttcgtcgtgcgagcggtgcggaggaagcgggcgaggaacgttgcgcggaagatgcggcggcgtatgatccgagcgcggtgggtcaaatgagcccggatgcggcgcgtgtgctgagcgaactgctggagggtgcgggtcgtcgtcgtgcgtgccgtgcgatgcgtccgaagaccgcgggtcgtcgtaacgacctgcacgacgatcgtaccgcgagcgcgcacaagaccagccgtcagaaacgtcgtgcggagtacgcgcgtgtgcaagaactgtataagaaatgccgtagccgtgcggcggcggaagtgatcgatggtgcgtgcggtggcgttggtcacagcctggaggaaatggaaacctactggcgtccgattctggaacgtgtgagcgacgcgccgggtccgaccccggaggcgctgcacgcgctgggtcgtgcggaatggcacggtggcaaccgtgattatacccagctgtggaagccgatcagcgttgaggaaattaaagcgagccgtttcgactggcgtaccagcccgggtccggatggtatccgtagcggccagtggcgtgcggtgccggttcacctgaaggcggaaatgttcaacgcgtggatggcgcgtggcgagatcccggaaattctgcgtcaatgccgtaccgtgtttgttccgaaagttgagcgtccgggtggcccgggtgaataccgtccgatcagcattgcgagcatcccgctgcgtcacttccacagcattctggcgcgtcgtctgctggcgtgctgcccgccggacgcgcgtcagcgtggctttatctgcgcggatggtaccctggagaacagcgcggtgctggacgcggttctgggtgatagccgtaagaaactgcgtgaatgccacgtggcggttctggacttcgcgaaggcgtttgataccgtgagccacgaggcgctggttgaactgctgcgtctgcgtggcatgccggagcagttctgcggttacattgcgcacctgtatgacaccgcgagcaccaccctggcggtgaacaacgaaatgagcagcccggtgaaagttggccgtggtgttcgtcaaggcgacccgctgagcccgatcctgtttaacgtggttatggatctgattctggcgagcctgccggagcgtgtgggttaccgtctggagatggaactggttagcgcgctggcgtatgcggacgatctggtgctgctggcgggcagcaaggttggtatgcaggaaagcatcagcgcggtggactgcgttggccgtcaaatgggtctgcgtctgaactgccgtaaaagcgcggtgctgagcatgatcccggatggtcaccgtaagaaacaccactacctgaccgagcgtaccttcaacattggtggcaagccgctgcgtcaggtgagctgcgttgaacgttggcgttatctgggcgtggactttgaggcgagcggttgcgttaccctggaacacagcatcagcagcgcgctgaacaacattagccgtgcgccgctgaaaccgcagcaacgtctggagatcctgcgtgcgcacctgattccgcgtttccagcacggctttgttctgggtaacatcagcgacgatcgtctgcgtatgctggatgtgcagattcgtaaggcggttggtcaatggctgcgtctgccggcggacgtgccgaaagcgtactatcacgcggcggttcaagatggtggcctggcgatcccgagcgtgcgtgcgaccatcccggacctgattgttcgtcgttttggtggcctggatagcagcccgtggagcgtggcgcgtgcggcggcgaagagcgacaaaattcgtaagaaactgcgttgggcgtggaagcagctgcgtcgtttcagccgtgtggatagcaccacccaacgtccgagcgttcgtctgttttggcgtgagcacctgcacgcgagcgttgacggtcgtgagctgcgtgaaagcacccgtaccccgaccagcaccaaatggattcgtgaacgttgcgcgcagattaccggtcgtgatttcgtgcaatttgttcacacccacatcaacgcgctgccgagccgtattcgtggcagccgtggccgtcgtggtggcggtgagagcagcctgacctgccgtgcgggttgcaaagtgcgtgaaaccaccgcgcacatcctgcagcaatgccaccgtacccacggcggtcgtatcctgcgtcacaacaagattgtgagcttcgttgcgaaggcgatggaggaaaacaaatggaccgtggagctggaaccgcgtctgcgtaccagcgttggcctgcgtaaaccggacatcattgcgagccgtgatggcgtgggtgttatcgtggacgttcaggtggttagcggtcaacgtagcctggatgagctgcaccgtgaaaagcgtaacaaatacggcaaccacggtgagctggttgagctggttgcgggccgtctgggtctgccgaaagcggagtgcgtgcgtgcgaccagctgcaccattagctggcgtggcgtttggagcctgaccagctataaagagctgcgtagcatcattggtctgcgtgaaccgaccctgcagatcgtgccgattctggcgctgcgtggcagccacatgaactggacccgttttaaccaaatgaccagcgtgatgggtggcggtgttggtggtggaggtagcgggggcagtggagggatggggagcgactacaaagaccatgacggtgattataaagatcatgacatcgattacaaggatgacgatgacaagaagtaataa

SEQ ID NO: 36 - protein, 1081 aa, synthetic construct
MKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGGGGSGGSGGMGSDYKDHDGDYKDHDIDYKDDDDKK

SEQ ID NO: 37 - RNA, 3634 nt, synthetic construct
  source 1..3634: mol_type = other RNA
aggaataaactagtattcttctggtccccacagactcagagagaacccgccaccatggattacaaggatgacgacgataagggatccaagaaaagcaacaaggagaaccgtccggaagcgagcggtctgccgctggagagcgaacgtaccggcgataacccgaccgtgcgtggtagcgcgggtgcggacccggttggtcaggatgcgccgggttggacctgccaattctgcgagcgtacctttagcaccaaccgtggtctgggcgtgcacaagcgtcgtgcgcacccggttgaaaccaacaccgacgcggcgccgatgatggtgaaacgtcgttggcacggcgaggaaatcgatctgctggcgcgtaccgaggcgcgtctgctggcggaacgtggccagtgcagcggtggcgacctgttcggcgcgctgccgggttttggtcgtaccctggaggcgattaaaggtcaacgtcgtcgtgaaccgtatcgtgcgctggttcaggcgcatctggcgcgttttggtagccaaccgggtccgagcagcggtggctgcagcgcggagccggattttcgtcgtgcgagcggtgcggaggaagcgggcgaggaacgttgcgcggaagatgcggcggcgtatgatccgagcgcggtgggtcaaatgagcccggatgcggcgcgtgtgctgagcgaactgctggagggtgcgggtcgtcgtcgtgcgtgccgtgcgatgcgtcccaaaaccgcgggtcgtcgtaacgacctgcacgacgatcgtaccgcgagcgcgcacaagaccagccgtcagaaacgtcgtgcggagtacgcgcgtgtgcaagaactgtataagaaatgccgtagccgtgcggcggcggaagtgatcgatggtgcgtgcggtggcgttggtcacagcctggaggaaatggaaacctactggcgtccgattctggaacgtgtgagcgacgcgccgggtccgaccccggaggcgctgcacgcgctgggtcgtgcggaatggcacggtggcaaccgtgattatacccagctgtggaagccgatcagcgttgaggaaattaaagcgagccgtttcgactggcgtaccagcccgggtccggatggtatccgtagcggccagtggcgtgcggtgccggttcacctgaaggcggaaatgttcaacgcgtggatggcgcgtggcgagatcccggaaattctgcgtcaatgccgtaccgtgtttgttccgaaagttgagcgtccgggtggcccgggtgaataccgtccgatcagcattgcgagcatcccgctgcgtcacttccacagcattctggcgcgtcgtctgctggcgtgctgcccgccggacgcgcgtcagcgtggctttatctgcgcggatggtaccctggagaacagcgcggtgctggacgcggttctgggtgatagccgtaagaaactgcgtgaatgccacgtggcggttctggacttcgcgaaggcgtttgataccgtgagccacgaggcgctggttgaactgctgcgtctgcgtggcatgccggagcagttctgcggttacattgcgcacctgtatgacaccgcgagcaccaccctggcggtgaacaacgaaatgagcagcccggtgaaagttggccgtggtgttcgtcaaggcgacccgctgagcccgatcctgtttaacgtggttatggatctgattctggcgagcctgccggagcgtgtgggttaccgtctggagatggaactggttagcgcgctggcgtatgcggacgatctggtgctgctggcgggcagcaaggttggtatgcaggaaagcatcagcgcggtggactgcgttggccgtcaaatgggtctgcgtctgaactgccgtaaaagcgcggtgctgagcatgatcccggatggtcaccgtaagaaacaccactacctgaccgagcgtaccttcaacattggtggcaagccgctgcgtcaggtgagctgcgttgaacgttggcgttatctgggcgtggactttgaggcgagcggttgcgttaccctggaacacagcatcagcagcgcgctgaacaacattagccgtgcgccgctgaaaccgcagcaacgtctggagatcctgcgtgcgcacctgattccgcgtttccagcacggctttgttctgggtaacatcagcgacgatcgtctgcgtatgctggatgtgcagattcgtaaggcggttggtcaatggctgcgtctgccggcggacgtgccgaaagcgtactatcacgcggcggttcaagatggtggcctggcgatcccgagcgtgcgtgcgaccatcccggacctgattgttcgtcgttttggtggcctggatagcagcccgtggagcgtggcgcgtgcggcggcgaagagcgacaaaattcgtaagaaactgcgttgggcgtggaagcagctgcgtcgtttcagccgtgtggatagcaccacccaacgtccgagcgttcgtctgttttggcgtgagcacctgcacgcgagcgttgacggtcgtgagctgcgtgaaagcacccgtaccccgaccagcaccaaatggattcgtgaacgttgcgcgcagattaccggtcgtgatttcgtgcaatttgttcacacccacatcaacgcgctgccgagccgtattcgtggcagccgtggccgtcgtggtggcggtgagagcagcctgacctgccgtgcgggttgcaaagtgcgtgaaaccaccgcgcacatcctgcagcaatgccaccgtacccacggcggtcgtatcctgcgtcacaacaagattgtgagcttcgttgcgaaggcgatggaggaaaacaaatggaccgtggagctggaaccgcgtctgcgtaccagcgttggcctgcgtaaaccggacatcattgcgagccgtgatggcgtgggtgttatcgtggacgttcaggtggttagcggtcaacgtagcctggatgagctgcaccgtgaaaagcgtaacaaatacggcaaccacggtgagctggttgagctggttgcgggccgtctgggtctgccgaaagcggagtgcgtgcgtgcgaccagctgcaccattagctggcgtggcgtttggagcctgaccagctataaagagctgcgtagcatcattggtctgcgtgaaccgaccctgcagatcgtgccgattctggcgctgcgtggcagccacatgaactggacccgttttaaccaaatgaccagcgtgatgggtggcggtgttggttagtgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 38 - DNA, 3411 bp, synthetic construct
  source 1..3411: mol_type = other DNA
atggattacaaggatgacgacgataagggtaccgcattcaacattccttaccctatcggtcgtgttgattgtgaagtgtgccatgaggatataggtcagatgaacctgccgattcgtgaacacgcagtgagcgtgcacaacatcaataaattcaactacctgtgtcagctgtgcagcaaaagctacgacaccatcaactccgttaaggcccattatgttgcatgtcgtcgtcagaagaatgcttctagtacgaccgcggttcccaccaacgtgattaataacaaccagctggcaattaacacaaaccaagtgattagccgtaatccgctacagtgcgtggaatgcctgatgaagcaggtggatttctacgccaaggacaccaaagccctggttacccacatgcggaccaaacatgccgccgcctacgaagagtccaagaaagttgccaccagaagagttgcatggagcccggatgaggaccagattctggcagaactggaagtgaagctgaaaaagatccagaaaggacagctgctgagccgtctggtggtcgaatataacaaatgcgccgataaaagcaaagcaccgagcaggtcgaaagatgccattcgcacgcggagacaacagcacgactacaaactgctgcttagaagcctgcagagccaacagcctcctgtgggtagcgaggatagtgattccgacatcagctccagcaataataatccgcttacaaccacccataatgttaccccgacccctgacagcagcaatgttgtgctgctgatccaaaagattcgggaaagcgtggacagcatcgtgaaaattacaaatctgaaactgaacaccaatatgctgaatgcagcaagcgcatttatcaaccagaacaataacatggacccgttggagttaagcatgagaggcattgaggaggacgttaaagccattagagataaagaactacagaaaccgacacgtaatgtccctagtagcaccaccagcagaaagccgaccagaaatgccaagcggctggaaaaaagcaagaagtatggttattatcagcatctttattacaacaacaaaaagaaactggtggctgagattctggatggcgagacatctggcgccaagcctccgcctatgaatctggttgaggattattatcgtaatatttggagcagatctaccattgatgattctcccgtcaacaatatcaaaactgttaattccgattctatctttgccccaatttctcgtgatgaaatcaaactagctctgagcaataccaagaaagattccgccgcaggacctgatgccgttaccattaaagaagcaaaagctataatcgataatctgtacgtggcctataatatctggctgggtgttcagggcattccggaacaactgaagctgaataaaaccatcctgattccgaaaggcaacagcgacctgagcctgctgaaaaattggcgtccgattaccatctcttccattattctgcgtgtttataatcgtctgctggcctatagaatgaataaaatcttcaaaacaaatgacaagcaagttggtttcaagcctgttaatggctgcggtattaacattagctggctgcatagcctgctgaagcatgcacgtctgaataaaaatagcatctacgcttgtctggttgacgtgtccaaggcctttgatagcgtgagccaccagagcatcgtgagagctctgaccatgaatggagcaccgtctctgctggttaaactcattatggatcagtacactaatgttaataccgtaatcacatgtagcggtagcatcagcaacaagattaatattagcagcggtgtcaagcaaggtgatcctctgagttctctgctgttcaacttggttattgatgaactgttcgacgtgatcaaggatcagtatggatatactatcgacaacattgggaccaccaatgccagatgctttgcagatgatctgaccctgatctctagcagccgtatgggcatgaacaaactgctggaactgacaacaaagttcttcaaggaacgtggtctgaatgttaaccccagcaagtgcatgtccatcggtatgagcaagggttacaagggtaaaaaaagcaaaattgaaagcgagccactgtttagcattaccgacgcacagatccccatgctgggctatattgacaaaacaacacgttatctgggtgtaaacttcacttccattggcgcaatcgacgccaagaggattaaaaaggacctgcaggatactctggacaagctggaacacctgaaactcaaagcacagtgtaaaatggacctgctacgtacctacatgatccctcggtttatgtttcagctcatacacaccgagctgtatcctaagctgctgatcaaaatggatatcctcatcagaaagctggctaaacgtatacttcatctgcctatcagcaccagcagcgaatttttttatctgccgtttaaagaaggcggactgcagctgacaagcctgaaagaagcagtgggtctggccaaaattaaactgcataaaaaaattatgtcctcaaacgacccgatgctgtgctatctgatcgagagccagcgtagtcgtattgttgaacacttcatgaaggatctgaagctgggcgacagcctgaccctgaacgagatgaataatattaaagaatgttttatgaaagagaagcgaattagcttcgcccagaaaatccacggcgtgggctttgaagttttcagctccagcccgttaaccaaccagtggatcaacggtgagatcaaaaccatgaccacaaaaacctatataaacagcatcaagttgcgtaccaacaccctggaaacccgcgtaacgacctcacgtggtctgaacatcatcaaaacctgtcggagatgtcatgttgccgatgaaagtctgatgcacgtgctgcagtgttgtagctcaacaaagggcctccggtacagccgtcatcataaaatttgcgcaaaagtggccaacaaactggtgatgaacggctatggtgtgtttcgtgaaaagagctatcctgatcctaacaatagcggtagctacctgcgtcccgatatcatcgccgtgaagaacggtcatgtgattgtgctggatgtgaccgtagtttacgaggtgaccggtgcaacctttatcaacgcctaccagaccaaaatcaacaagtacaacgcaattatggtgcagatcgaacagatgtttaattgtgtgaatggcgagctacatggcttagtcattggctcccgaggcagcatacaccacagccagctgcacatctggcaccagatgggttttagcagtattgaactgaaatatgtggcaatcggctgtatggaagattctctgcgcattatgagcaccttcagcaaggccatcacctgataa

SEQ ID NO: 39 - protein, 1135 aa, synthetic construct
MDYKDDDDKGTAFNIPYPIGRVDCEVCHEDIGQMNLPIREHAVSVHNINKFNYLCQLCSKSYDTINSVKAHYVACRRQKNASSTTAVPTNVINNNQLAINTNQVISRNPLQCVECLMKQVDFYAKDTKALVTHMRTKHAAAYEESKKVATRRVAWSPDEDQILAELEVKLKKIQKGQLLSRLVVEYNKCADKSKAPSRSKDAIRTRRQQHDYKLLLRSLQSQQPPVGSEDSDSDISSSNNNPLTTTHNVTPTPDSSNVVLLIQKIRESVDSIVKITNLKLNTNMLNAASAFINQNNNMDPLELSMRGIEEDVKAIRDKELQKPTRNVPSSTTSRKPTRNAKRLEKSKKYGYYQHLYYNNKKKLVAEILDGETSGAKPPPMNLVEDYYRNIWSRSTIDDSPVNNIKTVNSDSIFAPISRDEIKLALSNTKKDSAAGPDAVTIKEAKAIIDNLYVAYNIWLGVQGIPEQLKLNKTILIPKGNSDLSLLKNWRPITISSIILRVYNRLLAYRMNKIFKTNDKQVGFKPVNGCGINISWLHSLLKHARLNKNSIYACLVDVSKAFDSVSHQSIVRALTMNGAPSLLVKLIMDQYTNVNTVITCSGSISNKINISSGVKQGDPLSSLLFNLVIDELFDVIKDQYGYTIDNIGTTNARCFADDLTLISSSRMGMNKLLELTTKFFKERGLNVNPSKCMSIGMSKGYKGKKSKIESEPLFSITDAQIPMLGYIDKTTRYLGVNFTSIGAIDAKRIKKDLQDTLDKLEHLKLKAQCKMDLLRTYMIPRFMFQLIHTELYPKLLIKMDILIRKLAKRILHLPISTSSEFFYLPFKEGGLQLTSLKEAVGLAKIKLHKKIMSSNDPMLCYLIESQRSRIVEHFMKDLKLGDSLTLNEMNNIKECFMKEKRISFAQKIHGVGFEVFSSSPLTNQWINGEIKTMTTKTYINSIKLRTNTLETRVTTSRGLNIIKTCRRCHVADESLMHVLQCCSSTKGLRYSRHHKICAKVANKLVMNGYGVFREKSYPDPNNSGSYLRPDIIAVKNGHVIVLDVTVVYEVTGATFINAYQTKINKYNAIMVQIEQMFNCVNGELHGLVIGSRGSIHHSQLHIWHQMGFSSIELKYVAIGCMEDSLRIMSTFSKAIT

SEQ ID NO: 40 - DNA, 3405 bp, synthetic construct
  source 1..3405: mol_type = other DNA
atggattacaaggatgacgacgataagggtaccgtgattatacacccgggcaccgatggtatttggtgttgtaccgaatgtgtggaaatccataatagcggcaaggatctgaagcgacacctggccaaacgccatccctcggtcaccatctccggctacaagtgcaacctgtgtccgtttgttagcgagcggcaactgagtgttggtacacatttgcgttactgtcgtggtgttaaggaagttgtgaaaagagaattcgcatgtgcatcctgttcctttagcagcgacaccttcagcggccttcaggtgcacatgcagcgtaagcacatcgcagagtggaatgatcagctgaaggaaaagacagagttcgcctggaccgaccgggaactccgtgaactggctgaaaaggagctgaccacaccgtccttccggtacaacaagatcttctacgccgctctgggcacctccaggacctacgatgcagtgcggaaaattcgctataatgatcgatacaagagcgccatcgccgaaatgagaagccagattgcagatgcagcagcagccgcccaagaacgtgatgtggagcgtggtctcgtgagcgcccacagcgaccgtggaaaagagatgctgcctgttgttgaaaccaaaagcgacatccaggttaataacgatatcaaaaaagacattgagctgacccctaatagccgccagaaacagaccaatctggccctggccagacctgccgtgatcgaggtggaagaggatctgggcagacaggacgtgaagcagtacctggcgagcctgagacaggatgattacaccagccctgctgagcgtagcatttttgcatactgccgcgaagaaacaaactggagcgccacgaagcggcaagtgctgaaaataagtcggaccaccagaggtctgcgccagccgaaaaaagtgcgtccatttgagtttccggagggcttcaaacccaacagaaatatgaggaagtggcgcaaatatcgttttctgcaggaatgctaccgtgaaaaacgtgcagaaacagtgagcaaaatcctggacggcaccttcattgacgaacccgaggaagaaattcgtcctgaactggaggaggttcagagaatgtacatcgaccgtctggaaaagcgtacacagctggataccaccaaaatcgttcagacagatgaagtgttctgcctgcagagctacggcagaatcacgatcggtgaagtgagagatgctctgggcgcaagcaaaaaggacagcgcatccggtccggatgggctgctgctgcaagacgtgcggaggctgggacctctgctgctgtgcaatatcttcaacatgtggtatctgcacggcatcccggtggaagagaacagatgtaggacaatcctgctgtataaaagcggtgatcgacatctggccagcaactaccgtccggttaccatcggcaacatgctgaaccgcctgtatgcaaaaatatgggacaaaagaattagaaaaaatgtgcgtctgcatgttagacagaaagccttcatcccggttgatggatgttttgagaatgtaaagaccattcagtgcgttctacagagctatcgtaagcgcaaattagaacacaacgtggtttttattgatctggccaaagcatttgataccgtgctgcatgacagcatccggaaagccctgtggcgtaagggggtgcctagcggcgtggtgaaggttgttgatagcctgtacgccggagccgtaacatctatcagcgttggcaagaccaaaacgagaagcatttgcatcaacagcggtgtcaaacagggctgccctttgtcaccgctgctgtttaatctgattctggatgagttagcagaacggattgaagccacaggctgcggccttgatcttgacggccacgttctgtctagcatggcctttgccgacgactatgttctgctggctaaggatagcgtggaaatgaacgaactgattcgcgtttgttctaccttcttcaaagagaagggcttgtctgtgaatcctggcaaatgtcagagcctgcgcgtgctgccagtaaaagagaaaaaacgcagcatgaaggtactggtgcgtcctcatcgttggtggcgtattaaggaccaggatgtggatattcccagcatgacctacgattctctcggcaagtatctgggggttagcatcgaccccaccggtaaaatcgccctgcctattgaggaatggaagaactggatgaccaaactgaaagagtgcaagctgaaaccggaacagaaagtgaagattctgaaagaagtggtgtgtagcagagtcaattatgttctgcgtatgagcgaatgtggtattagcgaactgcgtagctggaccagatttgttagaaattgggcaaaaaacatcatccatctgcctacgtggtgcagcagcgattggattcattccattaaaggtctgggtattcctgatgtgtctaagggcattgttatccaacggatgagagctagtgagaagatgagcaccagcgaagatggcatcgttcgtgttgtgggagcacgcctggttcagaagaatcgtgttctgtgggaaaaagccggttttgaaggcattgaactgaaagctgcacgtagacactgcgaagttgagagactgaacaacattggcaatatcaccaacggagtagccctgaagaccatcgcagccgtgtcctctgtgaaccgatactggatgattgaggacaatctgaaaagcggtaataaaatcctggtgtggaaggcaatggcaggtgctatcccgaccaaaatcaatctgtcacggggcgttgccgaccagacccttaagaaatgtcgacgttgtggtctaacagcggaaaccgatggtcatattctggctggctgccacaccagctctgatgcatatagcaaacgacataatatgctgtgtgataaactggccaaagagctgaaactgaatggcggtccgaatcggcgtgtttggcgggaacggacctgctttaccagcacaggtcgtcgttatcgtccggatattattgtaaaagatgatagcaaaatcacagtcatcgacatgacctgcccgtatgaaaaatccgaaggtcatctgatccagtgtgaaagcgcgaaagtgaccaaatatgaacctctgaaactggataagtattggacaagagagctggaaggcgccaatggtattgtggcagaaaaagttgagctgatgggcctggccatcggtgcaatcggcaccatcatgcgttctaccctgcgcaaactgtgtgaactgaagagcggtagaattgtgagacgtctgcagatgattgcatgtaacaacagcgcccagatcatcaagggtcatttgtcgcgcgccacacgccgtaatctgagatgataa

SEQ ID NO: 41 - protein, 1133 aa, synthetic construct
MDYKDDDDKGTVIIHPGTDGIWCCTECVEIHNSGKDLKRHLAKRHPSVTISGYKCNLCPFVSERQLSVGTHLRYCRGVKEVVKREFACASCSFSSDTFSGLQVHMQRKHIAEWNDQLKEKTEFAWTDRELRELAEKELTTPSFRYNKIFYAALGTSRTYDAVRKIRYNDRYKSAIAEMRSQIADAAAAAQERDVERGLVSAHSDRGKEMLPVVETKSDIQVNNDIKKDIELTPNSRQKQTNLALARPAVIEVEEDLGRQDVKQYLASLRQDDYTSPAERSIFAYCREETNWSATKRQVLKISRTTRGLRQPKKVRPFEFPEGFKPNRNMRKWRKYRFLQECYREKRAETVSKILDGTFIDEPEEEIRPELEEVQRMYIDRLEKRTQLDTTKIVQTDEVFCLQSYGRITIGEVRDALGASKKDSASGPDGLLLQDVRRLGPLLLCNIFNMWYLHGIPVEENRCRTILLYKSGDRHLASNYRPVTIGNMLNRLYAKIWDKRIRKNVRLHVRQKAFIPVDGCFENVKTIQCVLQSYRKRKLEHNVVFIDLAKAFDTVLHDSIRKALWRKGVPSGVVKVVDSLYAGAVTSISVGKTKTRSICINSGVKQGCPLSPLLFNLILDELAERIEATGCGLDLDGHVLSSMAFADDYVLLAKDSVEMNELIRVCSTFFKEKGLSVNPGKCQSLRVLPVKEKKRSMKVLVRPHRWWRIKDQDVDIPSMTYDSLGKYLGVSIDPTGKIALPIEEWKNWMTKLKECKLKPEQKVKILKEVVCSRVNYVLRMSECGISELRSWTRFVRNWAKNIIHLPTWCSSDWIHSIKGLGIPDVSKGIVIQRMRASEKMSTSEDGIVRVVGARLVQKNRVLWEKAGFEGIELKAARRHCEVERLNNIGNITNGVALKTIAAVSSVNRYWMIEDNLKSGNKILVWKAMAGAIPTKINLSRGVADQTLKKCRRCGLTAETDGHILAGCHTSSDAYSKRHNMLCDKLAKELKLNGGPNRRVWRERTCFTSTGRRYRPDIIVKDDSKITVIDMTCPYEKSEGHLIQCESAKVTKYEPLKLDKYWTRELEGANGIVAEKVELMGLAIGAIGTIMRSTLRKLCELKSGRIVRRLQMIACNNSAQIIKGHLSRATRRNLR

SEQ ID NO: 42 - DNA, 3087 bp, synthetic construct
  source 1..3087: mol_type = other DNA
atggattacaaggatgacgacgataagggtaccaataccctgggagaaaacgtggacgcctcagccaccacctcctctgcaaaccctctgtctcaggaaccgggctggtgcgagagctgtagcaaactgttcaagtcccagcgtggtctgcgggtacaccagcgcagcaagcaccccgaactgtaccacagtcagaatcagccgctgccacgttcaaaagcacgttggagcgacgaagaaatggttatatttgcccgtgaagagattgctaatcgaaaaatcagattcattaatcagcatctgcacaaagtgtttccgcaccgtaccctggagagcatcaagggcctgaggggtaaaaacgttagatatgctcgtatcatggccgacctggaagccgagatgaccagccagccagaagcagcaacctcactgtgtaccgagacaagcgagaacctggcaagcagcaatgttctgcctcagacccgtggctgggcagagaacctggttgaaaatatcgatactgcccatctggccaacctgggtccgctgagccagtttgagcccgggaaaccgtcgtccagtaccaaagaagccattaacaccgaatataatgattggatctctaagtggctgcctagcggtgccgcccacagagaaagacgcgccaacccccccagcacgaagctgaatgcacgtgcaaccagaagactgcaatacagtagaatccagaatctgtacaaactgaacagatctgcctgtgcacaagaggttctgtccggtgcctggaaagttcagagcggagaactgaatttgaaagaggttcagccgttttgggaaaagatgttccggaaagaaagcgcaaaagacagacggaagcctaagcccacaggtgaagtgctgtggggtctgatggaaccgctgaccatcgcagaagttggcagcacactgaagtccacgacccctagcgcaccaggtccggataaactaacactggatggtgttaagcgtatcccgatcgcggaactggttagccactacaatctgtggctgtatgcaggttatcagcctgaaggcctgcgggaaggcatcaccacactgattccgaaaattaaaggcacaagagatcccgcgaaactgcgtccgatcaccgtgagtagtttcatctgccggatctttcatcgttgtctggctcagcgtatggaaaccagcctgcctctgggtgaacgtcaaaaagcattcagaaaggtggacggcatttgtcataatatctggtccctgcggagcctgatccacaacagcaaagacaatctgaaagagctgaacattacctttctggatgttcgcaaagccttcgacagcatctctcataagagcctgggaattgccgcggcaaggctgggcctgcctcctccgctaattacttacatcagcaacctgtatccgaattgcagcaccaaacttaaggtgaatggcaaaatttccaaacccatagaggtccgtcgtggcgtgcgacagggtgatcctctgagtcctctgctgtttaatgccgtgatggattgggccctgagcgagctggaccctagagtaggtgttcagatcggcgaacagcggattaaccacctggcctttgcagatgatattattctggttagcagtaccaagattggcatggtgagctctatcaataccctgagccgtcatctggccaaaagtggactggagatcagtgccggtaaagaaggaaaatcggccagcatggctattgttgtcgatggtaaaaagaagatgtggaccgttgatccgctgcccagatttaaagtgaatagccaaaaaattccggcactgagcatcacccagcagtacaagtacctggggattaatatcgatgcacagggcgcccggaacgacgccgccagaattctgaccgagggcctagccgaactgagccgtgctccgttaaaaccgcagcagagactgtatctgctccgcgtgcatctgttacccaagctgcagcatggtctggtgctctctagctgcgccaagcgtgctctgacctacttagacaagagcgttcgttcagccatcaggagatggctgaccctgccgaaagatacaccgacagctttttatcatgcaaaagcatgcgacggaggtctgggtataacccggctggaacataccattcctatcctaaagcgtaacagaatgatgaagctgaccttaagcgaagatccggttatcatggaactcgtgaagctcacatacttcaccaatttattacacaagtacagcaacgtgaaactgctgaacagctggcctgtgaccgataaggatagcctggcacgggctgaggcaagcatgctgcacacctctgtggatggccggggcctgagcaactgcagcgatgtgcctagacagagcgattgggttaccaatggtgctagcctgctgagcggcagagattttattggtgcaatcaaagttcgtggtaatctgctgcctaccaaagtgagcgcagctcgtggacgtcaacgtgaaattacctgcgattgttgtcgtcgtcctgagagtctgggccacatcctacagacatgtccgcggacctggggccctcgcattagccggcacgatagcctgctgaagagagttcgcaaccaggcctgccttaaaaactggacccctatcatcgaaccgagcattcctacaaacattggtttacggagaccggatctggtgctggcgaaaggcaatatcgctttcctggtggacgccaccgtggtggccgataatgcaaacatgcagctgcagcatgaagccaaagtggaaaaatataacaacagcgacatcaaggaatggatcaaagttcattgccccggcgtcgacgaggttcgtgtgaccagcctgacagctaattggcggggctgtctgtatggcggtagcgctagcttcctaaccgaggacctgggtctgccgaaggcggagctcagcctgctcagcgccaagattaacgagaaaggctactacctgtggtgtgcacattatagaggcaccgcaagactgtggaatcgtcctctgagatcctgataa

SEQ ID NO: 43 - protein, 1027 aa, synthetic construct
MDYKDDDDKGTNTLGENVDASATTSSANPLSQEPGWCESCSKLFKSQRGLRVHQRSKHPELYHSQNQPLPRSKARWSDEEMVIFAREEIANRKIRFINQHLHKVFPHRTLESIKGLRGKNVRYARIMADLEAEMTSQPEAATSLCTETSENLASSNVLPQTRGWAENLVENIDTAHLANLGPLSQFEPGKPSSSTKEAINTEYNDWISKWLPSGAAHRERRANPPSTKLNARATRRLQYSRIQNLYKLNRSACAQEVLSGAWKVQSGELNLKEVQPFWEKMFRKESAKDRRKPKPTGEVLWGLMEPLTIAEVGSTLKSTTPSAPGPDKLTLDGVKRIPIAELVSHYNLWLYAGYQPEGLREGITTLIPKIKGTRDPAKLRPITVSSFICRIFHRCLAQRMETSLPLGERQKAFRKVDGICHNIWSLRSLIHNSKDNLKELNITFLDVRKAFDSISHKSLGIAAARLGLPPPLITYISNLYPNCSTKLKVNGKISKPIEVRRGVRQGDPLSPLLFNAVMDWALSELDPRVGVQIGEQRINHLAFADDIILVSSTKIGMVSSINTLSRHLAKSGLEISAGKEGKSASMAIVVDGKKKMWTVDPLPRFKVNSQKIPALSITQQYKYLGINIDAQGARNDAARILTEGLAELSRAPLKPQQRLYLLRVHLLPKLQHGLVLSSCAKRALTYLDKSVRSAIRRWLTLPKDTPTAFYHAKACDGGLGITRLEHTIPILKRNRMMKLTLSEDPVIMELVKLTYFTNLLHKYSNVKLLNSWPVTDKDSLARAEASMLHTSVDGRGLSNCSDVPRQSDWVTNGASLLSGRDFIGAIKVRGNLLPTKVSAARGRQREITCDCCRRPESLGHILQTCPRTWGPRISRHDSLLKRVRNQACLKNWTPIIEPSIPTNIGLRRPDLVLAKGNIAFLVDATVVADNANMQLQHEAKVEKYNNSDIKEWIKVHCPGVDEVRVTSLTANWRGCLYGGSASFLTEDLGLPKAELSLLSAKINEKGYYLWCAHYRGTARLWNRPLRS

SEQ ID NO: 44 - DNA, 3492 bp, synthetic construct
  source 1..3492: mol_type = other DNA
atggggagcgactacaaagaccatgacggtgattataaagatcatgacatcgattacaaggatgacgatgacaagaagggtggaggtagcgggggcagtggagggcccaagaagaagaggaaggtgatgtgcgtgaccgagggtgaatgcctgccgccgaccgacaactgcaacccgagcgtggagaaccagctgccgtgcgtgaccgagggtcgcttcgaacgtgtgggcagcctggtgaccgtgcgtctgccatttcgcaaggtggcctgcgacctgtgcagcaaggagttcctgacctacagcaaatttgctgtgcaccaggccaacttccacaacagcgaaacccaggcttgctgcacctattgcggcaagagcgatggcaaccaccacagcattgcttgccatgtgccaaaatgcccgtggcgtcgcaccgtgaccttcgccgccaacctgagcaactttctgtgcgacctgtgcaacgatagctttaagaccaaaagcggtctgagccagcacaagcgccacaaacacccgtgcagccgtaatgccgaacgcatcctgagcctgggcgtgcgtaccccaagcgcccgtccacgtcaggtggtgtggagcgaggaagagacccgcaccctgcgtgaggtggaagtggtgtacagcggtcagaagaacattaacgtgctgtgcgccggtcacctgccaggcaagaccagcaaacaagtgagcgacaaacgtcgcgatctgcaccgcattcgtagcagcaacgtgcacggtaccccgaccacccagagccgtggcgacccggtggagcaggtggaagagtacgaagagctggattgggagggtatgcacccgttcccggacccggatagcaagttttgcagctatctggaccagctgcgcgatcagaaaggcctgaccgagccggtgtggcaggagatcgaaattgtggctcaggagtgggtggaaaacctggcccacgtgcagagcagctggaaccacgagcgtaccaccaagcaggtgccagaaaataataccccggctcgtcgcccgttcaagcgtcgcctgcaccgtgtggagcgttacaaacgctttcagcgtatgtatgacctgcagcgcaagcgtctggccgaagagatcctggatggccgtgaagcggtgacctgcaacctgaagaaagaagagattaaagaccactacgatcaggtgtatggtgtgagcaacgaccgtgtgagcctggatgattgcccgcgtccgccgggtgccaacaacaccgacctgctgaagccgttcaccccgaccgaggtgatggatagcctgcagggtatgaagaacggtgctccgggcccggataaaatcaccctgccgtttctgcagaagcgtctgaaaaacggtattcacgtgagcctggccaacgtgttcaacctgtggcagtttagcggccgtatcccggagtgcatgaaaagcaaccgcagcgtgctgattccgaagggcaaaagcaacctgcgcgacgtgcgtaactggcgcccgatcaccattagcagcatcgtgctgcgtctgtacacccgcattctggctcgtcgcctggaacgtgccgtgcagatcaatccacgtcagcgtggttttgtgccacaggcgggctgccgcgataacatttttctgctgcagagcgcgatgcgtcgcgctaagcgtaaaggtaccctggctctgggcctgctggacctgagcaaggccttcgataccgtgggccacaaacacctgctgaccagcctggagcgtttcgctgtgcacccgcactttgtgcgcatcgtggaagacatgtacagcggttgcagcaccagctttcgtgtgggcagccagagcacccgtccgattgtgctgatgcgcggtgtgaagcagggcgacccgatgagcccgatcctgtttaacatcgccctggacccgctgctgcgtcagctggaagaggaaagccgtggtttcatgtttcgtgagggtcaggccccggtgagcagcctggcttatgccgacgatatggctctgctggccaaggaccacgctagcctgcagagcatgctgggtaccgtggataaattctgcagcggcaacggtctgggcctgaacatcgcgaagagcgccggtctgctgattcgcggcgctaacaaaacctttaccgtgaacgactgcccgagctggctggtgaacggcgagaccctgccgatgatcggcccggaacagacctaccgttatctgggtgctagcatctgcccgtggaccggtattaacagcggcccggtgaagccgaccctggagaaatggatcgccaacattaccgaaagcccgctgaagccacaccagcgtgtggacattctgtgcaaatacgcgctgccgcgtctgttctatcagctggagctgggtaccctgaactttaaggaactgaaagaactggatagcatggtgaagcaggcggtgaagcgttggtgccatctgccagcttgcaccgccgatggtctgctgtacagccgtcaccgtgatggcggtctggccgtggtgaagctggaaagcctggtgccgtgcctgaagatcaaaaccaacctgcgtctggtgcacagcaccgacccggtgattagcagcctggccgagagcgatggtctggtgggtgctatcgaaggtattgctcagaaagcgggtctgccaatcccaaccccagaccagcgtagcggtacctaccacagcaactggcgcgatatggagcgtcgcagctgggaacgtctggccctgcacggtcagggtgtggagctgttcaaaggtagccgtagcgcgaaccactggctgccacgtccagtgggcatgaagccgcaccactgggtgaaatgcctggcgatgcgtgctaacgtgtatccgaccaagcgtggtctgagccgcggcaacctgagcaagaacaaagacagcgccaaatgccgtggttgcaccagcatgcgtgagaccctgtgccacctgagcggccagtgcccgaagctgaaaagcatgcgtatccgtcgccacaacaaaatctgcgagcacctgattgcggaagctagcttcaagggttggaaagtgctgcaggagccgaccctggtgaccgacaacggcgaacgtcgccgtccggatctgatctttcaccgtgacgataaggcggtggtggtggacgtgaccgtgcgctacgagattagcaaggataccctgcgtgaagcctacgcgagcaaagtgcgccgttatggctgcctgaccgagcagatcaaagacctgaccggtgcgaccagcgtggtgttccacggctttccgatgggtgcgcgtggcgcctggttcccagaaagcagcgacgtgatggctgatctgaacatccgtagcaagtacttcgaggaatttctgtgccgccgtaccattctgtataccctggatctgctgtggaaaagcaacaacgagcagtatctggaacgtctggcgccgtaataa

SEQ ID NO: 45 - protein, 1162 aa, synthetic construct
MGSDYKDHDGDYKDHDIDYKDDDDKKGGGSGGSGGPKKKRKVMCVTEGECLPPTDNCNPSVENQLPCVTEGRFERVGSLVTVRLPFRKVACDLCSKEFLTYSKFAVHQANFHNSETQACCTYCGKSDGNHHSIACHVPKCPWRRTVTFAANLSNFLCDLCNDSFKTKSGLSQHKRHKHPCSRNAERILSLGVRTPSARPRQVVWSEEETRTLREVEVVYSGQKNINVLCAGHLPGKTSKQVSDKRRDLHRIRSSNVHGTPTTQSRGDPVEQVEEYEELDWEGMHPFPDPDSKFCSYLDQLRDQKGLTEPVWQEIEIVAQEWVENLAHVQSSWNHERTTKQVPENNTPARRPFKRRLHRVERYKRFQRMYDLQRKRLAEEILDGREAVTCNLKKEEIKDHYDQVYGVSNDRVSLDDCPRPPGANNTDLLKPFTPTEVMDSLQGMKNGAPGPDKITLPFLQKRLKNGIHVSLANVFNLWQFSGRIPECMKSNRSVLIPKGKSNLRDVRNWRPITISSIVLRLYTRILARRLERAVQINPRQRGFVPQAGCRDNIFLLQSAMRRAKRKGTLALGLLDLSKAFDTVGHKHLLTSLERFAVHPHFVRIVEDMYSGCSTSFRVGSQSTRPIVLMRGVKQGDPMSPILFNIALDPLLRQLEEESRGFMFREGQAPVSSLAYADDMALLAKDHASLQSMLGTVDKFCSGNGLGLNIAKSAGLLIRGANKTFTVNDCPSWLVNGETLPMIGPEQTYRYLGASICPWTGINSGPVKPTLEKWIANITESPLKPHQRVDILCKYALPRLFYQLELGTLNFKELKELDSMVKQAVKRWCHLPACTADGLLYSRHRDGGLAVVKLESLVPCLKIKTNLRLVHSTDPVISSLAESDGLVGAIEGIAQKAGLPIPTPDQRSGTYHSNWRDMERRSWERLALHGQGVELFKGSRSANHWLPRPVGMKPHHWVKCLAMRANVYPTKRGLSRGNLSKNKDSAKCRGCTSMRETLCHLSGQCPKLKSMRIRRHNKICEHLIAEASFKGWKVLQEPTLVTDNGERRRPDLIFHRDDKAVVVDVTVRYEISKDTLREAYASKVRRYGCLTEQIKDLTGATSVVFHGFPMGARGAWFPESSDVMADLNIRSKYFEEFLCRRTILYTLDLLWKSNNEQYLERLAP

SEQ ID NO: 46 - DNA, 3039 bp, synthetic construct
  source 1..3039: mol_type = other DNA
atggattacaaggatgacgacgataagggtaccatgcaccaggtgcggaacgccgatgccaatctgcagaccatgttcccctgcagagagtgtgagcgcagctttcggacaaagatcggactgggagtgcacatgaggcacaggcacaaggacgagctggataccgcccggagaagggtggacgtgaaggccagatggaatgaggaggagctgtccatgatggccaggaaggagctggagctgaccgcaaacggagagcgcttcatcaataagaagctggccgagatctttacaaacagaagcgtggatgccatcaagaagtgcagacagagggacaattacaaggccaagatcgagcagctgcagggacaggcagcactgatctccgaggcaaacgagccacctaccacacagaggaggccatctctgagcgagctggaggtgaccccaagctcctctcactccgtgccaatcgcaccaccacctatccactctgacgatatcctgctgcaggagctgcagggcatgagccctgtggccgtgagaaggtcctggagagtggaggtgctgcagagcatcatcgatagggcacacatctccggcaaggaggcaaccctgcagtgtctgagcaactacctgctggagatcttccctaacagaaatgacaggccaagctccgcaacagtgccagcacgccggcctaggaatagaaggatctctcgccggcagcagtacgcccgctgcatcaagagcctgctggacggaaccgatgagtccgcactgcctaaccagtctatcatggagccatattggaggcaggtcatgacccagccatccccctctctgtgttccaacacagtgcctcggaagggcaatatgcaggagggcgtgtggtctccaatcaccagcagagatctgcaggtgcacaaggtgcccctgacatctagccctggcccagacggcatcacctcccagacagccagatctatcccaatcggcatcatgctgaggatcgtgaatctgatcctgtggtgcggcgatctgccagtgcctttccgcatggcaaggaccatctttatccctaagacagtgagagccaacaggccacaggacttccgcccaatcagcgtgccctccatcgtggtgcgccagctgaatgccatcctggcctctcggctgaccgcagccgtgagctgggaccccaggcagaggggctttctgcctaccgacggatgcgccgataacgccacaatcgtggacctggtgctgcgcgatcaccacaagcggtacgccagctgttatatcgccaccctggatgtgagcaaggcctttgactccgtggcccacgatgccgtgtttaacaccgtgacagcctacggcgcccccaagagcttcgtggactacgtgagaaggtggtattccggcggcggcacatatttcaatggcggcgattggagatccgaggagtttgtgccagcaaggggagtgaagcagggcgacccactgtcccccgtgctgtttaacctgatcatcgatcgcctgctgcggtctctgcctaaggacatcggcgtgcacgtgggcaacgccaaagtgaatgcctgtgccttcgccgacgatctgatgctgtttgccagcacccctaagggcctgcaggagctgctgaacaccacagtgaagttcctgtcctctgtgggcctgaccctgaatgccgacaagtgctttacaatctctatcaagggccagccaaagcagaaggtgaccgtggtggagcagaggacattctgtatcggcagagccagggtgcagctgaagcgctccgaggagtggaagtacctgggcatccactttacagcagatggaagggcaaggtataacccaagcgaggacatcggccctaagctggagaggctgatgcagtcccctctgaagccacagcagaagctgttcgcactgaggaccgtgctggtgcctcagctgtaccacaagctgacactgggcagcgtggcactgggagtgctgaggaagtgcgataagctggtgagatctttcgccaggaagctgctgggactgccactggacgtgagcgtggccttttatcacgcaccacactcttgtggaggactgggcatcccaagcgtgcgctggatcgcacccatgctgagaaccaagaggctggccggcatcaattggccacacctggagcagtccgaggtggcctctgcctttctgagcgaggagctgaggagggcaagagatagggcaaaggcaggagtgaacgagctgctgagccagcccaagatcgacacctactgggccgatcggctgtatacatccgtggacggcaatggcctgagagaggcaagaagatacgcaccacagcacggatgggtgtcccagcctacccggctgatgtctggcaaggcctatcgcacaggcatccagctgcggatcaacgccctgcctacccgctctcggaccacaagaggcaggcacgagatgaacaggcagtgcagggcaggatgtgatgcaccaagccacaatcacgtgctgcagagatgccacaggacacacggctctcgcgtgagccggcacaatggcgtggtgagctacctgaagaagggcctggagaccagaggctacacagtgtattccgagcagtctctgcacggccagaaccgcgtgtataagcccgatatcgtggccttccggcacgactccaccatcgtggtggatgcccaggtggtgacagacggactggacctggatagggcacaccagtctaaggtggagatctacaatcgccaggacctgctgaccacactgaggtccgtgtatagggcaagggagaacatcgaggtggtgtctgccaccctgaattggagaggcatctggagctttcagtccatcaccagactgaggacactgggcatcctgaccgccggcgactccaacgtgatcagctccagagtggtgtctggcagggtgtacagcttcaagacattcatgtttcacgccggctttcaccggggcatggcctag

SEQ ID NO: 47 - protein, 1012 aa, synthetic construct
MDYKDDDDKGTMHQVRNADANLQTMFPCRECERSFRTKIGLGVHMRHRHKDELDTARRRVDVKARWNEEELSMMARKELELTANGERFINKKLAEIFTNRSVDAIKKCRQRDNYKAKIEQLQGQAALISEANEPPTTQRRPSLSELEVTPSSSHSVPIAPPPIHSDDILLQELQGMSPVAVRRSWRVEVLQSIIDRAHISGKEATLQCLSNYLLEIFPNRNDRPSSATVPARRPRNRRISRRQQYARCIKSLLDGTDESALPNQSIMEPYWRQVMTQPSPSLCSNTVPRKGNMQEGVWSPITSRDLQVHKVPLTSSPGPDGITSQTARSIPIGIMLRIVNLILWCGDLPVPFRMARTIFIPKTVRANRPQDFRPISVPSIVVRQLNAILASRLTAAVSWDPRQRGFLPTDGCADNATIVDLVLRDHHKRYASCYIATLDVSKAFDSVAHDAVFNTVTAYGAPKSFVDYVRRWYSGGGTYFNGGDWRSEEFVPARGVKQGDPLSPVLFNLIIDRLLRSLPKDIGVHVGNAKVNACAFADDLMLFASTPKGLQELLNTTVKFLSSVGLTLNADKCFTISIKGQPKQKVTVVEQRTFCIGRARVQLKRSEEWKYLGIHFTADGRARYNPSEDIGPKLERLMQSPLKPQQKLFALRTVLVPQLYHKLTLGSVALGVLRKCDKLVRSFARKLLGLPLDVSVAFYHAPHSCGGLGIPSVRWIAPMLRTKRLAGINWPHLEQSEVASAFLSEELRRARDRAKAGVNELLSQPKIDTYWADRLYTSVDGNGLREARRYAPQHGWVSQPTRLMSGKAYRTGIQLRINALPTRSRTTRGRHEMNRQCRAGCDAPSHNHVLQRCHRTHGSRVSRHNGVVSYLKKGLETRGYTVYSEQSLHGQNRVYKPDIVAFRHDSTIVVDAQVVTDGLDLDRAHQSKVEIYNRQDLLTTLRSVYRARENIEVVSATLNWRGIWSFQSITRLRTLGILTAGDSNVISSRVVSGRVYSFKTFMFHAGFHRGMA

SEQ ID NO: 48 - DNA, 3198 bp, synthetic construct
  source 1..3198: mol_type = other DNA
atggggagcgactacaaagaccatgacggtgattataaagatcatgacatcgattacaaggatgacgatgacaagaagggtggaggtagcgggggcagtggagggcccaagaagaagaggaaggtgatgaccacccgcccgagcgttgacatcttcccagaggaccaatacgaaccaaatgccgcagcgaccctgagccgtgtgccgtgtaccgtgtgcggtcgtagcttcaacagcaaacgtggtctgggtgtgcacatgcgcagccgtcacccagacgagctggatgaggaacgtcgccgtgtggacatcaaggcccgttggagcgatgaggaaaaatggatgatggctcgcaaggaggtggaactgaccgccaacggctgcaagcacattaacaaacagctggcggtgtactttgctaaccgtagcgtggaagccatcaagaaactgcgccagcgtggtgactataaggagaaaatcgaacagattcgtggtcagagcgccctggctccagaggtggctaacctgaccatccgccgtcgcccgagccgcagcgaacaggatcaccaggtgaccaccagcgaaaccaccccgattaccccgtttgagcagagcaaccgtgaaatcctgcgtaccctgcgcggttacagcccggtggaatgccacagcaaatggcgtgctcaggagctgcagaccatcattgaccgcgcccacctggagggcaaggaaaccaccctgcagtgcctgagcctgtacctgctgggtatctttccggcgcagggcgtgcgtcacaccctgacccgcccgccacgtcgcccgcgtaaccgtcgcgagagccgtcgccagcagtatgctgtggtgcagcgtaactgggacaagcacaaaggtcgctgcatcaagagcctgctgaacggcaccgatgaaagcgtgatgccgagccaggagatcatggtgccgtattggcgtgaagtgatgacccagccaagcccaagcagctgcagcggtgaagtgatccagatggaccacagcctggaacgcgtgtggagcgcgatcaccgagcaggatctgcgtgctagccgcgtgagcctgagcagcagcccaggtccagacggtatcaccccaaagagcgcccgtgaggtgccaagcggtatcatgctgcgcattatgaacctgatcctgtggtgcggcaacctgccgcacagcattcgtctggcccgcaccgtgttcatcccgaagaccgtgaccgcgaaacgtccgcaggattttcgcccaatcagcgtgccaagcgtgctggtgcgtcagctgaacgcgattctggctacccgcctgaacagcagcatcaactgggacccgcgtcagcgtggtttcctgccaaccgacggttgcgctgataacgctaccattgtggatctggtgctgcgtcacagccacaaacacttccgcagctgctacatcgccaacctggacgtgagcaaggcgtttgatagcctgagccacgctagcatctacgacaccctgcgtgcttatggtgccccgaagggctttgtggattacgtgcagaacacctatgagggtggtggcaccagcctgaacggtgacggctggagcagcgaggaattcgtgccagctcgtggtgtgaaacagggcgacccgctgagcccgatcctgttcaacctggttatggatcgtctgctgcgcaccctgccgagcgaaattggtgcgaaagtgggcaacgctatcaccaacgccgcggctttcgccgacgatctggtgctgtttgcggaaacccgtatgggtctgcaggtgctgctggacaaaaccctggatttcctgagcattgtgggcctgaagctgaacgccgacaaatgctttaccgtgggtatcaagggccagccgaagcagaaatgcaccgtgctggaagcgcagagcttctacgtgggtagcagcgaaattccgagcctgaaacgtaccgacgagtggaagtatctgggtatcaactttaccgctaccggtcgcgtgcgttgcaacccagcggaggatatcggcccgaaactgcagcgtctgaccaaggcgccgctgaaaccgcagcagcgtctgttcgctctgcgcaccgtgctgattccgcagctgtaccacaagctggccctgggtagcgtggcgattggcgtgctgcgtaagaccgacaaactgatccgctactatgtgcgtcgctggctgaacctgccgctggatgtgccaattgccttcgtgcacgctccaccaaagagcggtggcctgggcatcccgagcctgcgttgggtggctccaatgctgcgtctgcgtcgcctgagcaacatcaaatggccgcacctgacccagaacgaggtggctagcagctttctggaggctgaaaagcagcgtgctcgtgaccgcctgctggctgagcagaacgaactgctgagccgtccggcgattgaaaagtactgggctaacaaactgtatctgagcgtggatggtagcggcctgcgtgagggtggtcactacggtccacagcacggttgggtgagccagccgacccgcctgctgaccggtaaagaatatatggacggcattcgtctgcgcatcaacgctctgccaaccaagagccgtaccacccgtggtcgccacgagctggaacgtcagtgccgtgctggttgcgatgctccagaaaccaccaaccacatcatgcagaaatgctaccgtagccacggtcgtcgcgtggctcgtcacaactgcgtggtgaaccgtattaagcgcggtctggaggaacgtggctgcgttgtgatcgtggagccgagcctgcagtgcgaaagcggtctgaacaaaccggacctggtggctctgcgccagaaccacatcgacgtgattgatacccagatcgtgaccgatggccacagcatggacgatgcccaccagcgtaagattaaccgttacgaccgtccagatatccgtaccgagctgcgtcgccgtttcgaagctgctggcgacattgagtttcacagcgcgaccctgaactggcgtggtatttggagcggccagagcgtgaagcgcctgatcgctaaaggtctgctgagcaagtatgatagccacatcattagcgtgcaggttatgcgtggctccctgggttgcttcaaacagttcatgtatctgagcggcttttcccgtgattggacctaataa

SEQ ID NO: 49 - protein, 1064 aa, synthetic construct
MGSDYKDHDGDYKDHDIDYKDDDDKKGGGSGGSGGPKKKRKVMTTRPSVDIFPEDQYEPNAAATLSRVPCTVCGRSFNSKRGLGVHMRSRHPDELDEERRRVDIKARWSDEEKWMMARKEVELTANGCKHINKQLAVYFANRSVEAIKKLRQRGDYKEKIEQIRGQSALAPEVANLTIRRRPSRSEQDHQVTTSETTPITPFEQSNREILRTLRGYSPVECHSKWRAQELQTIIDRAHLEGKETTLQCLSLYLLGIFPAQGVRHTLTRPPRRPRNRRESRRQQYAVVQRNWDKHKGRCIKSLLNGTDESVMPSQEIMVPYWREVMTQPSPSSCSGEVIQMDHSLERVWSAITEQDLRASRVSLSSSPGPDGITPKSAREVPSGIMLRIMNLILWCGNLPHSIRLARTVFIPKTVTAKRPQDFRPISVPSVLVRQLNAILATRLNSSINWDPRQRGFLPTDGCADNATIVDLVLRHSHKHFRSCYIANLDVSKAFDSLSHASIYDTLRAYGAPKGFVDYVQNTYEGGGTSLNGDGWSSEEFVPARGVKQGDPLSPILFNLVMDRLLRTLPSEIGAKVGNAITNAAAFADDLVLFAETRMGLQVLLDKTLDFLSIVGLKLNADKCFTVGIKGQPKQKCTVLEAQSFYVGSSEIPSLKRTDEWKYLGINFTATGRVRCNPAEDIGPKLQRLTKAPLKPQQRLFALRTVLIPQLYHKLALGSVAIGVLRKTDKLIRYYVRRWLNLPLDVPIAFVHAPPKSGGLGIPSLRWVAPMLRLRRLSNIKWPHLTQNEVASSFLEAEKQRARDRLLAEQNELLSRPAIEKYWANKLYLSVDGSGLREGGHYGPQHGWVSQPTRLLTGKEYMDGIRLRINALPTKSRTTRGRHELERQCRAGCDAPETTNHIMQKCYRSHGRRVARHNCVVNRIKRGLEERGCVVIVEPSLQCESGLNKPDLVALRQNHIDVIDTQIVTDGHSMDDAHQRKINRYDRPDIRTELRRRFEAAGDIEFHSATLNWRGIWSGQSVKRLIAKGLLSKYDSHIISVQVMRGSLGCFKQFMYLSGFSRDWT

SEQ ID NO: 50 - DNA, 3579 bp, synthetic construct
  source 1..3579: mol_type = other DNA
atggattacaaggatgacgacgataagggtacccatatggccggtcctagcaccagatctagaaggaagggctgcctgccccctgtggacttcgagaaccctaagaagagaacaagactcatggccaagatgacaaatggcaaccctaccagccacgtgccgtgccccgcaccctgcagcaatggccacgagggcggcggaagagtggctgtgatcgagggccggcttccagaactgagcggcagcagaattagcgggattcagcctgccctacctgtggaaaccagcttcgtgggacaaagcaccggcagaggggccgacggagatgccaacgccaacagcagccctcccagccccaacctgggcggcagcgtgggcatggtgcctgcagtgcgcgatggcacacctcctctgggccggcctggcgaggaccacagccgggaatgcgccggcggcaacacccctctgtggatgctggaggatagcttccggtgcgactactgccctagagagttcggaacaagagccggaagaagcctgcacatgcggcgggctcacctggctgagtacgacggcgctggattttgctggggcgaaagactgtccgagttcgccgctacccggctgtggtctaccgaggaaaccaagaagctggctgtcttttgtgaacgcggtgtgcccagcccttctgagtgcagggccatcgccgccagcctcggcgccggtaagacacaccaccaggtgaggagcaagtgccgactggtgtttgaggctatcagacggagagagctgctggaagtggccgccgccaccgagagactggaaaagagcgcacggcggaagcagcctgccgtgcccccagcacccgtgcacggcgtgagaggcgtgcttagaggcctgctgggcaagagagtgccgcgggagggtggcaccacaggaagcacctccgcccgtattgtgagaagggacgactgccggcagggcgctgtggcctccgccagcttgaacctgatccggcggctgggccgcaaggctacaggaagaagcggacgtaggagagtcctgggcagacctcccagaatggatgtgcgccggagcgtgcgtatgagaagaatgcggcggttcctgtacagactggctcggctgggctgggccaagctggccatgttcgtgttggatggccagatgggcgcttcttgtcctgttccactggtggaagtgagcgccgtgttcagagagcggtggagcatcgtcagagccttcctgggcctgggacagttcggcggcttcggcaccgccgacaacgcgggctttggcaagttgatagaccctgcggaggtgagagctcacctgcagagcatcaaaaacagaagcagccctggccctgacggcatcaccaaggtggccctgtctaagtgggaccctgagggaatcaagctggcccacatgtacagcacctggctggtgtctgctggcatcccaaaggtgttcaagaaatgcagaacaacactgatccctaagaccggcgacgtgtccctgcatggcgacgtggggcagtggcggcctatcacaatcgcctccctggtgctgagactgtactcccgcatcctgaccgagagaatgaccgtggcctgccctagccatcctagacaaagaggattcatcgccagccccggctgttctgaaaacctgatgctgcttgaaggctgtatgagcctgagcaaggccggcaacggctccctggctgttgtgtttgtggacttcgccaaggctttcgacaccgtgagccacgagcacctgctgagcgttttagttcagaagggcctggaccagcacatggtggaactgatcaaggacagctatgagaacagcgtgaccaaggtgcattgccaggagggatgctctaccgacatcgccatgaaagtgggcgtgaaacagggcgatagtatgtctccactgctgttcaacctggccctagaccctctgatccagcagctggaaagagaaggcaggggcttccccgtgaacggaaagtctatcaccgccatggcctttgccgatgacctggccatcgtgagcgacagctgggagggcatgagagccaatctggacatcctggtggacttctgtgagctgaccggaatgcggacccagcccagcaagtgccacggtttcctgattgaaaagagcggctcccgtagctacaaggtgaacagatgcgagccttggctgctgaacgataccgccctgcacatggtgggccccaaggagtccatcaagtacctgggggtgcaagtgaatccctggacaggcatcttcgccgaagataccgtggctaagctgagacagtgggtggtggccatcagcaagacacctctgagacctctggataaggttagcctgctgtgtcagttcgccgtgcctagggtgatcttcgtggccgaccactgtatgctgagcgccaaagccctgacagagatggacagaagcatccggcaggccgtaaagagatggctgcacctagctcggtgtaccaccaacggcctgctctacagccggaagtcatccggcggcctgggcatccctaaactgagcatgatcgtccctgcgatgcaggccagacggctgctggggctgagccgctctaaagacgagactgtaagatggatgtttctggaaaccacagatcacgtggccttcgagagagcctggcttcgggccggcggaagccccgacgaggtgccagagctgggccctgatctggtggaaggatctccagccgaaggcaatgccgatcccgtgtctacagtgagacccagaaagagaatcgtgccatgtgactggagacaagtggagttcgacagatgggccggccaactggttcagggaaagggaatcagaacattcgaggccgataagatctctaattgctggctgtatgactaccctcctaacaagctcaagcctggcgatttcaccgccgccgtgcagctgagagccaacgtgtaccccacaagagagctcgccggcagaggcagaaccgacacaatcgacgtgtgctgccggcactgcggcgaggctcctgaaacctgttggcacatcctggccctgtgccccaaggtgaagcggtgcagaatccagagacaccacaaggtctgccaggtgctggtggccgaggccgagagacacggctgggaagtggaaagagagaaacggtggatgctgcccagcggcgaatgcgtggcccccgatctgatctgctggctggacgaactggcactgatcgtggacgtaaccgttagatacgagttcgacgaggaatccctggaaagagccagaatcgagaaagaatgcaagtaccggcccctgatccccgtgatcagagcctctcgtgtgcagaccaagaaagtcacagtttacggctttcctctgggcgcccgtggtaagtggcctgccaaaaacgagctgctgctggccgacctgggcctgtcaaaggccaggaccagatctttcgccaaacttctgtcccggagagtgcttctgcacagcctggatgtcatgagaaccttcatgcgctag

SEQ ID NO: 51 - protein, 1192 aa, synthetic construct
MDYKDDDDKGTHMAGPSTRSRRKGCLPPVDFENPKKRTRLMAKMTNGNPTSHVPCPAPCSNGHEGGGRVAVIEGRLPELSGSRISGIQPALPVETSFVGQSTGRGADGDANANSSPPSPNLGGSVGMVPAVRDGTPPLGRPGEDHSRECAGGNTPLWMLEDSFRCDYCPREFGTRAGRSLHMRRAHLAEYDGAGFCWGERLSEFAATRLWSTEETKKLAVFCERGVPSPSECRAIAASLGAGKTHHQVRSKCRLVFEAIRRRELLEVAAATERLEKSARRKQPAVPPAPVHGVRGVLRGLLGKRVPREGGTTGSTSARIVRRDDCRQGAVASASLNLIRRLGRKATGRSGRRRVLGRPPRMDVRRSVRMRRMRRFLYRLARLGWAKLAMFVLDGQMGASCPVPLVEVSAVFRERWSIVRAFLGLGQFGGFGTADNAGFGKLIDPAEVRAHLQSIKNRSSPGPDGITKVALSKWDPEGIKLAHMYSTWLVSAGIPKVFKKCRTTLIPKTGDVSLHGDVGQWRPITIASLVLRLYSRILTERMTVACPSHPRQRGFIASPGCSENLMLLEGCMSLSKAGNGSLAVVFVDFAKAFDTVSHEHLLSVLVQKGLDQHMVELIKDSYENSVTKVHCQEGCSTDIAMKVGVKQGDSMSPLLFNLALDPLIQQLEREGRGFPVNGKSITAMAFADDLAIVSDSWEGMRANLDILVDFCELTGMRTQPSKCHGFLIEKSGSRSYKVNRCEPWLLNDTALHMVGPKESIKYLGVQVNPWTGIFAEDTVAKLRQWVVAISKTPLRPLDKVSLLCQFAVPRVIFVADHCMLSAKALTEMDRSIRQAVKRWLHLARCTTNGLLYSRKSSGGLGIPKLSMIVPAMQARRLLGLSRSKDETVRWMFLETTDHVAFERAWLRAGGSPDEVPELGPDLVEGSPAEGNADPVSTVRPRKRIVPCDWRQVEFDRWAGQLVQGKGIRTFEADKISNCWLYDYPPNKLKPGDFTAAVQLRANVYPTRELAGRGRTDTIDVCCRHCGEAPETCWHILALCPKVKRCRIQRHHKVCQVLVAEAERHGWEVEREKRWMLPSGECVAPDLICWLDELALIVDVTVRYEFDEESLERARIEKECKYRPLIPVIRASRVQTKKVTVYGFPLGARGKWPAKNELLLADLGLSKARTRSFAKLLSRRVLLHSLDVMRTFMR

SEQ ID NO: 52 - DNA, 3336 bp, synthetic construct
  source 1..3336: mol_type = other DNA
atggattacaaggatgacgacgataagggtaccatgaacatccccacccctgtgggcatcgaggtgctgctgcctgaccctaatcctaactgccctctgtgcggcgtccaggtgggcaaggtgggaatgctgggcaaacacttcgccagccgccacgccagagtggccgtgtcttataagtgtcggcgctgtggcaggacaaacagcaactcccattctatcagctgtcacgtgcctaagtgcaagggactcggcctggaagagacaggaagaaaggaaggcgaatttggctgcgagctgtgttcaggacggttcagaaccggcgtggggctgacccaacaccggcggctggctcaccctgtggaatggaacatggaaaaggttggctctacccagcctagaggaggcctgaagggcacaaagctgtggtccgtggacgagctgaaaagcctgatcgagctatccgtgcggttcgctggcgaacccaatcataagatcaacaagctgatcgctgatgaacttggtagcggcaagaccgccgagcaggtcagaagcaagcgccggctgctgcaaaaggagatggccagcaacggcccccccatcgctgccgctgaagagagcggcacctctcgggcccaggccgatccgatcgtgccccagagccctcctaacgtggccaagacacggctgatggaggccatcaacaccgagggcggcaccgagggcgagaacgagcggagagccatcgccgacctgatcagagacgtggaccagaaccccggcctgatcgagtctagcgccctggagctgatccagagactgggaaagggcgccaccagaaatagagccgaaaacggcagagagatcaccagagctaagaaaggatgggaaagacggctgagccagaagcgcagagaattcagagtgcaccagagactctacgagaaggacccaaccaagctggcagcgctggtgctggacggcgatgcctgtgctgagtgcaccatccctgtgacccaaatcgacgaagaatacagacggcgctgggaggccggcggcgagttccacgggctgggcaacttcagatctgccggaggcgccgataatagcgagtttcacaaccccatcctggccaccgaggtgtccgagaacctgatcaagatgaaaaacggctctgcccctggccctgatggcatctccaagaaggccctgcttcaatgggaccctgccggcgaacaactggctagactctacactgcctggctggtgcacggcgtgatcccacgggccttcaaggaatgtagaaccaagctgctgcccaagagcggtgacgcggatgagctgaaggacgtcaacggctggagacctatcaccatcggctccctagtgctgagacttttcagcagaatcctgacaatgcggctgaccagagcctgcacaatgaaccctagacagagaggttttctggcctctagcaatggttgcgccgacaatctgatggtgctgaatggcgtgatcaagcggagccgggaagaacgggctcctctggccgtcgtgttcgtggacttcgccaaggcctttgatagcatctcacacgagcactttctgcgggtgctggagcagcggcagctggatagacatgtgatcgaactgattaagaacagctacgtggactgcgtgacaagagtgggatctggcggcgaacgcaccagccccatcaacatgaaagtgggagtcaaacagggcgacccaatgagccccctgctgttcaacctggctatggaccctttgatccaagccctggaggacggccagagcggcctgtcttgggatggcagatccatcagcaccctggccttcgccgacgacttagtgctgctgagcagcagcgtgcggggcatgagaagaaacctggagatcctggaagaattctgccagctgaccggcctgaaggtgcagcctaagaagtgccacggcttctactacgacaagggcgtgatcaacgactgtgagccttggaccatcggcggcaccagcatccacctggtgggccctggcgagagcgtgaagtacctgggagttcaggtgggccccagaagaggcatcacaatgcctgatctctcgccacagctgcaggagtggatcgtgcgcatcaataaggcaccactgaagcctacgcagagggtgaaagtgctgaatagcttcgccctgcctcgactaatctaccaggctgaccactgcgacgtgcagggcaccgccctggccacactggacggaatggtgagaaaggccgtgaaaacatggctgcacctggcccctagcacttgtaacggcctgctgtacagcagaaatagagacagcggcctgggcatcgtgagactggaaagactgatccctagcctgcaggtcaagagaatccacagactgtcccggagccaggacctgatgacccggttaattaccgtgaaaaccgtcacccagtctgagtggcagcgcaagtggcaaatggtcggcggcgatcctgacaacagtccagtgttggaagtcgaggagggattccccgtggacggcagaagcctggttgtgaccgattggaggcggggagagaacgaggcctggctggccctccaggtgcagggcatgggcgctggtgagtttaagggcgataaaatcagcaacagctggctgttcgaccccactaaggtgggcttcagacagcggcactacatcgccgccctggcccttcggaccaacacctaccccacaggcgagttcctggcacgcggcagagataaggccggcgccgcatgcagacggtgccaggcccggctggagacatgcagccacatactcgggcagtgccccttcgtgaaaggcagcagaatcagacggcaccacagaatctgcgaactgctggccaacgaagccgagagagtgggctggaaggctaccagagagttcagagtgattacacctgagggcggagtacggattccagacctggtgtgcaccaagggcgataaggccctggtgctggacgtgacaattagattcgagatggcccctgacaccctgttgcgcgctgccgccgaaaaggtgaaccactaccagcctgtcgcctctcagattgccagcatggtgggcgctaaagaagtgaaagtcatgggattccctatcggcgccagaggaaagtggccttcttgcaacaacaaagtgctgacagagctgggcgttaccgtgggcagacgggccacctttgccagactggtgagtagacgctccttgctgtatagcttggatatcctgcgggctttcgtgtgcgagcccgccctgtag

SEQ ID NO: 53 - protein, 1111 aa, synthetic construct
MDYKDDDDKGTMNIPTPVGIEVLLPDPNPNCPLCGVQVGKVGMLGKHFASRHARVAVSYKCRRCGRTNSNSHSISCHVPKCKGLGLEETGRKEGEFGCELCSGRFRTGVGLTQHRRLAHPVEWNMEKVGSTQPRGGLKGTKLWSVDELKSLIELSVRFAGEPNHKINKLIADELGSGKTAEQVRSKRRLLQKEMASNGPPIAAAEESGTSRAQADPIVPQSPPNVAKTRLMEAINTEGGTEGENERRAIADLIRDVDQNPGLIESSALELIQRLGKGATRNRAENGREITRAKKGWERRLSQKRREFRVHQRLYEKDPTKLAALVLDGDACAECTIPVTQIDEEYRRRWEAGGEFHGLGNFRSAGGADNSEFHNPILATEVSENLIKMKNGSAPGPDGISKKALLQWDPAGEQLARLYTAWLVHGVIPRAFKECRTKLLPKSGDADELKDVNGWRPITIGSLVLRLFSRILTMRLTRACTMNPRQRGFLASSNGCADNLMVLNGVIKRSREERAPLAVVFVDFAKAFDSISHEHFLRVLEQRQLDRHVIELIKNSYVDCVTRVGSGGERTSPINMKVGVKQGDPMSPLLFNLAMDPLIQALEDGQSGLSWDGRSISTLAFADDLVLLSSSVRGMRRNLEILEEFCQLTGLKVQPKKCHGFYYDKGVINDCEPWTIGGTSIHLVGPGESVKYLGVQVGPRRGITMPDLSPQLQEWIVRINKAPLKPTQRVKVLNSFALPRLIYQADHCDVQGTALATLDGMVRKAVKTWLHLAPSTCNGLLYSRNRDSGLGIVRLERLIPSLQVKRIHRLSRSQDLMTRLITVKTVTQSEWQRKWQMVGGDPDNSPVLEVEEGFPVDGRSLVVTDWRRGENEAWLALQVQGMGAGEFKGDKISNSWLFDPTKVGFRQRHYIAALALRTNTYPTGEFLARGRDKAGAACRRCQARLETCSHILGQCPFVKGSRIRRHHRICELLANEAERVGWKATREFRVITPEGGVRIPDLVCTKGDKALVLDVTIRFEMAPDTLLRAAAEKVNHYQPVASQIASMVGAKEVKVMGFPIGARGKWPSCNNKVLTELGVTVGRRATFARLVSRRSLLYSLDILRAFVCEPAL

SEQ ID NO: 54 - DNA, 3132 bp, synthetic construct
  source 1..3132: mol_type = other DNA
atggattacaaggatgacgacgataagggtacccatatgagtcagaagagaagacccgaaaaagccgtccccgatgaaggagccaccgcccacgatgtcgcccagccagacaagagtaaatgtagcgtgtgcggcgagacattcaagggcccagccagcgtgaccatgcacatggtgaagaagcaccccgtggagtttaatgagctgaagatggccaagaagcccgtgcctaagaaggtgagatggtccgaggaggagatcttccagctggccaggacagaggcagagctgaccctgcagggcgtgcgcttcatcaacgtggagctgcagaagatctttcctgccagagagatcgagggcatcaagggccagaggaagctggccaagtacaaggagctggtgaaggaccagctggatgagatcggcagggccccaaatccccctgagcaggagatcggcgaggacgtgccaagccccttcaaggcatggctggagctgctgctggccctgccaaagacccccaatgattttctggagcacaagctggacaacatcatcgtgcaggccctgaaggaggacgtgaactctgatcaggtgttcaacgatctgaatagctacctgaagctgatcctggagccatctggaagggcaaagagcgtgcccggagagatcatccacggcgacccttctggcagcgccaagacctccgtgacaaaggcccctaagccagccacagtgagctcctctcggaagaagcggagagatgccgagtttgccagaatccagcggctgtataggaagaaccgcacatcttgcatcaataccatcctggacggcaacaccagagagcacgaggcccccaagaatatggagggcttctggcgcgagatctttgagcgggagagccctgacgatccagacgatcccgatatcttcctggaggaggaggcctccgacatctggaagtacatctctttttatgagatgtgcaacctgtatccaccaccttccacagcaccaggacctgacggcttcagctccaaggatctgaggcgcatgacccccagggtgctgaacaagatcctgaatctgctgctgcacctgcgcgatctgcctcagatcctgaagagccaccggacagtgctgatcccaaagacagatctgcctaccaagccaggcgacttccgccctatcaccatcagcaacatcctggtgcgccacctgaacaagatcctggccaatcgggtgtcccacctgatccccatcaatgagcggcagaaggcctttctgcctatcgatggctgcgccgagaacatcttcacactggactttatcctgcaccacgccagaaccaagatcaagtccctgtctatggccatcctggacatcagcaaggccttcgatagcgtgtcccaccactccatctttcgggccctgagagaggccaggtgcccaatcggcttcatcaagtttatcgagaattgctacggcggctgtttcaccaagctgttttgtggcggcgtgaagtatccctctgaggtgagcatgaataggggcgtgaagcagggcgatcccctgtctcccgtgctgttcaacctggtcatcgacggcctgatccgccagatcccaagcgccctgggctttaacgtgtctgaccaggtgaaggtgagctgcatcgcctacgccgacgatctgatcctgatcgccaccacacgggccggcctgaagacactgctggatctgaccaatagctatctggccaagagaggcctgtccctgaaccccgacaagtgttccgccctgtctatcgtggccagcggcaagcagaagctggtgtacatcgcctctagcgagcacttcgatctggccggccagaagatgagaaacctgaatgtgggcgactcctggaggtatctgggcatccagttttctcacctgggaagggcagagaaggtgacaccagatctgacctgtctgatcaatagactgcagaaggcccctctgaagctgcagcagaagctgtacgccctgagaatctatctgatccccaggctgatccacggcctgacactgtccaagaccaacctgggcgagctgaagaccctggacaagctgatccgcaagtacatccgggcctggctgcacctgccagacgatacacccatgggctacttctatacccctctgaaggcaggaggcctgggcctgccaagcctgcggctggtcatcctgaacaatcgcctggagagaatcctgagaatgaaggcctcccaggatatcatcgtgaggacaatcgccgagtctgagacactgggcgtggagatccgcaagctgcacgacctgctgtccatcgatggcacaatcctggacacctccgtgaagatccactctttttgggccgagagactgtactcctcttatgacggcaagtgcctgtgcaactctgccaatttcccacccggcaataagtggatcggcgaggatagcctgaaccagcggtcccacatctttgccgactgcctgaagctgagaatcaacgccctgcctacacggtccagaaccgcaaggggccgccctctgaaggataagccatgcagagccggctgtaggaattctgacggcgtgaaagtgatcgagacactgaaccacatcacccaggtgtgcgagaggacacacggagcaagggtgaagaggcacgacaggctggtggatttcgcagtgaagggcctgcagcggccccacagggtggtgctgaaggagccccactacaagaccgtgaatggcgtgaggaagcccgacatcgtgatcaagatccctgaccacacatatatctgcgatttccaggtggtgagcgacacctcctgtctggagctggagtttcgcaagaaggccctgaagtacgccgaggataagggcctgtgcgaccagctgaccagggatcacccaggagagctgagcttcaccgccatcaccttcaacacccgcggcctgatcgccaagagctccgtgacagccctgaggaagctgggaatgcctccacggtccatcatgaccctgcagaagatctgtatggaggggtcactggaaatctggagaatcttcaatcagactactgctatggcacgcaactag

SEQ ID NO: 55 - protein, 1043 aa, synthetic construct
MDYKDDDDKGTHMSQKRRPEKAVPDEGATAHDVAQPDKSKCSVCGETFKGPASVTMHMVKKHPVEFNELKMAKKPVPKKVRWSEEEIFQLARTEAELTLQGVRFINVELQKIFPAREIEGIKGQRKLAKYKELVKDQLDEIGRAPNPPEQEIGEDVPSPFKAWLELLLALPKTPNDFLEHKLDNIIVQALKEDVNSDQVFNDLNSYLKLILEPSGRAKSVPGEIIHGDPSGSAKTSVTKAPKPATVSSSRKKRRDAEFARIQRLYRKNRTSCINTILDGNTREHEAPKNMEGFWREIFERESPDDPDDPDIFLEEEASDIWKYISFYEMCNLYPPPSTAPGPDGFSSKDLRRMTPRVLNKILNLLLHLRDLPQILKSHRTVLIPKTDLPTKPGDFRPITISNILVRHLNKILANRVSHLIPINERQKAFLPIDGCAENIFTLDFILHHARTKIKSLSMAILDISKAFDSVSHHSIFRALREARCPIGFIKFIENCYGGCFTKLFCGGVKYPSEVSMNRGVKQGDPLSPVLFNLVIDGLIRQIPSALGFNVSDQVKVSCIAYADDLILIATTRAGLKTLLDLTNSYLAKRGLSLNPDKCSALSIVASGKQKLVYIASSEHFDLAGQKMRNLNVGDSWRYLGIQFSHLGRAEKVTPDLTCLINRLQKAPLKLQQKLYALRIYLIPRLIHGLTLSKTNLGELKTLDKLIRKYIRAWLHLPDDTPMGYFYTPLKAGGLGLPSLRLVILNNRLERILRMKASQDIIVRTIAESETLGVEIRKLHDLLSIDGTILDTSVKIHSFWAERLYSSYDGKCLCNSANFPPGNKWIGEDSLNQRSHIFADCLKLRINALPTRSRTARGRPLKDKPCRAGCRNSDGVKVIETLNHITQVCERTHGARVKRHDRLVDFAVKGLQRPHRVVLKEPHYKTVNGVRKPDIVIKIPDHTYICDFQVVSDTSCLELEFRKKALKYAEDKGLCDQLTRDHPGELSFTAITFNTRGLIAKSSVTALRKLGMPPRSIMTLQKICMEGSLEIWRIFNQTTAMARN

SEQ ID NO: 56 - DNA, 3096 bp, synthetic construct
  source 1..3096: mol_type = other DNA
atggattacaaggatgacgacgataagggtaccatgagcggcaagagctccaagcctcgcaccgtgtctagcggctcctctagccaggagacacccccttccggctctaatgcctgcgacatctgtggcaagtgcttcatgaagcccgtgggcctgtctctgcacatgagcaaggtgcaccctacccagtaccacgcccgcctggagaagaaccagccaaaggccaagaagttccggtggacagacgaggatctgtattttctggccaagaaggaggcagagctgctgctgctgggaggcatcaagtttatgaataaggagctggccgagttctttcccgagaagtctgtggatcagatcaagggccagcggagaagcgagacctacaagcagcaggtggtgagcatccactccgagctgctgaagctgcaggcagtggcagactccccaccaccttctcgcatccccgcaaaggaggtgtccgcatggctggatttcctgctggccctgcctaagaccaagaataagttttctgaggacaagctggatcagctgatcagaaccgcacaggagggaacaccagtgctgaacgacctggatctgtatctgagggaggtgctggtgcagccaaccagacagggagagaggcaggcaaagccactgccaccccctaagtcctctcgggagaagagggaccgcgagtacgccagagtgcagaatttctataggaagaacaagaccgcctgtgtgaacgccatcctggatggcaataagaagtgcgagaacaagatccctgacatcgatgagttctggaaggccatctttgagagccagtccccaccagacgcagagcccgtgagctacgtggtggatgaggagcctaagaatatctggtcttggatcagcttctttgagatgaacagaaattatcccgacacctctacaagcccaggacctgatggagtgaccgcaaggatgctgagatccatccctgccagggtgctgaacaagctgctgaatctgctgctgttcatcgaggacctgcccgccgtgtttaagtgtcaccgcaccgtgctgatccctaaggtggataacccagcactgccaggagagttccggccaatcacaatcagctccatcatcgtgagacagctgaataagatcatcgcagcaagggtgagcgagggagtgcctatcaacccacgccagaaggcctttcggcagatcgacggctgcgccgagaacgtgttcctgctggactttatcctgagggatgccaagaccaagatcaagagcctgtctctggccacagtggacatcaagaaggccttcgatagcgtgtcccaccacagcatctttagggcaatcaggggagcaaggtgtccagagaacctggtgaattacatccagaattcttatagcggctgcaccacacagatctccgtgggcggctccatctctaccacaaagatcctgatgaacagaggcgtgaagcagggcgatcccctgagccctgtgctgttcaacctggtcatcaacgagatcatcaggaagctgcctgcctccatcggctacccaatcaattccgagctgtctatcaactgcatcgcctatgccgacgatctgatcctggtggccaataccagagagggcctgaagctgctgctgaacctgctgaatgaggagctgcccaagaggggcctggagctgaacgcctctaagtgttttggcctgagcctgacagccctgggcaagctgaagaagacccacctgtgcacaagcgaccagctggatctgcacggcaccctgatcaagaatctgacagccgaggagagctgggtgtacctgggcgtgccattctcccacatcggccgcagcaagtccttttctcccgacctggaggccctgctgaacaagctgcagaagtccccactgaagctgcagcagaagctgttcgccctgcgcgtgtatctgatcccaaggctgctgcacggactggtgctgtctagggtggcaatcggcgagctgaagatcatggacaagctgatcctgaagcacctgagggtgtggctgaggctgccaaaggatacccccctgggcttcttttactccccagtgaagctgggcggcctgggcatcaagaacctgcgcacaaatgtgctgaagtgtagaaagcagaggatcgagcgcatgctggtgtccccagacgatgtggtgcggctggtggcagagtctgagatctttctgaaggagaccgacaagctgaaggatctgctgacaatcaatggcatgtgcctggacaccagaaacgtgcctaggacaggcaagaacaataagttctggtccgagagactgtacacctcttttgacggcaagacactggcctacagcgagtatttcacccagggcggcggctggatcagagaggataagatcctgcagccagcccacgtgtttgccgagtgtatcaagctgcggatcaacgccctgcctaccaagagcagagtggcccacggcaggccaacaaaggaccggtcctgtagagccggctgcctggatgtgcagaaggtgcctgccatcgagaccatcaatcacatcgcccaggtgtgcccaagaacacacggagcaaggatcaagaggcacgacaggctggtgcagttcctgtccctgaacctgcgcaagaatcctaagcggaacgtgctggtggagtacaattttagaaccgtggccggcatcaggaagccagacatcatcgtgatcgaggatacaagggcagcaatcctggacgtgcaggtggtgggcgattctagcaacctggagatggagtacctggagaagagcagaaagtattccaatgatgccaccctgtctatgaggatcaacgccctgcagaagctgtatcccaccgtgacaagcctgaccttccacgccgtgaccttcaacaataggggcctgatcgccaagtctacagtggcagcactgaggatgctgggagtgcctccacggtgtatcatgatcctgtgcgtgatcagcctggagaagaccctggaagtgtggcgcatgttcaaccagagcacagcctccgcccggaagtag

SEQ ID NO: 57 - protein, 1031 aa, synthetic construct
MDYKDDDDKGTMSGKSSKPRTVSSGSSSQETPPSGSNACDICGKCFMKPVGLSLHMSKVHPTQYHARLEKNQPKAKKFRWTDEDLYFLAKKEAELLLLGGIKFMNKELAEFFPEKSVDQIKGQRRSETYKQQVVSIHSELLKLQAVADSPPPSRIPAKEVSAWLDFLLALPKTKNKFSEDKLDQLIRTAQEGTPVLNDLDLYLREVLVQPTRQGERQAKPLPPPKSSREKRDREYARVQNFYRKNKTACVNAILDGNKKCENKIPDIDEFWKAIFESQSPPDAEPVSYVVDEEPKNIWSWISFFEMNRNYPDTSTSPGPDGVTARMLRSIPARVLNKLLNLLLFIEDLPAVFKCHRTVLIPKVDNPALPGEFRPITISSIIVRQLNKIIAARVSEGVPINPRQKAFRQIDGCAENVFLLDFILRDAKTKIKSLSLATVDIKKAFDSVSHHSIFRAIRGARCPENLVNYIQNSYSGCTTQISVGGSISTTKILMNRGVKQGDPLSPVLFNLVINEIIRKLPASIGYPINSELSINCIAYADDLILVANTREGLKLLLNLLNEELPKRGLELNASKCFGLSLTALGKLKKTHLCTSDQLDLHGTLIKNLTAEESWVYLGVPFSHIGRSKSFSPDLEALLNKLQKSPLKLQQKLFALRVYLIPRLLHGLVLSRVAIGELKIMDKLILKHLRVWLRLPKDTPLGFFYSPVKLGGLGIKNLRTNVLKCRKQRIERMLVSPDDVVRLVAESEIFLKETDKLKDLLTINGMCLDTRNVPRTGKNNKFWSERLYTSFDGKTLAYSEYFTQGGGWIREDKILQPAHVFAECIKLRINALPTKSRVAHGRPTKDRSCRAGCLDVQKVPAIETINHIAQVCPRTHGARIKRHDRLVQFLSLNLRKNPKRNVLVEYNFRTVAGIRKPDIIVIEDTRAAILDVQVVGDSSNLEMEYLEKSRKYSNDATLSMRINALQKLYPTVTSLTFHAVTFNNRGLIAKSTVAALRMLGVPPRCIMILCVISLEKTLEVWRMFNQSTASARK

SEQ ID NO: 58 - RNA, 54 nt, synthetic construct
  source 1..54: mol_type = other RNA
aggaataaactagtattcttctggtccccacagactcagagagaacccgccacc

SEQ ID NO: 59 - RNA, 412 nt, synthetic construct
  source 1..412: mol_type = other RNA
tgatgacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 60 - RNA, 361 nt, synthetic construct
  source 1..361: mol_type = other RNA
ggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggt

SEQ ID NO: 61 - RNA, 291 nt, synthetic construct
  source 1..291: mol_type = other RNA
ggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaa

SEQ ID NO: 62 - RNA, 403 nt, synthetic construct
  source 1..403: mol_type = other RNA
gactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttg

SEQ ID NO: 63 - RNA, 403 nt, synthetic construct
  source 1..403: mol_type = other RNA
gactctcttaagggaagaccccgcccatgaggcttggagagtgtgatcctgatcagatcacacttgaaaagttatgctgagtacgtccgcgtcgtgagagtcggtaactgtcccaggatggtctgggataggttaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttg

SEQ ID NO: 64 - RNA, 145 nt, synthetic construct
  source 1..145: mol_type = other RNA
gactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagca

SEQ ID NO: 65 - RNA, 83 nt, synthetic construct
  source 1..83: mol_type = other RNA
gactctcttaaggtgctgagtacgtccgcgtcgtgagagtcggtaactgtcccaggatggtctgggataggctaaactcagca

SEQ ID NO: 66 - RNA, 201 nt, synthetic construct
  source 1..201: mol_type = other RNA
ggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatt

SEQ ID NO: 67 - RNA, 108 nt, synthetic construct
  source 1..108: mol_type = other RNA
ggcgggagtaactatgactctcttaaggagtcatagagccagaacctcctcgtggtcccgctgggcacagggattaatttttctgtggcaaatttgactggcttcaga

SEQ ID NO: 68 - RNA, 748 nt, synthetic construct
  source 1..748: mol_type = other RNA
ggcgggagtaactatgactctcttaaggcgacttgagaaggtctggttacaactgggcatagctgcagagatcgcgcctcctcgtggccccgctggtaagcccttaacagggtgactaagtcgatctctgccccagtccaggagccgctgggtttcaccagcccagcgattccttccaaattcggtgaaacaaattcctcggtaaaagccgcgtggcttattgcctgaaacctggccccccggtttcagacaggggcaaagagttcggaagtggactgaccacccaccccgaacccgagagcgaatctggtcatgacccaactgtcccaaatcctggtccgtctcttggagcgggggaaggtgcacagccactacccttactcagggtatcggtgggcacccaaacctgtgaagaggactttataacatctagaccaaccaaattacccggaattgaatcagaattaggcccgctggtgaagttttctttagaggtttacaggtcagatcttaagggggatgtgcaatttgaggggattcattttccagataattggggggtactggaggggtttcctgaggtgtacgaacaactggcaccacagccaaacgggggagacgagttaaatcatagtctcccaggggacagggagggggatgtacttgagaaggatagcagcgaaaaggagaaggaggctgcaccagaggcattgccctcagtgcaaagggcccgcagtgaacagttgcc

SEQ ID NO: 69 - RNA, 748 nt, synthetic construct
  source 1..748: mol_type = other RNA
ggcgggagtaactatgactctcttaaggctggggaccgtggttacaacccgggcttagctgcagagacagtacctccccgtggttcccgccggaccccgtaacatcgggtgactgaatctgtctctgccccgggagtagttcctccttgccctattgaccagcggtcgccggctgctcaatagtattctaggcgtgaaatatagcgatagtcctagtggttgtcttactgggccatagccccttgcttcaggggtcattcgcgaagtctctcaggagaactgggggtggtgttcttctgggtatagctaaaccccctagactgtgtccgatccatggggtcctggatcgtgaatttcgtttcggtggcgactcagacgggagaattccctgtggatacggccaggagggcacctgtgccggtaacatcataccctgagtcggaatgccacntaccgttgcccctgacattttgtaactcggatgtgactatttggggaggggttcgccctgaaccggtggactgcttgggwgatcttccrgaggygtatgatgcactcccaggggtggctgggcctcgggaaycggtgggtgggagcccgccgggrgaaggggtcaggtcgccagggattgcgtcrccctctggtactgcggtccaacatgattttgggagtcccatcctcgtaccgggagccgaagccgccgaggtntctaccccggtagtgaaggttccncaagaccatccagcat

SEQ ID NO: 70 - RNA, 284 nt, synthetic construct
  source 1..284: mol_type = other RNA
ggggatctggggtaattgcgagcagagggggagtatttttctgtaattcgtaagtcatatcatatggtgtgcggaaggggaattttactctgtaactcacaagtctctcctttactcaagtcgactcaaaacctcctcgtggtggtccccggtaatgctaaacttgtttagcagctaatttgagcggcaaaaacttttccgatgggctggttacccagaggaaatttactcatattggaactacgaacacaaataacgagcctcggatatctttacacaatctg

SEQ ID NO: 71 - RNA, 171 nt, synthetic construct
  source 1..171: mol_type = other RNA
ggtctggcatttgatcgtttccgcctcctcgtggcgccagactgggtaagctgatttatcagtgagctaagagaaacgatcaccgcaggagtccatctacctgcgctgcacgtgatttcattgtgcacttggcgagttatcccttgtggagctcgctcgttagcttttgag

SEQ ID NO: 72 - RNA, 188 nt, synthetic construct
  source 1..188: mol_type = other RNA
ggggtagcaattgatcgattcccgcctcctcgtggcgctaccctgggataacctcaaagaaatttgacggtaaagctaagaggaattgatcacaccgtgacgaatatcatcagtcacagctgcacgaatccagatagatatataacaggcgagtaattcttttcgaagctcgtcagtaatcttcccag

SEQ ID NO: 73 - RNA, 258 nt, synthetic construct
  source 1..258: mol_type = other RNA
ggcgggagtaactatgactctcttaaggtagtccaaaagccttgtcatcttaggatgacgcgcaggtttttgagatgtatatttttatagtagtagtaaaatacgacttgttaaattcttaagataaagagtcatagtagcaagtgaagaccttttcgtggttcctgtcataagtaccctacggggaaaccaacttcacccctgcgaacatggagcttagaggttaccagcctcctcgctcgtcaattagcgaagagc

SEQ ID NO: 74 - RNA, 570 nt, synthetic construct
  source 1..570: mol_type = other RNA
ggtaactatgactctcttaacgacggtgaaccaccttgtcgcggtgtaagagctttagtgtctcgaacaagaaatagcttgtgtgctgtccttctgggcggtgcacatacttcttaacctcccgaggccatgccggcgggggctttagcccccggcaggttttaccatgccggacgggttcgagaggtagaggccaaactaagagttcaccagcagacttcgcacgcggctggccactggccgaagtttaaacaacagggccgcatcttcccaaactcaatatatggtgttaagtgaaccgtgccgatgggtgaatggccatgggtaagttggtctctaacggtccttgtggagaagtggcgacccttcacaatacttcagccttaccctatgcccgggcaacttagggtggacgtatatctcccgcggaaaacatcttatctcatggataaaaatatatacgaaaacactacttctccaggagggggaccgctttgcggtgagaagacgcatcgttccgatgtgatcatccctccaccggggttcgccccgtcgacggacactgcttcc

SEQ ID NO: 75 - RNA, 235 nt, synthetic construct
  source 1..235: mol_type = other RNA
ggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgaccttggtcggcgaaacagccc

SEQ ID NO: 76 - RNA, 81 nt, synthetic construct
  source 1..81: mol_type = other RNA
ggcgggagtaactatgactctcttaagggctgtacctccacgtggtcccgctggtaacgaccttggtcggcgaaacagccc

SEQ ID NO: 77 - RNA, 370 nt, synthetic construct
  source 1..370: mol_type = other RNA
ggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgaccaggaacaccggtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggt

SEQ ID NO: 78 - RNA, 304 nt, synthetic construct
  source 1..304: mol_type = other RNA
ggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacacgacataggagtcggtcggctaaatcagcccgcccaccatctgggatatggttgaccgtctaaccccagtactcaggtcacaaacaaa

SEQ ID NO: 79 - RNA, 370 nt, synthetic construct
  source 1..370: mol_type = other RNA
ggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgaccaggaacaccggtcggcaaaatcagcccgcccaccatctgggatatggttgaccgtctaaccccagtactcaggtcacaaacaaaatgggaacagatacagtgtatgtcggccaggactacccttctggcttatcaaaacgggtaccagcacggt

SEQ ID NO: 80 - RNA, 369 nt, synthetic construct
  source 1..369: mol_type = other RNA
ggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgaccaggaacaccggtcggctaaacagcccgcccaccatctgggatatggttgaccgtctaaccccagtactcaggtcacaaacaaaatgggaacagatacagtgtatgtcggccaggactacccttctggcttatcaaaacgggtaccagcacggt

SEQ ID NO: 81 - RNA, 372 nt, synthetic construct
  source 1..372: mol_type = other RNA
ggcgggagtaactatgactctcttaaggcgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgaccaggaacaccggtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggt

SEQ ID NO: 82 - RNA, 371 nt, synthetic construct
  source 1..371: mol_type = other RNA
ggcgggagtaactatgactctcttaagcgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgaccaggaacaccggtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggt

SEQ ID NO: 83 - RNA, 369 nt, synthetic construct
  source 1..369: mol_type = other RNA
ggcgggagtaactatgactctcttacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgaccaggaacaccggtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggt

SEQ ID NO: 84 - RNA, 341 nt, synthetic construct
  source 1..341: mol_type = other RNA
gactctcttaaggtgctgagtacgtccgcgtcgtgagagtcggtaactgtcccaggatggtctgggataggctaaactcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttg

SEQ ID NO: 85 - RNA, 77 nt, synthetic construct
  source 1..77: mol_type = other RNA
gactctcttaagggctgagtacctccgcgtggtgagagtcggtaacagccattgcactccggctggcgaaactcagc

SEQ ID NO: 86 - RNA, 198 nt, synthetic construct
  source 1..198: mol_type = other RNA
ggcgggagtaactatgactctcttaaggagtcatagagccagaacctcctcgtggtcccgctgggcacagaggaacaccctgtggcaaatttgactggcttcagagagcgtttttcgaagtggactgtgtgactgcgttccccccttagttgctatatccgcttcgattaacatctcacctcgacgtataagatcatt

SEQ ID NO: 87 - RNA, 201 nt, synthetic construct
  source 1..201: mol_type = other RNA
ggcgggagtaactatgactctcttaaggagtcatagagccagaacctcctcgtggtcccgctgggcacagggattaatttttctgtggtaaatttgactggcttcagagagcgtttttcgaagtggactgtgtgactgcgttccccccttagttgctatatccgcttcgattaacatctcacctcgacgtataagatcatt

SEQ ID NO: 88 - RNA, 99 nt, synthetic construct
  source 1..99: mol_type = other RNA
ggcgggagtaactatgactctcttaaggagtcatagagccagaacctcctcgtggtcccgctggacacagaggaacaccctgtggcaaatttgactggc

SEQ ID NO: 89 - RNA, 99 nt, synthetic construct
  source 1..99: mol_type = other RNA
ggcgggagtaactatgactctcttaaggagtcatagagccagaacctcctcgtggtcccgccggacacagaggaacaccctgtggcaaatttgactggc

SEQ ID NO: 90 - RNA, 97 nt, synthetic construct
  source 1..97: mol_type = other RNA
ggcgggagtaactatgactctcttaaggagtcatagagcagaacctcctcgtggtcccgctggacacagaggaacaccctgtggcaaatttgctggc

SEQ ID NO: 91 - RNA, 98 nt, synthetic construct
  source 1..98: mol_type = other RNA
ggcgggagtaactatgactctcttaagagtcatagagccagaacctcctcgtggtcccgctggacacagaggaacaccctgtggcaaatttgactggc

SEQ ID NO: 92 - RNA, 97 nt, synthetic construct
  source 1..97: mol_type = other RNA
ggcgggagtaactatgactctcttaaagtcatagagccagaacctcctcgtggtcccgctggacacagaggaacaccctgtggcaaatttgactggc

SEQ ID NO: 93 - RNA, 95 nt, synthetic construct
  source 1..95: mol_type = other RNA
ggcgggagtaactatgactctcttagtcatagagccagaacctcctcgtggtcccgctggacacagaggaacaccctgtggcaaatttgactggc

SEQ ID NO: 94 - RNA, 97 nt, synthetic construct
  source 1..97: mol_type = other RNA
ggcgggagtaactatgactctcttaaggagtcatagagcagaacccccccacggtcccgccggacacagaggaacaccctgtggcaaatttgctggc

SEQ ID NO: 95 - RNA, 100 nt, synthetic construct
  source 1..100: mol_type = other RNA
ggcgggagtaactatgactctcttaaggagtcatagagcagaacccccccacggtcccgccgggcaacacagaggaacaccctgtggcaaatttgctggc

SEQ ID NO: 96 - RNA, 100 nt, synthetic construct
  source 1..100: mol_type = other RNA
ggcgggagtaactatgactctcttaaggagtcatagagcagagcccccccacggccccgccgggcaacacagaggaacaccctgtggcaaatttgctggc

SEQ ID NO: 97 - RNA, 100 nt, synthetic construct; identical to SEQ ID NO: 96
  source 1..100: mol_type = other RNA

SEQ ID NO: 98 - RNA, 109 nt, synthetic construct
  source 1..109: mol_type = other RNA
ggcgggagtaactatgactctcttaaggagtcatagagccagaacctcctcgtggtcccgctgggcacagggattaatttttctgtggcaaatttgactggcttctaga

SEQ ID NO: 99 - RNA, 137 nt, synthetic construct
  source 1..137: mol_type = other RNA
gggtaaacggcgggagtaactatgactctcttaaggagtcatagagccagaacctcctcgtggtcccgctgggcacagggattaatttttctgtggcaaatttgactggcttctagaaggtcgaccagatgtccgag

SEQ ID NO: 100 - DNA, 115 bp, synthetic construct
  source 1..115: mol_type = other DNA
gggtaaacggcgggagtaactatgactctcttaaagtcatagagccagaacctcctcgtggtcccgctgggcacagggattaatttttctgtggcaaatttgactggcttctaga

SEQ ID NO: 101 - DNA, 90 bp, synthetic construct
  source 1..90: mol_type = other DNA
ggcgggagtaactatgactctcttaaggtcccagcctcctcgtggctcccgccggtaacagccattgcactccggctggcgaatgggacc

SEQ ID NO: 102 - DNA, 77 bp, synthetic construct
  source 1..77: mol_type = other DNA
gactctcttaaggggtcccagcctcctcgtggcgagagtcggtaacagccattgcactccggctggcgaatgggacc

SEQ ID NO: 103 - DNA, 76 bp, synthetic construct
  source 1..76: mol_type = other DNA
gactctcttaaggggtcccagccccctcacggcgagagtcggtaacacagaggaacaccctgtggcgaatgggacc

SEQ ID NO: 104 - DNA, 85 bp, synthetic construct
  source 1..85: mol_type = other DNA
gtaactatgactctcttaaggggtcccagcctcctcgtggctagttacggtaacagccattgcactccggctggcgaatgggacc

SEQ ID NO: 105 - DNA, 73 bp, synthetic construct
  source 1..73: mol_type = other DNA
ggccggcatggtcccagcctcctcgtggcgccggccggtaacagccattgcactccggctggcgaatgggacc

SEQ ID NO: 106 - DNA, 113 bp, synthetic construct
  source 1..113: mol_type = other DNA
ggcgggagtaactatgactctcttaaaaaagagaatcatagaacgtcagcagcctcctcgcggccccgccggtaagattccgaaaggaatcgcgaatgctgacgaaagtcttc

SEQ ID NO: 107 - DNA, 113 bp, synthetic construct
  source 1..113: mol_type = other DNA
ggcgggagtaactatgactctcttaaaaaagagaatcatagaacgtcagcagccccctcacggccccgccggtaagattccgaaaggaatcgcgaatgctgacgaaagtcttc

SEQ ID NO: 108 - DNA, 116 bp, synthetic construct
  source 1..116: mol_type = other DNA
ggcgggagtaactatgactctcttaaaaaagagaatcatagaacgtcagcagccccctcacggccccgccggtaacacagaggaacaccctgtggcgaatgctgacgaaagtcttc

SEQ ID NO: 109 - DNA, 83 bp, synthetic construct
  source 1..83: mol_type = other DNA
gactctcttaaggcgtcagcagcctcctcgcggccccgccggtaagattccgaaaggaatcgcgaatgctgacgaaagtcttc

SEQ ID NO: 110 - DNA, 83 bp, synthetic construct
  source 1..83: mol_type = other DNA
gactctcttaaggcgtcagcagccccctcacggccccgccggtaagattccgaaaggaatcgcgaatgctgacgaaagtcttc

SEQ ID NO: 111 - DNA, 86 bp, synthetic construct
  source 1..86: mol_type = other DNA
gactctcttaaggcgtcagcagccccctcacggccccgccggtaagacagaggaacaccctgtcgcgaatgctgacgaaagtcttc

SEQ ID NO: 112 - DNA, 108 bp, synthetic construct
  source 1..108: mol_type = other DNA
ggcgggagtaactatgactctcttaaggaagagaatcatagaacgtcagcagccccctcacggccccgccggtaacacagaggaacaccctgtggcgaatgctgacga

SEQ ID NO: 113 - DNA, 108 bp, synthetic construct
  source 1..108: mol_type = other DNA
ggcgggagtaactatgactctcttaagaaagagaatcatagaacgtcagcagccccctcacggccccgccggtaacacagaggaacaccctgtggcgaatgctgacga

SEQ ID NO: 114 - DNA, 108 bp, synthetic construct
  source 1..108: mol_type = other DNA
ggcgggagtaactatgactctcttacaaaagagaatcatagaacgtcagcagccccctcacggccccgccggtaacacagaggaacaccctgtggcgaatgctgacga

SEQ ID NO: 115 - DNA, 78 bp, synthetic construct
  source 1..78: mol_type = other DNA
gactctcttaaggacgcagcagccccctcacggcgagagtcggtaacacagaggaacaccctgtggcgaatgctgcga

SEQ ID NO: 116 - DNA, 78 bp, synthetic construct
  source 1..78: mol_type = other DNA
gactctcttaaggacgcagcagccccctcacggcgagagttggtaacacagaggaacaccctgtggcgaatgctgcga

SEQ ID NO: 117 - DNA, 77 bp, synthetic construct
  source 1..77: mol_type = other DNA
gactctcttaaaacgcagcagccccctcacggcgagagtcggtaacacagaggaacaccctgtggcgaatgctgcga

SEQ ID NO: 118 - DNA, 77 bp, synthetic construct
  source 1..77: mol_type = other DNA
gactctcttaaaacgcagcagccccctcacggcgagagttggtaacacagaggaacaccctgtggcgaatgctgcga

SEQ ID NO: 119 - DNA, 78 bp, synthetic construct
  source 1..78: mol_type = other DNA
gactctcttaaggacgacgcggcccccacacggcgagagtcggcaacacagaggaacaccctgtggcgaacgcgtcga

SEQ ID NO: 120 - DNA, 110 bp, synthetic construct
  source 1..110: mol_type = other DNA
ggcgggagtaactatgactctcttaaggaaaagagaatcatagaacgtcagcagcctcctcgcggccccgccggtaacacagaggaacaccctgtggcgaatgctgacga

SEQ ID NO: 121 - DNA, 114 bp, synthetic construct
  source 1..114: mol_type = other DNA
ggcgggagtaactatgactctcttaaaaaagagaatcatagaacgtcagcagcctcctcgcggccccgccggtaacacagaggaacaccctgtggcgaatgctgacgatctaga

SEQ ID NO: 122 - DNA, 112 bp, synthetic construct
  source 1..112: mol_type = other DNA
ggcgggagtaactatgactctcaacaaagagaatcatagaacgtcagcagcctcctcgcggccccgccggtaacacagaggaacaccctgtggcgaatgctgacgatctaga

SEQ ID NO: 123 - DNA, 102 bp, synthetic construct
  source 1..102: mol_type = other DNA
ggcgggagtaactatgacaacagtcatagaacgtcagcagcctcctcgcggccccgccggtaacacagaggaacaccctgtggcgaatgctgacgatctaga

SEQ ID NO: 124 - DNA, 98 bp, synthetic construct
  source 1..98: mol_type = other DNA
ggcgggagtaactaaacatagttagaacgtcagcagcctcctcgcggccccgccggtaacacagaggaacaccctgtggcgaatgctgacgatctaga

SEQ ID NO: 125 - DNA, 101 bp, synthetic construct
  source 1..101: mol_type = other DNA
ggcgggagtaactatgactctcttaaggaaaagagaatcatagaacgtcagcagcctcctcgcggccccgccggtaacacagaggaacaccctgtggcgaa

SEQ ID NO: 126 - DNA, 110 bp, synthetic construct
  source 1..110: mol_type = other DNA
ggcgggagtaactatgactctcttaaggaaaagagaatcatagaacgtcagcagcctcctcgcggccccgccggtaacacagaggaacaccctgtggagaatgctgacga

SEQ ID NO: 127 - DNA, 55 bp, synthetic construct
  source 1..55: mol_type = other DNA
ggcgggagtaactatgactctcttaacaaagagagaatagtaactcccgtctaga

SEQ ID NO: 128 - DNA, 116 bp, synthetic construct
  source 1..116: mol_type = other DNA
ggcgggagtaactatgactctcttaaggaaaagagaatcatagaacgtcagcagcctcctcgcggccccgccggtaacacagaggaacaccctgtggagaatgctgacgatctaga

SEQ ID NO: 129 - DNA, 124 bp, synthetic construct
  source 1..124: mol_type = other DNA
gggtaaacggcgggagtaactatgactctcttaaggaaaagagaatcatagaacgtcagcagcctcctcgcggccccgccggtaacacagaggaacaccctgtggcgaatgctgacgatctaga

SEQ ID NO: 130 - DNA, 124 bp, synthetic construct
  source 1..124: mol_type = other DNA
gggtaaacggcgggagtaactatgactctcttaagaaaagagaatcatagaacgtcagcagcctcctcgcggccccgccggtaacacagaggaacaccctgtggcgaatgctgacgattctaga

SEQ ID NO: 131 - DNA, 123 bp, synthetic construct
  source 1..123: mol_type = other DNA
gggtaaacggcgggagtaactatgactctcttagcaaagagaatcatagaacgtcagcagcctcctcgcggccccgccggtaacacagaggaacaccctgtggcgaatgctgacgattctaga

SEQ ID NO: 132 - DNA, 123 bp, synthetic construct
  source 1..123: mol_type = other DNA
gggtaaacggcgggagtaactatgactctcttgaaaaagagaatcatagaacgtcagcagcctcctcgcggccccgccggtaacacagaggaacaccctgtggcgaatgctgacgattctaga

SEQ ID NO: 133 - DNA, 121 bp, synthetic construct
  source 1..121: mol_type = other DNA
gggtaaacggcgggagtaactatgactctctgaaaagagaatcatagaacgtcagcagcctcctcgcggccccgccggtaacacagaggaacaccctgtggcgaatgctgacgattctaga

SEQ ID NO: 134 - DNA, 125 bp, synthetic construct
  source 1..125: mol_type = other DNA
gggtaaacggcgggagtaactatgactctcttaaggtaaagagaatcatagaacgtcagcagcctcctcgcggccccgccggtaacacagaggaacaccctgtggcgaatgctgacgattctaga

SEQ ID NO: 135 - DNA, 128 bp, synthetic construct
  source 1..128: mol_type = other DNA
gggtaaacggcgggagtaactatgactctcttaaggtagctaagagaatcatagaacgtcagcagcctcctcgcggccccgccggtaacacagaggaacaccctgtggcgaatgctgacgattctaga

SEQ ID NO: 136 - DNA, 124 bp, synthetic construct; identical to SEQ ID NO: 129
  source 1..124: mol_type = other DNA

SEQ ID NO: 137 - DNA, 135 bp, synthetic construct
  source 1..135: mol_type = other DNA
gggtaaacggcgggagtaactatgactctcttaaggaaaagagaatcatagaaactcccgaacgtcagcagcctcctcgcggctttacccggtaacacagaggaacaccctgtggcgaatgctgacgattctaga

SEQ ID NO: 138 - DNA, 111 bp, synthetic construct
  source 1..111: mol_type = other DNA
ggagtaactatgactctcttaaggaagagaatcatcaacgtcagcagcctcctcgcggcttactccggtaacacagaggaacaccctgtggcgaatgctgacgattctaga

SEQ ID NO: 139 - DNA, 105 bp, synthetic construct
  source 1..105: mol_type = other DNA
gtaactatgactctcttaaggaagagagtaaacgtcagcagcctcctcgcggctagttacggtaacacagaggaacaccctgtggcgaatgctgacgattctaga

SEQ ID NO: 140 - DNA, 86 bp, synthetic construct
  source 1..86: mol_type = other DNA
gactctcttaaggcgtcagcagcctcctcgcggctagttacggtaacacagaggaacaccctgtggcgaatgctgacgattctaga

SEQ ID NO: 141 - DNA, 91 bp, synthetic construct
  source 1..91: mol_type = other DNA
ggccggcatggtcccagcctcctcgctggcgccggctgggcaacattccgaggggaccgtcccctcggtaatggcgaatgggacctctaga

SEQ ID NO: 142 - DNA, 132 bp, synthetic construct
  source 1..132: mol_type = other DNA
gggtaaacggcgggagtaactatgactctcttaacaccgaactgttagagaatcatagaacgtcagcagcctcctcgcggccccgccggtaacacagaggaacaccctgtggcgaatgctgacgattctaga

SEQ ID NO: 143 - DNA, 113 bp, synthetic construct
  source 1..113: mol_type = other DNA
ggcgggagtaactatgactctcttaaaaaagagaatcatagaacgtcagcggcctccacgcggccccgccggaacgcagaggaacaccctgcggcgaacgctgacgatctaga

SEQ ID NO: 144 - DNA, 341 bp, synthetic construct
  source 1..341: mol_type = other DNA
gactctcttaaggtgctgagtacgtccgcgtcgtgagagtcggtaactgtcccaggatggtctgggataggctaaactcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttg

SEQ ID NO: 145 - DNA, 121 bp, synthetic construct
  source 1..121: mol_type = other DNA
gggtaaacggcgggagtaactatgactctcttaaaaaagagaatcatagaacgtcagcggcctccacgcggccccgccggaacgcagaggaacaccctgcggcgaacgctgacgctctaga

SEQ ID NO: 146 - DNA, 112 bp, synthetic construct
  source 1..112: mol_type = other DNA
ggcgggagtaactatgactctcttaaaaaagagaatcatagaccgtcagcggcctccacgcggccccgccggaacgcagaggaacaccctgcggcgaacgctgacgtctaga

SEQ ID NO: 147 - DNA, 122 bp, synthetic construct
  source 1..122: mol_type = other DNA
gggtaaacggcgggagtaactatgactctcttaaaaaagagaatcatagaacgtcagcggcccccacacggccccgccggaacgcagaggaacaccctgcggcgaacgctgacgattctaga

SEQ ID NO: 148 - DNA, 120 bp, synthetic construct
  source 1..120: mol_type = other DNA
gggtaaacggcgggagtaactatgactctcttaaaaaagagaatcatagaccgtcagcggcccccacacggccccgccggaacgcagaggaacaccctgcggcgaacgctgacgtctaga

SEQ ID NO: 149 - DNA, 112 bp, synthetic construct
  source 1..112: mol_type = other DNA
ggcgggagtaactatgactctcttaaaaaagagaatcatagaccgtcagcggcccccacacggccccgccggaacgcagaggaacaccctgcggcgaacgctgacgtctaga

SEQ ID NO: 150 - DNA, 113 bp, synthetic construct
  source 1..113: mol_type = other DNA
ggcgggagtaactatgactctcttaacaaagagaatcatagaacgtcagcggcctccacgcggccccgccggaacgcagaggaacaccctgcggcgaacgctgacgctctaga

SEQ ID NO: 151 - DNA, 113 bp, synthetic construct
  source 1..113: mol_type = other DNA
ggcgggagtaactatgactctcttaacaaagagaatcatagaacgtcagcggcccccacacggccccgccggaacgcagaggaacaccctgcggcgaacgctgacggtctaga

SEQ ID NO: 152 - DNA, 121 bp, synthetic construct
  source 1..121: mol_type = other DNA
gggtaaacggcgggagtaactatgactctcttaacaaagagaatcatagaacgtcagcggcccccacacggccccgccggaacgcagaggaacaccctgcggcgaacgctgacggtctaga

SEQ ID NO: 153 - DNA, 121 bp, synthetic construct
  source 1..121: mol_type = other DNA
gggtaaacggcgggagtaactatgactctcttaacaaagagaatcatagaacgtctgcggcccccacacggccccgccggaacgcagaggaacaccctgcggcgaacgcagacggtctaga

SEQ ID NO: 154 - RNA, 111 nt, synthetic construct
  source 1..111: mol_type = other RNA
tgagggggacagctgggagtctcggcatgattacaaatcttgcgctgcactcggatgtcgtccccgtgacggacacattaatccggaaagcgagtggtgactcgcctcaag

SEQ ID NO: 155 - RNA, 638 nt, synthetic construct
  source 1..638: mol_type = other RNA
taaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaagg

SEQ ID NO: 156 - RNA, 353 nt, synthetic construct
  source 1..353: mol_type = other RNA
taggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaata

SEQ ID NO: 157 - RNA, 328 nt, synthetic construct
  source 1..328: mol_type = other RNA
taattcaggttatttagatgcttagtttttgtacctttcttgttttgtttaggattttgatagtgttagtatttttatatttttgtacgattgcataatgttcttttttatacagttctgttttaataaaatagacgatagctagagacgttagggcagccacaagccagttaggtagcggatagtaggtaggaacagacttttactatttcataacgcgtcaattaccacctgatttggaccaattcacgggatttgtccaaggtggacgggccacctttacttaacccggaaaaggaacatatataatttatgtgtgttcgataaa

SEQ ID NO: 158 - RNA, 292 nt, synthetic construct
  source 1..292: mol_type = other RNA
tagggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaa

SEQ ID NO: 159 - RNA, 398 nt, synthetic construct
  source 1..398: mol_type = other RNA
tagggggcttggcatttctcattgcctgctcctgaaaggatatgggtcctgcgtcgcgtggtaggcagacccattcgtccgagtagggggcttggcagtntccattgcctgtgcccgaaaggacgtgggtcatctggtctgtctgcctacacctctctagacttgtaacatctagtctgtcaacaagatcaaaattcttcacacagacgaccgagcttgctcagtcttcctgtacccgcagaattttgctcttgctctcctttggctgtgtcctggacgtgggactattccatctcgtcccaaatgccgcgtccaattataccggatttgacaaagcggacggcccgctttataagccggaaaaggtgccttgtaaaattgcaaggttcattaaatag

SEQ ID NO: 160 - RNA, 696 nt, synthetic construct
  source 1..696: mol_type = other RNA
tgacctgaacaaaacgtgttgtcttgtcttgtctaaaactatttattcgaaataaggggaggctaactgcctgcaagttgaacgcgaaagttagaccttcccacctaaagcccaaaagtgatcggggaatgaatccgcgggtgaccccagagttgggtaaacccttgaaacgttggagaagcggaagagagtcccgccaccgagcatcgagtgctgcggcgcccgaatgaaaccgatcgcggatggtgcaagtcgtaggacggggcacgacctaagcctctgtcacggcggcgaagccaggaatcaccatgcaaaggtgtgaactggggcggatacctccacggggtttccctgggcatcgcgcgagcgatggccaaagtccgctttctcagctacaaaacaaaaatggtatgagacttcgttaacactaatttttccgagcctagcaggctcccttgacaacgcttatgaatctggaaaaggacacaaagtggaaaaagcgctgatggtggacaaaagtcagttgagacttgatatcagttgttttgactaagaattttattatcgttgacttttaaatattttattattgactgttaatatactgacttgggaccaagtcatctctgttacccggtaccggttcctgtcatcaaaccggaaagtccgtcccacgtaatgtggtagacgcaggag

SEQ ID NO: 161 - RNA, 174 nt, synthetic construct
  source 1..174: mol_type = other RNA
tagggttcctccaccctccggctgacgaacagctggtaaacgggggggcggtggggtgcctctccagccgactgatacaggagtaagggacggtggggtcgcatccaggaagcgcagcaccgcgatgccgaaactgatgtgcagtataacacagaaagcctaaagggccaaaag

SEQ ID NO: 162 - RNA, 148 nt, synthetic construct
  source 1..148: mol_type = other RNA
taaattttgtctctttccccaatgatgtctactagcacgctgccgaagctagatagattgaggaatctgcgtaatctgtaatgattacgcctcatgggcatctatcggtagcgtcgaccctgacgttaaattgggtaataagaaatat

SEQ ID NO: 163 - RNA, 251 nt, synthetic construct
  source 1..251: mol_type = other RNA
tgagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaa

SEQ ID NO: 164 - RNA, 258 nt, synthetic construct
  source 1..258: mol_type = other RNA
tagctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaa

SEQ ID NO: 165 - RNA, 1155 nt, synthetic construct
  source 1..1155: mol_type = other RNA
ggaggggagtaggtctctactctgacccgaagggcccccccgtttcagacctgattctaggctacctgtgcctaattgggggggtcccaaagagatgttgtctgttgtagaagggtttgcgccactgactgcacggaagggtgggcctcgacaggtaggggttacatgactccgtgctgctcagcagacccgcgcctctgagaccgggtagggctacttgaacaagcgacgccctggtgtatgtccgtatcctaacctggtttgggaaagccgataccggcaatgcccgccacaggtgtcgcgcaccccacgggatgacgtatgggccccgggggacctcatggatactccactggacttgcacaatcctggtgtactggatgcagcgacgttggtgacataagcaatcgctaagtcggggtaggggaggtggggacctcggcacggctgtaggaacgggtgtatgggctccggcagccgtcgtcactcccatacaacacaggggctgcatcctggtggccggtgctagttggttctggaagcccgcccgggctggttcgcagaagcagggtgcgcccagggtaggtttggtatatctgggtccggtgcgatacctatcgatgggcagcgagggccgcctcgtgacgcgctgtgtggagctggagccggcctgggtatgaacagttcttgcggatgtggcgtagctagatagtacccgtggttgtgggcgtggtgtcgaccaaatgttgtcctgtgtgcacataggccaagggttacgtgggtggcagtcagaagcacccgcacctggaagtgattgccccgggatcccggctctctgtgaagagctaccttgaggaaaggtgttccgctggaactcaagaccctacagtaggggatatcaactggctttgaggtgctgtgattccggaaccagggcgagggcgagtacttagagcatgtccaaaagcccggggaacgttccgggggcctgcttgggtcgttggacccacatccgtaaaacgatggatctcgcgtcggcgctcgggagaacttcccgcatgaacgctgattgcatgtgagaacgcccccacggcggcggggcaggcgctccccctgggtgtaaggctcgggggggtcacggctccgctctaaaag

SEQ ID NO: 166 - RNA, 345 nt, synthetic construct
  source 1..345: mol_type = other RNA
tgataatcgctccatcctgcaactaattatgaatgcaaatctgttaagtgacattagtgatacttacctgatacttaccctggtatttatttgacctatacttaccctggtatctacctgacatatatttatctaaccacctacctatgatgactcccgcggaaactctcacttaccttattacccacttggtcttttattttctcgttccttattactttgttcctttggtgtagggttctctggtttttggaacggcttccttagccggaattttgtctgatgtatcttgcttgtgtccttgaaatatacgacccaggcttgcgtcatttaggctctgggaaa

SEQ ID NO: 167 - RNA, 314 nt, synthetic construct
  source 1..314: mol_type = other RNA
tagatgactgccctacccctttgctgccgaagaactactgaagactattgaactaaccaactgtgttaagtaagaactaatgcctctttttccctgcatgtatcccctgatcagtgacttattttcttttcctgttgcgccctttgtttagttatttcctttaattactagaattatcttttcgttctccgtctaattgctttttcggtgtaggaacggctacctaaagctggaagtgggaagtgttttcaatgtactttgtgattatagaaatatatgacccgaggtgcattgtttggcatttcctcgagaaa

SEQ ID NO: 168 - RNA, 120 nt, synthetic construct
  source 1..120: mol_type = other RNA
tgacagtaatatgaaaacatcacatctgaccggcacagaatcaccatgccgtaatgcacccaactaaggattccaatgggtaaaaaaaaaaaaaaaaaaaaaaaaaaaaaaaaaaaaaaa

SEQ ID NO: 169 - RNA, 256 nt, synthetic construct
  source 1..256: mol_type = other RNA
tagctaaatcgtttggttcaaaacatttgcttgctgtcttggcataacatcaataaaggcataaacatcgcaaaataatggttataattaaatggctatgaggatggttttagtacgtaggcgttgcggaacttcggttcatatagagcaatgaatcgtgcatgctaggaaaactgaccacacacagtgttggcagacctagtatctttcgaagatttccatacctccgcgatcaaaaaaaaaaaaaaaaaaaaaa

SEQ ID NO: 170 - RNA, 161 nt, synthetic construct
  source 1..161: mol_type = other RNA
tagcaccgaggtttactttcctcattgaaagtcaactacactggccaacatctattgtgtacaaagtaaagggaaaatagtcatatgacgtgccatccaaatatggcattgttgtgtgggtaaattcatttcctacaaaaaaaaaaaaaaaaaaaaaaaaa

SEQ ID NO: 171 - RNA, 152 nt, synthetic construct
  source 1..152: mol_type = other RNA
tagcagctctgaaagcgtatcctacaaacgctgctaacttgcacaacatcttaaaaagttataaagaaagcacacgggtgtcttagcttatcagatatctctggtatagcgagtcatatagcgcaacaaaaaaaaaaaaaaaaaaaaaaaaa

SEQ ID NO: 172 - RNA, 306 nt, synthetic construct
  source 1..306: mol_type = other RNA
tgaactagtctccttcttctattagtcagtctaattaatttttcttacattctacatctagttccattattaaattggtatgatcagtgctatctctgctacactcaatgcttaatcgtatgttattgacagtctgacacttgattactcttacgacatatgcactgtttgcttcagagaaaccactgttcatatagtgaagttcctcagttttctgttgatatattcttctttcattctcgcttctccttttctactgtgttctttttatcagttttttgtggaaaaattgagaataaataaagt

SEQ ID NO: 173 - RNA, 510 nt, synthetic construct
  source 1..510: mol_type = other RNA
tgataaatgccaaaagttgcttgggctaaatgatacgtacgctagaaaaagcgacttgctgcacggatgacggttcatcagagcccgatatgtgcatgtcaaggcggcagggagaatcactagtgtagctgttctttccattacgacttacgcggttaacgtggcacgatagatttacaccaggaaataatacgtgaagggttccaccatatactggagtttagatctatgagggaaacatttgtaataagtcagtctggtaacctggcgccgctgttgagtcaaattaactatgtcaatactcattaagttatcgactttgatatggcatggggtgattccgcgttatatcaaagtcaaacatgatgattgcaatgagaaactaccacgcttggtcacgtttgtgaggagaacatctcattcaagcctcccggatgtcggcacccgctgacatcttctggcttatgaaaattttcattaatttttgtaagtcatgggcggcttgaaagc

SEQ ID NO: 174 - DNA, 176 bp, synthetic construct
  source 1..176: mol_type = other DNA
gcttgctgccttggcataacatcaataaaggcataaacttcccaaaagaggatgcttttagtacgtaggcgttgcggaacttcggttcagatagagcaatgaatcgtgcatgctaggaacacctagtatctttgaagatttgcatcctccgggaacaaaaaaaaaaaaaaaaaaaa

SEQ ID NO: 175 - DNA, 176 bp, synthetic construct
  source 1..176: mol_type = other DNA
gcttgctgccttggcataacatcaataaaggcataaacttcccaaaagaggatgcttttagtacgtaggcgttgcggaacttcggttcagatagagcaatgaatcgtgcatgctaggaacacctagtatctttgaagatttgcatcctccgggaacaaaagaaaagaaaagaaaaa

SEQ ID NO: 176 - DNA, 217 bp, synthetic construct
  source 1..217: mol_type = other DNA
gacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaa

SEQ ID NO: 177 - DNA, 98 bp, synthetic construct
  source 1..98: mol_type = other DNA
ccggacttgtcatgatctcccagacttgtccaaggtggacgggccacctttacttaacccggaaaaggaacatatattaattatatgtgttcggaaaa

SEQ ID NO: 178 - DNA, 68 bp, synthetic construct
  source 1..68: mol_type = other DNA
caaggtggacgggccacctttacttaacccggaaaaggaacatatattaattatatgtgttcggaaaa

SEQ ID NO: 179 - DNA, 32 bp, synthetic construct
  source 1..32: mol_type = other DNA
ggcgggagtaactatgactctcttaaggtagc

SEQ ID NO: 180 - DNA, 30 bp, synthetic construct
  source 1..30: mol_type = other DNA
ggcgggagtaactatgactctcttaaggta

SEQ ID NO: 181 - DNA, 28 bp, synthetic construct
  source 1..28: mol_type = other DNA
ggcgggagtaactatgactctcttaagg

SEQ ID NO: 182 - DNA, 27 bp, synthetic construct
  source 1..27: mol_type = other DNA
ggcgggagtaactatgactctcttaag

SEQ ID NO: 183 - DNA, 26 bp, synthetic construct
  source 1..26: mol_type = other DNA
ggcgggagtaactatgactctcttaa

SEQ ID NO: 184 - DNA, 25 bp, synthetic construct
  source 1..25: mol_type = other DNA
ggcgggagtaactatgactctctta

SEQ ID NO: 185 - DNA, 24 bp, synthetic construct
  source 1..24: mol_type = other DNA
ggcgggagtaactatgactctctt

SEQ ID NO: 186 - DNA, 23 bp, synthetic construct
  source 1..23: mol_type = other DNA
ggcgggagtaactatgactctct

SEQ ID NO: 187 - DNA, 22 bp, synthetic construct
  source 1..22: mol_type = other DNA
ggcgggagtaactatgactctc

SEQ ID NO: 188 - DNA, 18 bp, synthetic construct
  source 1..18: mol_type = other DNA
ggcgggagtaactatgac

SEQ ID NO: 189 - DNA, 14 bp, synthetic construct
  source 1..14: mol_type = other DNA
ggcgggagtaacta

SEQ ID NO: 190 - DNA, 24 bp, synthetic construct
  source 1..24: mol_type = other DNA
ggagtaactatgactctcttaagg

SEQ ID NO: 191 - DNA, 22 bp, synthetic construct
  source 1..22: mol_type = other DNA
ggagtaactatgactctcttaa

SEQ ID NO: 192 - DNA, 21 bp, synthetic construct
  source 1..21: mol_type = other DNA
ggagtaactatgactctctta

SEQ ID NO: 193 - DNA, 21 bp, synthetic construct
  source 1..21: mol_type = other DNA
gtaactatgactctcttaagg

SEQ ID NO: 194 - DNA, 19 bp, synthetic construct
  source 1..19: mol_type = other DNA
gtaactatgactctcttaa

SEQ ID NO: 195 - DNA, 21 bp, synthetic construct
  source 1..21: mol_type = other DNA
gtaactatgactctcttaagg

SEQ ID NO: 196 - DNA, 21 bp, synthetic construct
  source 1..21: mol_type = other DNA
gtaactatgactctcttaagg

SEQ ID NO: 197 - DNA, 36 bp, synthetic construct
  source 1..36: mol_type = other DNA
gggtaaacggcgggagtaactatgactctcttaagg

SEQ ID NO: 198 - DNA, 34 bp, synthetic construct
  source 1..34: mol_type = other DNA
gggtaaacggcgggagtaactatgactctcttaa

SEQ ID NO: 199 - DNA, 33 bp, synthetic construct
  source 1..33: mol_type = other DNA
gggtaaacggcgggagtaactatgactctctta

SEQ ID NO: 200 - DNA, 34 bp, synthetic construct; identical to SEQ ID NO: 198
  source 1..34: mol_type = other DNA

SEQ ID NO: 201 - DNA, 36 bp, synthetic construct; identical to SEQ ID NO: 197
  source 1..36: mol_type = other DNA

SEQ ID NO: 202 - DNA, 33 bp, synthetic construct; identical to SEQ ID NO: 199
  source 1..33: mol_type = other DNA

SEQ ID NO: 203 - DNA, 32 bp, synthetic construct
  source 1..32: mol_type = other DNA
gggtaaacggcgggagtaactatgactctctt

SEQ ID NO: 204 - skipped

SEQ ID NO: 205 - DNA, 24 bp, synthetic construct
  source 1..24: mol_type = other DNA
gggagtaactatgactctcttaag

SEQ ID NO: 206 - DNA, 264 bp, synthetic construct
  source 1..264: mol_type = other DNA
tggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggt

SEQ ID NO: 207 - DNA, 93 bp, synthetic construct
  source 1..93: mol_type = other DNA
gagcgtttttcgaagtggactgtgtgactgcgttccccccttagttgctatatccgcttcgattaacatctcacctcgacgtataagatcatt

SEQ ID NO: 208-217 - skipped (10 entries)

SEQ ID NO: 218 - DNA, 100 bp, synthetic construct
  source 1..100: mol_type = other DNA
gaaattaatacgactcactatagggagaactcactatttgttttcgcgcccagttgcaaaaagtgtcggccaccatgggaacagatacagtgtatgtcgg

SEQ ID NO: 219 - DNA, 128 bp, synthetic construct
  source 1..128: mol_type = other DNA
  misc_feature 1..64: note = TOP
  misc_feature 65..128: note = BOTTOM
caagcgcgggtaaacggcgggagtaactatgactctcttaaggtagccaaatgcctcgtcatctagatgacgaggcatttggctaccttaagagagtcatagttactcccgccgtttacccgcgcttg

SEQ ID NO: 220 - RNA, 168 nt, synthetic construct
  source 1..168: mol_type = other RNA
  misc_feature 1..84: note = TOP
  misc_feature 85..168: note = BOTTOM
caagcgcgggtaaacggcgggagtaactatgactctcttaaggtagccaaatgcctcgtcatctaattagtgacgcgcatgaatattcatgcgcgtcactaattagatgacgaggcatttggctaccttaagagagtcatagttactcccgccgtttacccgcgcttg

SEQ ID NO: 221 - RNA, 150 nt, synthetic construct
  source 1..150: mol_type = other RNA
  misc_feature 1..75: note = TOP
  misc_feature 76..150: note = BOTTOM
caaatcgatcgtcaaacatgcgttaaggggcgaaagactaatcgaaccatctagtagctggttccgtccgaagttaacttcggacggaaccagctactagatggttcgattagtctttcgccccttaacgcatgtttgacgatcgatttg

SEQ ID NO: 222 - RNA, 128 nt, synthetic construct
  source 1..128: mol_type = other RNA
  misc_feature 1..64: note = TOP
  misc_feature 65..128: note = BOTTOM
tttgggttccggggggagtatggttgcaaagctgaaacttaaaggaattgacggaaaggagtggccactcctttccgtcaattcctttaagtttcagctttgcaaccatactccccccggaacccaaa

SEQ ID NO: 223 - RNA, 128 nt, synthetic construct
  source 1..128: mol_type = other RNA
  misc_feature 1..64: note = TOP
  misc_feature 65..128: note = BOTTOM
tttggattccgggggtagtatggttgcaaagctgaaacttaaacggaagggcaccaccaggagtactcctggtggtgcccttccgtttaagtttcagctttgcaaccatactacccccggaatccaaa

SEQ ID NO: 224 - RNA, 128 nt, synthetic construct
  source 1..128: mol_type = other RNA
  misc_feature 1..64: note = TOP
  misc_feature 65..128: note = BOTTOM
caagcgcgggtaaacggcgggagtaactatgactctctggctaccttaaaatgcctcgtcatctagatgacgaggcattttaaggtagccagagagtcatagttactcccgccgtttacccgcgcttg

SEQ ID NO: 225 - DNA, 137 bp, synthetic construct
  source 1..137: mol_type = other DNA
tgagggggacagctgggagtctcggcatgattacaaatcttgcgctgcactcggatgtcgtccccgtgacggacacattaatccggaaagcgagtggtgactcgcctcaagtagcaaaaaaaaaaaaaaaaaaaaaa

SEQ ID NO: 226 - DNA, 155 bp, synthetic construct
  source 1..155: mol_type = other DNA
tgagggggacagctgggagtctcggcatgattacaaatcttgcgctgcactcggatgtcgtccccgtgacggacacattaatccggaaagcgagtggtgactcgcctcaagtagcaaaaaaaaaaaaaaaaaaaaaaaaaaaaaaaaaaaaaaaa

SEQ ID NO: 227 - DNA, 175 bp, synthetic construct
  source 1..175: mol_type = other DNA
tgagggggacagctgggagtctcggcatgattacaaatcttgcgctgcactcggatgtcgtccccgtgacggacacattaatccggaaagcgagtggtgactcgcctcaagtagcaaaaaaaaaaaaaaaaaaaaaaaaaaaaaaaaaaaaaaaaaaaaaaaaaaaaaaaaaaaa

SEQ ID NO: 228 - RNA, 153 nt, synthetic construct
  source 1..153: mol_type = other RNA
tgagggggacagctgggagtctcggcatgattacaaatcttgcgctgcactcggatgtcgtccccgtgacggacacattaatccggaaagcgagtggtgactcgcctcaagtagccaaatgcctcgtcatcaaaaaaaaaaaaaaaaaaaaaa

SEQ ID NO: 229 - RNA, 171 nt, synthetic construct
  source 1..171: mol_type = other RNA
tgagggggacagctgggagtctcggcatgattacaaatcttgcgctgcactcggatgtcgtccccgtgacggacacattaatccggaaagcgagtggtgactcgcctcaagtagccaaatgcctcgtcatcaaaaaaaaaaaaaaaaaaaaaaaaaaaaaaaaaaaaaaaa

SEQ ID NO: 230 - RNA, 191 nt, synthetic construct
  source 1..191: mol_type = other RNA
tgagggggacagctgggagtctcggcatgattacaaatcttgcgctgcactcggatgtcgtccccgtgacggacacattaatccggaaagcgagtggtgactcgcctcaagtagccaaatgcctcgtcatcaaaaaaaaaaaaaaaaaaaaaaaaaaaaaaaaaaaaaaaaaaaaaaaaaaaaaaaaaaaa

SEQ ID NO: 231 - DNA, 664 bp, synthetic construct
  source 1..664: mol_type = other DNA
taaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagcaaaaaaaaaaaaaaaaaaaaaa

SEQ ID NO: 232 - DNA, 379 bp, synthetic construct
  source 1..379: mol_type = other DNA
taggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagcaaaaaaaaaaaaaaaaaaaaaa

SEQ ID NO: 233 - DNA, 397 bp, synthetic construct
  source 1..397: mol_type = other DNA
taggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagcaaaaaaaaaaaaaaaaaaaaaaaaaaaaaaaaaaaaaaaa

SEQ ID NO: 234 - DNA, 417 bp, synthetic construct
  source 1..417: mol_type = other DNA
taggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagcaaaaaaaaaaaaaaaaaaaaaaaaaaaaaaaaaaaaaaaaaaaaaaaaaaaaaaaaaaaa

SEQ ID NO: 235 - RNA, 403 nt, synthetic construct
  source 1..403: mol_type = other RNA
taggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagccaaatgaaaaaaaaaaaaaaaaaaaaaaaaaaaaaaaaaaaaaaaa

SEQ ID NO: 236 - RNA, 385 nt, synthetic construct
  source 1..385: mol_type = other RNA
taggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagccaaatgaaaaaaaaaaaaaaaaaaaaaa

SEQ ID NO: 237 - RNA, 423 nt, synthetic construct
  source 1..423: mol_type = other RNA
taggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagccaaatgaaaaaaaaaaaaaaaaaaaaaaaaaaaaaaaaaaaaaaaaaaaaaaaaaaaaaaaaaaaa

SEQ ID NO: 238 - DNA, 318 bp, synthetic construct
  source 1..318: mol_type = other DNA
tagggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 239 - RNA, 302 nt, synthetic construct
  source 1..302: mol_type = other RNA
tagggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagccaaatg

SEQ ID NO: 240 - RNA, 324 nt, synthetic construct
  source 1..324: mol_type = other RNA
tagggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagccaaatgaaaaaaaaaaaaaaaaaaaaaa

SEQ ID NO: 241 - RNA, 324 nt, synthetic construct; identical to SEQ ID NO: 240
  source 1..324: mol_type = other RNA

SEQ ID NO: 242 - RNA, 312 nt, synthetic construct
  source 1..312: mol_type = other RNA
tagggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagccaaatgcctcgtcatc

SEQ ID NO: 243 - RNA, 327 nt, synthetic construct
  source 1..327: mol_type = other RNA
tagggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagccaaatgcctcgtcatcaaaaaaaaaaaaaaa

SEQ ID NO: 244 - RNA, 334 nt, synthetic construct
  source 1..334: mol_type = other RNA
tagggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagccaaatgcctcgtcatcaaaaaaaaaaaaaaaaaaaaaa

SEQ ID NO: 245 - DNA, 354 bp, synthetic construct
  source 1..354: mol_type = other DNA
taattcaggttatttagatgcttagtttttgtacctttcttgttttgtttaggattttgatagtgttagtatttttatatttttgtacgattgcataatgttcttttttatacagttctgttttaataaaatagacgatagctagagacgttagggcagccacaagccagttaggtagcggatagtaggtaggaacagacttttactatttcataacgcgtcaattaccacctgatttggaccaattcacgggatttgtccaaggtggacgggccacctttacttaacccggaaaaggaacatatataatttatgtgtgttcgataaatagcaaaaaaaaaaaaaaaaaaaaaa

SEQ ID NO: 246 - DNA, 424 bp, synthetic construct
  source 1..424: mol_type = other DNA
tagggggcttggcatttctcattgcctgctcctgaaaggatatgggtcctgcgtcgcgtggtaggcagacccattcgtccgagtagggggcttggcagtntccattgcctgtgcccgaaaggacgtgggtcatctggtctgtctgcctacacctctctagacttgtaacatctagtctgtcaacaagatcaaaattcttcacacagacgaccgagcttgctcagtcttcctgtacccgcagaattttgctcttgctctcctttggctgtgtcctggacgtgggactattccatctcgtcccaaatgccgcgtccaattataccggatttgacaaagcggacggcccgctttataagccggaaaaggtgccttgtaaaattgcaaggttcattaaatagtagcaaaaaaaaaaaaaaaaaaaaaa

SEQ ID NO: 247 - DNA, 277 bp, synthetic construct
  source 1..277: mol_type = other DNA
tgagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagcaaaaaaaaaaaaaaaaaaaaaa

SEQ ID NO: 248 - DNA, 284 bp, synthetic construct
  source 1..284: mol_type = other DNA
tagctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaatagcaaaaaaaaaaaaaaaaaaaaaa

SEQ ID NO: 249 - RNA, 300 nt, synthetic construct
  source 1..300: mol_type = other RNA
tagctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaatagccaaatgcctcgtcatcaaaaaaaaaaaaaaaaaaaaaa

SEQ ID NO: 250 - RNA, 278 nt, synthetic construct
  source 1..278: mol_type = other RNA
tagctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaatagccaaatgcctcgtcatc

SEQ ID NO: 251 - DNA, 262 bp, synthetic construct
  source 1..262: mol_type = other DNA
tagctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaatagc

SEQ ID NO: 252 - RNA, 196 nt, synthetic construct
  source 1..196: mol_type = other RNA
gcttgctgccttggcataacatcaataaaggcataaacttcccaaaagaggatgcttttagtacgtaggcgttgcggaacttcggttcagatagagcaatgaatcgtgcatgctaggaacacctagtatctttgaagatttgcatcctccgggaacaaaaaaaaaaaaaaaaaaaatagccaaatgcctcgtcatc

SEQ ID NO: 253 - RNA, 196 nt, synthetic construct
  source 1..196: mol_type = other RNA
gcttgctgccttggcataacatcaataaaggcataaacttcccaaaagaggatgcttttagtacgtaggcgttgcggaacttcggttcagatagagcaatgaatcgtgcatgctaggaacacctagtatctttgaagatttgcatcctccgggaacaaaagaaaagaaaagaaaaatagccaaatgcctcgtcatc

SEQ ID NO: 254 - DNA, 667 bp, synthetic construct
  source 1..667: mol_type = other DNA
gggagtaactatgactctcttaaggtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagc

SEQ ID NO: 255 - DNA, 673 bp, synthetic construct
  source 1..673: mol_type = other DNA
gggagtaactatgactctcttaaggtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagccaaatg

SEQ ID NO: 256 - DNA, 689 bp, synthetic construct
  source 1..689: mol_type = other DNA
gggagtaactatgactctcttaaggtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagcaaaaaaaaaaaaaaaaaaaaaa

SEQ ID NO: 257 - DNA, 1057 bp, synthetic construct
  source 1..1057: mol_type = other DNA
gggagtaactatgactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttgtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagc

SEQ ID NO: 258 - DNA, 1063 bp, synthetic construct
  source 1..1063: mol_type = other DNA
gggagtaactatgactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttgtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagccaaatg

SEQ ID NO: 259 - DNA, 1079 bp, synthetic construct
  source 1..1079: mol_type = other DNA
gggagtaactatgactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttgtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagcaaaaaaaaaaaaaaaaaaaaaa

SEQ ID NO: 260 - DNA, 1075 bp, synthetic construct
  source 1..1075: mol_type = other DNA
gggagtaactatgactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttgtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggaaaaaaaaaaaaaaaaaaaaaa

SEQ ID NO: 261 - DNA, 1085 bp, synthetic construct
  source 1..1085: mol_type = other DNA
gggagtaactatgactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttgtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagccaaatgaaaaaaaaaaaaaaaaaaaaaa

SEQ ID NO: 262 - DNA, 664 bp, synthetic construct; identical to SEQ ID NO: 231
  source 1..664: mol_type = other DNA

SEQ ID NO: 263 - RNA, 391 nt, synthetic construct
  source 1..391: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagagcggccgctgagggggacagctgggagtctcggcatgattacaaatcttgcgctgcactcggatgtcgtccccgtgacggacacattaatccggaaagcgagtggtgactcgcctcaagtagcaaaaaaaaaaaaaaaaaaaaaa

SEQ ID NO: 264 - RNA, 633 nt, synthetic construct
  source 1..633: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagagcggccgctaggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagcaaaaaaaaaaaaaaaaaaaaaa

SEQ ID NO: 265 - RNA, 918 nt, synthetic construct
  source 1..918: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagagcggccgctaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagcaaaaaaaaaaaaaaaaaaaaaa

SEQ ID NO: 266 - RNA, 551 nt, synthetic construct
  source 1..551: mol_type = other RNA
gggagacggtcaaccgcgtaggagcggtgaccggaattcggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttctagagcggccgctgagggggacagctgggagtctcggcatgattacaaatcttgcgctgcactcggatgtcgtccccgtgacggacacattaatccggaaagcgagtggtgactcgcctcaagtagcaaaaaaaaaaaaaaaaaaaaaa

SEQ ID NO: 267 - RNA, 793 nt, synthetic construct
  source 1..793: mol_type = other RNA
gggagacggtcaaccgcgtaggagcggtgaccggaattcggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttctagagcggccgctaggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagcaaaaaaaaaaaaaaaaaaaaaa

SEQ ID NO: 268 - RNA, 1078 nt, synthetic construct
  source 1..1078: mol_type = other RNA
gggagacggtcaaccgcgtaggagcggtgaccggaattcggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttctagagcggccgctaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagcaaaaaaaaaaaaaaaaaaaaaa

SEQ ID NO: 269 - DNA, 1835 bp, synthetic construct
  source 1..1835: mol_type = other DNA
ccaatgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 270 - DNA, 5444 bp, synthetic construct
  source 1..5444: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccgaattctgcagatatccagcacagtggcggccgctcgagtctagagggcccgattacaaggatgacgacgataagtgataaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 271 - DNA, 26 bp, synthetic construct
  source 1..26: mol_type = other DNA
cagcactagatttttggggttgaatg

SEQ ID NO: 272 - RNA, 163 nt, synthetic construct
  source 1..163: mol_type = other RNA
atacccgcttaattcattcagatctgtaatagaactgtcattcaaccccaaaaatctagtgctgatataaccttcaccaattaggttcaaataagtggtaatgcgggacaaaagactatcgacatttgatacactatttatcaatggatgtcttatttttttt

SEQ ID NO: 273 - DNA, 2246 bp, synthetic construct
  source 1..2246: mol_type = other DNA
agtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaa

SEQ ID NO: 274 - DNA, 3926 bp, synthetic construct
  source 1..3926: mol_type = other DNA
gctgggcgcgcgccgcggctggacgaggcgcgcgccccccccacgcccggggcacccccctcgcggccctcccccgccccacccgcgcgcgccgctcgctccctccccaccccgcgccctctctctctctctctcccccgctccccgtcctcccccctccccgggggagcgccgcgtgggggcgcggcggggggagaagggtcggggcggcaggggccgcgcggcggccgccggggcggccggcgggggcaggtccccgcgaggggggccccggggacccggggggccggcggcggcgcggactctggacgcgagccgggcccttcccgtggatcgccccagctgcggcgggcgtcgcggccgcccccggggagcccggcggcggcgcggcgcgccccccacccccaccccacgtctcggtcgcgcgcgcgtccgctgggggcgggagcggtcgggcggcggcggtcggcgggcggcggggcggggcggttcgtccccccgccctacccccccggccccgtccgccccccgttcccccctcctcctcggcgcgcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgcggccctgcgggccgccggtgaaataccactactctgatcgttttttcactgacccggtgaggcgggggggcgagcccgaggggctctcgcttctggcgccaagcgcccgcccggccgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcgccaagcgttggattgttcacccactaatagggaacgtgagctgggtttagaccgtcgtgagacaggttagttttaccctactgatgatgtgttgttgccatggtaatcctgctcagtacgagaggaaccgcaggttcagaca

SEQ ID NO: 275 - DNA, 799 bp, synthetic construct
  source 1..799: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 276 - DNA, 213 bp, synthetic construct
  source 1..213: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 277 - DNA, 228 bp, synthetic construct
  source 1..228: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcgtacggccaccatg

SEQ ID NO: 278 - DNA, 511 bp, synthetic construct
  source 1..511: mol_type = other DNA
ttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacct

SEQ ID NO: 279 - DNA, 122 bp, synthetic construct
  source 1..122: mol_type = other DNA
aacttgtttattgcagcttataatggttacaaataaagcaatagcatcacaaatttcacaaataaagcatttctttcactgcattctagttgtggtttgtccaaactcatcaatgtatctta

SEQ ID NO: 280 - DNA, 49 bp, synthetic construct
  source 1..49: mol_type = other DNA
aaataaaatacgaaatgaattcgtaatcatgtgtgttggtttcttgtgt

SEQ ID NO: 281 - DNA, 216 bp, synthetic construct
  source 1..216: mol_type = other DNA
ctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 282 - DNA, 634 bp, synthetic construct
  source 1..634: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggataggggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgcttgtctcgctccggggaacgctcggaaactcccggccgccgccacccgcgtctgttctgttacacaagggaagaaaagccgctgccgcactccgagtgtcgtacggccacc

SEQ ID NO: 283 - DNA, 324 bp, synthetic construct
  source 1..324: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattgcgtctatatatgcagaggccgaggccgcctcggcctctgagctattccagaagtagtgaggaggccgtacggccacc

SEQ ID NO: 284 - DNA, 744 bp, synthetic construct
  source 1..744: mol_type = other DNA
atggccccaaagaagaagcggaaggtcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 285 - protein, 247 aa, synthetic construct
MAPKKKRKVVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 286 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 287 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 288 - DNA, 1434 bp, synthetic construct
  source 1..1434: mol_type = other DNA
atggtgtccaaaggcgaggaagtgatcaaggaatttatgagattcaaggtcagaatggaaggatctatgaacggccacgagttcgagatcgagggagaaggagaaggcagaccctacgagggcacccaaaccgctaaattaaaagtgacaaagggcggacctctgccgttcgcctgggatatcctgtccccccagttcatgtacggcagcaaggcctacgtgaagcaccctgctgacatccccgattacaaaaagctgtcttttcctgagggctttaagtgggagagagtgatgaacttcgaggacggaggcctggtgaccgttacccaagacagcagcctgcaagatggaacactgatctacaaagtgaagatgcggggcactaatttccctccagacggccccgtgatgcagaagaaaaccatgggctgggaggcctctacagaacggctgtaccccagagacggagtgctgaagggcgagattcaccaggcactgaagctgaaagatggcggacactacctggtggagttcaagaccatttacatggccaagaagcctgtgcagctgcctggctactactacgtggacaccaagctggacatcacctcccacaatgaggattataccatcgtggaacagtacgagagaagcgagggcagacaccacctgttcctcggccacggcacaggctccaccggctctggcagcagcggcacagccagcagcgaggacaacaacatggccgtgatcaaggagttcatgcggttcaaggtgcggatggaaggcagtatgaacggccatgagttcgaaatcgagggcgaaggcgaaggcagaccttacgagggcacacagacagctaagctgaaggtgaccaagggaggccctctgccattcgcctgggacatcctgagccctcagtttatgtatggcagcaaggcctacgtgaagcaccccgccgacatccccgactacaagaagctgagcttccctgagggtttcaaatgggaaagggtgatgaacttcgaggacggcggactggtgacagtgacccaggacagcagcctgcaggatggcaccctgatctataaggtgaagatgagaggcaccaactttccacctgacgggcccgtgatgcagaaaaaaaccatgggatgggaggccagcaccgagcggctgtaccctagagatggcgttctgaagggcgagatccaccaggccctgaagctgaaggacggcggccactacctggtggagttcaagaccatctacatggccaagaaacctgtgcagctgcctggatactactacgtggacacaaagctggacatcacctctcacaacgaggactataccatcgtcgagcagtacgaacgcagcgaaggcagacaccatctgttcctgtacggtatggacgagctgtacaagtaatga

SEQ ID NO: 289 - protein, 476 aa, synthetic construct
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 290 - RNA, 711 nt, synthetic construct
  source 1..711: mol_type = other RNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 291 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 292 - DNA, 3408 bp, synthetic construct
  source 1..3408: mol_type = other DNA
atggtgatgcctcgagcaccccggtgcagagccgtgcggagcctgctgcggtctcactacagagaggtcctccccctggccacgttcgtgcggcgtctgggccctcagggctggcgactggtgcagcgcggcgaccccgccgcattccgcgccctggtggcccagtgcctggtgtgtgtgccttgggatgccagacctcctcctgcggctccttcttttagacaggtgtcttgtctgaaggagctggtggcccgggtgctgcagcggctgtgcgagcggggagctaagaatgtgttagctttcggttttgccctgctggacggcgccagaggcggaccacctgaagccttcactacctcggtgagaagctacctgcctaacaccgtgacagacgccctgcggggcagcggcgcctggggcctgctactgagacgggtgggagatgatgtgctggtgcacctgctggccagatgcgccctctttgtgctggtggcccctagctgtgcctaccaggtgtgtggccctcccctgtaccagctgggcgccgccacgcaggccagaccccctccgcacgccagcggccctaggagacgtctgggctgcgaaagagcttggaaccacagtgtgcgagaagccggcgtccccctgggcctccctgcccctggagccagaagaagaggaggctccgccagcagaagcctcccgctgcccaagcggcctagacggggcgccgcccctgaacccgagcggaccccggtcggccagggcagttgggcccaccccggaaggaccagaggacctagcgaccggggcttctgcgtggtctccccagcccggccagccgaggaagcaaccagcctggagggcgccctgagcggcaccagacactcccacccttccgttggcagacagcaccacgccggcccaccaagcaccagcagacctccgcgaccttgggacacaccctgtcctcctgtgtacgccgagaccaagcacttcctgtactcctctggcgataaggaacagctccggcctagcttcctgctgagctctctgagacccagcttgaccggcgcccgcagactcgtggaaacaatcttcctgggcagcagaccttggatgccaggcacccctagacggctgcctagactgccccagagatactggcagatgcgtccactgtttttggaactgctgggcaaccacgctcagtgtccttacggcgttctgctgaaaacccactgccccctgagagccgctgtcaccccagccgccggcgtgtgcgccagagaaaagcctcagggaagcgtggctgctcctgaggaagaggatacagaccctagacgcctggtgcaactgctgcgccaacatagcagcccttggcaggtgtacggcttcgtcagagcttgtctgagaaggctggtgcccccgggactgtggggctccagacacaacgagcggagattcctgcggaacacaaagaagttcatcagcctgggcaagcacgccaagctgtctctgcaggagttgacctggaagatgagcgtgagggattgcgcgtggctgagaagatcccctggagtgggctgtgtgccagccgccgagcacagactgcgagaggaaatcctggccaaattcctgcactggctgatgagcgtatatgtggtggaactgctgcggagcttcttctacgtgaccgagacaacatttcagaagaacagacttttcttctaccggaagtctgtatggagcaagctgcagtccatcggaatcagacagcacctgaagagagttcagctgcgggaactgagcgaggcagaggtgaggcagcatagagaggctagacctgctctgcttaccagcagactgagattcatcccaaaacctgatggcctgagacctatcgtgaatatggactacgtggtgggcgcccgaaccttccggcgtgagaagcgggctgaaagactgacatcccgggtgaaggccctgttcagcgtgctgaactacgagagagctcgtcggcctgggctactgggcgccagcgtgctgggcctggatgacatccacagagcctggagaacattcgtgctgagagtgagggctcaagatcccccaccggagctgtacttcgttaaggtggacgtgacaggcgcatacgacaccatccctcaggacagactgaccgaggtgattgccagcattatcaagccccagaacacctattgcgtgagacggtacgccgtggtgcagaaagccgcacatggccacgtgcggaaagccttcaagtctcacgtgtccaccctgaccgacctgcagccttacatgcggcaattcgtggcccacctccaggagacaagccctctgagagacgccgtggtgatcgagcagtccagcagccttaatgaggcctctagcggcctgttcgacgtgttcctgagatttatgtgccaccacgccgtgagaatcagaggcaaaagctacgtgcagtgccagggcatcccccaaggatctatcctgagcaccctgctgtgcagcctgtgctacggcgacatggaaaacaagctgttcgccggaatccggagagacggcctgctgctgcggctcgttgacgacttcctgctggtgacacctcacctgacacacgccaaaaccttcctcagaacactggttcggggcgtccccgagtacggctgcgtcgtgaacctgagaaagacagtggtgaacttccctgtggaagatgaggcactcggcggaacagcgttcgtgcagatgcccgcccacggcctgttcccctggtgcggcctgctgctggacaccagaaccctggaggtgcagagcgattacagcagctatgccagaaccagcatccgagccagcctaacctttaaccggggcttcaaagccggcagaaatatgagaagaaagctgttcggagtgctgaggcttaaatgccacagcctgttcctggacctgcaggtcaacagcctgcaaacagtgtgcaccaacatctacaagatcctgctgttacaagcctacagatttcacgcctgtgtgctgcagctgcctttccaccagcaggtgtggaagaaccctacgtttttcctgagagtgatctctgatacagccagtttgtgctacagcatcctgaaggccaagaacgccggcatgagcctgggagccaagggcgctgctgggcctctgccttctgaggccgtgcagtggctgtgccaccaggcctttctgctgaagctgaccagacatagagtgacctacgtgcctctcctgggcagcctgagaaccgcccagacccagctctctcggaagctgcctggcacaaccctgaccgccctggaagctgctgccaatcctgccctgcctagcgacttcaagaccatcctggactgataa

SEQ ID NO: 293 - protein, 1134 aa, synthetic construct
MVMPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 294 - DNA, 1369 bp, synthetic construct
  source 1..1369: mol_type = other DNA
atggtgatgagcaccgccgtgctggagaatcctggcctgggcagaaagctgtccgatttcggccaagagacaagctacattgaggataattgcaaccagaacggcgctatcagcctgatcttttctttgaaagaggaagtgggcgctctggccaaggtcctgaggctgttcgaggaaaacgacgtgaacctgacccatatcgaaagcagacccagccggctgaagaaagatgagtatgagttcttcacccacctggacaagagatctctccctgccctgacaaacatcatcaaaatcctaagacacgacattggcgccaccgtgcacgagctgtcaagagataagaagaaggacaccgtgccttggtttcctagaaccatccaggagctggatagattcgccaatcagatcctgtcttacggcgccgaactggacgccgaccaccccggcttcaaggaccctgtgtaccgggccagacgcaagcagttcgccgacatcgcctataactaccggcatggccagcctatccctagagtggaatacatggaagaagagaaaaaaacctggggcaccgttttcaagaccctgaaaagcctgtataagactcacgcctgctacgagtacaaccacatcttccctctgctggaaaagtactgtggctttcacgaggacaacatcccacagctggaggacgtgtcccagttcctgcagacctgtaccggcttccggctgcggcccgtggccggactgctgagcagccgggacttcctgggcggactggcttttagagtgttccactgcacacagtacatcagacacggctccaagcctatgtacacacctgagcccgacatctgccacgagctgctgggacacgtccctctgtttagcgatagatctttcgcccagtttagccaggagatcggcctggccagcttgggcgcccctgatgaatacatcgagaaactggctacaatctactggttcacagtggagttcggcctctgcaagcagggcgacagcatcaaggcctacggcgccggactgctttctagcttcggcgagctgcaatactgtctgtccgagaagcccaagctgctgcctctggaactggaaaagaccgccatccagaattacaccgtgaccgagttccagcccctgtactacgtggctgagagtttcaacgacgctaaggaaaaggtgcggaacttcgccgccacaatcccaagacctttcagcgtgcggtacgacccctacacccaaagaatcgaggtgctggacaacacccagcagctgaagatcctggcagatagcatcaacagcgagatcggaatcctgtgcagcgccctgcagaagattaagtgataag

SEQ ID NO: 295 - protein, 452 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 296 - RNA, 1805 nt, synthetic construct
  source 1..1805: mol_type = other RNA
tctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaagcttgccaccatggccccaaagaagaagcggaaggtcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgc

SEQ ID NO: 297 - RNA, 2313 nt, synthetic construct
  source 1..2313: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaa

SEQ ID NO: 298 - RNA, 2335 nt, synthetic construct
  source 1..2335: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaaaaaaaaaaaaaaaaaaaaaaaa

SEQ ID NO: 299 - RNA, 2317 nt, synthetic construct
  source 1..2317: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagc

SEQ ID NO: 300 - RNA, 2339 nt, synthetic construct
  source 1..2339: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 301 - RNA, 2333 nt, synthetic construct
  source 1..2333: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagccaaatgcctcgtcatc

SEQ ID NO: 302 - RNA, 2355 nt, synthetic construct
  source 1..2355: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagccaaatgcctcgtcatcaaaaaaaaaaaaaaaaaaaaaa

SEQ ID NO: 303 - RNA, 2093 nt, synthetic construct
  source 1..2093: mol_type = other RNA
aggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 304 - RNA, 2424 nt, synthetic construct
  source 1..2424: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaagcttgccaccatggccccaaagaagaagcggaaggtcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctaggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagcaaaaaaaaaaaaaaaaaaaaaa

SEQ ID NO: 305 - RNA, 2363 nt, synthetic construct
  source 1..2363: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaagcttgccaccatggccccaaagaagaagcggaaggtcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 306 - RNA, 2400 nt, synthetic construct
  source 1..2400: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctaggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaatatagcaaaaaaaaaaaaaaaaaaaaaa

SEQ ID NO: 307 - RNA, 2375 nt, synthetic construct
  source 1..2375: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctaattcaggttatttagatgcttagtttttgtacctttcttgttttgtttaggattttgatagtgttagtatttttatatttttgtacgattgcataatgttcttttttatacagttctgttttaataaaatagacgatagctagagacgttagggcagccacaagccagttaggtagcggatagtaggtaggaacagacttttactatttcataacgcgtcaattaccacctgatttggaccaattcacgggatttgtccaaggtggacgggccacctttacttaacccggaaaaggaacatatataatttatgtgtgttcgataaatagcaaaaaaaaaaaaaaaaaaaaaa

SEQ ID NO: 308 - RNA, 2330 nt, synthetic construct
  source 1..2330: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 309 - RNA, 2339 nt, synthetic construct; identical to SEQ ID NO: 300
  source 1..2339: mol_type = other RNA

SEQ ID NO: 310 - RNA, 2363 nt, synthetic construct; identical to SEQ ID NO: 305
  source 1..2363: mol_type = other RNA

SEQ ID NO: 311 - RNA, 2363 nt, synthetic construct
  source 1..2363: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcattgcggccgctgtggaattgtgagcgctcacaattccacaacttgtttattgcagcttataatggttacaaataaagcaatagcatcacaaatttcacaaataaagcatttttttcactgcattctagttgtggtttgtccaaactcatcaatgtatcttatcatgtctggatcaagcttttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcacgaccttccgcttcttctttggggccatggtggcaagcttccaacctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgtcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcacaatgccaggcgggccatttaccgtcattgacgtcaatagggggcgtacttggcatatgatacacttgatgtactgccaagtgggcagtttaccgtaaatactccacccattgacgtcaatggaaagtccctattggcgttactattgacgtcaatgggcgggggtcgttgggcggtcagccaggcgggccatttaccgtaagttatgtaacgtgtggaattgtgagcgctcacaattccacacggacaactggtcgacctcggacatctggtcgaccttctagatgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 312 - RNA, 2063 nt, synthetic construct
  source 1..2063: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctaagcttgccaccatggccccaaagaagaagcggaaggtcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttaacttgtttattgcagcttataatggttacaaataaagcaatagcatcacaaatttcacaaataaagcatttctttcactgcattctagttgtggtttgtccaaactcatcaatgtatcttatgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 313 - RNA, 2009 nt, synthetic construct
  source 1..2009: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcattgcggccgctgtggaattgtgagcgctcacaattccacaaaaacctcccacatctccccctgaacctgaaacataaaatgaatgcaattgttgttgttaacttgtttattgcagcttataatggttacaaataaagcaatagcatcacaaatttcacaaataaagctagatggttacaaataaagcaatagcatcacaaatttcacaaataaacaccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattataagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggccgtacgcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgccccggacaactggtcgacctcggacatctggtcgaccttctagatgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 314 - RNA, 5025 nt, synthetic construct
  source 1..5025: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgatgcctcgagcaccccggtgcagagccgtgcggagcctgctgcggtctcactacagagaggtcctccccctggccacgttcgtgcggcgtctgggccctcagggctggcgactggtgcagcgcggcgaccccgccgcattccgcgccctggtggcccagtgcctggtgtgtgtgccttgggatgccagacctcctcctgcggctccttcttttagacaggtgtcttgtctgaaggagctggtggcccgggtgctgcagcggctgtgcgagcggggagctaagaatgtgttagctttcggttttgccctgctggacggcgccagaggcggaccacctgaagccttcactacctcggtgagaagctacctgcctaacaccgtgacagacgccctgcggggcagcggcgcctggggcctgctactgagacgggtgggagatgatgtgctggtgcacctgctggccagatgcgccctctttgtgctggtggcccctagctgtgcctaccaggtgtgtggccctcccctgtaccagctgggcgccgccacgcaggccagaccccctccgcacgccagcggccctaggagacgtctgggctgcgaaagagcttggaaccacagtgtgcgagaagccggcgtccccctgggcctccctgcccctggagccagaagaagaggaggctccgccagcagaagcctcccgctgcccaagcggcctagacggggcgccgcccctgaacccgagcggaccccggtcggccagggcagttgggcccaccccggaaggaccagaggacctagcgaccggggcttctgcgtggtctccccagcccggccagccgaggaagcaaccagcctggagggcgccctgagcggcaccagacactcccacccttccgttggcagacagcaccacgccggcccaccaagcaccagcagacctccgcgaccttgggacacaccctgtcctcctgtgtacgccgagaccaagcacttcctgtactcctctggcgataaggaacagctccggcctagcttcctgctgagctctctgagacccagcttgaccggcgcccgcagactcgtggaaacaatcttcctgggcagcagaccttggatgccaggcacccctagacggctgcctagactgccccagagatactggcagatgcgtccactgtttttggaactgctgggcaaccacgctcagtgtccttacggcgttctgctgaaaacccactgccccctgagagccgctgtcaccccagccgccggcgtgtgcgccagagaaaagcctcagggaagcgtggctgctcctgaggaagaggatacagaccctagacgcctggtgcaactgctgcgccaacatagcagcccttggcaggtgtacggcttcgtcagagcttgtctgagaaggctggtgcccccgggactgtggggctccagacacaacgagcggagattcctgcggaacacaaagaagttcatcagcctgggcaagcacgccaagctgtctctgcaggagttgacctggaagatgagcgtgagggattgcgcgtggctgagaagatcccctggagtgggctgtgtgccagccgccgagcacagactgcgagaggaaatcctggccaaattcctgcactggctgatgagcgtatatgtggtggaactgctgcggagcttcttctacgtgaccgagacaacatttcagaagaacagacttttcttctaccggaagtctgtatggagcaagctgcagtccatcggaatcagacagcacctgaagagagttcagctgcgggaactgagcgaggcagaggtgaggcagcatagagaggctagacctgctctgcttaccagcagactgagattcatcccaaaacctgatggcctgagacctatcgtgaatatggactacgtggtgggcgcccgaaccttccggcgtgagaagcgggctgaaagactgacatcccgggtgaaggccctgttcagcgtgctgaactacgagagagctcgtcggcctgggctactgggcgccagcgtgctgggcctggatgacatccacagagcctggagaacattcgtgctgagagtgagggctcaagatcccccaccggagctgtacttcgttaaggtggacgtgacaggcgcatacgacaccatccctcaggacagactgaccgaggtgattgccagcattatcaagccccagaacacctattgcgtgagacggtacgccgtggtgcagaaagccgcacatggccacgtgcggaaagccttcaagtctcacgtgtccaccctgaccgacctgcagccttacatgcggcaattcgtggcccacctccaggagacaagccctctgagagacgccgtggtgatcgagcagtccagcagccttaatgaggcctctagcggcctgttcgacgtgttcctgagatttatgtgccaccacgccgtgagaatcagaggcaaaagctacgtgcagtgccagggcatcccccaaggatctatcctgagcaccctgctgtgcagcctgtgctacggcgacatggaaaacaagctgttcgccggaatccggagagacggcctgctgctgcggctcgttgacgacttcctgctggtgacacctcacctgacacacgccaaaaccttcctcagaacactggttcggggcgtccccgagtacggctgcgtcgtgaacctgagaaagacagtggtgaacttccctgtggaagatgaggcactcggcggaacagcgttcgtgcagatgcccgcccacggcctgttcccctggtgcggcctgctgctggacaccagaaccctggaggtgcagagcgattacagcagctatgccagaaccagcatccgagccagcctaacctttaaccggggcttcaaagccggcagaaatatgagaagaaagctgttcggagtgctgaggcttaaatgccacagcctgttcctggacctgcaggtcaacagcctgcaaacagtgtgcaccaacatctacaagatcctgctgttacaagcctacagatttcacgcctgtgtgctgcagctgcctttccaccagcaggtgtggaagaaccctacgtttttcctgagagtgatctctgatacagccagtttgtgctacagcatcctgaaggccaagaacgccggcatgagcctgggagccaagggcgctgctgggcctctgccttctgaggccgtgcagtggctgtgccaccaggcctttctgctgaagctgaccagacatagagtgacctacgtgcctctcctgggcagcctgagaaccgcccagacccagctctctcggaagctgcctggcacaaccctgaccgccctggaagctgctgccaatcctgccctgcctagcgacttcaagaccatcctggactgataagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 315 - RNA, 6778 nt, synthetic construct
  source 1..6778: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgatgcctcgagcaccccggtgcagagccgtgcggagcctgctgcggtctcactacagagaggtcctccccctggccacgttcgtgcggcgtctgggccctcagggctggcgactggtgcagcgcggcgaccccgccgcattccgcgccctggtggcccagtgcctggtgtgtgtgccttgggatgccagacctcctcctgcggctccttcttttagacaggtgtcttgtctgaaggagctggtggcccgggtgctgcagcggctgtgcgagcggggagctaagaatgtgttagctttcggttttgccctgctggacggcgccagaggcggaccacctgaagccttcactacctcggtgagaagctacctgcctaacaccgtgacagacgccctgcggggcagcggcgcctggggcctgctactgagacgggtgggagatgatgtgctggtgcacctgctggccagatgcgccctctttgtgctggtggcccctagctgtgcctaccaggtgtgtggccctcccctgtaccagctgggcgccgccacgcaggccagaccccctccgcacgccagcggccctaggagacgtctgggctgcgaaagagcttggaaccacagtgtgcgagaagccggcgtccccctgggcctccctgcccctggagccagaagaagaggaggctccgccagcagaagcctcccgctgcccaagcggcctagacggggcgccgcccctgaacccgagcggaccccggtcggccagggcagttgggcccaccccggaaggaccagaggacctagcgaccggggcttctgcgtggtctccccagcccggccagccgaggaagcaaccagcctggagggcgccctgagcggcaccagacactcccacccttccgttggcagacagcaccacgccggcccaccaagcaccagcagacctccgcgaccttgggacacaccctgtcctcctgtgtacgccgagaccaagcacttcctgtactcctctggcgataaggaacagctccggcctagcttcctgctgagctctctgagacccagcttgaccggcgcccgcagactcgtggaaacaatcttcctgggcagcagaccttggatgccaggcacccctagacggctgcctagactgccccagagatactggcagatgcgtccactgtttttggaactgctgggcaaccacgctcagtgtccttacggcgttctgctgaaaacccactgccccctgagagccgctgtcaccccagccgccggcgtgtgcgccagagaaaagcctcagggaagcgtggctgctcctgaggaagaggatacagaccctagacgcctggtgcaactgctgcgccaacatagcagcccttggcaggtgtacggcttcgtcagagcttgtctgagaaggctggtgcccccgggactgtggggctccagacacaacgagcggagattcctgcggaacacaaagaagttcatcagcctgggcaagcacgccaagctgtctctgcaggagttgacctggaagatgagcgtgagggattgcgcgtggctgagaagatcccctggagtgggctgtgtgccagccgccgagcacagactgcgagaggaaatcctggccaaattcctgcactggctgatgagcgtatatgtggtggaactgctgcggagcttcttctacgtgaccgagacaacatttcagaagaacagacttttcttctaccggaagtctgtatggagcaagctgcagtccatcggaatcagacagcacctgaagagagttcagctgcgggaactgagcgaggcagaggtgaggcagcatagagaggctagacctgctctgcttaccagcagactgagattcatcccaaaacctgatggcctgagacctatcgtgaatatggactacgtggtgggcgcccgaaccttccggcgtgagaagcgggctgaaagactgacatcccgggtgaaggccctgttcagcgtgctgaactacgagagagctcgtcggcctgggctactgggcgccagcgtgctgggcctggatgacatccacagagcctggagaacattcgtgctgagagtgagggctcaagatcccccaccggagctgtacttcgttaaggtggacgtgacaggcgcatacgacaccatccctcaggacagactgaccgaggtgattgccagcattatcaagccccagaacacctattgcgtgagacggtacgccgtggtgcagaaagccgcacatggccacgtgcggaaagccttcaagtctcacgtgtccaccctgaccgacctgcagccttacatgcggcaattcgtggcccacctccaggagacaagccctctgagagacgccgtggtgatcgagcagtccagcagccttaatgaggcctctagcggcctgttcgacgtgttcctgagatttatgtgccaccacgccgtgagaatcagaggcaaaagctacgtgcagtgccagggcatcccccaaggatctatcctgagcaccctgctgtgcagcctgtgctacggcgacatggaaaacaagctgttcgccggaatccggagagacggcctgctgctgcggctcgttgacgacttcctgctggtgacacctcacctgacacacgccaaaaccttcctcagaacactggttcggggcgtccccgagtacggctgcgtcgtgaacctgagaaagacagtggtgaacttccctgtggaagatgaggcactcggcggaacagcgttcgtgcagatgcccgcccacggcctgttcccctggtgcggcctgctgctggacaccagaaccctggaggtgcagagcgattacagcagctatgccagaaccagcatccgagccagcctaacctttaaccggggcttcaaagccggcagaaatatgagaagaaagctgttcggagtgctgaggcttaaatgccacagcctgttcctggacctgcaggtcaacagcctgcaaacagtgtgcaccaacatctacaagatcctgctgttacaagcctacagatttcacgcctgtgtgctgcagctgcctttccaccagcaggtgtggaagaaccctacgtttttcctgagagtgatctctgatacagccagtttgtgctacagcatcctgaaggccaagaacgccggcatgagcctgggagccaagggcgctgctgggcctctgccttctgaggccgtgcagtggctgtgccaccaggcctttctgctgaagctgaccagacatagagtgacctacgtgcctctcctgggcagcctgagaaccgcccagacccagctctctcggaagctgcctggcacaaccctgaccgccctggaagctgctgccaatcctgccctgcctagcgacttcaagaccatcctggactgataagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgcgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcgtacggccaccatggtgtccaaaggcgaggaagtgatcaaggaatttatgagattcaaggtcagaatggaaggatctatgaacggccacgagttcgagatcgagggagaaggagaaggcagaccctacgagggcacccaaaccgctaaattaaaagtgacaaagggcggacctctgccgttcgcctgggatatcctgtccccccagttcatgtacggcagcaaggcctacgtgaagcaccctgctgacatccccgattacaaaaagctgtcttttcctgagggctttaagtgggagagagtgatgaacttcgaggacggaggcctggtgaccgttacccaagacagcagcctgcaagatggaacactgatctacaaagtgaagatgcggggcactaatttccctccagacggccccgtgatgcagaagaaaaccatgggctgggaggcctctacagaacggctgtaccccagagacggagtgctgaagggcgagattcaccaggcactgaagctgaaagatggcggacactacctggtggagttcaagaccatttacatggccaagaagcctgtgcagctgcctggctactactacgtggacaccaagctggacatcacctcccacaatgaggattataccatcgtggaacagtacgagagaagcgagggcagacaccacctgttcctcggccacggcacaggctccaccggctctggcagcagcggcacagccagcagcgaggacaacaacatggccgtgatcaaggagttcatgcggttcaaggtgcggatggaaggcagtatgaacggccatgagttcgaaatcgagggcgaaggcgaaggcagaccttacgagggcacacagacagctaagctgaaggtgaccaagggaggccctctgccattcgcctgggacatcctgagccctcagtttatgtatggcagcaaggcctacgtgaagcaccccgccgacatccccgactacaagaagctgagcttccctgagggtttcaaatgggaaagggtgatgaacttcgaggacggcggactggtgacagtgacccaggacagcagcctgcaggatggcaccctgatctataaggtgaagatgagaggcaccaactttccacctgacgggcccgtgatgcagaaaaaaaccatgggatgggaggccagcaccgagcggctgtaccctagagatggcgttctgaagggcgagatccaccaggccctgaagctgaaggacggcggccactacctggtggagttcaagaccatctacatggccaagaaacctgtgcagctgcctggatactactacgtggacacaaagctggacatcacctctcacaacgaggactataccatcgtcgagcagtacgaacgcagcgaaggcagacaccatctgttcctgtacggtatggacgagctgtacaagtaatgattaattaaaataaaagatctttatgttcattagatctgtgtgttggtttttgtgtgtgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 316 - RNA, 2985 nt, synthetic construct
  source 1..2985: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgatgagcaccgccgtgctggagaatcctggcctgggcagaaagctgtccgatttcggccaagagacaagctacattgaggataattgcaaccagaacggcgctatcagcctgatcttttctttgaaagaggaagtgggcgctctggccaaggtcctgaggctgttcgaggaaaacgacgtgaacctgacccatatcgaaagcagacccagccggctgaagaaagatgagtatgagttcttcacccacctggacaagagatctctccctgccctgacaaacatcatcaaaatcctaagacacgacattggcgccaccgtgcacgagctgtcaagagataagaagaaggacaccgtgccttggtttcctagaaccatccaggagctggatagattcgccaatcagatcctgtcttacggcgccgaactggacgccgaccaccccggcttcaaggaccctgtgtaccgggccagacgcaagcagttcgccgacatcgcctataactaccggcatggccagcctatccctagagtggaatacatggaagaagagaaaaaaacctggggcaccgttttcaagaccctgaaaagcctgtataagactcacgcctgctacgagtacaaccacatcttccctctgctggaaaagtactgtggctttcacgaggacaacatcccacagctggaggacgtgtcccagttcctgcagacctgtaccggcttccggctgcggcccgtggccggactgctgagcagccgggacttcctgggcggactggcttttagagtgttccactgcacacagtacatcagacacggctccaagcctatgtacacacctgagcccgacatctgccacgagctgctgggacacgtccctctgtttagcgatagatctttcgcccagtttagccaggagatcggcctggccagcttgggcgcccctgatgaatacatcgagaaactggctacaatctactggttcacagtggagttcggcctctgcaagcagggcgacagcatcaaggcctacggcgccggactgctttctagcttcggcgagctgcaatactgtctgtccgagaagcccaagctgctgcctctggaactggaaaagaccgccatccagaattacaccgtgaccgagttccagcccctgtactacgtggctgagagtttcaacgacgctaaggaaaaggtgcggaacttcgccgccacaatcccaagacctttcagcgtgcggtacgacccctacacccaaagaatcgaggtgctggacaacacccagcagctgaagatcctggcagatagcatcaacagcgagatcggaatcctgtgcagcgccctgcagaagattaagtgataagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 317 - RNA, 4738 nt, synthetic construct
  source 1..4738: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgatgagcaccgccgtgctggagaatcctggcctgggcagaaagctgtccgatttcggccaagagacaagctacattgaggataattgcaaccagaacggcgctatcagcctgatcttttctttgaaagaggaagtgggcgctctggccaaggtcctgaggctgttcgaggaaaacgacgtgaacctgacccatatcgaaagcagacccagccggctgaagaaagatgagtatgagttcttcacccacctggacaagagatctctccctgccctgacaaacatcatcaaaatcctaagacacgacattggcgccaccgtgcacgagctgtcaagagataagaagaaggacaccgtgccttggtttcctagaaccatccaggagctggatagattcgccaatcagatcctgtcttacggcgccgaactggacgccgaccaccccggcttcaaggaccctgtgtaccgggccagacgcaagcagttcgccgacatcgcctataactaccggcatggccagcctatccctagagtggaatacatggaagaagagaaaaaaacctggggcaccgttttcaagaccctgaaaagcctgtataagactcacgcctgctacgagtacaaccacatcttccctctgctggaaaagtactgtggctttcacgaggacaacatcccacagctggaggacgtgtcccagttcctgcagacctgtaccggcttccggctgcggcccgtggccggactgctgagcagccgggacttcctgggcggactggcttttagagtgttccactgcacacagtacatcagacacggctccaagcctatgtacacacctgagcccgacatctgccacgagctgctgggacacgtccctctgtttagcgatagatctttcgcccagtttagccaggagatcggcctggccagcttgggcgcccctgatgaatacatcgagaaactggctacaatctactggttcacagtggagttcggcctctgcaagcagggcgacagcatcaaggcctacggcgccggactgctttctagcttcggcgagctgcaatactgtctgtccgagaagcccaagctgctgcctctggaactggaaaagaccgccatccagaattacaccgtgaccgagttccagcccctgtactacgtggctgagagtttcaacgacgctaaggaaaaggtgcggaacttcgccgccacaatcccaagacctttcagcgtgcggtacgacccctacacccaaagaatcgaggtgctggacaacacccagcagctgaagatcctggcagatagcatcaacagcgagatcggaatcctgtgcagcgccctgcagaagattaagtgataagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgcgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcgtacggccaccatggtgtccaaaggcgaggaagtgatcaaggaatttatgagattcaaggtcagaatggaaggatctatgaacggccacgagttcgagatcgagggagaaggagaaggcagaccctacgagggcacccaaaccgctaaattaaaagtgacaaagggcggacctctgccgttcgcctgggatatcctgtccccccagttcatgtacggcagcaaggcctacgtgaagcaccctgctgacatccccgattacaaaaagctgtcttttcctgagggctttaagtgggagagagtgatgaacttcgaggacggaggcctggtgaccgttacccaagacagcagcctgcaagatggaacactgatctacaaagtgaagatgcggggcactaatttccctccagacggccccgtgatgcagaagaaaaccatgggctgggaggcctctacagaacggctgtaccccagagacggagtgctgaagggcgagattcaccaggcactgaagctgaaagatggcggacactacctggtggagttcaagaccatttacatggccaagaagcctgtgcagctgcctggctactactacgtggacaccaagctggacatcacctcccacaatgaggattataccatcgtggaacagtacgagagaagcgagggcagacaccacctgttcctcggccacggcacaggctccaccggctctggcagcagcggcacagccagcagcgaggacaacaacatggccgtgatcaaggagttcatgcggttcaaggtgcggatggaaggcagtatgaacggccatgagttcgaaatcgagggcgaaggcgaaggcagaccttacgagggcacacagacagctaagctgaaggtgaccaagggaggccctctgccattcgcctgggacatcctgagccctcagtttatgtatggcagcaaggcctacgtgaagcaccccgccgacatccccgactacaagaagctgagcttccctgagggtttcaaatgggaaagggtgatgaacttcgaggacggcggactggtgacagtgacccaggacagcagcctgcaggatggcaccctgatctataaggtgaagatgagaggcaccaactttccacctgacgggcccgtgatgcagaaaaaaaccatgggatgggaggccagcaccgagcggctgtaccctagagatggcgttctgaagggcgagatccaccaggccctgaagctgaaggacggcggccactacctggtggagttcaagaccatctacatggccaagaaacctgtgcagctgcctggatactactacgtggacacaaagctggacatcacctctcacaacgaggactataccatcgtcgagcagtacgaacgcagcgaaggcagacaccatctgttcctgtacggtatggacgagctgtacaagtaatgattaattaaaataaaagatctttatgttcattagatctgtgtgttggtttttgtgtgtgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 318 - RNA, 6633 nt, synthetic construct
  source 1..6633: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatgcaaatcgagctgtccacttgcttcttcctgtgcctgctgcgcttttgtttcagtgctacccgcagatactacctgggagccgtggagctttcttgggactatatgcagagcgatctgggcgagctgccagtcgacgccagatttccacctagagtgcctaagagtttccccttcaacacctctgtggtgtacaagaaaaccctgtttgtggagttcaccgaccacctgttcaatatcgctaagcctcggcctccttggatgggcctgctgggccccaccattcaggctgaagtgtacgacaccgtggtgatcaccctgaagaacatggcctcccaccctgtctctctgcacgccgtcggcgtgtcgtactggaaggccagcgagggcgctgaatacgacgaccagaccagccaaagagagaaggaggatgataaggtgttcccaggtggcagccacacctacgtgtggcaggtgctgaaagagaacggcccaatggcctcagaccctctgtgcctgacatacagctatttatcccacgttgacctggtgaaggacctgaacagcggactgatcggcgccctgctggtgtgcagagaaggctccctggccaaggaaaagacacagacactacacaagttcatcttactgtttgccgtgttcgacgagggcaagagctggcacagcgagaccaagaatagcctgatgcaagacagagatgccgcctccgctagagcgtggcctaaaatgcacaccgtgaacggctacgtcaacagatctctgcccggcctgattggctgtcaccggaagtccgtgtactggcacgtgatcggaatgggcacaaccccggaggtgcactcaatcttcctggaaggccacacgttcctggtgagaaaccaccggcaggcctcgctcgagatctcccctatcacatttctgaccgcccagaccttgctcatggacctgggccagtttctgcttttctgccacatcagctcccaccagcacgacggcatggaggcttacgtgaaggtggattcttgtcctgaggagcctcagctgagaatgaagaataatgaggaagccgaagattacgacgacgacctgacggacagcgagatggatgttgtgaggtttgacgatgataatagcccttcctttatccagatccggtcagtggcgaagaaacaccctaagacatgggtgcactacatcgccgctgaggaggaggattgggactacgcccccctggtgctggcccctgacgaccggagctacaaatctcagtacctgaacaacggccctcaaagaatcggcagaaagtacaagaaggtgagattcatggcttatacagatgaaacatttaaaaccagagaagctatccagcatgagagcggaatcctgggacctctgctgtacggcgaggtgggcgacaccctgctgatcatctttaagaaccaggccagcagaccttacaatatctaccctcacggcattacagacgtgagacctctgtacagccggcggctgcccaagggcgtgaagcacctgaaggacttcccaatcctgcctggagagatcttcaagtacaagtggaccgtgacagtggaagatggccctaccaagtctgacccccggtgcctgacccggtactactcgagttttgtgaacatggagcgcgacctggcctctggactcatcggcccattgctgatctgctacaaagagtccgtggaccagagaggcaaccagatcatgagcgacaagcgtaacgtgatcctgttcagcgtgtttgatgagaatagaagctggtatctgaccgagaacatccagcggttcctccccaaccctgccggcgtgcagctggaggaccctgagttccaggccagcaacatcatgcactctattaacggctacgtgttcgactccctgcagctaagcgtttgcctgcacgaggtggcttactggtacatcctgagcatcggcgcccagaccgacttcctgtccgtgttcttctcaggctacacattcaaacataagatggtgtatgaggacaccctgaccctgttccccttttccggagagaccgtgttcatgagcatggagaaccctggcctttggatcctgggctgtcacaatagcgactttcggaaccggggaatgaccgccctgctgaaagtcagctcttgcgacaagaacaccggcgactactacgaggacagctatgaggacatctccgcctacctgctgagcaagaacaacgccatcgagcctcggtcgttcagccagaactctaggcaccccagcacccggcagaagcaattcaacgcaaccaccatccctgaaaacgacatcgaaaagaccgatccttggttcgctcacagaaccccaatgcccaagatccaaaacgtgtcctcttctgacctgctgatgctgctgagacagagcccaacccctcacggcctgagcctgtctgacctgcaggaggccaaatacgagactttctctgacgatcctagccccggggccatcgacagcaacaacagcctgagcgagatgactcatttccggcctcagctccaccactctggagacatggttttcacacctgaaagcggcctgcagctgagactgaacgagaagctaggcaccaccgctgccaccgaactgaagaagctggacttcaaggtaagcagcaccagcaacaacctgatcagcaccatcccttctgacaaccttgccgccggcaccgacaacacaagcagcctgggccctccttcaatgcctgtgcactacgacagccagctggataccacactgtttggaaagaaatctagccctctgaccgaatctggcggccctctgagcctgagcgaagagaacaacgatagcaaactgctggagagcggcctgatgaacagccaggagtcatcttggggcaagaacgtgagcagtaccagggagatcacccggacaaccctgcagagcgatcaggaggagatcgattatgatgacactatcagcgtggagatgaagaaagaggacttcgacatctacgacgaggacgaaaaccaaagccctcggagcttccagaagaaaaccagacactatttcatcgccgctgtggagagactgtgggactacggcatgagctcctctccccacgtgctgcggaaccgggcccagagcggatctgttcctcagttcaagaaagtcgtgttccaagagttcaccgacggctcttttacccagcctctgtatagaggcgaattaaacgaacacctgggcctgctgggcccttacatcagagcagaagttgaggataacatcatggtgaccttcagaaatcaggcctctcggccctactctttctactcctcgctcatcagctacgaggaggaccagaggcaaggcgccgaacccagaaagaacttcgtgaagcccaacgaaaccaagacttacttttggaaggtgcaacaccacatggcccctaccaaagacgagtttgattgcaaggcctgggcctacttcagcgatgtggacctggaaaaggatgtccatagcggcctgattggccccttgctggtgtgccacacaaacacactcaatcctgcccacgggagacaggtgaccgtgcaggagttcgccctgttcttcaccatcttcgacgagacaaagagctggtacttcacagaaaacatggaaagaaattgcagagccccttgcaacattcagatggaggatcccaccttcaaggaaaattacagattccacgccatcaacggatacatcatggacacactgccaggactggttatggcccaggatcagagaatccggtggtacctgctctctatgggctcaaacgagaatatccactccatccactttagcggccacgtgttcaccgtgagaaagaaagaggagtacaagatggccctgtacaatctgtaccccggcgtgttcgagacagtggaaatgctgccaagcaaggccgggatctggcgggtggaatgtcttattggcgagcacctgcacgccggaatgagcacactgtttctggtatacagcaacaagtgccagacacccctgggcatggcttctggtcacatcagagattttcagatcacagcctctggtcagtacggacagtgggctcccaagctggccagactgcactactccggctccatcaacgcctggagcacaaaagagcctttcagctggatcaaggtcgatctgctggctccaatgatcatccacggaatcaagacccagggcgcccggcagaaattctcgagcctctacatctcgcagttcattatcatgtacagcctggacggaaagaagtggcagacctacagaggaaatagtaccggcaccctgatggtgttcttcggcaatgtggacagctcaggaattaagcacaacatcttcaatcctccaatcatagccagatacatcagacttcatcctacccactactctatcagaagcacgctgagaatggagtggatgggatgtgacctgaacagctgcagcatgcctcttggcatggaatctaaagccatctctgatgcccagatcaccgcttctagttacttcaccaacatgttcgccacatggtcccctagcaaggccagactgcacctgcagggcaggtctaatgcctggcggcctcaggtgaacaaccccaaggaatggctgcaggtcgatttccagaaaaccatgaaagtgaccggagtgaccacccagggcgtgaaaagcctgctgaccagcatgtacgtgaaggagttcctgatctctagctcccaggatggccaccagtggaccctgttctttcagaacggcaaagtgaaagtgttccaaggcaaccaggacagcttcaccccagtcgtgaacagcctggatccacctctccttaccagatacctgagaatccatccccagtcctgggtccaccagatcgccctgcggatggaggtgctgggctgcgaggcccaagacctgtactaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 319 - RNA, 8386 nt, synthetic construct
  source 1..8386: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatgcaaatcgagctgtccacttgcttcttcctgtgcctgctgcgcttttgtttcagtgctacccgcagatactacctgggagccgtggagctttcttgggactatatgcagagcgatctgggcgagctgccagtcgacgccagatttccacctagagtgcctaagagtttccccttcaacacctctgtggtgtacaagaaaaccctgtttgtggagttcaccgaccacctgttcaatatcgctaagcctcggcctccttggatgggcctgctgggccccaccattcaggctgaagtgtacgacaccgtggtgatcaccctgaagaacatggcctcccaccctgtctctctgcacgccgtcggcgtgtcgtactggaaggccagcgagggcgctgaatacgacgaccagaccagccaaagagagaaggaggatgataaggtgttcccaggtggcagccacacctacgtgtggcaggtgctgaaagagaacggcccaatggcctcagaccctctgtgcctgacatacagctatttatcccacgttgacctggtgaaggacctgaacagcggactgatcggcgccctgctggtgtgcagagaaggctccctggccaaggaaaagacacagacactacacaagttcatcttactgtttgccgtgttcgacgagggcaagagctggcacagcgagaccaagaatagcctgatgcaagacagagatgccgcctccgctagagcgtggcctaaaatgcacaccgtgaacggctacgtcaacagatctctgcccggcctgattggctgtcaccggaagtccgtgtactggcacgtgatcggaatgggcacaaccccggaggtgcactcaatcttcctggaaggccacacgttcctggtgagaaaccaccggcaggcctcgctcgagatctcccctatcacatttctgaccgcccagaccttgctcatggacctgggccagtttctgcttttctgccacatcagctcccaccagcacgacggcatggaggcttacgtgaaggtggattcttgtcctgaggagcctcagctgagaatgaagaataatgaggaagccgaagattacgacgacgacctgacggacagcgagatggatgttgtgaggtttgacgatgataatagcccttcctttatccagatccggtcagtggcgaagaaacaccctaagacatgggtgcactacatcgccgctgaggaggaggattgggactacgcccccctggtgctggcccctgacgaccggagctacaaatctcagtacctgaacaacggccctcaaagaatcggcagaaagtacaagaaggtgagattcatggcttatacagatgaaacatttaaaaccagagaagctatccagcatgagagcggaatcctgggacctctgctgtacggcgaggtgggcgacaccctgctgatcatctttaagaaccaggccagcagaccttacaatatctaccctcacggcattacagacgtgagacctctgtacagccggcggctgcccaagggcgtgaagcacctgaaggacttcccaatcctgcctggagagatcttcaagtacaagtggaccgtgacagtggaagatggccctaccaagtctgacccccggtgcctgacccggtactactcgagttttgtgaacatggagcgcgacctggcctctggactcatcggcccattgctgatctgctacaaagagtccgtggaccagagaggcaaccagatcatgagcgacaagcgtaacgtgatcctgttcagcgtgtttgatgagaatagaagctggtatctgaccgagaacatccagcggttcctccccaaccctgccggcgtgcagctggaggaccctgagttccaggccagcaacatcatgcactctattaacggctacgtgttcgactccctgcagctaagcgtttgcctgcacgaggtggcttactggtacatcctgagcatcggcgcccagaccgacttcctgtccgtgttcttctcaggctacacattcaaacataagatggtgtatgaggacaccctgaccctgttccccttttccggagagaccgtgttcatgagcatggagaaccctggcctttggatcctgggctgtcacaatagcgactttcggaaccggggaatgaccgccctgctgaaagtcagctcttgcgacaagaacaccggcgactactacgaggacagctatgaggacatctccgcctacctgctgagcaagaacaacgccatcgagcctcggtcgttcagccagaactctaggcaccccagcacccggcagaagcaattcaacgcaaccaccatccctgaaaacgacatcgaaaagaccgatccttggttcgctcacagaaccccaatgcccaagatccaaaacgtgtcctcttctgacctgctgatgctgctgagacagagcccaacccctcacggcctgagcctgtctgacctgcaggaggccaaatacgagactttctctgacgatcctagccccggggccatcgacagcaacaacagcctgagcgagatgactcatttccggcctcagctccaccactctggagacatggttttcacacctgaaagcggcctgcagctgagactgaacgagaagctaggcaccaccgctgccaccgaactgaagaagctggacttcaaggtaagcagcaccagcaacaacctgatcagcaccatcccttctgacaaccttgccgccggcaccgacaacacaagcagcctgggccctccttcaatgcctgtgcactacgacagccagctggataccacactgtttggaaagaaatctagccctctgaccgaatctggcggccctctgagcctgagcgaagagaacaacgatagcaaactgctggagagcggcctgatgaacagccaggagtcatcttggggcaagaacgtgagcagtaccagggagatcacccggacaaccctgcagagcgatcaggaggagatcgattatgatgacactatcagcgtggagatgaagaaagaggacttcgacatctacgacgaggacgaaaaccaaagccctcggagcttccagaagaaaaccagacactatttcatcgccgctgtggagagactgtgggactacggcatgagctcctctccccacgtgctgcggaaccgggcccagagcggatctgttcctcagttcaagaaagtcgtgttccaagagttcaccgacggctcttttacccagcctctgtatagaggcgaattaaacgaacacctgggcctgctgggcccttacatcagagcagaagttgaggataacatcatggtgaccttcagaaatcaggcctctcggccctactctttctactcctcgctcatcagctacgaggaggaccagaggcaaggcgccgaacccagaaagaacttcgtgaagcccaacgaaaccaagacttacttttggaaggtgcaacaccacatggcccctaccaaagacgagtttgattgcaaggcctgggcctacttcagcgatgtggacctggaaaaggatgtccatagcggcctgattggccccttgctggtgtgccacacaaacacactcaatcctgcccacgggagacaggtgaccgtgcaggagttcgccctgttcttcaccatcttcgacgagacaaagagctggtacttcacagaaaacatggaaagaaattgcagagccccttgcaacattcagatggaggatcccaccttcaaggaaaattacagattccacgccatcaacggatacatcatggacacactgccaggactggttatggcccaggatcagagaatccggtggtacctgctctctatgggctcaaacgagaatatccactccatccactttagcggccacgtgttcaccgtgagaaagaaagaggagtacaagatggccctgtacaatctgtaccccggcgtgttcgagacagtggaaatgctgccaagcaaggccgggatctggcgggtggaatgtcttattggcgagcacctgcacgccggaatgagcacactgtttctggtatacagcaacaagtgccagacacccctgggcatggcttctggtcacatcagagattttcagatcacagcctctggtcagtacggacagtgggctcccaagctggccagactgcactactccggctccatcaacgcctggagcacaaaagagcctttcagctggatcaaggtcgatctgctggctccaatgatcatccacggaatcaagacccagggcgcccggcagaaattctcgagcctctacatctcgcagttcattatcatgtacagcctggacggaaagaagtggcagacctacagaggaaatagtaccggcaccctgatggtgttcttcggcaatgtggacagctcaggaattaagcacaacatcttcaatcctccaatcatagccagatacatcagacttcatcctacccactactctatcagaagcacgctgagaatggagtggatgggatgtgacctgaacagctgcagcatgcctcttggcatggaatctaaagccatctctgatgcccagatcaccgcttctagttacttcaccaacatgttcgccacatggtcccctagcaaggccagactgcacctgcagggcaggtctaatgcctggcggcctcaggtgaacaaccccaaggaatggctgcaggtcgatttccagaaaaccatgaaagtgaccggagtgaccacccagggcgtgaaaagcctgctgaccagcatgtacgtgaaggagttcctgatctctagctcccaggatggccaccagtggaccctgttctttcagaacggcaaagtgaaagtgttccaaggcaaccaggacagcttcaccccagtcgtgaacagcctggatccacctctccttaccagatacctgagaatccatccccagtcctgggtccaccagatcgccctgcggatggaggtgctgggctgcgaggcccaagacctgtactaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgcgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcgtacggccaccatggtgtccaaaggcgaggaagtgatcaaggaatttatgagattcaaggtcagaatggaaggatctatgaacggccacgagttcgagatcgagggagaaggagaaggcagaccctacgagggcacccaaaccgctaaattaaaagtgacaaagggcggacctctgccgttcgcctgggatatcctgtccccccagttcatgtacggcagcaaggcctacgtgaagcaccctgctgacatccccgattacaaaaagctgtcttttcctgagggctttaagtgggagagagtgatgaacttcgaggacggaggcctggtgaccgttacccaagacagcagcctgcaagatggaacactgatctacaaagtgaagatgcggggcactaatttccctccagacggccccgtgatgcagaagaaaaccatgggctgggaggcctctacagaacggctgtaccccagagacggagtgctgaagggcgagattcaccaggcactgaagctgaaagatggcggacactacctggtggagttcaagaccatttacatggccaagaagcctgtgcagctgcctggctactactacgtggacaccaagctggacatcacctcccacaatgaggattataccatcgtggaacagtacgagagaagcgagggcagacaccacctgttcctcggccacggcacaggctccaccggctctggcagcagcggcacagccagcagcgaggacaacaacatggccgtgatcaaggagttcatgcggttcaaggtgcggatggaaggcagtatgaacggccatgagttcgaaatcgagggcgaaggcgaaggcagaccttacgagggcacacagacagctaagctgaaggtgaccaagggaggccctctgccattcgcctgggacatcctgagccctcagtttatgtatggcagcaaggcctacgtgaagcaccccgccgacatccccgactacaagaagctgagcttccctgagggtttcaaatgggaaagggtgatgaacttcgaggacggcggactggtgacagtgacccaggacagcagcctgcaggatggcaccctgatctataaggtgaagatgagaggcaccaactttccacctgacgggcccgtgatgcagaaaaaaaccatgggatgggaggccagcaccgagcggctgtaccctagagatggcgttctgaagggcgagatccaccaggccctgaagctgaaggacggcggccactacctggtggagttcaagaccatctacatggccaagaaacctgtgcagctgcctggatactactacgtggacacaaagctggacatcacctctcacaacgaggactataccatcgtcgagcagtacgaacgcagcgaaggcagacaccatctgttcctgtacggtatggacgagctgtacaagtaatgattaattaaaataaaagatctttatgttcattagatctgtgtgttggtttttgtgtgtgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 320 - RNA, 5991 nt, synthetic construct
  source 1..5991: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatgcaaatcgagctgtccacttgcttcttcctgtgcctgctgcgcttttgtttcagtgctacccgcagatactacctgggagccgtggagctttcttgggactatatgcagagcgatctgggcgagctgccagtcgacgccagatttccacctagagtgcctaagagtttccccttcaacacctctgtggtgtacaagaaaaccctgtttgtggagttcaccgaccacctgttcaatatcgctaagcctcggcctccttggatgggcctgctgggccccaccattcaggctgaagtgtacgacaccgtggtgatcaccctgaagaacatggcctcccaccctgtctctctgcacgccgtcggcgtgtcgtactggaaggccagcgagggcgctgaatacgacgaccagaccagccaaagagagaaggaggatgataaggtgttcccaggtggcagccacacctacgtgtggcaggtgctgaaagagaacggcccaatggcctcagaccctctgtgcctgacatacagctatttatcccacgttgacctggtgaaggacctgaacagcggactgatcggcgccctgctggtgtgcagagaaggctccctggccaaggaaaagacacagacactacacaagttcatcttactgtttgccgtgttcgacgagggcaagagctggcacagcgagaccaagaatagcctgatgcaagacagagatgccgcctccgctagagcgtggcctaaaatgcacaccgtgaacggctacgtcaacagatctctgcccggcctgattggctgtcaccggaagtccgtgtactggcacgtgatcggaatgggcacaaccccggaggtgcactcaatcttcctggaaggccacacgttcctggtgagaaaccaccggcaggcctcgctcgagatctcccctatcacatttctgaccgcccagaccttgctcatggacctgggccagtttctgcttttctgccacatcagctcccaccagcacgacggcatggaggcttacgtgaaggtggattcttgtcctgaggagcctcagctgagaatgaagaataatgaggaagccgaagattacgacgacgacctgacggacagcgagatggatgttgtgaggtttgacgatgataatagcccttcctttatccagatccggtcagtggcgaagaaacaccctaagacatgggtgcactacatcgccgctgaggaggaggattgggactacgcccccctggtgctggcccctgacgaccggagctacaaatctcagtacctgaacaacggccctcaaagaatcggcagaaagtacaagaaggtgagattcatggcttatacagatgaaacatttaaaaccagagaagctatccagcatgagagcggaatcctgggacctctgctgtacggcgaggtgggcgacaccctgctgatcatctttaagaaccaggccagcagaccttacaatatctaccctcacggcattacagacgtgagacctctgtacagccggcggctgcccaagggcgtgaagcacctgaaggacttcccaatcctgcctggagagatcttcaagtacaagtggaccgtgacagtggaagatggccctaccaagtctgacccccggtgcctgacccggtactactcgagttttgtgaacatggagcgcgacctggcctctggactcatcggcccattgctgatctgctacaaagagtccgtggaccagagaggcaaccagatcatgagcgacaagcgtaacgtgatcctgttcagcgtgtttgatgagaatagaagctggtatctgaccgagaacatccagcggttcctccccaaccctgccggcgtgcagctggaggaccctgagttccaggccagcaacatcatgcactctattaacggctacgtgttcgactccctgcagctaagcgtttgcctgcacgaggtggcttactggtacatcctgagcatcggcgcccagaccgacttcctgtccgtgttcttctcaggctacacattcaaacataagatggtgtatgaggacaccctgaccctgttccccttttccggagagaccgtgttcatgagcatggagaaccctggcctttggatcctgggctgtcacaatagcgactttcggaaccggggaatgaccgccctgctgaaagtcagctcttgcgacaagaacaccggcgactactacgaggacagctatgaggacatctccgcctacctgctgagcaagaacaacgccatcgagcctcggtcgttcagccagaacccccctgtgctgaagagacaccagagggagatcacccggacaaccctgcagagcgatcaggaggagatcgattatgatgacactatcagcgtggagatgaagaaagaggacttcgacatctacgacgaggacgaaaaccaaagccctcggagcttccagaagaaaaccagacactatttcatcgccgctgtggagagactgtgggactacggcatgagctcctctccccacgtgctgcggaaccgggcccagagcggatctgttcctcagttcaagaaagtcgtgttccaagagttcaccgacggctcttttacccagcctctgtatagaggcgaattaaacgaacacctgggcctgctgggcccttacatcagagcagaagttgaggataacatcatggtgaccttcagaaatcaggcctctcggccctactctttctactcctcgctcatcagctacgaggaggaccagaggcaaggcgccgaacccagaaagaacttcgtgaagcccaacgaaaccaagacttacttttggaaggtgcaacaccacatggcccctaccaaagacgagtttgattgcaaggcctgggcctacttcagcgatgtggacctggaaaaggatgtccatagcggcctgattggccccttgctggtgtgccacacaaacacactcaatcctgcccacgggagacaggtgaccgtgcaggagttcgccctgttcttcaccatcttcgacgagacaaagagctggtacttcacagaaaacatggaaagaaattgcagagccccttgcaacattcagatggaggatcccaccttcaaggaaaattacagattccacgccatcaacggatacatcatggacacactgccaggactggttatggcccaggatcagagaatccggtggtacctgctctctatgggctcaaacgagaatatccactccatccactttagcggccacgtgttcaccgtgagaaagaaagaggagtacaagatggccctgtacaatctgtaccccggcgtgttcgagacagtggaaatgctgccaagcaaggccgggatctggcgggtggaatgtcttattggcgagcacctgcacgccggaatgagcacactgtttctggtatacagcaacaagtgccagacacccctgggcatggcttctggtcacatcagagattttcagatcacagcctctggtcagtacggacagtgggctcccaagctggccagactgcactactccggctccatcaacgcctggagcacaaaagagcctttcagctggatcaaggtcgatctgctggctccaatgatcatccacggaatcaagacccagggcgcccggcagaaattctcgagcctctacatctcgcagttcattatcatgtacagcctggacggaaagaagtggcagacctacagaggaaatagtaccggcaccctgatggtgttcttcggcaatgtggacagctcaggaattaagcacaacatcttcaatcctccaatcatagccagatacatcagacttcatcctacccactactctatcagaagcacgctgagaatggagtggatgggatgtgacctgaacagctgcagcatgcctcttggcatggaatctaaagccatctctgatgcccagatcaccgcttctagttacttcaccaacatgttcgccacatggtcccctagcaaggccagactgcacctgcagggcaggtctaatgcctggcggcctcaggtgaacaaccccaaggaatggctgcaggtcgatttccagaaaaccatgaaagtgaccggagtgaccacccagggcgtgaaaagcctgctgaccagcatgtacgtgaaggagttcctgatctctagctcccaggatggccaccagtggaccctgttctttcagaacggcaaagtgaaagtgttccaaggcaaccaggacagcttcaccccagtcgtgaacagcctggatccacctctccttaccagatacctgagaatccatccccagtcctgggtccaccagatcgccctgcggatggaggtgctgggctgcgaggcccaagacctgtactaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 321 - RNA, 7744 nt, synthetic construct
  source 1..7744: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatgcaaatcgagctgtccacttgcttcttcctgtgcctgctgcgcttttgtttcagtgctacccgcagatactacctgggagccgtggagctttcttgggactatatgcagagcgatctgggcgagctgccagtcgacgccagatttccacctagagtgcctaagagtttccccttcaacacctctgtggtgtacaagaaaaccctgtttgtggagttcaccgaccacctgttcaatatcgctaagcctcggcctccttggatgggcctgctgggccccaccattcaggctgaagtgtacgacaccgtggtgatcaccctgaagaacatggcctcccaccctgtctctctgcacgccgtcggcgtgtcgtactggaaggccagcgagggcgctgaatacgacgaccagaccagccaaagagagaaggaggatgataaggtgttcccaggtggcagccacacctacgtgtggcaggtgctgaaagagaacggcccaatggcctcagaccctctgtgcctgacatacagctatttatcccacgttgacctggtgaaggacctgaacagcggactgatcggcgccctgctggtgtgcagagaaggctccctggccaaggaaaagacacagacactacacaagttcatcttactgtttgccgtgttcgacgagggcaagagctggcacagcgagaccaagaatagcctgatgcaagacagagatgccgcctccgctagagcgtggcctaaaatgcacaccgtgaacggctacgtcaacagatctctgcccggcctgattggctgtcaccggaagtccgtgtactggcacgtgatcggaatgggcacaaccccggaggtgcactcaatcttcctggaaggccacacgttcctggtgagaaaccaccggcaggcctcgctcgagatctcccctatcacatttctgaccgcccagaccttgctcatggacctgggccagtttctgcttttctgccacatcagctcccaccagcacgacggcatggaggcttacgtgaaggtggattcttgtcctgaggagcctcagctgagaatgaagaataatgaggaagccgaagattacgacgacgacctgacggacagcgagatggatgttgtgaggtttgacgatgataatagcccttcctttatccagatccggtcagtggcgaagaaacaccctaagacatgggtgcactacatcgccgctgaggaggaggattgggactacgcccccctggtgctggcccctgacgaccggagctacaaatctcagtacctgaacaacggccctcaaagaatcggcagaaagtacaagaaggtgagattcatggcttatacagatgaaacatttaaaaccagagaagctatccagcatgagagcggaatcctgggacctctgctgtacggcgaggtgggcgacaccctgctgatcatctttaagaaccaggccagcagaccttacaatatctaccctcacggcattacagacgtgagacctctgtacagccggcggctgcccaagggcgtgaagcacctgaaggacttcccaatcctgcctggagagatcttcaagtacaagtggaccgtgacagtggaagatggccctaccaagtctgacccccggtgcctgacccggtactactcgagttttgtgaacatggagcgcgacctggcctctggactcatcggcccattgctgatctgctacaaagagtccgtggaccagagaggcaaccagatcatgagcgacaagcgtaacgtgatcctgttcagcgtgtttgatgagaatagaagctggtatctgaccgagaacatccagcggttcctccccaaccctgccggcgtgcagctggaggaccctgagttccaggccagcaacatcatgcactctattaacggctacgtgttcgactccctgcagctaagcgtttgcctgcacgaggtggcttactggtacatcctgagcatcggcgcccagaccgacttcctgtccgtgttcttctcaggctacacattcaaacataagatggtgtatgaggacaccctgaccctgttccccttttccggagagaccgtgttcatgagcatggagaaccctggcctttggatcctgggctgtcacaatagcgactttcggaaccggggaatgaccgccctgctgaaagtcagctcttgcgacaagaacaccggcgactactacgaggacagctatgaggacatctccgcctacctgctgagcaagaacaacgccatcgagcctcggtcgttcagccagaacccccctgtgctgaagagacaccagagggagatcacccggacaaccctgcagagcgatcaggaggagatcgattatgatgacactatcagcgtggagatgaagaaagaggacttcgacatctacgacgaggacgaaaaccaaagccctcggagcttccagaagaaaaccagacactatttcatcgccgctgtggagagactgtgggactacggcatgagctcctctccccacgtgctgcggaaccgggcccagagcggatctgttcctcagttcaagaaagtcgtgttccaagagttcaccgacggctcttttacccagcctctgtatagaggcgaattaaacgaacacctgggcctgctgggcccttacatcagagcagaagttgaggataacatcatggtgaccttcagaaatcaggcctctcggccctactctttctactcctcgctcatcagctacgaggaggaccagaggcaaggcgccgaacccagaaagaacttcgtgaagcccaacgaaaccaagacttacttttggaaggtgcaacaccacatggcccctaccaaagacgagtttgattgcaaggcctgggcctacttcagcgatgtggacctggaaaaggatgtccatagcggcctgattggccccttgctggtgtgccacacaaacacactcaatcctgcccacgggagacaggtgaccgtgcaggagttcgccctgttcttcaccatcttcgacgagacaaagagctggtacttcacagaaaacatggaaagaaattgcagagccccttgcaacattcagatggaggatcccaccttcaaggaaaattacagattccacgccatcaacggatacatcatggacacactgccaggactggttatggcccaggatcagagaatccggtggtacctgctctctatgggctcaaacgagaatatccactccatccactttagcggccacgtgttcaccgtgagaaagaaagaggagtacaagatggccctgtacaatctgtaccccggcgtgttcgagacagtggaaatgctgccaagcaaggccgggatctggcgggtggaatgtcttattggcgagcacctgcacgccggaatgagcacactgtttctggtatacagcaacaagtgccagacacccctgggcatggcttctggtcacatcagagattttcagatcacagcctctggtcagtacggacagtgggctcccaagctggccagactgcactactccggctccatcaacgcctggagcacaaaagagcctttcagctggatcaaggtcgatctgctggctccaatgatcatccacggaatcaagacccagggcgcccggcagaaattctcgagcctctacatctcgcagttcattatcatgtacagcctggacggaaagaagtggcagacctacagaggaaatagtaccggcaccctgatggtgttcttcggcaatgtggacagctcaggaattaagcacaacatcttcaatcctccaatcatagccagatacatcagacttcatcctacccactactctatcagaagcacgctgagaatggagtggatgggatgtgacctgaacagctgcagcatgcctcttggcatggaatctaaagccatctctgatgcccagatcaccgcttctagttacttcaccaacatgttcgccacatggtcccctagcaaggccagactgcacctgcagggcaggtctaatgcctggcggcctcaggtgaacaaccccaaggaatggctgcaggtcgatttccagaaaaccatgaaagtgaccggagtgaccacccagggcgtgaaaagcctgctgaccagcatgtacgtgaaggagttcctgatctctagctcccaggatggccaccagtggaccctgttctttcagaacggcaaagtgaaagtgttccaaggcaaccaggacagcttcaccccagtcgtgaacagcctggatccacctctccttaccagatacctgagaatccatccccagtcctgggtccaccagatcgccctgcggatggaggtgctgggctgcgaggcccaagacctgtactaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgcgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcgtacggccaccatggtgtccaaaggcgaggaagtgatcaaggaatttatgagattcaaggtcagaatggaaggatctatgaacggccacgagttcgagatcgagggagaaggagaaggcagaccctacgagggcacccaaaccgctaaattaaaagtgacaaagggcggacctctgccgttcgcctgggatatcctgtccccccagttcatgtacggcagcaaggcctacgtgaagcaccctgctgacatccccgattacaaaaagctgtcttttcctgagggctttaagtgggagagagtgatgaacttcgaggacggaggcctggtgaccgttacccaagacagcagcctgcaagatggaacactgatctacaaagtgaagatgcggggcactaatttccctccagacggccccgtgatgcagaagaaaaccatgggctgggaggcctctacagaacggctgtaccccagagacggagtgctgaagggcgagattcaccaggcactgaagctgaaagatggcggacactacctggtggagttcaagaccatttacatggccaagaagcctgtgcagctgcctggctactactacgtggacaccaagctggacatcacctcccacaatgaggattataccatcgtggaacagtacgagagaagcgagggcagacaccacctgttcctcggccacggcacaggctccaccggctctggcagcagcggcacagccagcagcgaggacaacaacatggccgtgatcaaggagttcatgcggttcaaggtgcggatggaaggcagtatgaacggccatgagttcgaaatcgagggcgaaggcgaaggcagaccttacgagggcacacagacagctaagctgaaggtgaccaagggaggccctctgccattcgcctgggacatcctgagccctcagtttatgtatggcagcaaggcctacgtgaagcaccccgccgacatccccgactacaagaagctgagcttccctgagggtttcaaatgggaaagggtgatgaacttcgaggacggcggactggtgacagtgacccaggacagcagcctgcaggatggcaccctgatctataaggtgaagatgagaggcaccaactttccacctgacgggcccgtgatgcagaaaaaaaccatgggatgggaggccagcaccgagcggctgtaccctagagatggcgttctgaagggcgagatccaccaggccctgaagctgaaggacggcggccactacctggtggagttcaagaccatctacatggccaagaaacctgtgcagctgcctggatactactacgtggacacaaagctggacatcacctctcacaacgaggactataccatcgtcgagcagtacgaacgcagcgaaggcagacaccatctgttcctgtacggtatggacgagctgtacaagtaatgattaattaaaataaaagatctttatgttcattagatctgtgtgttggtttttgtgtgtgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 322 - RNA, 2240 nt, synthetic construct
  source 1..2240: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggctctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 323 - RNA, 2240 nt, synthetic construct; identical to SEQ ID NO: 322
  source 1..2240: mol_type = other RNA

SEQ ID NO: 324 - RNA, 1838 nt, synthetic construct
  source 1..1838: mol_type = other RNA
gggtaaacggcgggagtaactatgactctcttaaggagtcatagagccagaacctcctcgtggtcccgctgggcacagggattaatttttctgtggcaaatttgactggctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggataggggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgcttgtctcgctccggggaacgctcggaaactcccggccgccgccacccgcgtctgttctgttacacaagggaagaaaagccgctgccgcactccgagtgtcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaataaaagatctttatgttcattagatctgtgtgttggtttttgtgtggctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 325 - RNA, 1529 nt, synthetic construct
  source 1..1529: mol_type = other RNA
gggtaaacggcgggagtaactatgactctcttaaggagtcatagagccagaacctcctcgtggtcccgctgggcacagggattaatttttctgtggcaaatttgactggctctagagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattgcgtctatatatgcagaggccgaggccgcctcggcctctgagctattccagaagtagtgaggaggccgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaataaaagatctttatgttcattagatctgtgtgttggtttttgtgtggctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 326 - RNA, 2330 nt, synthetic construct
  source 1..2330: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatttctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 327 - RNA, 1844 nt, synthetic construct
  source 1..1844: mol_type = other RNA
gggtaaacggcgggagtaactatgactctcttaaggagtcatagagccagaacctcctcgtggtcccgctgggcacagggattaatttttctgtggcaaatttgactggctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggataggggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgcttgtctcgctccggggaacgctcggaaactcccggccgccgccacccgcgtctgttctgttacacaagggaagaaaagccgctgccgcactccgagtgtcgtacggccaccatggtgagcaaaggcgaagaagataacatggcgattattaaagaatttatgcgctttaaagtgcatatggaaggcagcgtgaacggccatgaatttgaaattgaaggcgaaggcgaaggccgcccgtatgaaggcacccagaccgcgaaactgaaagtgaccaaaggcggcccgctgccgtttgcgtgggatattctgagcccgcagtttatgtatggcagcaaagcgtatgtgaaacatccggcggatattccggattatctgaaactgagctttccggaaggctttaaatgggaacgcgtgatgaactttgaagatggcggcgtggtgaccgtgacccaggatagcagcctgcaggatggcgaatttatttataaagtgaaactgcgcggcaccaactttccgagcgatggcccggtgatgcagaaaaaaaccatgggctgggaagcgagcagcgaacgcatgtatccggaagatggcgcgctgaaaggcgaaattaaacagcgcctgaaactgaaagatggcggccattatgatgcggaagtgaaaaccacctataaagcgaaaaaaccggtgcagctgccgggcgcgtataacgtgaacattaaactggatattaccagccataacgaagattataccattgtggaacagtatgaacgcgcggaaggccgccatagcaccggcggcatggatgaactgtataaatagtgaaagcttaataaaagatctttatgttcattagatctgtgtgttggtttttgtgtggcggccgctagggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 328 - RNA, 1535 nt, synthetic construct
  source 1..1535: mol_type = other RNA
gggtaaacggcgggagtaactatgactctcttaaggagtcatagagccagaacctcctcgtggtcccgctgggcacagggattaatttttctgtggcaaatttgactggctctagagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattgcgtctatatatgcagaggccgaggccgcctcggcctctgagctattccagaagtagtgaggaggccgtacggccaccatggtgagcaaaggcgaagaagataacatggcgattattaaagaatttatgcgctttaaagtgcatatggaaggcagcgtgaacggccatgaatttgaaattgaaggcgaaggcgaaggccgcccgtatgaaggcacccagaccgcgaaactgaaagtgaccaaaggcggcccgctgccgtttgcgtgggatattctgagcccgcagtttatgtatggcagcaaagcgtatgtgaaacatccggcggatattccggattatctgaaactgagctttccggaaggctttaaatgggaacgcgtgatgaactttgaagatggcggcgtggtgaccgtgacccaggatagcagcctgcaggatggcgaatttatttataaagtgaaactgcgcggcaccaactttccgagcgatggcccggtgatgcagaaaaaaaccatgggctgggaagcgagcagcgaacgcatgtatccggaagatggcgcgctgaaaggcgaaattaaacagcgcctgaaactgaaagatggcggccattatgatgcggaagtgaaaaccacctataaagcgaaaaaaccggtgcagctgccgggcgcgtataacgtgaacattaaactggatattaccagccataacgaagattataccattgtggaacagtatgaacgcgcggaaggccgccatagcaccggcggcatggatgaactgtataaatagtgaaagcttaataaaagatctttatgttcattagatctgtgtgttggtttttgtgtggcggccgctagggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 329 - RNA, 2934 nt, synthetic construct
  source 1..2934: mol_type = other RNA
gggtaaacggcgggagtaactatgactctcttaaggagtcatagagccagaacctcctcgtggtcccgctgggcacagggattaatttttctgtggcaaatttgactggctctagactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattgcgtctatatatgcagaggccgaggccgcctcggcctctgagctattccagaagtagtgaggaggccgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaataaaagatctttatgttcattagatctgtgtgttggtttttgtgtggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggataggggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgcttgtctcgctccggggaacgctcggaaactcccggccgccgccacccgcgtctgttctgttacacaagggaagaaaagccgctgccgcactccgagtgtcgtacggccaccatggtgagcaaaggcgaagaagataacatggcgattattaaagaatttatgcgctttaaagtgcatatggaaggcagcgtgaacggccatgaatttgaaattgaaggcgaaggcgaaggccgcccgtatgaaggcacccagaccgcgaaactgaaagtgaccaaaggcggcccgctgccgtttgcgtgggatattctgagcccgcagtttatgtatggcagcaaagcgtatgtgaaacatccggcggatattccggattatctgaaactgagctttccggaaggctttaaatgggaacgcgtgatgaactttgaagatggcggcgtggtgaccgtgacccaggatagcagcctgcaggatggcgaatttatttataaagtgaaactgcgcggcaccaactttccgagcgatggcccggtgatgcagaaaaaaaccatgggctgggaagcgagcagcgaacgcatgtatccggaagatggcgcgctgaaaggcgaaattaaacagcgcctgaaactgaaagatggcggccattatgatgcggaagtgaaaaccacctataaagcgaaaaaaccggtgcagctgccgggcgcgtataacgtgaacattaaactggatattaccagccataacgaagattataccattgtggaacagtatgaacgcgcggaaggccgccatagcaccggcggcatggatgaactgtataaatagtgaaagcttaataaaagatctttatgttcattagatctgtgtgttggtttttgtgtggcggccgcggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 330 - RNA, 2168 nt, synthetic construct
  source 1..2168: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggctctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 331 - RNA, 2049 nt, synthetic construct
  source 1..2049: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggctctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 332 - RNA, 2019 nt, synthetic construct
  source 1..2019: mol_type = other RNA
gggagacggtcaaccgcgtaggagcggtgaccggaattcggcgggagtaactatgactctcttaaggagtcatagagccagaacctcctcgtggtcccgctgggcacagggattaatttttctgtggcaaatttgactggctctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgacaaggtggacgggccacctttacttaacccggaaaaggaacatatattaattatatgtgttcggaaaatagcaaaaaaaaaaaaaaaaaaaaaa

SEQ ID NO: 333 - DNA, 36 bp, synthetic construct
  source 1..36: mol_type = other DNA
aggtcgaccagatgtccgaggtcgaccagttgtccg

SEQ ID NO: 334 - DNA, 26 bp, synthetic construct
  source 1..26: mol_type = other DNA
tagcaaaaaaaaaaaaaaaaaaaaaa

SEQ ID NO: 335 - DNA, 29 bp, synthetic construct
  source 1..29: mol_type = other DNA
catcatggattaggatcggaagacccccg

SEQ ID NO: 336 - DNA, 29 bp, synthetic construct
  source 1..29: mol_type = other DNA
gtacgccggcgaaattggatcagtagatg

SEQ ID NO: 337 - DNA, 28 bp, synthetic construct
  source 1..28: mol_type = other DNA
gagaaacagacgggcctgatctacaccc

SEQ ID NO: 338 - DNA, 34 bp, synthetic construct
  misc_feature 1..34: note = 3'UTR
  source 1..34: mol_type = other DNA
ctatctgaaccgaagttccgcaacgcctacgtac

SEQ ID NO: 339 - DNA, 33 bp, synthetic construct
  source 1..33: mol_type = other DNA
cactgcgtgtggtcagttttcctagcatgcacg

SEQ ID NO: 340 - DNA, 42 bp, synthetic construct
  source 1..42: mol_type = other DNA
gatgttatgccaagacagcaagcaaatgttttgaaccaaacg

SEQ ID NO: 341 - DNA, 28 bp, synthetic construct
  source 1..28: mol_type = other DNA
ttgaggcgagtcaccactcgctttccgg

SEQ ID NO: 342 - DNA, 29 bp, synthetic construct
  source 1..29: mol_type = other DNA
gtgtccgtcacggggacgacatccgagtg

SEQ ID NO: 343 - DNA, 35 bp, synthetic construct
  source 1..35: mol_type = other DNA
ctcctgaccaactagctcactgactaattttaaac

SEQ ID NO: 344 - DNA, 34 bp, synthetic construct
  source 1..34: mol_type = other DNA
ccacttattctacacctctcatgtctcttcaccg

SEQ ID NO: 345 - DNA, 27 bp, synthetic construct
  source 1..27: mol_type = other DNA
ctagcagccgacttagaactggtgcgg

SEQ ID NO: 346 - DNA, 28 bp, synthetic construct
  source 1..28: mol_type = other DNA
cttcgtcttcggaatccatgtccatagc

SEQ ID NO: 347 - DNA, 34 bp, synthetic construct
  source 1..34: mol_type = other DNA
ccaggggaatccgactgtttaattaaaacaaagc

SEQ ID NO: 348 - DNA, 30 bp, synthetic construct
  source 1..30: mol_type = other DNA
gcgactcgcatcactgactttaattggttg

SEQ ID NO: 349 - RNA, 21 nt, synthetic construct
  source 1..21: mol_type = other RNA
cgcgcttcgtatttcagaatt

SEQ ID NO: 350 - RNA, 21 nt, synthetic construct
  source 1..21: mol_type = other RNA
ttctgaaatacgaagcgcgtg

SEQ ID NO: 351 - RNA, 21 nt, synthetic construct
  source 1..21: mol_type = other RNA
tgacctctccaaaccctcttt

SEQ ID NO: 352 - RNA, 21 nt, synthetic construct
  source 1..21: mol_type = other RNA
agagggtttggagaggtcatg

SEQ ID NO: 353 - RNA, 21 nt, synthetic construct
  source 1..21: mol_type = other RNA
gggagcacctgaatcctaatt

SEQ ID NO: 354 - RNA, 21 nt, synthetic construct
  source 1..21: mol_type = other RNA
ttaggattcaggtgctcccgg

SEQ ID NO: 355 - RNA, 21 nt, synthetic construct
  source 1..21: mol_type = other RNA
ggatattactttcgtgtaatt

SEQ ID NO: 356 - RNA, 21 nt, synthetic construct
  source 1..21: mol_type = other RNA
ttacacgaaagtaatatccaa

SEQ ID NO: 357 - RNA, 21 nt, synthetic construct
  source 1..21: mol_type = other RNA
cgtcgattcccagatcttatt

SEQ ID NO: 358 - RNA, 21 nt, synthetic construct
  source 1..21: mol_type = other RNA
taagatctgggaatcgacgaa

SEQ ID NO: 359 - RNA, 21 nt, synthetic construct
  source 1..21: mol_type = other RNA
gaatcgcaaggatatgatatt

SEQ ID NO: 360 - RNA, 21 nt, synthetic construct
  source 1..21: mol_type = other RNA
tatcatatccttgcgattctc